(19) United States
(12) Patent Application Publication
(10) Pub. No.: US 2017/0291890 A1
(43) Pub. Date: Oct. 12, 2017

(54) HETEROCYCLE SUBSTITUTED AMINO-PYRIDINE COMPOUNDS AND METHODS OF USE THEREOF

(71) Applicant: Epizyme, Inc., Cambridge, MA (US)

(72) Inventor: John Emmerson Campbell, Cambridge, MA (US)

(21) Appl. No.: 15/511,779

(22) PCT Filed: Sep. 17, 2015
(86) PCT No.: PCT/US15/50813

§ 371 Date: Mar. 16, 2017

Related U.S. Application Data
(60) Provisional application No. 62/051,889, filed on Sep. 17, 2014.

(51) Int. Cl. C07D 405/14; C07D 401/14; C07D 401/12

(52) U.S. Cl. CPC C07D 405/14; C07D 401/12; C07D 401/14

(57) ABSTRACT

The present disclosure relates to heterocycle substituted amino-pyridine compounds. The present disclosure also relates to pharmaceutical compositions containing these compounds and methods of treating cancer by administering these compounds and pharmaceutical compositions to subjects in need thereof. The present disclosure also relates to the use of such compounds for research or other non-therapeutic purposes.

### RELATED APPLICATIONS

This application is a continuation of U.S. Patent Application No. 62/051,889, filed Sep. 17, 2014, the disclosure of which is incorporated herein by reference in its entirety.

### BACKGROUND

There is an ongoing need for new agents for treating cancer and neoplastic diseases.

### SUMMARY

In one aspect, the present disclosure features a heterocycle substituted amino-pyridine compound of Formula (III) below or a pharmaceutically acceptable salt thereof:

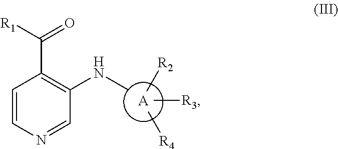

wherein

ring A is 5-membered heteroaryl or 5-membered heterocycloalkyl;

R₁ is hydroxyl, C₁-C₆ alkoxyl, or mono- or di-C₁-C₆-alkylamino and said C₁-C₆ alkoxyl, or mono- or di-C₁-C₆-alkylamino is optionally substituted with one or more substituents selected from the group consisting of halo, hydroxyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl;

R₂ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₂ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₂ is attached to the pyrazolyl via a carbon ring atom thereof;

R₃ is H, halo, cyano, azido, oxo, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more -Q₁-T₁, wherein Q₁ is a bond, C(O), a C₁-C₆ alkyl linker, or a 4- to 6-membered heterocycloalkyl linker and T₁ is selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when T₁ is C₁-C₆ alkyl, C₁-C₆ alkoxyl,C(O)O—C₁-C₆ alkyl, mono-C₁-C₆ alkylamino, di-C₁-C₆ alkylamino, C₃-C₈ cycloalkyl, C₆-C₁₀ aryl, 4 to 12-membered heterocycloalkyl, or 5- or 6-membered heteroaryl, T₁ is optionally substituted with one or more substituents selected from the group consisting of halo, C₁-C₄ alkyl, and C₆-C₁₀ aryl; and

R₄ is H, C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and when R₄ is not H, R₄ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₄ is attached to the pyrazolyl via a carbon ring atom thereof, and when neither R₂ nor R₄ is H, only one of R₂, R₃, and R₄ is aryl or heteroaryl.

One subset of the compounds of Formula (III) includes those of Formula (IIIa) or (IIIb):

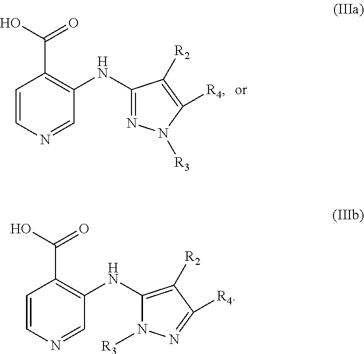

Another subset of the compounds of Formula (III) includes those of Formula (IIIc) or (IIId):

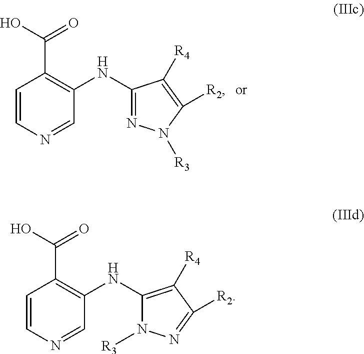

Still another subset of the compounds of Formula (III) includes those of Formula (IIIe) or (IIIf):

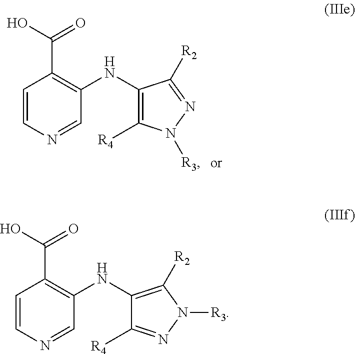

Another subset of the compounds of Formula (III) includes those of Formula (IIIg), (IIIh) (IIIi) or (IIIj):

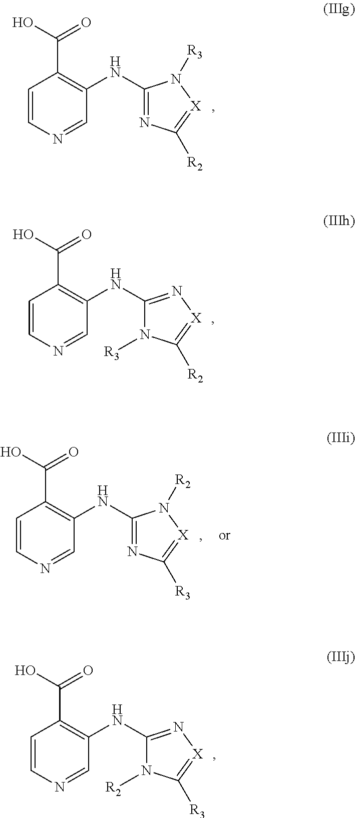

wherein X is N or CR₄.

Yet another subset of the compounds of Formula (III) includes those of Formula (IIIk) or (IIIl):

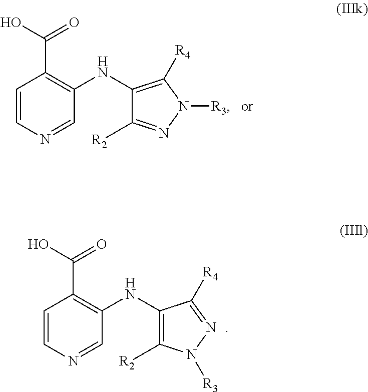

Yet another subset of the compounds of Formula (III) includes those of Formula (I):

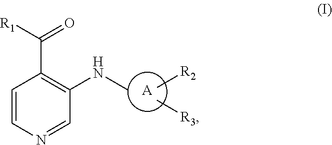

wherein

ring A is 5-membered heteroaryl or 5-membered heterocycloalkyl;

R₁ is hydroxyl or C₁-C₆ alkoxyl;

R₂ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₂ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₂ is attached to the pyrazolyl via a carbon ring atom thereof; and

R₃ is H, halo, cyano, azido, oxo, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more substituents selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

One subset of the compounds of Formula (I) includes those of Formula (Ia) or (Ib):

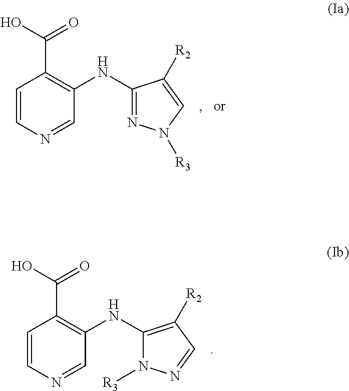

Another subset of the compounds of Formula (I) includes those of Formula (Ic) or (Id):

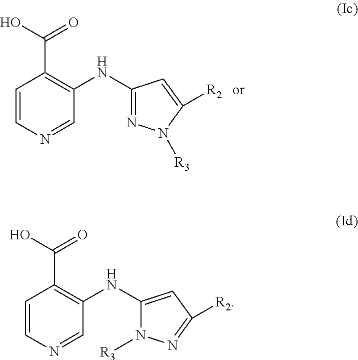

Still another subset of the compounds of Formula (I) includes those of Formula (Ie) or (If):

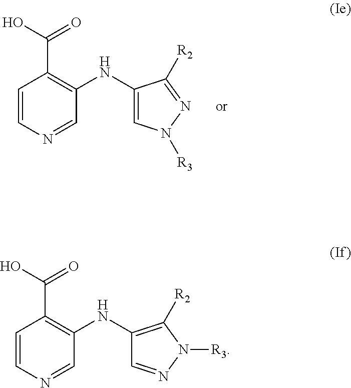

Yet another subset of the compounds of Formula (I) includes those of Formula (Ig) or (Ih):

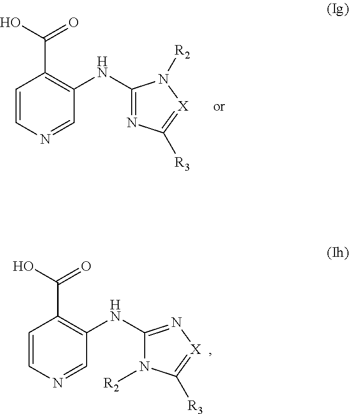

wherein X is N or CH.

The present disclosure also provides pharmaceutical compositions comprising one or more pharmaceutically acceptable carriers and one or more compounds selected from those of any of the Formulae described herein.

The present disclosure also provides compounds as described in any Formula herein or a pharmaceutically acceptable salt thereof for use in a method of treating cancer such as prostate cancer, breast cancer, bladder cancer, lung cancer, gastric cancer, or melanoma.

In addition, the present disclosure provides a method of treating cancer in a subject in need thereof by administering to the subject a therapeutically effective amount of a compound of Formula (II) or a pharmaceutically acceptable salt thereof:

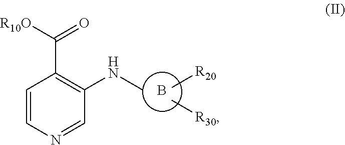

wherein, ring B is pyrazolyl, ring B is pyrazolyl;

R₁₀ is H or C₁-C₆ alkyl;

R₂₀ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, C₆-C₁₀ aryl or 5- or 6-membered heteroaryl and R₂₀ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, and R₂₀ is attached to ring B via a nitrogen ring atom thereof; and

R₃₀ is H, halo, cyano, azido, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more substituents selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

Another aspect of this disclosure is a method of treating or preventing a disorder that is regulated by histone methylation and/or demethylation by modulating the activity of a demethylase comprising a Jumonji C (JmjC) domain (e.g., JHDM proteins, JMJD2 proteins and JARID1 proteins). For example, the disorder is a JMJD2-mediated and/or JARID1-mediated disorder. The method includes administering to a subject in need thereof a therapeutically effective amount of one or more compounds selected from those of any of the Formulae described herein. The JMJD2-mediated and/or JARID1-mediated disorder is a disease, disorder, or condition that is mediated at least in part by the activity of JMJD2 and/or JARID1. In one embodiment, the JMJD2-mediated and/or JARID1-mediated disorder is related to an increased JMJD2 and/or JARID1 activity. In one embodiment, the JMJD2-mediated and/or JARID1-mediated disorder is a cancer, such as prostate cancer, breast cancer, bladder cancer, lung cancer, gastric cancer, or melanoma.

Unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models. Methods described herein may be used to identify suitable candidates for treating or preventing JMJD2 and/or JARID1-mediated disorders.

Further, the compounds or methods described herein can be used for research (e.g., studying enzymes) and other non-therapeutic purposes.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the invention will be apparent from the following detailed description and claims.

### DETAILED DESCRIPTION

The present disclosure provides novel heterocycle substituted amino-pyridine compounds, synthetic methods for making the compounds, pharmaceutical compositions containing them and various uses of the compounds.

In one aspect, the present disclosure features a heterocycle substituted amino-pyridine compound of Formula (III) below or a pharmaceutically acceptable salt thereof:

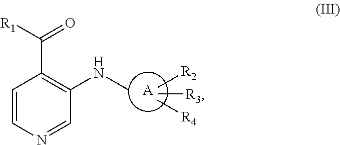

wherein

ring A is 5-membered heteroaryl or 5-membered heterocycloalkyl;

R₁ is hydroxyl, C₁-C₆ alkoxyl, or mono- or di-C₁-C₆-alkylamino and said C₁-C₆ alkoxyl, or mono- or di-C₁-C₆-alkylamino is optionally substituted with one or more substituents selected from the group consisting of halo, hydroxyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl;

R₂ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₂ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₂ is attached to the pyrazolyl via a carbon ring atom thereof;

R₃ is H, halo, cyano, azido, oxo, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more -Q₁-T₁, wherein Q₁ is a bond, C(O), a C₁-C₆ alkyl linker, or a 4- to 6-membered heterocycloalkyl linker and T₁ is selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when T₁ is C₁-C₆ alkyl, C₁-C₆ alkoxyl,C(O)O—C₁-C₆ alkyl, mono-C₁-C₆ alkylamino, di-C₁-C₆ alkylamino, C₃-C₈ cycloalkyl, C₆-C₁₀ aryl, 4 to 12-membered heterocycloalkyl, or 5- or 6-membered heteroaryl, T₁ is optionally substituted with one or more substituents selected from the group consisting of halo, C₁-C₄ alkyl, and C₆-C₁₀ aryl; and

R₄ is H, C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and when R₄ is not H, R₄ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₄ is attached to the pyrazolyl via a carbon ring atom thereof, and when neither R₂ nor R₄ is H, only one of R₂, R₃, and R₄ is aryl or heteroaryl.

In another aspect, the present disclosure provides the compounds of Formula (I):

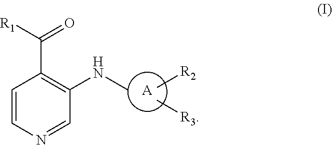

In this Formula,

ring A is 5-membered heteroaryl or 5-membered heterocycloalkyl;

R₁ is hydroxyl or C₁-C₆ alkoxyl;

R₂ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₂ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₂ is attached to the pyrazolyl via a carbon ring atom thereof; and

R₃ is H, halo, cyano, azido, oxo, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more substituents selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

The compounds of Formula (III) or (I) can have one or more of the following features when applicable:

For example, ring A is a nitrogen-containing heteroaryl, such as pyrazolyl, imidazolyl, pyrrolyl, triazolyl, oxazolyl, oxadiazolyl, thiazolyl, isoxazolyl, isothiazolyl, thiadiazolyl, or tetrazolyl.

For example, ring A is pyrazolyl.

For example, ring A is imidazolyl.

For example, ring A is a nitrogen-containing heterocycloalkyl such as pyrrolidinyl, 3-pyrrolinyl, 2-pyrrolinyl, imidazolinyl, imidazolidinyl, 2-pyrazolinyl, and pyrazolidinyl.

For example, ring A is furyl, thienyl, 1,3-dioxolanyl, dihydrofuranyl, dihydrothiophenyl, tetrahydrofuranyl or tetrahydrothiophenyl.

For example, R₁ is hydroxyl.

For example, R₂ is attached to ring A via a carbon ring atom thereof.

For example, R₂ is phenyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂ is pyridinyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂ is unsubstituted phenyl.

For example, R₂ is unsubstituted pyridinyl.

For example, R₂ is C₁-C₆ alkyl, C₂-C₆ alkenyl, C₂-C₆ alkynyl, or C₃-C₈ cycloalkyl, each of which is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂ is C₁-C₆ alkyl substituted with C₃-C₈ cycloalkyl, C₆-C₁₀ aryl, 4 to 12-membered heterocycloalkyl, or 5- or 6-membered heteroaryl. For example, R₂ is benzyl.

For example, R₂ is C₂-C₆ alkyl substituted with C₃-C₈ cycloalkyl, C₆-C₁₀ aryl, 4 to 12-membered heterocycloalkyl, or 5- or 6-membered heteroaryl.

For example, R₂ is C₁-C₆ alkyl substituted with one or more substituents selected from the group consisting of halo, hydroxyl, C(O)OH, C(O)O—C₁-C₆ alkyl, cyano, C₁-C₆ alkoxyl, amino, mono-C₁-C₆ alkylamino, and di-C₁-C₆ alkylamino.

For example, R₂ is C₃-C₈ cycloalkyl optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂ is unsubstituted C₁-C₆ alkyl, C₂-C₆ alkenyl, C₂-C₆ alkynyl, or C₃-C₈ cycloalkyl. For example, R₂ is cyclopentyl or cyclohexyl.

For example, R₂ is optionally substituted C₁-C₆ alkoxyl, C₆-C₁₀ aryloxy, amino, mono-C₁-C₆ alkylamino, or di-C₁-C₆ alkylamino.

For example, R₂ is oxo when ring A is 5-membered heterocycloalkyl. For example, R₂ together with ring A is furanone.

For example, R₃ is oxo when ring A is 5-membered heterocycloalkyl. For example, only one of R₂ and R₃ is oxo.

For example, R₃ is attached to ring A via a carbon ring atom thereof.

For example, R₃ is attached to ring A via a nitrogen ring atom thereof when ring A is a nitrogen-containing heteroaryl or heterocycloalkyl.

For example, R₃ is C₁-C₆ alkyl optionally substituted with one or more -Q₁-T₁.

For example, R₃ is unsubstituted C₁-C₆ alkyl, e.g., methyl.

For example, R₃ is C₁-C₆ alkyl, C₂-C₆ alkenyl, C₂-C₆ alkynyl, or C₃-C₈ cycloalkyl, each of which is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₃ is C₁-C₆ alkyl substituted with C₃-C₈ cycloalkyl, C₆-C₁₀ aryl, 4 to 12-membered heterocycloalkyl, or 5- or 6-membered heteroaryl, each of which is further optionally substituted with one or more halo or C₁-C₄ alkyl. For example, R₃ is benzyl.

For example, R₃ is C₂-C₆ alkyl substituted with C₃-C₈ cycloalkyl, C₆-Cₘ aryl, 4 to 12-membered heterocycloalkyl, or 5- or 6-membered heteroaryl.

For example, R₃ is C₁-C₆ alkyl substituted with one or more substituents selected from the group consisting of halo, hydroxyl, C(O)OH, C(O)O—C₁-C₆ alkyl, cyano, C₁-C₆ alkoxyl, amino, mono-C₁-C₆ alkylamino, and di-C₁-C₆ alkylamino.

For example, R₃ is C₃-C₈ cycloalkyl optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₃ is unsubstituted C₂-C₆ alkenyl, C₂-C₆ alkynyl, or C₃-C₈ cycloalkyl. For example, R₃ is cyclopentyl or cyclohexyl.

For example, R₃ is optionally substituted C₁-C₆ alkoxyl, C₆-C₁₀ aryloxy, amino, mono-C₁-C₆ alkylamino, or di-C₁-C₆ alkylamino.

For example, R₃ is H.

For example, R₃ is azido.

For example, R₃ is cyano.

For example, R₃ is C(O)H.

For example, R₃ is ORₐ or —C(O)Rₐ.

For example, Rₐ is C₁-C₆ alkyl or 4 to 7-membered heterocycloalkyl (e.g., azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, and morpholinyl, and the like), each of which is optionally substituted with one or more -Q₁-T₁.

For example, R₃ is 4 to 7-membered heterocycloalkyl (e.g., azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, and morpholinyl, and the like), each of which is optionally substituted with one or more -Q₁-T₁.

For example, R₃ is piperidinyl, 2,2,6,6-tetramethyl-piperidinyl, 1,2,3,6-tetrahydropyridinyl, 2,2,6,6-tetramethyl-1,2,3,6-tetrahydropyridinyl, piperazinyl, morpholinyl, tetrahydro-2H-pyranyl, 3,6-dihydro-2H-pyranyl, or pyrrolidinyl, each of which is optionally substituted with one or more -Q₁-T₁.

For example, R₃ is 1,4-dioxaspiro[4.5]decan-8-yl.

For example, R₃ is 1,2,3,4-tetrahydroisoquinolinyl, e.g., 1,2,3,4-tetrahydroisoquinolin-5-yl.

For example, R₃ is 4 to 10-membered heterocycloalkyl optionally substituted with a substituent selected from phenyl, benzyl, C(O)-phenyl, C₁-C₆ alkyl, C₁-C₆ haloalkyl, C₃-C₈ cycloalkyl, C₃-C₈ cycloalkyl-C₁-C₆ alkyl, C₁-C₆ alkyl-C₃-C₈ cycloalkyl, 4 to 7-membered heterocycloalkyl (e.g., azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, and morpholinyl, and the like), 4 to 7-membered heterocycloalkyl-C₁-C₆ alkyl or C₁-C₆ alkyl-4 to 7-membered heterocycloalkyl.

For example, R₃ is —NRₐR_{b}, —C(O)Rₐ, —C(O)ORₐ, —C(O)NRₐR_{b}, —NR_{b}C(O)Rₐ, —SRₐ, —S(O)₂Rₐ, or —S(O)₂NRₐR_{b}.

For example, each of Rₐ and R_{b}, independently, is H, optionally substituted C₁-C₆ alkyl or optionally substituted C₃-C₈ cycloalkyl.

For example, one of Rₐ and R_{b} is H.

For example, Rₐ and R_{b}, together with the N atom to which they are attached, form a 4 to 7-membered heterocycloalkyl ring having 0 or 1 additional heteroatoms to the N atom (e.g., azetidinyl, pyrrolidinyl, imidazolidinyl, pyrazolidinyl, oxazolidinyl, isoxazolidinyl, triazolidinyl, piperidinyl, 1,2,3,6-tetrahydropyridinyl, piperazinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, and morpholinyl, and the like) and the ring is optionally substituted with one or more -Q₁-T₁.

For example, R₃ is phenyl optionally substituted with one substituent selected from the group consisting of halo, hydroxyl, C(O)OH, C(O)O—C₁-C₆ alkyl, C(O)NH₂, C(O)NH(C₁-C₆ alkyl), C(O)N(C₁-C₆ alkyl)₂, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₃ is pyridinyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₃ is unsubstituted phenyl.

For example, R₃ is unsubstituted pyridinyl.

For example, Q₁ is a bond.

For example, Q₁ is C(O). For example, Q₁ is C(O) and T₁ is amino, mono-C₁-C₆ alkylamino, di-C₁-C₆ alkylamino, C₃-C₈ cycloalkyl, or C₆-C₁₀ aryl.

For example, Q₁ is a C₁-C₆ alkyl linker, linear or branched.

For example, Q₁ is a 4 to 6-membered heterocycloalkyl linker (e.g., azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, and morpholinyl, and the like).

For example, T₁ is amino, mono-C₁-C₆ alkylamino, di-C₁-C₆ alkylamino, C₃-C₈ cycloalkyl, or C₆-C₁₀ aryl.

For example, T₁ is halo, hydroxyl, oxo, C(O)OH, C(O)O—C₁-C₆ alkyl, cyano, C₁-C₆ alkyl, or C₁-C₆ alkoxyl.

For example, T₁ is 4 to 12-membered heterocycloalkyl or 5- or 6-membered heteroaryl, each of which is optionally substituted with one or more substituents selected from the group consisting of halo, C₁-C₄ alkyl, and C₆-C₁₀ aryl.

For example, R₄ is H.

For example, R₄ is attached to ring A via a carbon ring atom thereof.

For example, R₄ is phenyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₄ is pyridinyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₄ is unsubstituted phenyl.

For example, R₄ is unsubstituted pyridinyl.

For example, R₄ is C₁-C₆ alkyl, C₂-C₆ alkenyl, C₂-C₆ alkynyl, or C₃-C₈ cycloalkyl, each of which is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₄ is C₁-C₆ alkyl substituted with C₃-C₈ cycloalkyl, C₆-C₁₀ aryl, 4 to 12-membered heterocycloalkyl, or 5- or 6-membered heteroaryl. For example, R₄ is benzyl.

For example, R₄ is C₂-C₆ alkyl substituted with C₃-C₈ cycloalkyl, C₆-C₁₀ aryl, 4 to 12-membered heterocycloalkyl, or 5- or 6-membered heteroaryl.

For example, R₄ is C₁-C₆ alkyl substituted with one or more substituents selected from the group consisting of halo, hydroxyl, C(O)OH, C(O)O—C₁-C₆ alkyl, cyano, C₁-C₆ alkoxyl, amino, mono-C₁-C₆ alkylamino, and di-C₁-C₆ alkylamino.

For example, R₄ is C₃-C₈ cycloalkyl optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₄ is unsubstituted C₁-C₆ alkyl, C₂-C₆ alkenyl, C₂-C₆ alkynyl, or C₃-C₈ cycloalkyl. For example, R₄ is cyclopentyl or cyclohexyl.

For example, R₄ is optionally substituted C₁-C₆ alkoxyl, C₆-C₁₀ aryloxy, amino, mono-C₁-C₆ alkylamino, or di-C₁-C₆ alkylamino.

For example, R₄ is oxo when ring A is 5-membered heterocycloalkyl. For example, R₂ together with ring A is furanone. For example, one or two of R₂, R₃ and R₄ are oxo when ring A is 5-membered heterocycloalkyl.

For example, one of R₂ and R₄ is C₁-C₆ alkyl (e.g., methyl), and the other is C₆-C₁₀ aryl (e.g., phenyl).

For example, each of R₂ and R₄ is independently C₁-C₆ alkyl (e.g., methyl).

For example, the compound is of Formula (Ia):

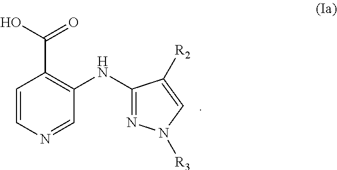

For example, the compound is of Formula (Ib):

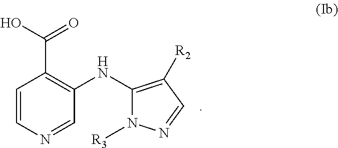

For example, the compound is of Formula (Ic):

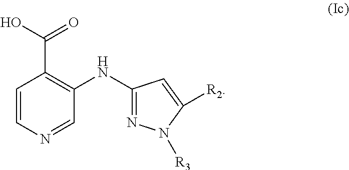

For example, the compound is of Formula (Id):

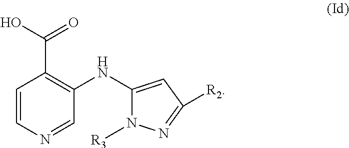

For example, the compound is of Formula (Ie):

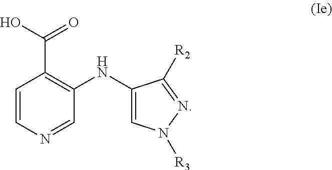

For example, the compound is of Formula (If):

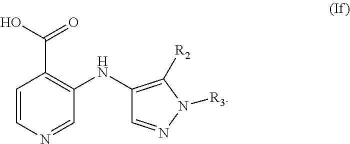

For example, the compound is of Formula (Ig):

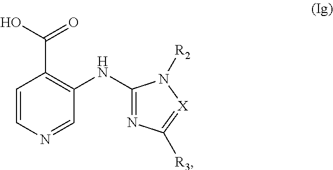

wherein X is N or CH.

For example, the compound is of Formula (Ih):

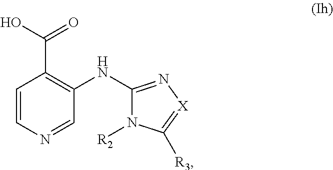

wherein X is N or CH.

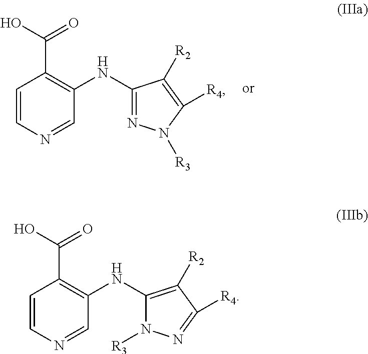

For example, the compound is of Formula (IIIc) or (IIId):

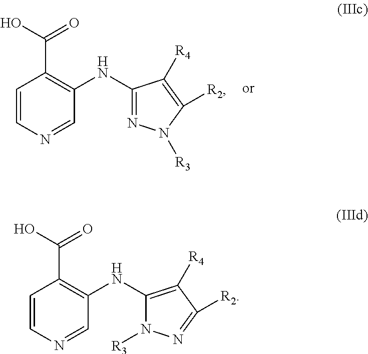

For example, the compound is of Formula (IIIe) or (IIIf):

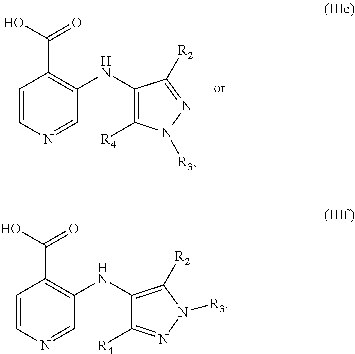

For example, the compound is of Formula (IIIg), (IIIh) (IIIi) or (IIIj):

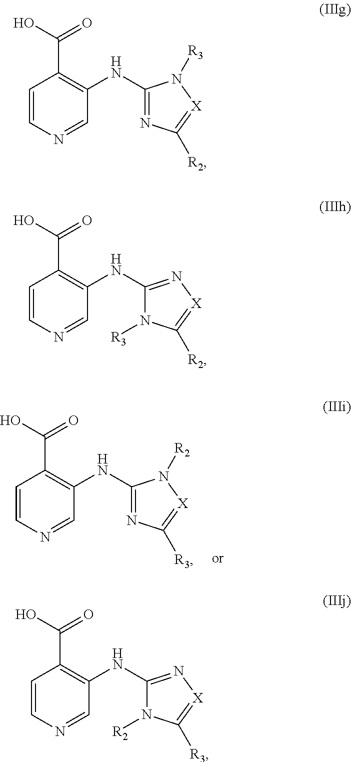

wherein X is N or CR₄.

For example, the compound is of Formula (IIIk) or (IIIl):

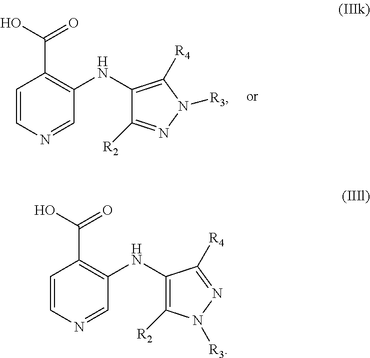

In yet another aspect, the disclosure features a compound of Formula (II) or a pharmaceutically acceptable salt thereof:

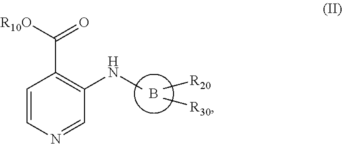

and use thereof for treating cancer.

In Formula (II),

ring B is pyrazolyl;
R₁₀ is H or C₁-C₆ alkyl;
R₂₀ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, C₆-C₁₀ aryl or 5- or 6-membered heteroaryl and R₂₀ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, and R₂₀ is attached to ring B via a nitrogen ring atom thereof; and
R₃₀ is H, halo, cyano, azido, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more substituents selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

The compounds of Formula (II) can have one or more of the following features when applicable:

For example, R₁₀ is H.

For example, R₂₀ is phenyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂₀ is pyridinyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂₀ is unsubstituted phenyl.

For example, R₂₀ is unsubstituted pyridinyl.

For example, R₂₀ is C₁-C₆ alkyl, C₂-C₆ alkenyl, C₂-C₆ alkynyl, or C₃-C₈ cycloalkyl, each of which is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂₀ is C₁-C₆ alkyl substituted with C₃-C₈ cycloalkyl, 4 to 12-membered heterocycloalkyl, or 5- or 6-membered heteroaryl.

For example, R₂₀ is C₁-C₆ alkyl substituted with C₆-C₁₀ aryl and R₃₀ is halo, cyano, azido, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}. For example, R₂₀ is benzyl.

For example, R₂₀ is C₂-C₆ alkyl substituted with C₃-C₈ cycloalkyl, C₆-C₁₀ aryl, 4 to 12-membered heterocycloalkyl, or 5- or 6-membered heteroaryl.

For example, R₂₀ is C₁-C₆ alkyl substituted with one or more substituents selected from the group consisting of halo, hydroxyl, C(O)OH, C(O)O—C₁-C₆ alkyl, cyano, C₁-C₆ alkoxyl, amino, mono-C₁-C₆ alkylamino, and di-C₁-C₆ alkylamino.

For example, R₂₀ is C₃-C₈ cycloalkyl optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂₀ is unsubstituted C₁-C₆ alkyl, C₂-C₆ alkenyl, C₂-C₆ alkynyl, or C₃-C₈ cycloalkyl. For example, R₂₀ is cyclopentyl or cyclohexyl.

For example, R₂₀ is optionally substituted C₁-C₆ alkoxyl, C₆-C₁₀ aryloxy, amino, mono-C₁-C₆ alkylamino, or di-C₁-C₆ alkylamino.

For example, R₃₀ is H.

For example, R₃₀ is C₁-C₆ alkyl, e.g., methyl.

For example, R₃₀ is azido.

For example, R₃₀ is cyano.

For example, R₃₀ is C(O)H.

For example, R₃₀ is ORₐ or —C(O)Rₐ.

For example, Rₐ is C₁-C₆ alkyl or 4 to 7-membered heterocycloalkyl (e.g., azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, and morpholinyl, and the like), each of which is optionally substituted.

For example, R₃₀ is 4 to 7-membered heterocycloalkyl (e.g., azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, and morpholinyl, and the like), each of which is optionally substituted.

For example, R₃₀ is piperidinyl, 2,2,6,6-tetramethyl-piperidinyl, 1,2,3,6-tetrahydropyridinyl, 2,2,6,6-tetramethyl-1,2,3,6-tetrahydropyridinyl, piperazinyl, morpholinyl, tetrahydro-2H-pyranyl, 3,6-dihydro-2H-pyranyl, or pyrrolidinyl, each of which is optionally substituted.

For example, R₃₀ is —NRₐR_{b}, —C(O)Rₐ, —C(O)ORₐ, —C(O)NRₐR_{b}, —NR_{b}C(O)Rₐ, —SRₐ, —S(O)₂Rₐ, or —S(O)₂NRₐR_{b}.

For example, each of Rₐ and R_{b}, independently, is H, optionally substituted C₁-C₆ alkyl or optionally substituted C₃-C₈ cycloalkyl.

For example, one of Rₐ and R_{b} is H.

For example, Rₐ and R_{b}, together with the N atom to which they are attached, form a 4 to 7-membered heterocycloalkyl ring having 0 or 1 additional heteroatoms to the N atom (e.g., azetidinyl, pyrrolidinyl, imidazolidinyl, pyrazolidinyl, oxazolidinyl, isoxazolidinyl, triazolidinyl, piperidinyl, 1,2,3,6-tetrahydropyridinyl, piperazinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, and morpholinyl, and the like) and the ring is optionally substituted.

For example, the compound of Formula (II) is of any one of Formulae (IIa)-(IIf):

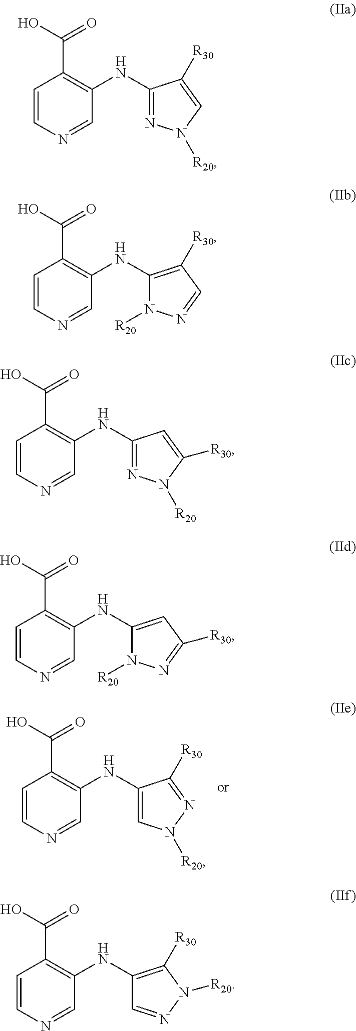

In yet another aspect, a subset of compounds of Formula (III) includes those wherein

ring A is pyrazolyl;

R₁ is hydroxyl or C₁-C₆ alkoxyl;

R₂ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₂ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein R₂ is attached to ring C via a carbon ring atom thereof; and

R₃ is H, halo, cyano, azido, oxo, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more -Q₁-T₁ wherein Q₁ is a bond, C₁-C₆ alkyl linker, or 4- to 6-membered heterocycloalkyl linker and T₁ is selected from the group consisting of halo, hydroxyl, oxo, C(O)OH, C(O)O—C₁-C₆ alkyl, —C(O)NRₐR_{b}, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and when T₁ is C₁-C₆ alkyl, C₁-C₆ alkoxyl,C(O)O—C₁-C₆ alkyl, mono-C₁-C₆ alkylamino, di-C₁-C₆ alkylamino, C₃-C₈ cycloalkyl, C₆-C₁₀ aryl, 4 to 12-membered heterocycloalkyl, or 5- or 6-membered heteroaryl, it is optionally substituted with one or more substituents selected from the group consisting of halo, C₁-C₄ alkyl, and C₆-C₁₀ aryl; and

R₄ is H, C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₄ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl wherein R₄ is attached to ring C via a carbon ring atom thereof, and when neither R₂ nor R₄ is H, only one of R₂, R₃, and R₄ is aryl or heteroaryl.

This subset can have one or more of the following features when applicable:

For example, ring A is a nitrogen-containing heteroaryl, such as pyrazolyl, imidazolyl, pyrrolyl, triazolyl, oxazolyl, oxadiazolyl, thiazolyl, isoxazolyl, isothiazolyl, thiadiazolyl, or tetrazolyl.

For example, R₁ is OH.

For example, R₂ is phenyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂ is unsubstituted phenyl.

For example, R₂ is C₁-C₆ alkyl, C₂-C₆ alkenyl, C₂-C₆ alkynyl, or C₃-C₈ cycloalkyl, each of which is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

For example, R₂ is C₁-C₆ alkyl substituted with C₃-C₈ cycloalkyl, C₆-C₁₀ aryl, 4 to 12-membered heterocycloalkyl, or 5- or 6-membered heteroaryl.

For example, R₂ is benzyl.

For example, R₂ is unsubstituted C₁-C₆ alkyl, e.g., methyl.

For example, R₃ is C₁-C₆ alkyl and R₃ is substituted with one or more -Q₁-T₁ wherein Q₁ is a bond, C₁-C₆ alkyl linker, or 4- to 6-membered heterocycloalkyl linker and T₁ is selected from the group consisting of halo, hydroxyl, oxo, C(O)OH, C(O)O—C₁-C₆ alkyl, —C(O)NRₐR_{b}, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; and wherein when T₁ is C₁-C₆ alkyl, C₁-C₆ alkoxyl,C(O)O—C₁-C₆ alkyl, mono-C₁-C₆ alkylamino, di-C₁-C₆ alkylamino, C₃-C₈ cycloalkyl, C₆-C₁₀ aryl, 4 to 12-membered heterocycloalkyl, or 5- or 6-membered heteroaryl, it is optionally substituted with one or more substituents selected from the group consisting of halo, C₁-C₄ alkyl, and C₆-C₁₀ aryl.

For example, R₃ is C₁-C₆ alkyl substituted with one or more -Q₁-T₁ wherein Q₁ is a bond and T₁ is selected from the group consisting of halo and dimethylamino.

For example, R₃ is C₁-C₆ alkyl substituted with one or more -Q₁-T₁ wherein Q₁ is a bond and T₁ is a 4- to 12-membered heterocycloalkyl optionally substituted with one or more substituents selected from the group consisting of halo, C₁-C₄ alkyl, and C₆-C₁₀ aryl.

For example, R₃ is (4,4-difluoropiperidin-1-yl)ethyl.

For example, R₃ is (4-methylpiperidin-1-yl)ethyl.

For example, R₃ is benzyl.

For example, R₃ is C₆-C₁₀ aryl or 4- to 12-membered heterocycloalkyl, and R₃ is substituted with one or more -Q₁-T₁ wherein Q₁ is a bond, C₁-C₆ alkyl linker, or 4- to 6-membered heterocycloalkyl linker and T₁ is selected from the group consisting of halo, hydroxyl, oxo, C(O)OH, C(O)O—C₁-C₆ alkyl, —C(O)NRₐR_{b}, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; wherein when T₁ is C₁-C₆ alkyl, C₁-C₆ alkoxyl,C(O)O—C₁-C₆ alkyl, mono-C₁-C₆ alkylamino, di-C₁-C₆ alkylamino, C₃-C₈ cycloalkyl, C₆-C₁₀ aryl, 4 to 12-membered heterocycloalkyl, or 5- or 6-membered heteroaryl, it is optionally substituted with one or more substituents selected from the group consisting of halo, C₁-C₄ alkyl, and C₆-C₁₀ aryl.

For example, R₃ is phenyl substituted with one or more -Q₁-T₁ wherein Q₁ is a bond and T₁ is selected from the group consisting of halo, C₁-C₆ alkyl, C₁-C₆ alkoxyl, cyano, and —C(O)NRₐR_{b}, wherein Rₐ is H and R_{b} is methyl.

For example, R₃ is unsubstituted phenyl.

For example, R₃ is C₃-C₈ cycloalkyl, and R₃ is substituted with one or more -Q₁-T₁ wherein Q₁ is a bond and T₁ is selected from the group consisting of halo, C₁-C₆ alkyl, C₆-C₁₀ aryl, C₃-C₈ cycloalkyl, and amino.

For example, R₃ is 4- to 12-membered heterocycloalkyl, and R₃ is substituted with one or more -Q₁-T₁ wherein Q₁ is a bond, C₁-C₆ alkyl linker, or 4- to 12-membered heterocycloalkyl linker, and T₁ is selected from the group consisting of C₁-C₆ alkyl, C₆-C₁₀ aryl, and C₃-C₈ cycloalkyl.

For example, R₃ is 1-(cyclopropylmethyl)piperidin-4-yl.

For example, R₃ is 1-cyclobutylpiperidin-4-yl.

For example, R₃ is 1-phenylpiperidin-4-yl.

For example, R₃ is 1-methylpiperidin-4-yl.

For example, R₃ is 4-methylpyrrolidin-3-yl.

For example, R₃ is an unsubstituted 4- to 12-membered heterocycloalkyl, e.g., piperidinyl, tetrahydropyranyl, azepanyl, and the like.

For example, R₃ is C₁-C₆ alkyl, e.g., methyl, ethyl, and the like.

For example, R₄ is H.

For example, R₄ is methyl.

Representative compounds of the present disclosure include compounds listed in Table 1 and salts or tautomers thereof

TABLE 1

| Compound | | |
|---|---|---|
| Number | Structure | Data |
| 1 | 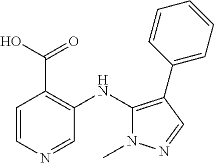 [SMILES: Cn1ncc(-c2ccccc2)c1Nc1cnccc1C(=O)O] | MS (M + 1)^{+:} 294.95 |
| 2 | 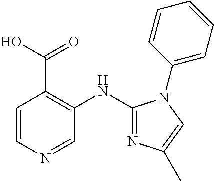 [SMILES: Cc1cn(-c2ccccc2)c(Nc2cnccc2C(=O)O)n1] | MS (M + 1)^{+:} 295.1 |
| 3 | 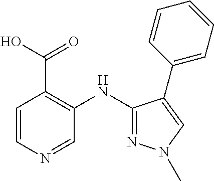 [SMILES: Cn1cc(-c2ccccc2)c(Nc2cnccc2C(=O)O)n1] | MS (M + 1)^{+:} 295.1 |
| 4 | 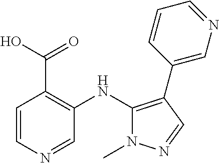 [SMILES: Cn1ncc(-c2cccnc2)c1Nc1cnccc1C(=O)O] | MS (M + 1)^{+:} 295.95 |
| 5 | 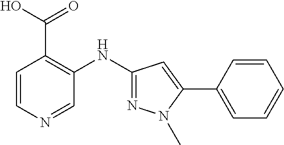 [SMILES: Cn1nc(Nc2cnccc2C(=O)O)cc1-c1ccccc1] | MS (M + 1)^{+:} 294.95 |
| 6 | 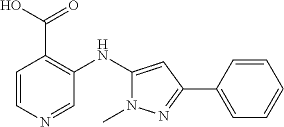 [SMILES: Cn1nc(-c2ccccc2)cc1Nc1cnccc1C(=O)O] | MS (M + 1)^{+:} 294.95 |
| 7 | 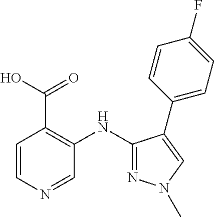 [SMILES: Cn1cc(-c2ccc(F)cc2)c(Nc2cnccc2C(=O)O)n1] | LCMS: 313.10 [M + 1]; ¹H NMR (400 MHz, DMSO-d6) δ 13.87 (s, 1H), 10.02 (s, 1H), 9.37 (s, 1H), 8.05 (d, J = 5.1 Hz, 1H), 7.97 (s, 1H), 7.69 (d, J = 5.0 Hz, 1H), 7.51 (dd, J = 8.4, 5.4 Hz, 2H), 7.23 (t, J = 8.7 Hz, 2H), 3.86 (s, 3H) ppm. |
| 8 | 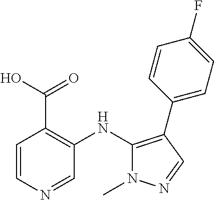 [SMILES: Cn1ncc(-c2ccc(F)cc2)c1Nc1cnccc1C(=O)O] | LCMS: 313.10 [M + 1]; ¹H NMR (400 MHz, DMSO-d6) δ 13.85 (s, 1H), 9.08 (s, 1H), 8.00 (d, J = 5.0 Hz, 1H), 7.93 (s, 1H), 7.68 (d, J = 5.0 Hz, 1H), 7.59 (s, 1H), 7.51 (dd, J = 8.5, 5.5 Hz, 2H), 7.12 (t, J = 8.7 Hz, 2H), 3.66 (s, 3H) ppm. |
| 9 | 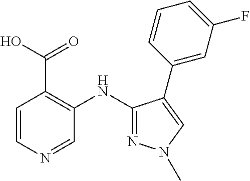 [SMILES: Cn1cc(-c2cccc(F)c2)c(Nc2cnccc2C(=O)O)n1] | LCMS: 313.10 [M + 1]; ¹H NMR (400 MHz, DMSO-d6) δ 10.17 (s, 1H), 9.40 (s, 1H), 8.11-8.02 (m, 2H), 7.76 (d, J = 5.0 Hz, 1H), 7.40 (td, J = 8.1, 6.2 Hz, 1H), 7.35-7.25 (m, 2H), 7.05 (td, J = 8.7, 2.5 Hz, 1H), 3.84 (s, 3H) ppm. |
| 10 | 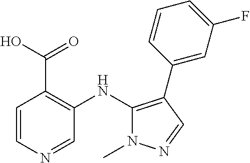 [SMILES: Cn1ncc(-c2cccc(F)c2)c1Nc1cnccc1C(=O)O] | LCMS: 313.10 [M + 1]; ¹H NMR (400 MHz, DMSO-d6) δ 13.95 (s, 1H), 9.09 (s, 1H), 8.01 (d, J = 6.0 Hz, 2H), 7.70 (d, J = 5.0 Hz, 1H), 7.59 (s, 1H), 7.37-7.25 (m, 3H), 6.96 (td, J = 8.6, 7.9, 2.8 Hz, 1H), 3.68 (s, 3H) ppm. |
| 11 | 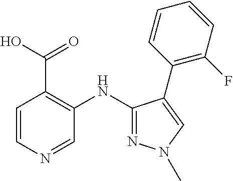 [SMILES: Cn1cc(-c2ccccc2F)c(Nc2cnccc2C(=O)O)n1] | LCMS: 312.90 [M + 1]; ¹H NMR (400 MHz, DMSO-d6) δ 10.02 (s, 1H), 9.50 (s, 1H), 8.07 (d, J = 5.6 Hz, 1H), 7.93 (s, 1H), 7.71 (d, J = 5.7 Hz, 1H), 7.50 (d, J = 7.7 Hz, 1H), 7.39-7.20 (m, 3H), 3.89 (s, 3H) ppm. |
| 12 | 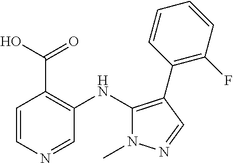 [SMILES: Cn1ncc(-c2ccccc2F)c1Nc1cnccc1C(=O)O] | LCMS: 312.90 [M + 1]; ¹H NMR (400 MHz, DMSO-d6) δ 9.08 (s, 1H), 7.98 (d, J = 5.1 Hz, 1H), 7.80 (d, J = 2.4 Hz, 1H), 7.69-7.60 (m, 2H), 7.43 (td, J = 7.7, 1.8 Hz, 1H), 7.28-7.05 (m, 3H), 3.70 (s, 3H) ppm. |
| 13 | 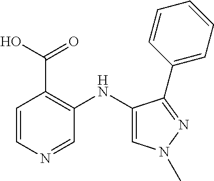 [SMILES: Cn1cc(Nc2cnccc2C(=O)O)c(-c2ccccc2)n1] | LCMS: 295.05 [M + 1]; ¹H NMR (400 MHz, DMSO-d6) δ 9.12 (s, 1H), 8.13- 7.82 (m, 4H), 7.79-7.56 (m, 2H), 7.47- 7.19 (m, 3H), 3.92 (s, 3H) ppm. |
| 14 | 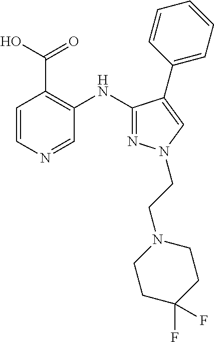 [SMILES: O=C(O)c1ccncc1Nc1nn(CCN2CCC(F)(F)CC2)cc1-c1ccccc1] | LCMS: 428.00 [M + 1]; ¹H NMR (400 MHz, Methanol-d4) δ 9.80 (s, 1H), 8.31 (d, J = 5.9 Hz, 1H), 8.13 (d, J = 5.7 Hz, 1H), 7.96 (d, J = 1.5 Hz, 1H), 7.51-7.36 (m, 4H), 7.31 (t, J = 7.3 Hz, 1H), 4.71 (t, J = 5.8 Hz, 2H), 3.85 (t, J = 5.9 Hz, 2H), 3.62 (s, 4H), 2.45 (tt, J = 11.5, 5.8 Hz, 4H) ppm. |
| 15 | 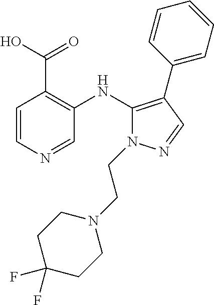 [SMILES: O=C(O)c1ccncc1Nc1c(-c2ccccc2)cnn1CCN1CCC(F)(F)CC1] | LCMS: 428.30 [M + 1]; ¹H NMR (400 MHz, Methanol-d4) δ 8.38 (d, J = 5.8 Hz, 1H), 8.13 (d, J = 5.9 Hz, 1H), 8.07 (s, 1H), 8.00 (d, J = 1.0 Hz, 1H), 7.49 (d, J = 7.5 Hz, 2H), 7.29 (t, J = 7.6 Hz, 2H), 7.20 (t, J = 7.6 Hz, 1H), 4.63 (t, J = 5.9 Hz, 2H), 3.79 (t, J = 5.9 Hz, 4H), 3.47 (s, 2H), 2.46 (s, 4H) ppm. |
| 16 | 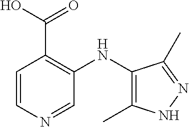 [SMILES: Cc1n[nH]c(C)c1Nc1cnccc1C(=O)O] | LCMS: 232.95 [M + 1]; ¹H NMR (400 MHz, Methanol-d4) δ 8.44 (d, J = 5.6 Hz, 1H), 8.22-8.14 (m, 2H), 2.31 (s, 6H) ppm. |
| 17 | 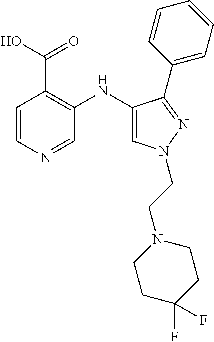 [SMILES: O=C(O)c1ccncc1Nc1cn(CCN2CCC(F)(F)CC2)nc1-c1ccccc1] | LCMS: 427.95 [M + 1]; ¹H NMR (400 MHz, Methanol-d4) δ 8.48 (s, 1H), 8.38-8.31 (m, 1H), 8.08-7.96 (m, 2H), 7.74 (d, J = 7.4 Hz, 2H), 7.32 (dq, J = 13.4, 7.1 Hz, 3H), 4.75 (t, J = 5.4 Hz, 2H), 4.00- 3.84 (m, 4H), 3.42 (s, 2H), 2.65-2.42 (m, 4H) ppm. |
| 18 | 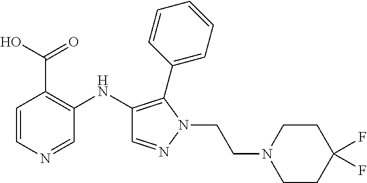 [SMILES: O=C(O)c1ccncc1Nc1cnn(CCN2CCC(F)(F)CC2)c1-c1ccccc1] | LCMS: 428.00 [M + 1]; ¹H NMR (400 MHz, DMSO-d6) δ 8.77 (d, J = 7.5 Hz, 1H), 8.08 (s, 1H), 7.85 (d, J = 5.0 Hz, 1H), 7.70 (s, 1H), 7.57-7.36 (m, 6H), 4.19 (t, J = 6.4 Hz, 2H), 2.73 (t, J = 6.4 Hz, 2H), 2.35-2.33 (m, 4H), 1.84- 1.74 (m, 4H) ppm. |
| 19 | 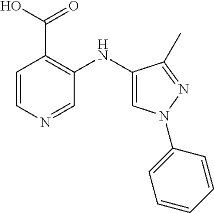 [SMILES: Cc1nn(-c2ccccc2)cc1Nc1cnccc1C(=O)O] | LCMS: 294.91 [M + 1]; ¹H NMR (400 MHz, DMSO-d6) δ 13.78 (brs, 1H), 8.90 (s, 1H), 8.66 (s, 1H), 8.27 (s, 1H), 7.99 (d, J = 5.1 Hz, 1H), 7.84 (d, J = 8.0 Hz, 2H), 7.68 (d, J = 5.1 Hz, 1H), 7.48 (t, J = 7.8 Hz, 2H), 7.27 (t, J = 7.4 Hz, 1H), 2.17 (s, 3H) ppm. |
| 20 | 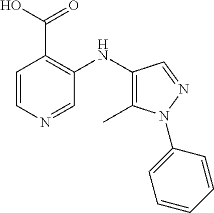 [SMILES: Cc1c(Nc2cnccc2C(=O)O)cnn1-c1ccccc1] | LCMS: 294.95 [M + 1]; ¹H NMR (400 MHz, DMSO-d6) δ 13.72 (brs, 1H), 8.74 (s, 1H), 8.12 (s, 1H), 7.95 (d, J = 5.1 Hz, 1H), 7.75 (s, 1H), 7.68-7.51 (m, 5H), 7.45 (t, J = 7.2 Hz, 1H), 2.21 (s, 3H) ppm. |
| 21 | 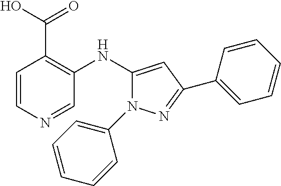 [SMILES: O=C(O)c1ccncc1Nc1cc(-c2ccccc2)nn1-c1ccccc1] | LCMS: 357.25 [M + 1]; ¹H NMR (400 MHz, DMSO-d6) δ 9.65 (s, 1H), 8.48 (s, 1H), 8.13 (d, J = 5.3 Hz, 1H), 7.97-7.90 (m, 2H), 7.80 (d, J = 5.2 Hz, 1H), 7.68 (d, J = 7.9 Hz, 2H), 7.55-7.32 (m, 6H), 7.08 (s, 1H) ppm. |
| 22 | 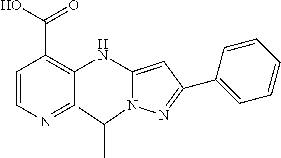 [SMILES: CC(C)n1nc(-c2ccccc2)cc1Nc1cnccc1C(=O)O] | LCMS: 323.00 [M + 1]; ¹H NMR (400 MHz, DMSO-d6) δ 9.49 (s, 1H), 8.22 (d, J = 4.0 Hz, 2H), 8.06 (d, J = 5.4 Hz, 1H), 7.86-7.79 (m, 2H), 7.41 (dd, J = 8.4, 7.0 Hz, 2H), 7.35-7.26 (m, 1H), 6.79 (s, 1H), 4.54 (hept, J = 6.6 Hz, 1H), 1.42 (d, J = 6.5 Hz, 6H) ppm. |
| 23 | 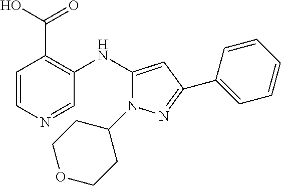 [SMILES: O=C(O)c1ccncc1Nc1cc(-c2ccccc2)nn1C1CCOCC1] | LCMS: 365.00 [M + 1]; ¹H NMR (400 MHz, DMSO-d6) δ 9.36 (s, 1H), 8.25 (s, 1H), 8.15 (d, J = 5.2 Hz, 1H), 7.85 (dd, J = 12.3, 6.4 Hz, 3H), 7.41 (t, J = 7.5 Hz, 2H), 7.31 (t, J = 7.4 Hz, 1H), 6.79 (s, 1H), 4.44 (td, J = 11.1, 5.4 Hz, 1H), 4.01-3.91 (m, 2H), 3.51-3.33 (m, 2H), 2.13 (qd, J = 12.4, 4.5 Hz, 2H), 1.85 (dd, J = 12.9, 4.3 Hz, 2H) ppm. |
| 24 | 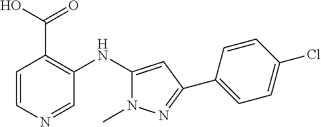 [SMILES: Cn1nc(-c2ccc(Cl)cc2)cc1Nc1cnccc1C(=O)O] | LCMS: 329.00 [M + 1]; ¹H NMR (400 MHz, DMSO-d6) δ 9.30 (s, 1H), 8.30 (s, 1H), 8.11 (d, J = 5.0 Hz, 1H), 7.85 (dd, J = 8.4, 2.2 Hz, 2H), 7.73 (d, J = 5.0 Hz, 1H), 7.46 (dd, J = 8.7, 2.3 Hz, 2H), 6.81 (s, 1H), 3.74 (s, 3H) ppm. |
| 25 | 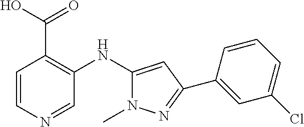 [SMILES: Cn1nc(-c2cccc(Cl)c2)cc1Nc1cnccc1C(=O)O] | LCMS: 329.00 [M + 1]; ¹H NMR (400 MHz, DMSO-d6) δ 13.99 (s, 1H), 9.36 (s, 1H), 8.34 (d, J = 1.7 Hz, 1H), 8.12 (dd, J = 5.1, 1.7 Hz, 1H), 7.88 (t, J = 1.9 Hz, 1H), 7.84-7.69 (m, 2H), 7.48- 7.31 (m, 2H), 6.89 (d, J = 1.9 Hz, 1H), 3.75 (d, J = 1.8 Hz, 3H) ppm. |
| 26 | 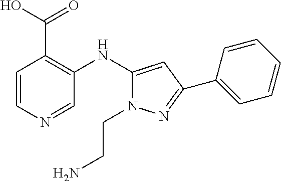 [SMILES: NCCn1nc(-c2ccccc2)cc1Nc1cnccc1C(=O)O] | LCMS: 323.82 [M + 1]; ¹H NMR (400 MHz, DMSO-d6) δ 9.33 (s, 1H), 8.36 (s, 1H), 8.27 (d, J = 6.1 Hz, 3H), 8.19 (d, J = 5.4 Hz, 1H), 7.99 (d, J = 5.4 Hz, 1H), 7.92-7.83 (m, 2H), 7.43 (t, J = 7.6 Hz, 2H), 7.33 (t, J = 7.4 Hz, 1H), 6.85 (s, 1H), 4.30 (t, J = 6.1 Hz, 2H), 3.32-3.27 (m, 2H) ppm. |
| 27 | 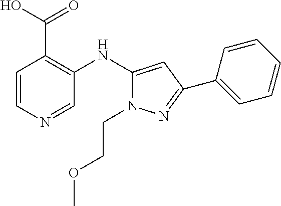 [SMILES: COCCn1nc(-c2ccccc2)cc1Nc1cnccc1C(=O)O] | LCMS: 338.94 [M + 1]; ¹H NMR (400 MHz, DMSO-d6) δ 8.59 (s, 1H), 8.46 (d, J = 5.6 Hz, m 1H), 8.25 (d, J = 6.4 Hz, 1H), 7.82 (d, J = 7.6 Hz, 2H), 7.46-7.32 (m, 3H), 6.82 (s, 1H), 4.37 (t, J = 4.8 Hz, 2H), 3.75 (t, J = 4.8 Hz, 2H), 3.35 (s, 3H) ppm. |
| 28 | 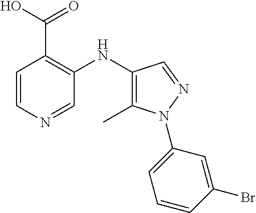 [SMILES: Cc1c(Nc2cnccc2C(=O)O)cnn1-c1cccc(Br)c1] | LCMS: 374.91 [M + 1]; ¹H NMR (400 MHz, DMSO-d6) δ 13.69 (brs, 1H), 8.68 (s, 1H), 8.13 (s, 1H), 7.96 (d, J = 5.0 Hz, 1H), 7.87 (d, J = 2.3 Hz, 1H), 7.78 (s, 1H), 7.69-7.54 (m, 3H), 7.51 (t, J = 8.0 Hz, 1H), 2.23 (s, 3H) ppm. |
| 29 | 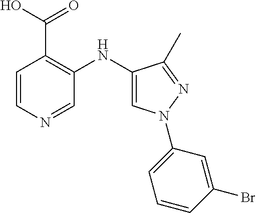 [SMILES: Cc1nn(-c2cccc(Br)c2)cc1Nc1cnccc1C(=O)O] | LCMS: 374.91 [M + 1]; ¹H NMR (400 MHz, DMSO-d6) δ 9.11 (s, 1H), 8.77 (s, 1H), 8.36 (s, 1H), 8.11-8.04 (m, 2H), 7.87 (dd, J = 5.7, 3.5 Hz, 2H), 7.45 (d, J = 6.8 Hz, 2H), 2.19 (s, 3H) ppm. |
| 30 | 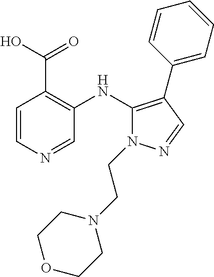 [SMILES: O=C(O)c1ccncc1Nc1c(-c2ccccc2)cnn1CCN1CCOCC1] | LCMS: 394.00 [M + 1]; ¹H NMR (400 MHz, Methanol-d4) δ 8.41 (d, J = 5.8 Hz, 1H), 8.15 (d, J = 5.8 Hz, 1H), 8.02 (d, J = 11.9 Hz, 2H), 7.53- 7.45 (m, 2H), 7.34-7.25 (m, 2H), 7.25-7.15 (m, 1H), 4.63 (t, J = 6.1 Hz, 2H), 4.12-4.04 (m, 2H), 3.91 (t, J = 12.4 Hz, 2H), 3.79-3.66 (m, 4H), 3.31-3.22 (m, 2H) ppm. |
| 31 | 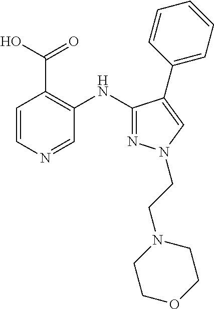 [SMILES: O=C(O)c1ccncc1Nc1nn(CCN2CCOCC2)cc1-c1ccccc1] | LCMS: 394.16 [M + 1]; ¹H NMR (400 MHz, Methanol-d4) δ 9.89 (s, 1H), 8.45 (d, J = 5.9 Hz, 1H), 8.25 (d, J = 5.9 Hz, 1H), 7.99 (s, 1H), 7.56-7.49 (m, 2H), 7.48-7.39 (m, 2H), 7.37-7.28 (m, 1H), 4.71 (t, J = 6.1 Hz, 2H), 4.06 (d, J = 13.1 Hz, 2H), 3.90-3.79 (m, 4H), 3.59 (d, J = 12.5 Hz, 2H), 3.38-3.22 (m, 2H) ppm. |
| 32 | 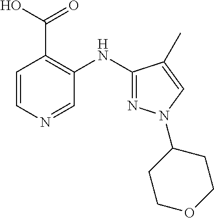 [SMILES: Cc1cn(C2CCOCC2)nc1Nc1cnccc1C(=O)O] | LCMS: 303.05 [M + 1]; ¹H NMR (400 MHz, DMSO-d6) δ 13.64 (s, 1H), 8.55 (s, 1H), 7.96-7.87 (m, 2H), 7.61 (d, J = 5.0 Hz, 1H), 7.47 (s, 1H), 4.40 (tt, J = 11.2, 4.2 Hz, 1H), 4.02-3.93 (m, 2H), 3.49 (td, J = 11.9, 2.0 Hz, 2H), 2.15 (s, 3H), 2.05 (qd, J = 12.4, 4.6 Hz, 2H), 1.83 (dt, J = 13.6, 3.1 Hz, 2H) ppm. |
| 33 | 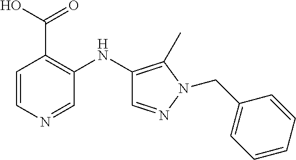 [SMILES: Cc1c(Nc2cnccc2C(=O)O)cnn1Cc1ccccc1] | LCMS: 309.05 [M + 1]; ¹H NMR (400 MHz, DMSO-d6) δ 13.65 (s, 1H), 8.64 (s, 1H), 7.98-7.86 (m, 2H), 7.61 (d, J = 5.0 Hz, 1H), 7.51 (s, 1H), 7.41-7.24 (m, 3H), 7.19-7.12 (m, 2H), 5.35 (s, 2H), 2.05 (s, 3H) ppm. |
| 34 | 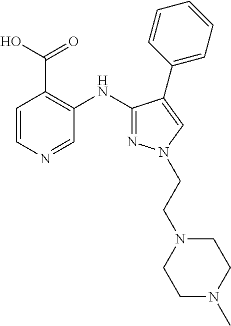 [SMILES: CN1CCN(CCn2cc(-c3ccccc3)c(Nc3cnccc3C(=O)O)n2)CC1] | LCMS: 407.15 [M + 1]; ¹H NMR (400 MHz, Methanol-d4) δ 8.40 (dd, J = 5.9, 1.2 Hz, 1H), 8.16-8.06 (m, 2H), 7.98 (d, J = 1.2 Hz, 1H), 7.52-7.44 (m, 2H), 7.29 (ddd, J = 7.8, 6.8, 1.3 Hz, 2H), 7.24- 7.15 (m, 1H), 4.58 (t, J = 5.8 Hz, 2H), 3.67-3.63 (m, 10H), 3.00 (s, J = 1.2 Hz, 3H) ppm. |
| 35 | 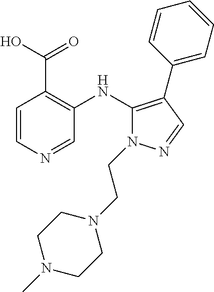 [SMILES: CN1CCN(CCn2ncc(-c3ccccc3)c2Nc2cnccc2C(=O)O)CC1] | LCMS: 407.21 [M + 1]; ¹H NMR (400 MHz, Methanol-d4) δ 9.91 (s, 1H), 8.45 (d, J = 5.8 Hz, 1H), 8.24 (s, 1H), 7.98 (d, J = 1.1 Hz, 1H), 7.57-7.49 (m, 2H), 7.43 (dd, J = 8.5, 6.9 Hz, 2H), 7.37-7.26 (m, 1H), 4.62 (t, J = 5.9 Hz, 2H), 3.70- 3.58 (m, 10H), 2.97 (s, 3H) ppm. |
| 36 | 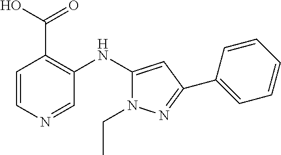 [SMILES: CCn1nc(-c2ccccc2)cc1Nc1cnccc1C(=O)O] | LCMS: 308.97 [M + 1]; ¹H NMR (400 MHz, Methanol-d4) δ 8.52-8.43 (m, 2H), 8.29 (d, J = 5.9 Hz, 1H), 7.87-7.78 (m, 2H), 7.48-7.32 (m, 3H), 6.83 (s, 1H), 4.22 (q, J = 7.2 Hz, 2H), 1.48 (t, J = 7.2 Hz, 3H) ppm. |
| 37 | 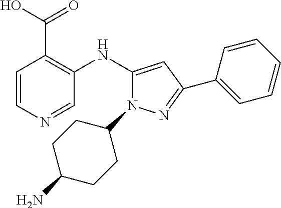 | LCMS: 378 [M + 1]; ¹H NMR (400 MHz, DMSO-d6) δ 9.25 (s, 1H), 8.26 (s, 1H), 8.11 (d, J = 5.0 Hz, 1H), 7.89 (d, J = 7.3 Hz, 5H), 7.73 (d, J = 5.0 Hz, 1H), 7.42 (t, J = 7.6 Hz, 2H), 7.31 (t, J = 7.4 Hz, 1H), 6.80 (s, 1H), 4.36 (q, J = 5.3 Hz, 1H), 3.27 (s, 1H), 2.09 (ddt, J = 24.9, 19.0, 7.7 Hz, 4H), 1.84 (ddd, J = 21.4, 11.6, 7.2 Hz, 4H) ppm. |
| 38 | 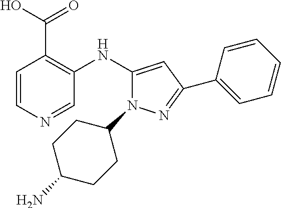 | LCMS: 378.10 [M + 1]; ¹H NMR (400 MHz, DMSO-d6) δ 9.25 (s, 1H), 8.26 (s, 1H), 8.12 (d, J = 5.1 Hz, 1H), 7.82 (d, J = 7.0 Hz, 5H), 7.74 (d, J = 5.1 Hz, 1H), 7.40 (t, J = 7.6 Hz, 2H), 7.30 (t, J = 7.4 Hz, 1H), 6.77 (s, 1H), 4.14 (tt, J = 10.4, 5.3 Hz, 1H), 3.23- 3.10 (m, 1H), 2.10-1.96 (m, 6H), 1.52-1.38 (m, 2H) ppm. |
| 39 | 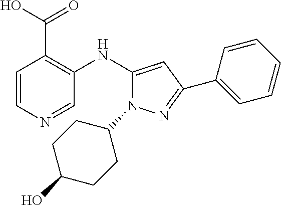 | LCMS: 379.00 [M + 1]; ¹H NMR (400 MHz, DMSO-d6) δ 9.29 (s, 1H), 8.28 (s, 1H), 8.11 (d, J = 5.1 Hz, 1H), 7.82 (d, J = 7.6 Hz, 2H), 7.73 (d, J = 5.0 Hz, 1H), 7.39 (t, J = 7.5 Hz, 2H), 7.29 (t, J = 7.4 Hz, 1H), 6.75 (s, 1H), 4.15-4.12 (m, 1H), 3.50-3.45 (m, 1H), 2.00-1.92 (m, 6H), 1.31-1.18 (m, 2H) ppm. |
| 40 | 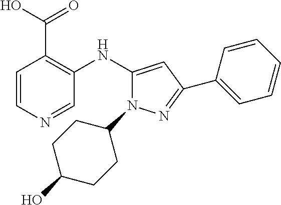 | LCMS: 379.04 [M + 1]; ¹H NMR (400 MHz, DMSO-d6) δ 9.34 (s, 1H), 8.28 (s, 1H), 8.13 (dd, J = 5.2, 2.2 Hz, 1H), 7.83 (t, J = 8.0 Hz, 3H), 7.40 (t, J = 7.8 Hz, 2H), 7.30 (dd, J = 8.3, 6.2 Hz, 1H), 6.76 (d, J = 1.8 Hz, 1H), 4.21-4.09 (m, 1H), 3.84 (s, 1H), 2.29 (q, J = 12.7, 11.2 Hz, 2H), 1.82-1.77 (m, 2H), 1.65-1.61 (m, 2H), 1.55-1.48 (m, 2H) ppm. |
| 41 | 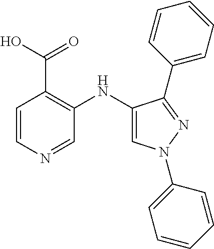 [SMILES: O=C(O)c1ccncc1Nc1cn(-c2ccccc2)nc1-c1ccccc1] | LCMS: 356.99 [M + 1]; ¹H NMR (400 MHz, DMSO-d6) δ 13.68 (s, 1H), 8.89 (s, 1H), 8.23 (s, 1H), 8.00 (s, 1H), 7.91 (d, J = 5.0 Hz, 1H), 7.59 (d, J = 5.0 Hz, 1H), 7.44-7.19 (m, 10H) ppm. |
| 42 | 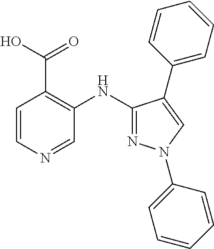 [SMILES: O=C(O)c1ccncc1Nc1nn(-c2ccccc2)cc1-c1ccccc1] | LCMS: 357 [M + 1]; ¹H NMR (400 MHz, DMSO-d6) δ 10.73 (s, 1H), 9.93 (s, 1H), 8.86 (s, 1H), 8.28 (d, J = 5.3 Hz, 1H), 8.01 (d, J = 5.4 Hz, 1H), 7.93 (d, J = 8.1 Hz, 2H), 7.69-7.27 (m, 8H) ppm. |
| 43 | 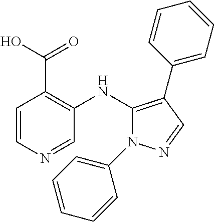 [SMILES: O=C(O)c1ccncc1Nc1c(-c2ccccc2)cnn1-c1ccccc1] | LCMS: 357.05 [M + 1]; ¹H NMR (400 MHz, Methanol-d4) δ 8.12 (s, 1H), 7.92-7.79 (m, 2H), 7.69 (s, 1H), 7.59 (ddt, J = 7.9, 3.1, 1.8 Hz, 4H), 7.49-7.27 (m, 6H), 7.26-7.16 (m, 1H) ppm. |
| 44 | 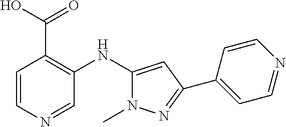 [SMILES: Cn1nc(-c2ccncc2)cc1Nc1cnccc1C(=O)O] | LCMS: 295.95 [M + 1]; ¹H NMR (400 MHz, DMSO-d6) δ 9.40 (s, 1H), 8.65-8.59 (m, 2H), 8.34 (s, 1H), 8.13 (d, J= 5.1 Hz, 1H), 7.90-7.83 (m, 2H), 7.74 (d, J = 5.1 Hz, 1H), 7.03 (s, 1H), 3.79 (s, 3H) ppm. |
| 45 | 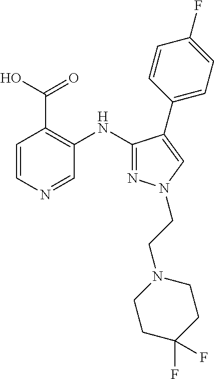 [SMILES: O=C(O)c1ccncc1Nc1nn(CCN2CCC(F)(F)CC2)cc1-c1ccc(F)cc1] | LCMS: 446 [M + 1]; ¹H NMR (400 MHz, DMSO-d6) δ 11.75 (brs, 1H), 10.28 (s, 1H), 9.53 (s, 1H), 8.19- 8.11 (m, 2H), 7.87 (d, J = 5.3 Hz, 1H), 7.60-7.50 (m, 2H), 7.33-7.22 (m, 2H), 4.66 (t, J = 6.5 Hz, 2H), 3.72 (q, J = 8.9, 7.7 Hz, 2H), 3.25 (s, 4H), 2.40 (s, 4H) ppm. |
| 46 | 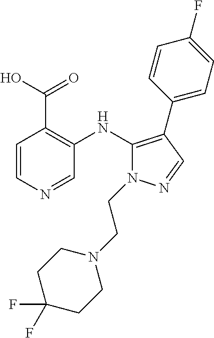 [SMILES: O=C(O)c1ccncc1Nc1c(-c2ccc(F)cc2)cnn1CCN1CCC(F)(F)CC1] | LCMS: 446 [M + 1]; ¹H NMR (400 MHz, DMSO-d6) δ 11.71 (s, 1H), 9.22 (s, 1H), 8.09-8.01 (m, 2H), 7.79 (d, J = 5.1 Hz, 1H), 7.69 (s, 1H), 7.56-7.46 (m, 2H), 7.18- 7.07 (m, 2H), 4.50 (t, J = 6.7 Hz, 2H), 3.65 (q, J = 7.2, 6.8 Hz, 4H), 3.22 (q, J = 7.0 Hz, 2H), 2.37 (s, 4H) ppm. |
| 47 | 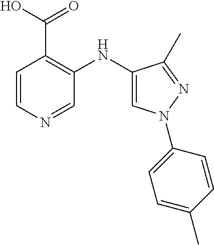 [SMILES: Cc1ccc(-n2cc(Nc3cnccc3C(=O)O)c(C)n2)cc1] | LCMS: 308.95 [M + 1]; ¹H NMR (400 MHz, DMSO-d6) δ 13.70 (s, 1H), 8.68 (s, 1H), 8.11 (s, 1H), 7.95 (d, J = 5.1 Hz, 1H), 7.72 (s, 1H), 7.65 (d, J = 5.1 Hz, 1H), 7.52- 7.43 (m, 2H), 7.38-7.31 (m, 2H), 2.38 (s, 3H), 2.18 (s, 3H) ppm. |
| 48 | 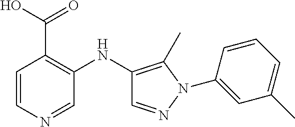 [SMILES: Cc1cccc(-n2ncc(Nc3cnccc3C(=O)O)c2C)c1] | LCMS: 308.97 [M + 1]; ¹H NMR (400 MHz, DMSO-d6) δ 13.69 (s, 1H), 8.68 (s, 1H), 8.11 (s, 1H), 7.95 (d, J = 5.1 Hz, 1H), 7.73 (s, 1H), 7.65 (d, J = 5.1 Hz, 1H), 7.47- 7.35 (m, 3H), 7.26 (d, J = 7.2 Hz, 1H), 2.40 (s, 3H), 2.19 (s, 3H) ppm. |
| 49 | 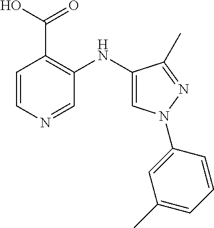 [SMILES: Cc1cccc(-n2cc(Nc3cnccc3C(=O)O)c(C)n2)c1] | LCMS: 308.90 [M + 1]; ¹H NMR (400 MHz, DMSO-d6) δ 13.80 (s, 1H), 9.10 (s, 1H), 8.62 (s, 1H), 8.27 (s, 1H), 7.98 (d, J = 5.0 Hz, 1H), 7.72-7.58 (m, 3H), 7.34 (t, J = 7.8 Hz, 1H), 7.08 (d, J = 7.5 Hz, 1H), 2.38 (s, 3H), 2.17 (s, 3H) ppm. |
| 50 | 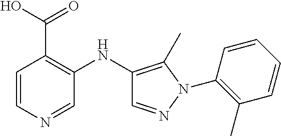 [SMILES: Cc1ccccc1-n1ncc(Nc2cnccc2C(=O)O)c1C] | LCMS: 308.97 [M + 1]; ¹H NMR (400 MHz, DMSO-d6) δ 13.72 (s, 1H), 8.67 (s, 1H), 8.09 (s, 1H), 7.94 (d, J = 5.0 Hz, 1H), 7.71 (s, 1H), 7.65 (d, J = 5.0 Hz, 1H), 7.44 (d, J = 2.7 Hz, 2H), 7.42-7.34 (m, 2H), 2.04 (s, 3H), 1.93 (s, 3H) ppm. |
| 51 | 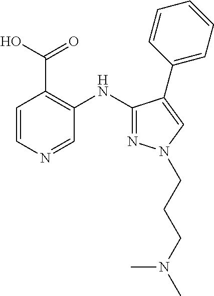 [SMILES: CN(C)CCCn1cc(-c2ccccc2)c(Nc2cnccc2C(=O)O)n1] | LCMS: 366.05 [M + 1]; ¹H NMR (400 MHz, DMSO-d6) δ 10.36 (s, 2H), 9.56 (s, 1H), 8.20-8.08 (m, 2H), 7.92 (d, J = 5.3 Hz, 1H), 7.56-7.48 (m, 2H), 7.42 (td, J = 7.9, 2.0 Hz, 2H), 7.29 (dd, J = 8.3, 6.4 Hz, 1H), 4.23 (m, 2H), 3.17-3.06 (m, 2H), 2.77 (d, 7= 4.8 Hz, 6H), 2.28 (t, J = 7.7 Hz, 2H) ppm. |
| 52 | 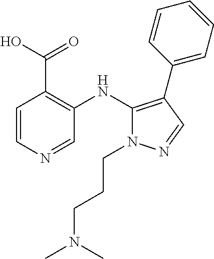 [SMILES: CN(C)CCCn1ncc(-c2ccccc2)c1Nc1cnccc1C(=O)O] | LCMS: 366 [M + 1]; ¹H NMR (400 MHz, DMSO-d6) δ 10.18 (s, 1H), 9.20 (s, 1H), 8.04 (d, J = 4.5 Hz, 2H), 7.80 (d, J = 5.1 Hz, 1H), 7.67 (s, 1H), 7.49 (d, J = 7.8 Hz, 2H), 7.28 (t, J = 7.6 Hz, 2H), 7.15 (t, J = 7.5 Hz, 1H), 4.07 (t, J = 6.4 Hz, 2H), 3.08 (dt, J = 9.6, 5.2 Hz, 2H), 2.69 (d, J = 4.8 Hz, 6H), 2.15 (dq, J = 14.0, 6.8 Hz, 2H) ppm. |
| 53 | 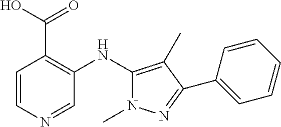 [SMILES: Cc1c(-c2ccccc2)nn(C)c1Nc1cnccc1C(=O)O] | LCMS: 309 [M + 1]; ¹H NMR (400 MHz, DMSO-d6) δ 9.05 (s, 1H), 8.16-8.09 (m, 1H), 7.91-7.80 (m, 2H), 7.71 (ddd, J = 8.3, 2.3, 1.3 Hz, 2H), 7.50-7.40 (m, 2H), 7.38-7.29 (m, 1H), 3.72-3.66 (m, 3H), 2.01 (d, J = 3.1 Hz, 3H) ppm. |
| 54 | 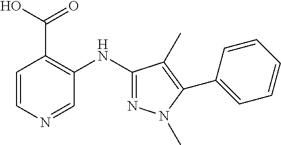 [SMILES: Cc1c(Nc2cnccc2C(=O)O)nn(C)c1-c1ccccc1] | LCMS: 308.90 [M + 1]; ¹H NMR (400 MHz, DMSO-d6) δ 9.95 (s, 1H), 9.51 (s, 1H), 8.12 (d, J = 5.2 Hz, 1H), 7.83 (d, J = 5.2 Hz, 1H), 7.59-7.41 (m, 5H), 3.70 (s, 3H), 1.92 (s, 3H) ppm. |
| 55 | 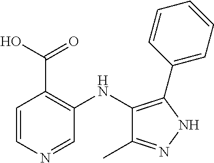 [SMILES: Cc1n[nH]c(-c2ccccc2)c1Nc1cnccc1C(=O)O] | LCMS: 295.05 [M + 1]; ¹H NMR (400 MHz, D₂O) δ 8.21 (d, J = 5.6 Hz, 1H), 7.99 (s, 1H), 7.87-7.81 (m, 1H), 7.73 (d, J = 6.4 Hz, 2H), 7.48 (t, J = 3.6 Hz, 3H), 2.29 (s, 3H) ppm. |
| 56 | 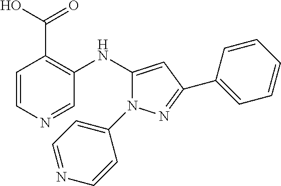 [SMILES: O=C(O)c1ccncc1Nc1cc(-c2ccccc2)nn1-c1ccncc1] | LCMS: 358 [M + 1]; ¹H NMR (400 MHz, DMSO-d6) δ 9.57 (s, 1H), 8.77-8.71 (m, 2H), 8.37 (s, 1H), 8.15 (d, J = 5.0 Hz, 1H), 8.06-7.95 (m, 4H), 7.74 (d, J = 5.0 Hz, 1H), 7.54-7.38 (m, 3H), 7.18 (s, 1H) ppm. |
| 57 | 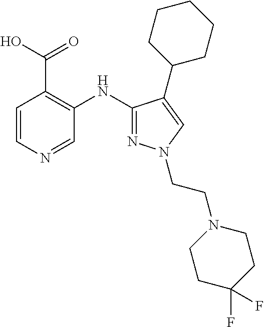 [SMILES: O=C(O)c1ccncc1Nc1nn(CCN2CCC(F)(F)CC2)cc1C1CCCCC1] | LCMS: 434 [M + 1]; ¹H NMR (400 MHz, Methanol-d4) δ 9.79 (s, 1H), 8.28 (d, J = 5.5 Hz, 1H), 8.18 (s, 1H), 7.56 (s, 1H), 4.61 (t, J = 5.9 Hz, 2H), 3.77 (t, J = 5.9 Hz, 2H), 3.54 (s, 4H), 2.43 (td, J = 13.9, 5.9 Hz, 5H), 1.99-1.90 (m, 2H), 1.86- 1.71 (m, 3H), 1.37 (tt, J = 24.7, 12.1 Hz, 5H) ppm. |
| 58 | 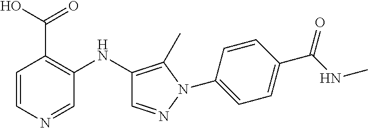 [SMILES: CNC(=O)c1ccc(-n2ncc(Nc3cnccc3C(=O)O)c2C)cc1] | LCMS: 352.05 [M + 1]; ¹H NMR (400 MHz, DMSO-d6) δ 8.71 (s, 1H), 8.56 (d, J = 4.9 Hz, 1H), 8.13 (s, 1H), 7.98 (dd, J = 10.4, 6.6 Hz, 3H), 7.80 (s, 1H), 7.69 (dd, J = 22.1, 6.7 Hz, 3H), 2.81 (d, J = 4.4 Hz, 3H), 2.26 (s, 3H) ppm. |
| 59 | 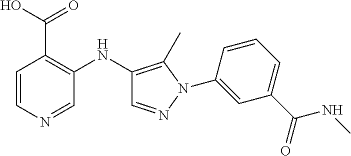 [SMILES: CNC(=O)c1cccc(-n2ncc(Nc3cnccc3C(=O)O)c2C)c1] | LCMS: 352.05 [M + 1]; ¹H NMR (400 MHz, DMSO-d6) δ 8.91 (s, 1H), 8.68-8.61 (m, 1H), 8.17 (s, 1H), 8.09-8.01 (m, 2H), 7.96-7.86 (m, 2H), 7.83-7.70 (m, 2H), 7.64 (t, J = 7.9 Hz, 1H), 2.81 (d, J = 4.5 Hz, 3H), 2.23 (s, 3H) ppm. |
| 60 | 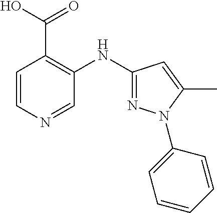 [SMILES: Cc1cc(Nc2cnccc2C(=O)O)nn1-c1ccccc1] | LCMS: 295.05 [M + 1]; ¹H NMR (400 MHz, DMSO-d6) δ 9.45 (s, 1H), 8.30 (s, 1H), 8.04 (d, J = 5.0 Hz, 1H), 7.64 (d, J = 5.0 Hz, 1H), 7.55 (d, J = 7.2 Hz, 2H), 7.44 (t, J = 7.8 Hz, 2H), 7.33 (t, J = 7.4 Hz, 1H), 6.31 (s, 1H), 2.25 (s, 3H) ppm. |
| 61 | 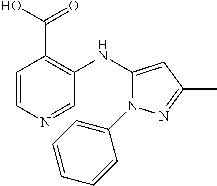 [SMILES: Cc1cc(Nc2cnccc2C(=O)O)n(-c2ccccc2)n1] | LCMS: 294.95 [M + 1]; ¹H NMR (400 MHz, DMSO-d6) δ 9.51-9.45 (m, 1H), 8.30 (s, 1H), 8.04 (d, J = 5.1 Hz, 1H), 7.64 (d, J = 5.0 Hz, 1H), 7.55 (d, J = 7.9 Hz, 1H), 7.38 (dt, J = 44.4, 7.6 Hz, 4H), 6.31 (s, 1H), 2.25 (s, 3H) ppm. |
| 62 | 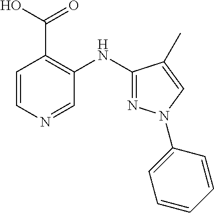 [SMILES: Cc1cn(-c2ccccc2)nc1Nc1cnccc1C(=O)O] | LCMS: 294.95 [M + 1]; ¹H NMR (400 MHz, DMSO-d6) δ 14.17 (brs, 1H), 10.20 (brs, 1H), 9.78 (s, 1H), 8.32 (s, 1H), 8.16 (d, J = 4.8 Hz, 1H), 7.79 (d, J = 7.0 Hz, 3H), 7.48 (t, J = 7.0 Hz, 2H), 7.22 (t, J = 7.0 Hz, 1H), 2.09 (s, 3H) ppm. |
| 63 | 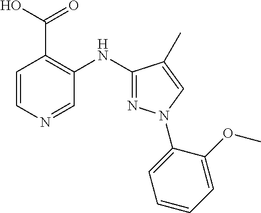 [SMILES: COc1ccccc1-n1cc(C)c(Nc2cnccc2C(=O)O)n1] | LCMS: 325.50 [M + 1]; ¹H NMR (400 MHz, DMSO-d6) δ 10.30 (s, 1H), 9.75 (s, 1H), 8.23 (d, J = 5.4 Hz, 1H), 8.07 (d, J = 1.1 Hz, 1H), 8.01 (d, J = 5.4 Hz, 1H), 7.72 (dd, J = 7.9, 1.6 Hz, 1H), 7.36-7.21 (m, 2H), 7.15-7.05 (m, 1H), 3.95 (s, 3H), 2.10 (s, 3H) ppm. |
| 64 | 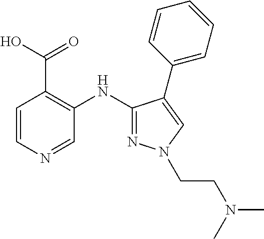 [SMILES: CN(C)CCn1cc(-c2ccccc2)c(Nc2cnccc2C(=O)O)n1] | LCMS: 352.05 [M + 1]; ¹H NMR (400 MHz, DMSO-d6) δ 10.23 (s, 2H), 9.50 (s, 1H), 8.16-8.06 (m, 2H), 7.73 (d, J = 5.1 Hz, 1H), 7.55-7.48 (m, 2H), 7.42 (t, J = 7.7 Hz, 2H), 7.34-7.24 (m, 1H), 4.57 (t, J = 6.3 Hz, 2H), 3.64 (t, J = 6.4 Hz, 2H), 2.83 (s, 6H) ppm. |
| 65 | 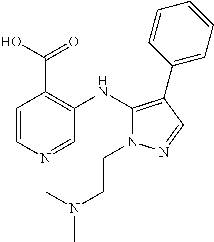 [SMILES: CN(C)CCn1ncc(-c2ccccc2)c1Nc1cnccc1C(=O)O] | LCMS: 352 [M + 1]; ¹H NMR (400 MHz, DMSO-d6) δ 10.36 (s, 1H), 9.32 (s, 1H), 8.13-8.05 (m, 2H), 7.92- 7.86 (m, 1H), 7.78 (d, J = 1.8 Hz, 1H), 7.52-7.45 (m, 2H), 7.32-7.23 (m, 2H), 7.20-7.11 (m, 1H), 4.44 (t, J = 6.3 Hz, 2H), 3.54 (d, J = 6.3 Hz, 2H), 2.87- 2.71 (m, 6H) ppm. |
| 66 | 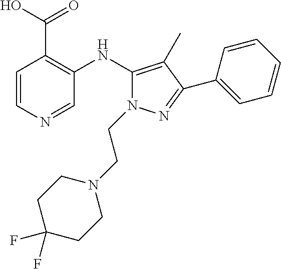 [SMILES: Cc1c(-c2ccccc2)nn(CCN2CCC(F)(F)CC2)c1Nc1cnccc1C(=O)O] | LCMS: 442.10 [M + 1]; ¹H NMR (400 MHz, Methanol-d4) δ 8.42 (d, J = 5.7 Hz, 1H), 8.26 (d, J = 5.6 Hz, 1H), 8.18 (s, 1H), 7.74 (d, J = 7.7 Hz, 2H), 7.43 (dt, J = 32.0, 7.2 Hz, 3H), 4.57 (t, J = 6.1 Hz, 2H), 3.77 (t, J = 6.3 Hz, 2H), 3.65-3.53 (m, 4H), 2.38- 2.45 (m, 4H), 2.10 (s, 3H) ppm. |
| 67 | 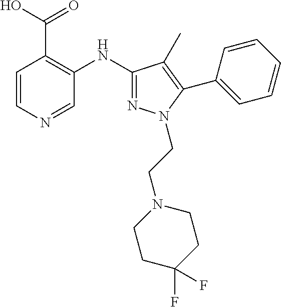 [SMILES: Cc1c(Nc2cnccc2C(=O)O)nn(CCN2CCC(F)(F)CC2)c1-c1ccccc1] | LCMS: 442.10 [M + 1]; ¹H NMR (400 MHz, DMSO-d6) δ 10.85 (brs, 1H), 10.05 (s, 1H), 9.60 (s, 1H), 8.15 (d, 5.2 Hz, 1H), 7.83 (d, J = 5.1 Hz, 1H), 7.63-7.46 (m, 5H), 4.39 (t, J = 6.8 Hz, 2H), 3.59-3.50 (m, 6H), 2.35-2.31 (m, 4H), 1.93 (s, 3H) ppm. |
| 68 | 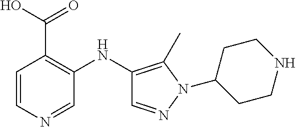 [SMILES: Cc1c(Nc2cnccc2C(=O)O)cnn1C1CCNCC1] | LCMS: 302.05 [M + 1]; ¹H NMR (400 MHz, Methanol-d4) δ 7.96-7.80 (m, 3H), 7.54 (s, 1H), 4.61 (td, J = 10.9, 5.5 Hz, 1H), 3.60 (dd, J = 10.4, 6.6 Hz, 2H), 3.29-3.20 (m, 2H), 2.44-2.28 (m, 4H), 2.25 (s, 3H) ppm. |
| 69 | 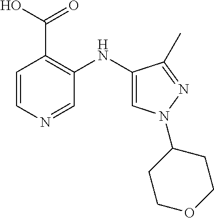 [SMILES: Cc1nn(C2CCOCC2)cc1Nc1cnccc1C(=O)O] | LCMS: 302.05 [M + 1]; ¹H NMR (400 MHz, Methanol-d4) δ 8.28 (d, J = 5.7 Hz, 1H), 8.10 (s, 1H), 8.05 (d, J = 5.6 Hz, 1H), 7.86 (s, 1H), 4.51 (tt, J = 9.9, 4.6 Hz, 1H), 3.58 (dt, J = 13.3, 3.9 Hz, 2H), 3.29-3.17 (m, 2H), 2.41-2.17 (m, 4H), 2.15 (s, 3H) ppm. |
| 70 | 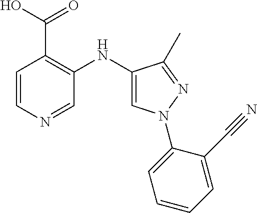 [SMILES: Cc1nn(-c2ccccc2C#N)cc1Nc1cnccc1C(=O)O] | LCMS: 320 [M + 1]; ¹H NMR (400 MHz, TFA) δ 8.83 (s, 1H), 8.03-7.85 (m, 3H), 7.70 (s, 1H), 7.47 (t, J = 7.1 Hz, 1H),7.24 (d, J = 9.2 Hz, 1H), 7.15 (t, J = 7.0 Hz, 1H), 2.56 (s, 3H) ppm. |
| 71 | 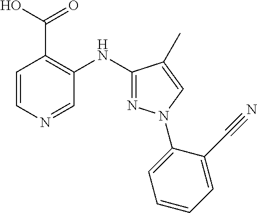 [SMILES: Cc1cn(-c2ccccc2C#N)nc1Nc1cnccc1C(=O)O] | LCMS: 320 [M + 1]; ¹H NMR (400 MHz, DMSO-d6) δ 10.44 (s, 1H), 9.90 (s, 1H), 8.31 (d, J = 1.1 Hz, 1H), 8.21 (d, J = 5.1 Hz, 1H), 7.96 (dd, J = 7.5, 1.1 Hz, 1H), 7.89- 7.76 (m, 3H), 7.45 (ddd, J = 8.1, 5.6, 2.9 Hz, 1H), 2.13 (d, J = 1.0 Hz, 3H) ppm. |
| 72 | 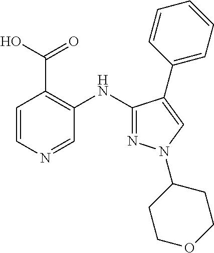 [SMILES: O=C(O)c1ccncc1Nc1nn(C2CCOCC2)cc1-c1ccccc1] | LCMS: 365.05 [M + 1]; ¹H NMR (400 MHz, DMSO-d6) δ 12.39 (s, 1H), 9.29 (s, 1H), 8.00 (s, 1H), 7.87 (d, J = 4.8 Hz, 1H), 7.69-7.57 (m, 3H), 7.35 (t, J = 7.8 Hz, 2H), 7.24- 7.15 (m, 1H), 4.33 (td, J = 10.8, 5.1 Hz, 1H), 4.03-3.95 (m, 2H), 3.59- 3.43 (m, 2H), 2.07-1.92 (m, 4H) ppm. |
| 73 | 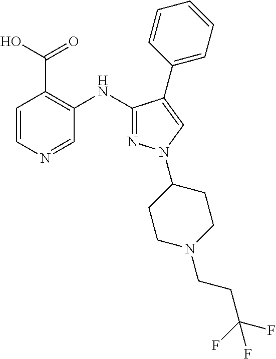 [SMILES: O=C(O)c1ccncc1Nc1nn(C2CCN(CCC(F)(F)F)CC2)cc1-c1ccccc1] | LCMS: 377 [M + 1]; ¹H NMR (400 MHz, DMSO-d6) δ 13.95 (s, 1H), 10.14 (s, 1H), 9.43 (s, 1H), 8.15-8.04 (m, 2H), 7.70 (d, J = 5.0 Hz, 1H), 7.54-7.46 (m, 2H), 7.41 (t, J = 7.8 Hz, 2H), 7.32- 7.22 (m, 1H), 4.40 (t, J = 6.8 Hz, 2H), 2.96 (qt, J = 11.3, 6.8 Hz, 2H) ppm. |
| 74 | 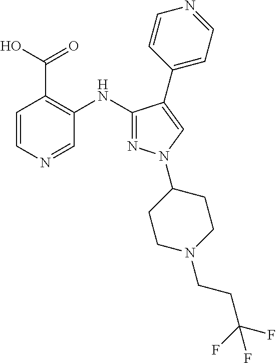 [SMILES: O=C(O)c1ccncc1Nc1nn(C2CCN(CCC(F)(F)F)CC2)cc1-c1ccncc1] | LCMS: 378 [M + 1]; ¹H NMR (400 MHz, DMSO-d6) δ 10.36 (s, 1H), 9.39 (s, 1H), 8.56 (d, J = 4.8 Hz, 2H), 8.38 (s, 1H), 8.11 (d, J = 4.9 Hz, 1H), 7.74 (d, J = 5.0 Hz, 1H), 7.58- 7.52 (m, 2H), 4.42 (t, J = 6.8 Hz, 2H), 2.97 (qt, J = 11.1, 6.8 Hz, 2H) ppm. |
| 75 | 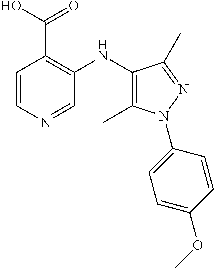 [SMILES: COc1ccc(-n2nc(C)c(Nc3cnccc3C(=O)O)c2C)cc1] | LCMS: 339.00 [M + 1]; ¹H NMR (400 MHz, DMSO-d6) δ 8.87 (s, 1H), 8.10-7.97 (m, 3H), 7.52-7.42 (m, 2H), 7.11-7.02 (m, 2H), 3.82 (s, 3H), 2.12 (s, 3H), 2.07 (s, 3H) ppm. |
| 76 | 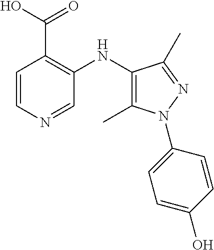 [SMILES: Cc1nn(-c2ccc(O)cc2)c(C)c1Nc1cnccc1C(=O)O] | LCMS: 325.00 [M + 1]; ¹H NMR (400 MHz, DMSO-d6) δ 13.65 (s, 1H), 9.72 (s, 1H), 8.52 (s, 1H), 7.96-7.87 (m, 2H), 7.64 (d, J = 5.1 Hz, 1H), 7.33 (d, J = 8.7 Hz, 2H), 6.91-6.78 (m, 2H), 2.06 (d, J = 19.9 Hz, 6H) ppm. |
| 77 | 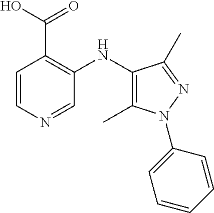 [SMILES: Cc1nn(-c2ccccc2)c(C)c1Nc1cnccc1C(=O)O] | LCMS: 309 [M + 1]; ¹H NMR (400 MHz, DMSO-d6) δ 8.95 (s, 1H), 8.09 (s, 2H), 8.02 (s, 1H), 7.62-7.49 (m, 4H), 7.47-7.37 (m, 1H), 2.19 (s, 3H), 2.09 (s, 3H) ppm. |
| 78 | 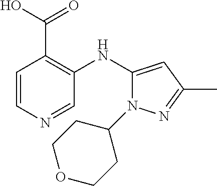 [SMILES: Cc1cc(Nc2cnccc2C(=O)O)n(C2CCOCC2)n1] | LCMS: 303.05 [M + 1]; ¹H NMR (400 MHz, DMSO-d6) δ 13.96 (s, 1H), 9.12 (s, 1H), 8.13-8.02 (m, 2H), 7.69 (d, J = 5.0 Hz, 1H), 6.02 (s, 1H), 4.27 (tt, J = 11.2, 4.8 Hz, 1H), 3.91 (dd, J = 11.9, 4.3 Hz, 2H), 3.37-3.31 (m, 2H), 2.17 (s, 3H), 2.02 (qd, J = 12.3, 4.5 Hz, 2H), 1.77-1.69 (m, 2H) ppm. |
| 79 | 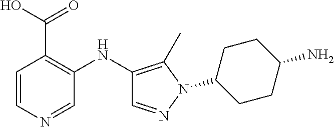 | LCMS: 316 [M + 1]; ¹H NMR (400 MHz, D₂O) δ 8.36 (d, J = 5.6 Hz, 1H), 8.08 (d, J = 6.0 Hz, 1H), 8.01 (s, 1H), 769 (s, 1H), 4.50-4.45 (m, 1H), 3.76-3.72 (m, 1H), 2.28 (s, 3H), 2.19-2.09 (m, 8H) ppm. |
| 80 | 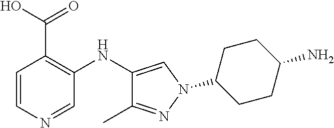 | LCMS: 316 [M + 1]; ¹H NMR (400 MHz, D₂O) δ 8.36 (d, J = 5.6 Hz, 1H), 8.08 (d, J = 6.0 Hz, 1H), 8.01 (s, 1H), 769 (s, 1H), 4.50-4.45 (m, 1H), 3.76-3.72 (m, 1H), 2.28 (s, 3H), 2.19-2.09 (m, 8H) ppm. |
| 81 | 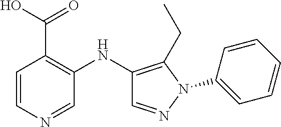 | LCMS: 309.05 [M + 1]; ¹H NMR (400 MHz, DMSO-d6) δ 13.71 (s, 1H), 8.69 (s, 1H), 8.14 (s, 1H), 7.95 (d, J = 5.1 Hz, 1H), 7.74 (s, 1H), 7.64 (d, J = 4.8 Hz, 1H), 7.57-7.42 (m, 5H), 2.64 (q, J = 7.5 Hz, 2H), 0.92 (t, J = 7.5 Hz, 3H) ppm. |
| 82 | 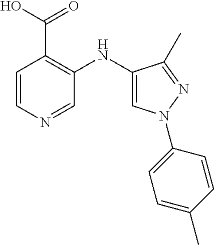 [SMILES: Cc1ccc(-n2cc(Nc3cnccc3C(=O)O)c(C)n2)cc1] | LCMS: 309.05 [M + 1]; ¹H NMR (400 MHz, DMSO-d6) δ 13.75 (s, 1H), 8.87 (s, 1H), 8.60 (s, 1H), 8.25 (s, 1H), 7.98 (d, J = 5.0 Hz, 1H), 7.69 (dd, J = 18.3, 6.5 Hz, 3H), 7.28 (d, J = 8.1 Hz, 2H), 2.33 (s, 3H), 2.15 (s, 3H) ppm. |
| 83 | 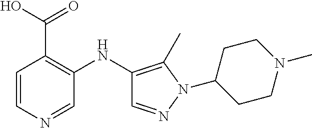 [SMILES: Cc1c(Nc2cnccc2C(=O)O)cnn1C1CCN(C)CC1] | LCMS: 316 [M + 1]; ¹H NMR (400 MHz, DMSO-d6) δ 10.70 (d, J = 11.2 Hz, 1H), 8.88 (s, 1H), 8.08-7.93 (m, 3H), 7.56 (d, J = 10.8 Hz, 1H), 4.47 (ddt, J = 11.5, 8.2, 4.0 Hz, 1H), 3.54-3.50 (m, 2H), 3.22-3.09 (m, 2H), 2.79 (dd, J = 20.3, 4.8 Hz, 3H), 2.47- 2.31 (m, 2H), 2.17 (s, 3H), 2.13-2.02 (m, 2H) ppm. |
| 84 | 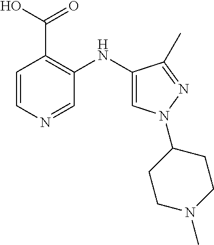 [SMILES: Cc1nn(C2CCN(C)CC2)cc1Nc1cnccc1C(=O)O] | LCMS: 316 [M + 1]; ¹H NMR (400 MHz, DMSO-d6) δ 10.82 (s, 1H), 8.97 (s, 1H), 8.05 (dd, J = 13.5, 5.3 Hz, 3H), 7.93 (s, 1H), 4.36 (tt, J = 10.2, 5.8 Hz, 1H), 3.56- 3.47 (m, 2H), 3.20-3.06 (m, 2H), 2.76 (dd, J = 7.6, 4.8 Hz, 3H), 2.32-2.17 (m, 4H), 2.06 (d, J = 9.4 Hz, 3H) ppm. |
| 85 | 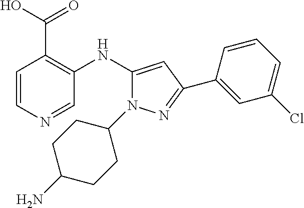 [SMILES: NC1CCC(n2nc(-c3cccc(Cl)c3)cc2Nc2cnccc2C(=O)O)CC1] | LCMS: 412.00 [M + 1]; ¹H NMR (400 MHz, DMSO-d6) δ 12.65 (s, 1H), 8.63 (s, 1H), 8.16 (d, J = 13.2 Hz, 3H), 8.04 (d, J = 4.8 Hz, 1H), 7.96 (t, J = 1.9 Hz, 1H), 7.85 (dt, J = 7.9, 1.3 Hz, 1H), 7.74 (d, J = 4.8 Hz, 1H), 7.42 (t, J = 7.8 Hz, 1H), 7.33 (ddd, J = 8.0, 2.2, 1.1 Hz, 1H), 6.85 (s, 1H), 4.44 (s, 1H), 3.42 (s, 1H), 2.32 (q, J = 11.8 Hz, 2H), 2.03 (d, J = 12.1 Hz, 2H), 1.91-1.86 (m, 4H) ppm. |
| 86 | 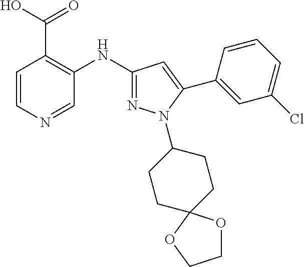 [SMILES: O=C(O)c1ccncc1Nc1cc(-c2cccc(Cl)c2)n(C2CCC3(CC2)OCCO3)n1] | LCMS: 455.00 [M+l]; 'H NMR (400 MHz, D₂O) δ 8.15 (s, 1H), 7.97 (d, J = 4.8 Hz, 1H), 7.67 (d, J = 4.4 Hz, 1H), 7.46 (s, 1H), 7.36-7.33 (m, 1H), 7.16- 7.13 (m, 2H), 6.15 (s, 1H), 4.15-4.10 (m, 1H), 3.99 (s, 4H), 1.98-1.95 (m, 2H), 1.85-1.80 (m, 4H), 1.71-1.68 (m, 2H) ppm. |
| 87 | 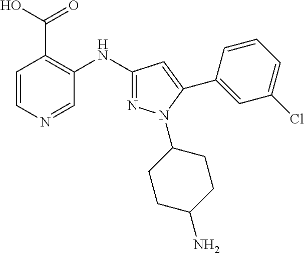 [SMILES: NC1CCC(n2nc(Nc3cnccc3C(=O)O)cc2-c2cccc(Cl)c2)CC1] | LCMS: 412.00 [M + 1]; ¹H NMR (400 MHz, DMSO-d6) δ 12.74 (s, 1H), 8.55 (s, 1H), 8.23 (s, 1H), 8.02 (d, J = 4.8 Hz, 1H), 7.91 (t, J = 1.8 Hz, 1H), 7.80 (dt, J = 7.7, 1.4 Hz, 1H), 7.74 (d, J = 4.8 Hz, 1H), 7.45-7.28 (m, 2H), 6.76 (s, 1H), 4.19 (tt, J = 10.2, 5.1 Hz, 1H), 3.21 (td, J = 11.2, 5.4 Hz, 1H), 2.16 (d, J = 12.1 Hz, 2H), 2.10-1.95 (m, 4H), 1.59 (qd, J = 12.3, 5.0 Hz, 2H) ppm. |
| 88 | 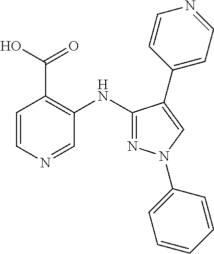 [SMILES: O=C(O)c1ccncc1Nc1nn(-c2ccccc2)cc1-c1ccncc1] | LCMS: 358.00 [M + 1]; ¹H NMR (400 MHz, DMSO-d6) δ 10.62 (s, 1H), 9.65 (s, 1H), 9.29 (s, 1H), 8.79 (d, J = 5.7 Hz, 2H), 8.24 (d, J = 5.1 Hz, 1H), 8.02 (s, 2H), 7.98-7.90 (m, 2H), 7.84 (d, J = 5.1 Hz, 1H), 7.60 (t, J = 7.8 Hz, 2H), 7.38 (t, J = 7.4 Hz, 1H), 7.21 (dd, J = 27.1, 7.5 Hz, 1H) ppm. |
| 89 | 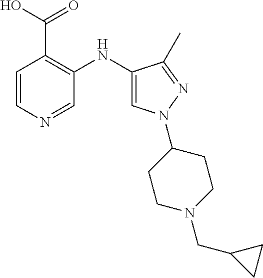 [SMILES: Cc1nn(C2CCN(CC3CC3)CC2)cc1Nc1cnccc1C(=O)O] | ESI-LCMS (m/z): 356.3 [M + 1]; ¹H-NMR (400 MHz, Methanol-d4): δ 7.86 (s, 1H), 7.83 (d, J = 5.2 Hz, 1H), 7.78 (d, J = 5.2 Hz, 1H), 7.72 (s, 1H), 4.45-4.33 (m, 1H), 3.73-3.64 (m, 2H), 3.10-3.00 (m, 2H), 2.96 (d, J = 7.2 Hz, 2H), 2.40-2.25 (m, 4H), 2.14 (s, 3H), 1.18-1.07 (m, 1H), 0.81-0.73 (m, 2H), 0.44-0.38 (m, 2H) ppm. |
| 90 | 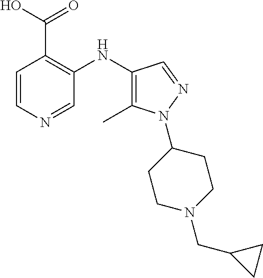 [SMILES: Cc1c(Nc2cnccc2C(=O)O)cnn1C1CCN(CC2CC2)CC1] | ESI-LCMS (m/z): 356.2 [M + 1]; ¹H-NMR (400 MHz, Methanol-d4): δ 7.82 (d, J = 5.2 Hz, 1H), 7.79 (s, 1H), 7.77 (d, J = 4.8 Hz, 1H), 7.48 (s, 1H), 4.61-4.50 (m, 1H), 3.85-3.75 (m, 2H), 3.26-3.16 (m, 2H), 3.08 (d, J = 7.2 Hz, 2H), 2.53-2.40 (m, 2H), 2.30-2.20 (m, 5H), 1.22-1.12 (m, 1H), 0.84-0.78 (m, 2H), 0.50-0.44 (m, 2H) ppm. |
| 91 | 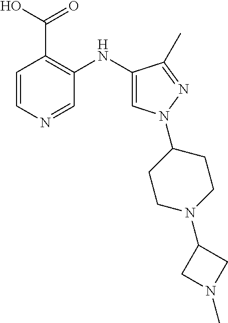 [SMILES: Cc1nn(C2CCN(C3CN(C)C3)CC2)cc1Nc1cnccc1C(=O)O] | ESI-LCMS (m/z): 371.3 [M + 1]; ¹H-NMR (400 MHz, Methanol-d4): δ 7.85 (s, 1H), 7.83-7.75 (m, 2H), 7.69 (s, 1H), 4.16-4.08 (m, 1H), 4.02-3.93 (m, 2H), 3.66-3.55 (m, 2H), 3.25-3.18 (m, 2H), 3.00-2.94 (m, 2H), 2.72 (s, 3H), 2.17-2.04 (m, 9H) ppm. |
| 92 | 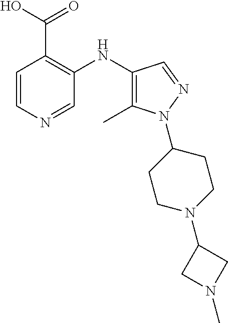 [SMILES: Cc1c(Nc2cnccc2C(=O)O)cnn1C1CCN(C2CN(C)C2)CC1] | ESI-LCMS (m/z): 371.1 [M + 1]; ¹H-NMR (400 MHz, Methanol-d4): δ 7.81-7.74 (m, 3H), 7.46 (s, 1H), 4.25-4.15 (m, 1H), 3.83-3.75 (m, 2H), 3.31-3.25 (m, 2H), 3.16-3.08 (m, 1H), 2.98-2.92 (m, 2H), 2.55 (s, 3H), 2.29-2.17 (m, 5H), 2.14-2.05 (m, 2H), 2.00-1.94 (m, 2H) ppm. |
| 93 | 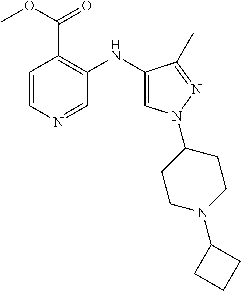 [SMILES: COC(=O)c1ccncc1Nc1cn(C2CCN(C3CCC3)CC2)nc1C] | ESI-LCMS: 370.3 [M + 1]; ¹H-NMR (400 MHz, Methanol-d4): δ 7.85 (s, 1H), 7.78 (d, J = 5.6 Hz, 1H), 7.66 (d, J = 5.2 Hz, 1H), 7.64 (s, 1H), 4.09-3.98 (m, 1H), 3.86 (s, 3H), 2.98-2.90 (m, 2H), 2.80-2.70 (m, 1H), 2.11-1.78 (m, 13H), 1.70-1.60 (m, 2H) ppm. |
| 94 | 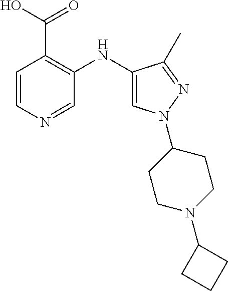 [SMILES: Cc1nn(C2CCN(C3CCC3)CC2)cc1Nc1cnccc1C(=O)O] | ESI-LCMS: 356.2 [M + 1]; ¹H-NMR (400 MHz, Methanol-d4): δ 7.86 (s, 1H), 7.83 (d, J = 5.2 Hz, 1H), 7.79 (d, J = 5.2 Hz, 1H), 7.71 (s, 1H), 4.42- 4.32 (m, 1H), 3.68-3.46 (m, 3H), 2.95-2.80 (m, 2H), 2.37-2.18 (m, 8H), 2.13 (s, 3H), 1.90-1.80 (m, 2H) ppm. |
| 95 | 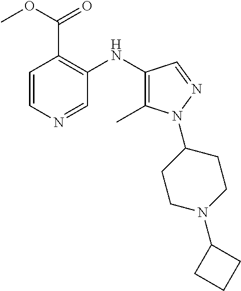 [SMILES: COC(=O)c1ccncc1Nc1cnn(C2CCN(C3CCC3)CC2)c1C] | ESI-LCMS: 370.2 [M + 1]; ¹H-NMR (400 MHz, Methanol-d4): δ 7.92 (s, 1H), 7.88 (d, J = 5.2 Hz, 1H), 7.86 (d, J = 5.2 Hz, 1H), 7.46 (s, 1H), 4.28- 4.18 (m, 1H), 3.97 (s, 3H), 3.12-3.03 (m, 2H), 2.90-2.82 (m, 1H), 2.30- 2.20 (m, 5H), 2.18-1.90 (m, 8H), 1.80-1.70 (m, 2H) ppm. |
| 96 | 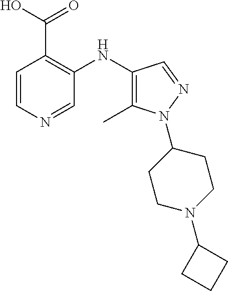 [SMILES: Cc1c(Nc2cnccc2C(=O)O)cnn1C1CCN(C2CCC2)CC1] | ESI-LCMS (m/z): 356.2 [M + 1]; ¹H NMR (400 MHz, DMSO-d6) δ 7.80 (s, 1H), 7.75 (d, J = 4.8 Hz, 1H), 7.58 (d, J = 4.8 Hz, 1H), 7.39 (s, 1H), 4.14-4.08 (m, 2H), 3.16 (d, J = 5.2 Hz, 2H), 2.98-2.91 (m, 2H), 2.12 (s, 3H), 2.04-1.94 (m, 4H), 1.90-1.80 (m, 4H), 1.68-1.58 (m, 2H) ppm. |
| 97 | 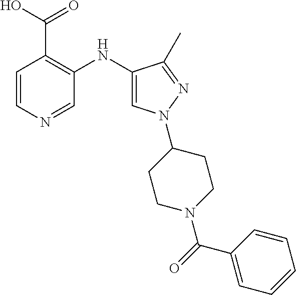 [SMILES: Cc1nn(C2CCN(C(=O)c3ccccc3)CC2)cc1Nc1cnccc1C(=O)O] | ESI-LCMS (m/z): 406.2 [M + 1]; ¹H-NMR (400 MHz, Methanol-d4): δ 7.96 (s, 1H), 7.93-7.85 (m, 2H), 7.80 (s, 1H), 7.53-7.45 (m, 5H), 4.83-4.75 (m, 1H), 4.50-4.39 (m, 1H), 3.94-3.85 (m, 1H), 3.36-3.32 (m, 1H), 3.10-3.02 (m, 1H), 2.30-1.95 (m, 7H) ppm. |
| 98 | 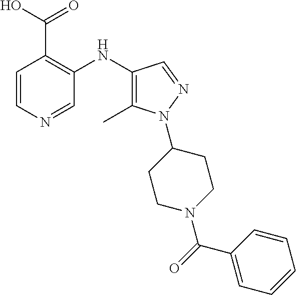 [SMILES: Cc1c(Nc2cnccc2C(=O)O)cnn1C1CCN(C(=O)c2ccccc2)CC1] | ESI-LCMS (m/z): 406.2 [M + 1]; ¹H-NMR (400 MHz, Methanol-d4): δ 8.27 (d, J = 5.6 Hz, 1H), 8.04 (s, 1H), 8.02 (d, J = 5.6 Hz, 1H), 7.57 (s, 1H), 7.55-7.45 (m, 5H), 4.88-4.78 (m, 1H), 4.63-4.54 (m, 1H), 3.96-3.85 (m, 1H), 3.43-3.32 (m, 1H), 3.15-3.05 (m, 1H), 2.28 (s, 3H), 2.25-2.08 (m, 3H), 2.00-1.90 (m, 1H) ppm. |
| 99 | 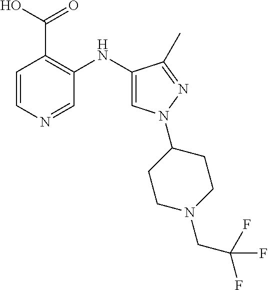 [SMILES: Cc1nn(C2CCN(CC(F)(F)F)CC2)cc1Nc1cnccc1C(=O)O] | ESI-LCMS (m/z): 384.2 [M + 1]; ¹H-NMR (400 MHz, Methanol-d4): δ 7.91 (s, 1H), 7.89-7.83 (m, 2H), 7.74 (s, 1H), 4.15-4.07 (m, 1H), 3.18-3.11 (m, 4H), 2.64-2.57 (m, 2H), 2.12-2.06 (m, 7H) ppm. |
| 100 | 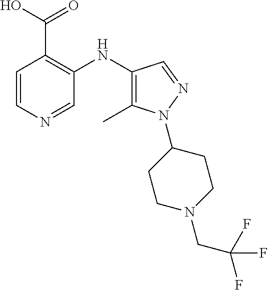 [SMILES: Cc1c(Nc2cnccc2C(=O)O)cnn1C1CCN(CC(F)(F)F)CC1] | ESI-LCMS (m/z): 384.2 [M + 1]; ¹H-NMR (400 MHz, Methanol-d4): δ 7.84-7.76 (m, 3H), 7.47 (s, 1H), 4.25-4.15 (m, 1H), 3.19-3.10 (m, 4H), 2.70-2.60 (m, 2H), 2.32-2.20 (m, 5H), 1.95-1.87 (m, 2H) ppm. |
| 101 | 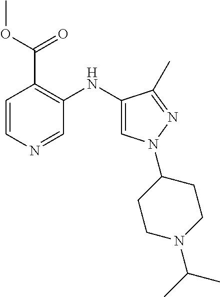 [SMILES: COC(=O)c1ccncc1Nc1cn(C2CCN(C(C)C)CC2)nc1C] | ESI-LCMS: 358.2 [M + 1]; ¹H-NMR (400 MHz, Methanol-d4): δ 7.99 (s, 1H), 7.92 (d, J = 5.2 Hz, 1H), 7.80 (d, J = 5.2 Hz, 1H), 7.77 (s, 1H), 4.47 (m, 1H), 3.98 (s, 3H), 3.70-3.60 (m, 3H), 3.30-3.22 (m, 2H), 2.44-2.30 (m, 4H), 2.13 (s, 3H), 1.42 (d, J = 6.8 Hz, 6H) ppm. |
| 102 | 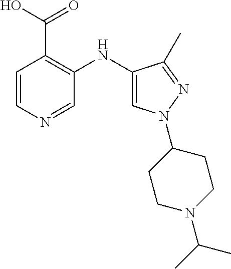 [SMILES: Cc1nn(C2CCN(C(C)C)CC2)cc1Nc1cnccc1C(=O)O] | ESI-LCMS: 344.32 [M + 1]; ¹H NMR (400 MHz, DMSO-d6) δ 9.93 (brs, 1H), 7.88 (s, 1H), 7.79 (d, J = 4.8 Hz, 1H), 7.75 (s, 1H), 7.61 (d, J = 4.8 Hz, 1H), 4.32-4.20 (m, 1H), 3.35-3.20 (m, 3H), 2.92-2.80 (m, 1H), 2.30-2.10 (m, 4H), 1.99 (s, 3H), 1.20 (d, J = 6.4 Hz, 6H) ppm. |
| 103 | 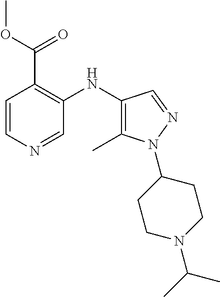 [SMILES: COC(=O)c1ccncc1Nc1cnn(C2CCN(C(C)C)CC2)c1C] | ESI-LCMS: 358.2 [M + 1]; ¹H-NMR (400 MHz, Methanol-d4): δ 7.87 (s, 1H), 7.83 (d, J = 5.2 Hz, 1H), 7.72 (d, J = 5.6 Hz, 1H), 7.41 (s, 1H), 4.21-4.11 (m, 1H), 3.92 (s, 3H), 3.10-3.11 (m, 2H), 2.85-2.75 (m, 1H), 2.48-2.36 (m, 2H), 2.26-2.12 (m, 5H), 1.96-1.92 (m, 2H), 1.09 (d, J = 6.8 Hz, 6H) ppm. |
| 104 | 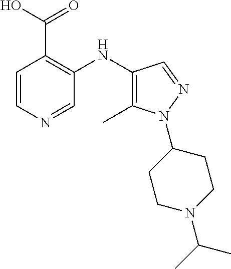 [SMILES: Cc1c(Nc2cnccc2C(=O)O)cnn1C1CCN(C(C)C)CC1] | ESI-LCMS: 344.3 [M + 1]; ¹H-NMR (400 MHz, DMSO-d6) δ 7.80 (s, 1H), 7.75 (d, J = 4.8 Hz, 1H), 7.59 (d, J = 5.2 Hz, 1H), 7.40 (s, 1H), 4.15-4.07 (m, 2H), 3.10-2.80 (m, 3H), 2.13 (s, 3H), 2.10-1.90 (m, 5H), 1.04 (br s, 6H) ppm. |
| 105 | 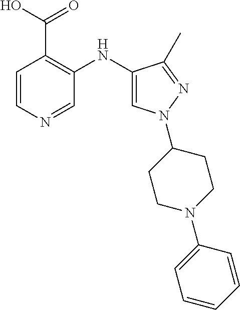 [SMILES: Cc1nn(C2CCN(c3ccccc3)CC2)cc1Nc1cnccc1C(=O)O] | ESI-LCMS (m/z): 378.2 [M + 1]; ¹H NMR (400 MHz, DMSO-d₆): δ 13.67 (brs, 1H), 8.70 (brs, 1H), 8.05 (s, 1H), 7.95-7.86 (m, 2H), 7.62 (d, J = 5.2 Hz, 1H), 7.26-7.18 (m, 2H), 7.01-6.96 (m, 2H), 6.80-6.74 (m, 1H), 4.31-4.20 (m, 1H), 3.87-3.80 (m, 2H), 2.90-2.80 (m, 2H), 2.13-1.98 (m, 7H) ppm. |
| 106 | 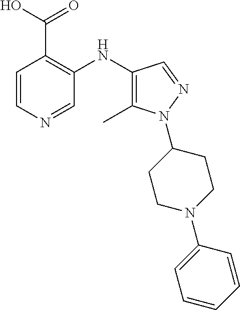 [SMILES: Cc1c(Nc2cnccc2C(=O)O)cnn1C1CCN(c2ccccc2)CC1] | ESI-LCMS (m/z): 378.1 [M + 1]; ¹H NMR (400 MHz, DMSO-d6): δ 8.80 (brs, 1H), 7.93 (s, 1H), 7.88 (d, J = 4.8 Hz, 1H), 7.61 (d, J = 4.8 Hz, 1H), 7.45 (s, 1H), 7.23 (t, J = 7.6 Hz, 2H), 6.99 (d, J = 8.4 Hz, 2H), 6.77 (t, J = 7.2 Hz, 1H), 4.42- 4.34 (m, 1H), 3.90-3.83 (m. 2H), 2.95- 2.85 (m, 2H), 2.20-2.09 (m, 5H), 1.97- 1.92 (m, 2H) ppm. |
| 107 | 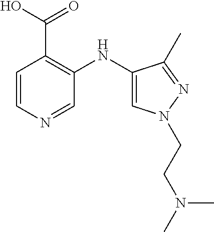 [SMILES: Cc1nn(CCN(C)C)cc1Nc1cnccc1C(=O)O] | ESI-LCMS (m/z): 290.1 [M + 1]; ¹H NMR (400 MHz, DMSO-d6): δ 9.92 (brs, 1H), 7.99 (s, 1H), 7.82 (d, J = 4.8 Hz, 1H), 7.80 (s, 1H), 7.63 (d, J = 4.0 Hz, 1H), 4.24-4.15 (m, 2H), 2.94-2.85 (m, 2H), 2.35 (s, 3H), 2.34 (s, 3H), 2.02 (s, 3H) ppm. |
| 108 | 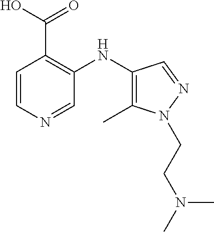 [SMILES: Cc1c(Nc2cnccc2C(=O)O)cnn1CCN(C)C] | ESI-LCMS (m/z): 290.1 [M + 1]; ¹H-NMR (400 MHz, CDCl₃): δ 9.12 (s, 1H), 7.96- 7.70 (m, 3H), 7.45 (s, 1H), 4.37 (brs, 2H), 3.24 (brs, 2H), 2.58 (s, 6H), 2.12 (s, 3H) ppm. |
| 109 | 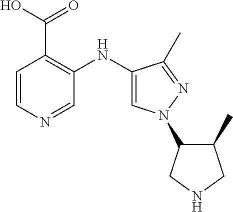 | ESI-LCMS (m/z): 302.2 [M + 1]; ¹H-NMR (400 MHz, Methanol-d₄) δ 7.88 (s, 1H), 7.82 (d, J = 5.0 Hz, 1H), 7.78 (d, J = 4.7 Hz, 1H), 7.71 (s, 1H), 4.86-4.82 (m, 1H), 3.75-3.60 (m, 2H), 3.44-3.37 (m, 1H), 3.17-3.05 (m, 1H), 2.71-2.59 (m, 1H), 2.15 (s, 3H), 0.77 (d, J = 6.8 Hz, 3H). |
| 110 | 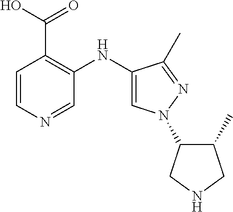 | ESI-LCMS (m/z): 302.2 [M + 1]; ¹H-NMR (400 MHz, Methanol-d₄) δ 7.88 (s, 1H), 7.82 (d, J = 5.0 Hz, 1H), 7.78 (d, J = 4.7 Hz, 1H), 7.71 (s, 1H), 4.86-4.82 (m, 1H), 3.75-3.60 (m, 2H), 3.44-3.37 (m, 1H), 3.17-3.05 (m, 1H), 2.71-2.59 (m, 1H), 2.15 (s, 3H), 0.77 (d, J = 6.8 Hz, 3H) ppm. |
| 111 | 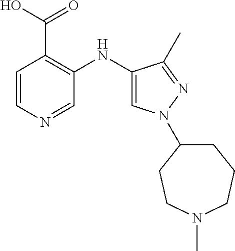 [SMILES: Cc1nn(C2CCCN(C)CC2)cc1Nc1cnccc1C(=O)O] | ESI-LCMS (m/z): 330.1 [M + 1]; ¹H-NMR (400 MHz, Methanol-d4) δ 7.79 (d, J = 5.2 Hz, 1H), 7.77 (s, 1H), 7.75 (d, J = 5.2 Hz, 1H), 7.46 (s, 1H), 4.61-4.52 (m, 1H), 3.10-3.02 (m, 1H), 2.95-2.80 (m, 3H), 2.52 (s, 3H), 2.40-2.30 (m, 1H), 2.25-2.10 (m, 6H), 2.05-1.95 (m, 1H), 1.90-1.80 (m, 1H) ppm. |
| 112 | 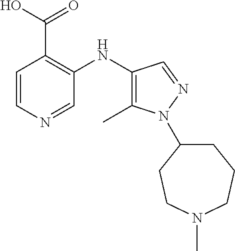 [SMILES: Cc1c(Nc2cnccc2C(=O)O)cnn1C1CCCN(C)CC1] | ESI-LCMS (m/z): 330.2 [M + 1]; ¹H-NMR (400 MHz, Methanol-d4) δ 7.85 (s, 1H), 7.81 (d, J = 4.8 Hz, 1H), 7.78 (d, J = 4.8 Hz, 1H), 7.69 (s, 1H), 4.51-4.44 (m, 1H), 3.25-3.18 (m, 1H), 3.06-2.95 (m, 3H), 2.64 (s, 3H), 2.35-2.15 (m, 4H), 2.13 (s, 3H), 2.06-1.96 (m, 1H), 1.93-1.80 (m, 1H) ppm. |
| 113 | 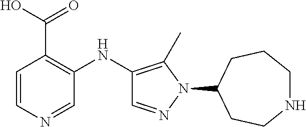 | ESI-LCMS (m/z): 316.3 [M + 1]; ¹H-NMR (400 MHz, Methanol-d4) δ 7.82-7.75 (m, 3H), 7.49 (s, 1H), 4.70-4.60 (m, 1H), 3.45-3.38 (m, 1H), 3.25-3.18 (m, 3H), 2.46-2.36 (m, 1H), 2.34-2.10 (m, 6H), 2.06-1.96 (m, 1H), 1.94-1.85 (m, 1H) ppm. |
| 114 | 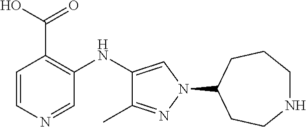 | ESI-LCMS (m/z): 316.2 [M + 1]; ¹H-NMR (400 MHz, Methanol-d4) δ 7.85 (s, 1H), 7.82 (d, J = 4.8, 1H),7.77 (d, J = 4.8, 1H), 7.68 (s, 1H), 4.55-4.45 (m, 1H), 3.48-3.42 (m, 1H), 3.36-3.20 (m, 3H), 2.40-2.33 (m, 2H), 2.32-2.02 (m, 6H), 1.98-1.86 (m, 1H) ppm. |
| 115 | 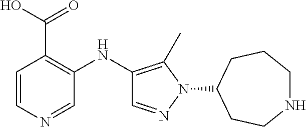 | ESI-LCMS (m/z): 316.2 [M + 1]; ¹H-NMR (400 MHz, Methanol-d4) δ 7.84-7.71 (m, 3H), 7.49 (s, 1H), 4.73-4.65 (m, 1H), 3.58-3.51 (m, 1H), 3.35-3.27 (m, 3H), 2.52-2.43 (m, 1H), 2.39-2.30 (m, 1H), 2.25-2.15 (m, 5H), 2.10-2.00 (m, 1H), 1.98-1.90 (m, 1H) ppm. |
| 116 | 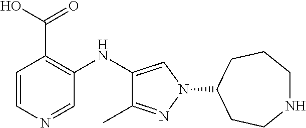 | ESI-LCMS (m/z): 316 [M + 1]; ¹H-NMR (400 MHz, Methanol-d4) δ 7.86 (s, 1H), 7.82 (d, J = 4.8 Hz, 1H), 7.77 (d, J = 4.8 Hz, 1H), 7.68 (s, 1H), 4.55-4.45 (m, 1H), 3.50-3.43 (m, 1H), 3.36-3.22 (m, 3H), 2.42-2.35 (m, 2H), 2.32-2.05 (m, 6H), 1.96-1.87 (m, 1H) ppm. |
| 117 | 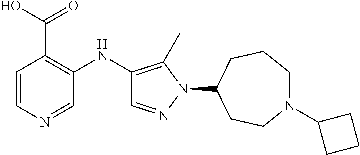 | ESI-LCMS (m/z): 370.2 [M + 1]; ¹H-NMR (400 MHz, Methanol-d4) δ 7.84-7.75 (m, 3H), 7.49 (s, 1H), 4.70-4.60 (m, 1H), 3.69-3.57 (m, 1H), 3.20-3.05 (m, 3H), 2.50-2.40 (m, 1H), 2.38-2.00 (m, 11H), 1.98-1.74 (m, 4H) ppm. |
| 118 | 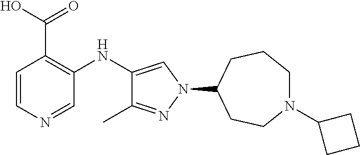 | ESI-LCMS (m/z): 370.3 [M + 1]; ¹H-NMR (400 MHz, Methanol-d4) δ 7.86 (s, 1H), 7.81 (d, J = 5.2 Hz, 1H), 7.77 (d, J = 5.2 Hz, 1H), 7.69 (s, 1H), 4.49-4.40 (m, 1H), 3.32-3.25 (m, 1H), 3.05-2.97 (m, 1H), 2.92-2.74 (m, 3H), 2.32-1.90 (m, 12H), 1.89-1.68 (m, 3H) ppm. |
| 119 | 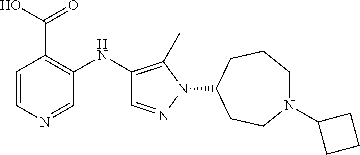 | ESI-LCMS (m/z): 370.2 [M + 1]; ¹H-NMR (400 MHz, Methanol-d4) δ 7.82-7.74 (m, 3H), 7.46 (s, 1H), 4.63-4.54 (m, 1H), 3.40-3.32 (m, 1H), 3.12-3.04 (m, 1H), 3.02-2.94 (m, 1H), 2.92-2.77 (m, 2H), 2.42-2.32 (m, 1H), 2.25-1.96 (m, 11H), 1.92-1.68 (m, 3H) ppm. |
| 120 | 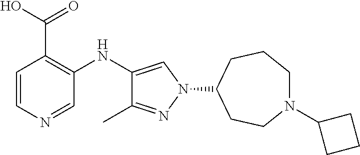 | ESI-LCMS (m/z): 370.3 [M + 1]; ¹H-NMR (400 MHz, Methanol-d4) δ 7.86 (s, 1H), 7.82 (d, J = 5.2 Hz, 1H), 7.78 (d, J = 52 Hz, 1H), 7.68 (s, 1H), 4.54-4.45 (m, 1H), 3.66-3.55 (m, 1H), 3.32-3.23 (m, 1H), 3.15-3.00 (m, 3H), 2.43-2.00 (m, 12H), 1.98-1.71 (m, 3H) ppm. |
| 121 | 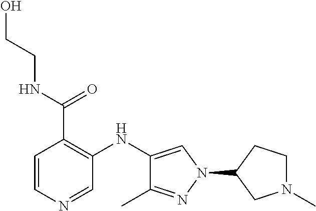 | ESI-LCMS (m/z): 345.2 [M + 1]; ¹H-NMR (400 MHz, Methanol-d₄) δ 7.94 (s, 1H), 7.89 (d, J = 5.2 Hz, 1H), 7.76 (s, 1H), 7.52 (d, J = 5.1 Hz, 1H), 4.88-4.83 (m, 1H), 3.74 (t, J = 5.7 Hz, 2H), 3.53 (t, J = 5.8 Hz, 2H), 3.10-3.00 (m, 1H), 2.96-2.87 (m, 2H), 2.75-2.65 (m, 1H), 2.56-2.46 (m, 1H), 2.44 (s, 3H), 2.29-2.16 (m, 1H), 2.11 (s, 3H) ppm. |
| 122 | 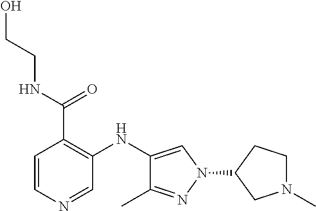 | ESI-LCMS (m/z): 345.2 [M + 1]; ¹H-NMR (400 MHz, Methanol-d₄) δ 7.94 (s, 1H), 7.90 (d, J = 5.2 Hz, 1H), 7.76 (s, 1H), 7.52 (d, J = 5.1 Hz, 1H), 4.88-4.84 (m, 1H), 3.74 (t, J = 5.8 Hz, 2H), 3.53 (t, J = 5.8 Hz, 2H), 3.07-3.01 (m, 1H), 2.94-2.85 (m, 2H), 2.74-2.65 (m, 1H), 2.54-2.45 (m, 1H), 2.43 (s, 3H), 2.28-2.17 (m, 1H), 2.11 (s, 3H) ppm. |
| 123 | 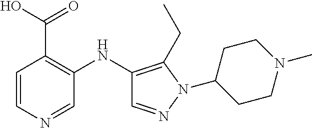 [SMILES: CCc1c(Nc2cnccc2C(=O)O)cnn1C1CCN(C)CC1] | ESI-LCMS (m/z): 330.2 [M + 1]; ¹H-NMR (400 MHz, DMSO-d6): δ 7.89 (s, 1H), 7.79 (d, J = 4.8 Hz, 1H), 7.59 (d, J = 4.8 Hz, 1H), 7.45 (s, 1H), 4.30-4.20 (m, 1H), 3.24-3.18 (m, 2H), 2.65-2.54 (m, 4H), 2.30-2.15 (m, 2H), 1.98-1.90 (m, 2H), 1.05 (t, J = 7.6 Hz, 3H) ppm. (NMe- piperidine protons obscured by solvent) |
| 124 | 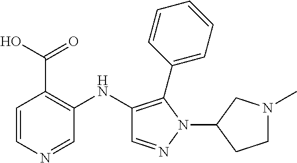 [SMILES: CN1CCC(n2ncc(Nc3cnccc3C(=O)O)c2-c2ccccc2)C1] | ESI-LCMS (m/z): 364.1 [M + 1]; ¹H-NMR (400 MHz, Methanol-d4): δ 8.05 (s, 1H), 7.85 (d, J = 5.2 Hz, 1H), 7.80 (s, 1H), 7.78 (d, J = 5.6 Hz, 1H), 7.55-7.45 (m, 3H), 7.47-7.39 (m, 2H), 5.30-5.20 (m, 1H), 4.20-3.80 (m, 4H), 3.09 (s, 3H), 2.80-2.60 (m, 1H), 2.50-2.40 (m, 1H) ppm. |
| 125 | 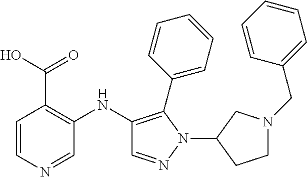 [SMILES: O=C(O)c1ccncc1Nc1cnn(C2CCN(Cc3ccccc3)C2)c1-c1ccccc1] | ESI-LCMS (m/z): 440.2 [M + 1]; ¹H-NMR (400 MHz, Methanol-d4) δ 7.94 (s, 1H), 7.76 (d, J = 5.2 Hz, 1H), 7.73 (s, 1H), 7.70 (d, J = 5.2 Hz, 1H), 7.56-7.52 (m, 2H), 7.50-7.33 (m, 8H), 5.22-5.12 (m, 1H), 4.45 (d, J = 12.8 Hz, 1H), 4.32 (d, J = 12.8 Hz, 1H), 3.71-3.55 (m, 3H), 3.41- 3.35 (m, 1H), 2.58-2.42 (m, 2H) ppm. |
| 126 | 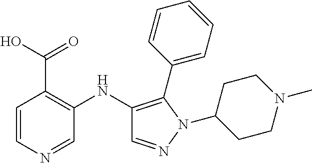 [SMILES: CN1CCC(n2ncc(Nc3cnccc3C(=O)O)c2-c2ccccc2)CC1] | ESI-LCMS (m/z): 378.1 [M + 1]; ¹H-NMR (400 MHz, Methanol-d4) δ 7.92 (s, 1H), 7.74 (d, J = 4.8 Hz, 1H), 7.68 (d, J = 4.4 Hz, 1H), 7.63 (s, 1H), 7.50-7.36 (m, 5H), 4.52-4.41 (m, 1H), 3.55-3.46 (m, 2H), 3.03-2.89 (m, 2H), 2.78 (s, 3H), 2.59- 2.42 (m, 2H), 2.18-2.11 (m, 2H) ppm. |
| 127 | 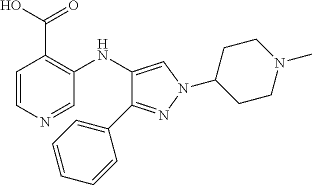 [SMILES: CN1CCC(n2cc(Nc3cnccc3C(=O)O)c(-c3ccccc3)n2)CC1] | ESI-LCMS (m/z): 378.2 [M + 1]; ¹H-NMR (400 MHz, DMSO-d₆): δ 10.10 (brs, 1H), 7.98 (s, 1H), 7.92 (s, 1H), 7.80 (d, J = 4.8 Hz, 1H), 7.75 (d, J = 8.0 Hz, 2H), 7.63 (d, J = 4.8 Hz, 1H), 7.30 (t, J = 8.0 Hz, 2H), 7.23 (t, J = 6.8 Hz, 1H), 4.41-4.30 (m, 1H), 3.40-3.20 (m, 2H), 2.80-2.65 (m, 2H), 2.57 (s, 3H), 2.26-2.10 (m, 4H) ppm. |
| 128 | 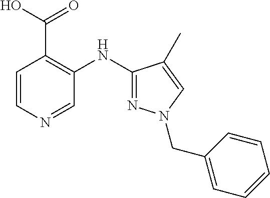 [SMILES: Cc1cn(Cc2ccccc2)nc1Nc1cnccc1C(=O)O] | ESI-LCMS (m/z): 309.1 [M + 1]; ¹H-NMR (400 MHz, DMSO-d6): δ 11.14 (brs, 1H), 9.35 (s, 1H), 7.93 (d, J = 4.8 Hz, 1H), 7.66 (d, J = 4.8 Hz, 1H), 7.54 (s, 1H), 7.38-7.31 (m, 2H), 7.30-7.20 (m, 4H), 5.20 (s, 2H), 1.96 (s, 3H) ppm. |
| 129 | 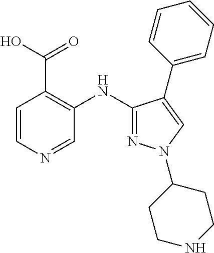 [SMILES: O=C(O)c1ccncc1Nc1nn(C2CCNCC2)cc1-c1ccccc1] | ESI-LCMS (m/z): 364.2 [M + 1]; ¹H-NMR (400 MHz, DMSO-d6): δ 12.30 (s, 1H), 9.37 (s, 1H), 9.01 (brs, 1H), 7.95 (s, 1H), 7.91 (d, J = 4.8 Hz, 1H), 7.69 (d, J = 4.8 Hz, 1H), 7.58 (d, J = 7.6 Hz, 2H), 7.35 (t, J = 7.6 Hz, 2H), 7.21 (t, J = 7.6 Hz, 1H), 4.43-4.36 (m, 1H), 3.45-3.35 (m, 2H), 3.13-3.02 (m, 2H), 2.30-2.17 (m, 4H) ppm. |
| 130 | 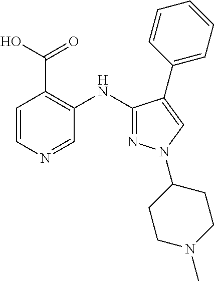 [SMILES: CN1CCC(n2cc(-c3ccccc3)c(Nc3cnccc3C(=O)O)n2)CC1] | ESI-LCMS (m/z): 378.2 [M + 1]; ¹H-NMR (400 MHz, Methanol-d4): δ 9.23 (s, 1H), 7.94 (d, J = 5.2 Hz, 1H), 7.86 (d, J = 4.8 Hz, 1H), 7.75 (s, 1H), 7.52 (d, J = 7.2 Hz, 2H), 7.36 (t, J = 7.6 Hz, 2H), 7.22 (t, J = 7.2 Hz, 1H), 4.45-4.35 (m, 1H), 3.62-3.54 (m, 2H), 3.22-3.12 (m, 2H), 2.89 (s, 3H), 2.40-2.28 (m, 4H) ppm. |
| 131 | 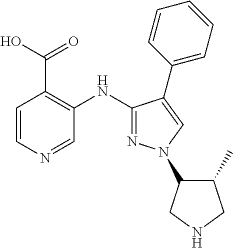 | ESI-LCMS (m/z): 364.2 [M + 1]; ¹H-NMR (400 MHz, Methanol-d4): δ 8.00-7.97 (m, 1H), 7.90 (d, J = 5.2 Hz, 1H), 7.83 (d, J = 5.2 Hz, 1H), 7.55 (s, 1H), 7.52 (d, J = 7.6 Hz, 2H), 7.25 (t, J = 7.2 Hz, 2H), 7.15 (t, J = 7.2 Hz, 1H), 4.84 (q, J = 6.4 Hz, 1H), 3.81-3.69 (m, 3H), 3.10-3.02 (m, 1H), 2.84-2.76 (m, 1H), 1.07 (d, J = 6.8 Hz, 3H) ppm. |
| 132 | 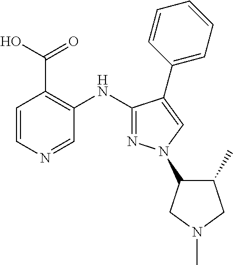 | ESI-LCMS (m/z): 378.2 [M + 1]; ¹H-NMR (400 MHz, Methanol-d4): δ 7.97 (brs, 1H), 7.88 (d, J = 4.8 Hz, 1H), 7.80 (d, J = 4.8 Hz, 1H), 7.53 (s, 1H), 7.50 (d, J = 7.6 Hz, 2H), 7.23 (t, J = 7.6 Hz, 2H), 7.13 (t, J = 7.2 Hz, 1H), 4.95-4.85 (m, 1H), 3.90- 3.75 (m, 3H), 3.10 (t, J = 10.4 Hz, 1H), 3.02 (s, 3H), 2.90-2.80 (m, 1H), 1.08 (d, J = 6.8 Hz, 3H) ppm. |
| 133 | 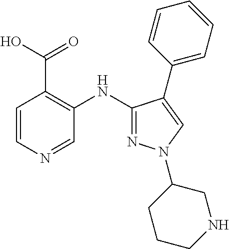 [SMILES: O=C(O)c1ccncc1Nc1nn(C2CCCNC2)cc1-c1ccccc1] | ESI-LCMS (m/z): 364.2 [M + 1]; ¹H-NMR (400 MHz, Methanol-d4): δ 7.99 (s, 1H), 7.89 (d, J = 4.8 Hz, 1H), 7.83-7.80 (m, 1H), 7.54 (s, 1H), 7.51 (d, J = 7.6 Hz, 2H), 7.25 (t, J = 7.6 Hz, 2H), 7.15 (t, J = 7.6 Hz, 1H), 4.81-4.75 (m, 1H), 3.62-3.50 (m, 2H), 3.32-3.12 (m, 2H), 2.20-2.02 (m, 3H), 1.90-1.78 (m, 1H) ppm. |
| 134 | 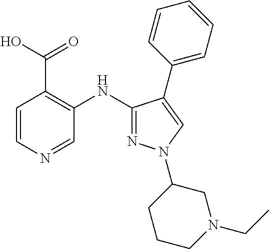 [SMILES: CCN1CCCC(n2cc(-c3ccccc3)c(Nc3cnccc3C(=O)O)n2)C1] | ESI-LCMS (m/z): 392.2 [M + 1]; ¹H-NMR (400 MHz, Methanol-d4): δ 7.99 (s, 1H), 7.88-7.85 (m, 1H), 7.76-7.65 (m, 1H), 7.55 (s, 1H), 7.50 (d, J = 7.2 Hz, 2H), 7.24 (t, J = 7.2 Hz, 2H), 7.15 (t, J = 7.2 Hz, 1H), 4.90-4.80 (m, 1H), 3.60-3.18 (m, 6H), 2.20-2.08 (m, 3H), 2.00-1.88 (m, 1H), 1.39-1.35 (t, J = 7.2 Hz, 3H) ppm. |
| 135 | 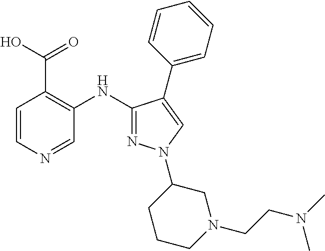 [SMILES: CN(C)CCN1CCCC(n2cc(-c3ccccc3)c(Nc3cnccc3C(=O)O)n2)C1] | ESI-LCMS (m/z): 435.3 [M + 1]; ¹H-NMR (400 MHz, Methanol-d4): δ 7.92 (s, 1H), 7.90 (d, J = 4.8 Hz, 1H), 7.85 (d, J = 4.8 Hz, 1H), 7.61-7.56 (m, 3H), 7.27 (t, J = 7.6 Hz, 2H), 7.16 (t, J = 7.6 Hz, 1H), 4.50-4.40 (m, 1H), 3.33-3.20 (m, 1H), 3.22-3.17 (m, 1H), 3.15-3.08 (m, 1H), 2.92-2.80 (m, 8H), 2.68-2.60 (m, 1H), 2.48-2.40 (m, 1H), 2.30-2.22 (m, 1H), 2.15-2.05 (m, 1H), 2.00-1.92 (m, 1H), 1.90-1.89 (m, 1H), 1.72-1.60 (m, 1H) ppm. |
| 136 | 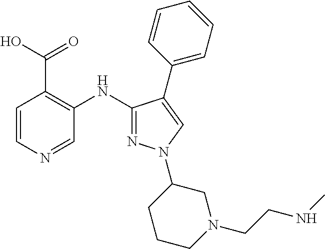 [SMILES: CNCCN1CCCC(n2cc(-c3ccccc3)c(Nc3cnccc3C(=O)O)n2)C1] | ESI-LCMS (m/z): 421.2 [M + 1]; ¹H-NMR (400 MHz, Methanol-d4): δ 7.91 (s, 1H), 7.89 (d, J = 4.8 Hz, 1H), 7.84 (d, J = 5.2 Hz, 1H), 7.57 (s, 1H), 7.56 (d, J = 8.0 Hz, 2H), 7.27 (t, J = 8.0 Hz, 2H), 7.16 (t, J = 7.2 Hz, 1H), 4.48-4.40 (m, 1H), 3.30-3.22 (m, 1H), 3.20-3.13 (m, 1H), 3.09-3.02 (m, 1H), 2.91-2.89 (m, 1H), 2.83-2.73 (m, 4H), 2.66-2.61 (m, 1H), 2.40-2.35 (m, 1H), 2.25-2.18 (m, 1H), 2.15-2.08 (m, 1H), 2.03-1.95 (m, 1H), 1.88-1.82 (m, 1H), 1.78-1.67 (m, 1H) ppm. |
| 137 | 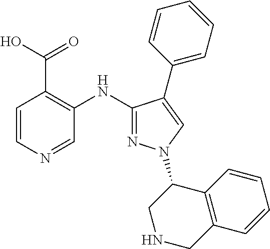 [SMILES: O=C(O)c1ccncc1Nc1nn([C@H]2CNCc3ccccc32)cc1-c1ccccc1] | ESI-LCMS (m/z): 412.2 [M + 1]; ¹H-NMR (400 MHz, Methanol-d4): δ 7.99 (s, 1H), 7.93 (d, J = 4.8 Hz, 1H), 7.85 (d, J = 5.2 Hz, 1H), 7.66 (s, 1H), 7.53 (d, J = 7.2 Hz, 2H), 7.16 (t, J = 7.6 Hz, 2H), 7.18-7.14 (m, 1H), 6.96-6.91 (m, 2H), 6.60-6.54 (m, 2H), 4.80-4.70 (m, 1H), 3.60 (t, J = 10.4 Hz, 1H), 3.54-3.40 (m, 2H), 3.05-2.95 (m, 1H) ppm. |
| 138 | 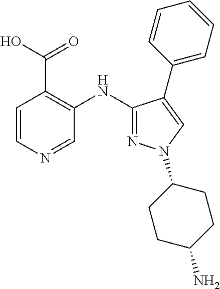 [SMILES: N[C@H]1CC[C@@H](n2cc(-c3ccccc3)c(Nc3cnccc3C(=O)O)n2)CC1] | ESI-LCMS (m/z): 378 [M + 1]; ¹H-NMR (400 MHz, Methanol-d4): δ 7.91 (d, J = 2.4 Hz, 1H), 7.88 (d, J = 5.2 Hz, 1H), 7.83 (d, J = 4.8 Hz, 1H), 7.52 (s, 1H), 7.51 (d, J = 7.2 Hz, 2H), 7.24 (t, J = 7.6 Hz, 2H), 7.13 (t, J = 7.2 Hz, 1H), 4.52- 4.45 (m, 1H), 3.47-3.43 (m, 1H), 2.26- 2.10 (m, 4H), 1.97-1.88 (m, 4H) ppm. |
| 139 | 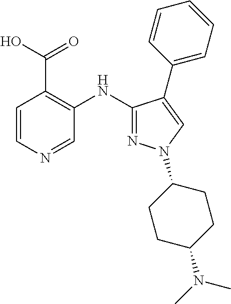 [SMILES: CN(C)[C@H]1CC[C@@H](n2cc(-c3ccccc3)c(Nc3cnccc3C(=O)O)n2)CC1] | ESI-LCMS (m/z): 406 [M + 1]; ¹H-NMR (400 MHz, Methanol-d4): δ 7.89-7.85 (m, 2H), 7.82 (d, J = 4.8 Hz, 1H), 7.54-7.49 (m, 3H), 7.23 (t, J = 7.6 Hz, 2H), 7.12 (t, J = 7.6 Hz, 1H), 4.60-4.54 (m, 1H), 3.10- 3.03 (m, 1H), 2.79 (s, 6H), 2.42-2.32 (m, 2H), 2.27-2.16 (m, 2H), 1.96-1.82 (m, 4H) ppm. |
| 140 | 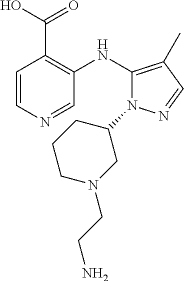 | ESI-LCMS (m/z): 345.3 [M + 1]; ¹H-NMR (400 MHz, Methanol-d4): δ 7.97 (d, J = 5.2 Hz, 1H), 7.85 (d, J = 5.2 Hz, 1H), 7.67 (s, 1H), 7.44 (s, 1H), 4.38-4.29 (m, 1H), 3.10-2.95 (m, 3H), 2.90-2.84 (m, 1H), 2.70-2.54 (m, 2H), 2.40-2.31 (m, 1H), 2.20-2.12 (m, 1H), 2.06-1.89 (m, 5H), 1.87-1.78 (m, 1H), 1.75-1.60 (m, 1H) ppm. |
| 141 | 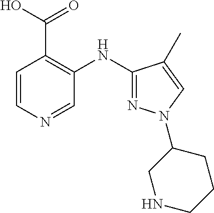 [SMILES: Cc1cn(C2CCCNC2)nc1Nc1cnccc1C(=O)O] | ESI-LCMS (m/z): 302 [M + 1]; ¹H-NMR (400 MHz, Methanol-d4): δ 9.21 (s, 1H), 7.93 (d, J = 5.2 Hz, 1H), 7.85 (d, J = 4.8 Hz, 1H), 7.41 (s, 1H), 4.30-4.22 (m, 1H), 3.48-3.42 (m, 1H), 3.24-3.12 (m, 2H), 2.93-2.84 (m, 1H), 2.25-2.19 (m, 1H), 2.17-2.10 (m, 1H), 2.09 (s, 3H), 2.01-1.96 (m, 1H), 1.82-1.73 (m, 1H) ppm. |
| 142 | 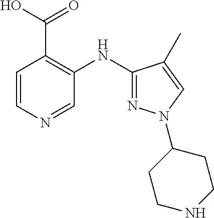 [SMILES: Cc1cn(C2CCNCC2)nc1Nc1cnccc1C(=O)O] | ESI-LCMS (m/z): 302.2 [M + 1]; ¹H-NMR (400 MHz, Methanol-d4): δ 7.95 (d, J = 4.8 Hz, 1H), 7.84 (d, J = 5.2 Hz, 1H), 7.61 (s, 1H), 7.46 (s, 1H), 4.50-4.40 (m, 1H), 3.40-3.34 (m, 2H), 3.01-2.94 (m, 2H), 2.25-2.17 (m, 2H), 2.03-1.95 (m, 2H), 1.89 (s, 3H) ppm. |
| 143 | 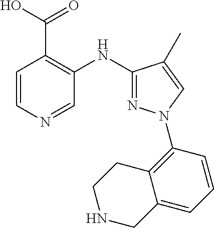 [SMILES: Cc1cn(-c2cccc3c2CCNC3)nc1Nc1cnccc1C(=O)O] | ESI-LCMS (m/z): 350.2 [M + 1]; ¹H-NMR (400 MHz, DMSO-d6): δ 12.47 (brs, 1H), 9.43 (s, 1H), 7.93 (d, J = 4.8 Hz, 1H), 7.73 (s, 1H), 7.69 (d, J = 4.8 Hz, 1H), 7.38 (t, J = 7.6 Hz, 1H), 7.33 (d, J = 6.8 Hz, 1H), 7.24 (d, J = 7.6 Hz, 1H), 4.37 (s, 2H), 3.40-3.30 (m, 2H), 3.07 (t, J = 6.0 Hz, 2H), 2.07 (s, 3H) ppm. |
| 144 | 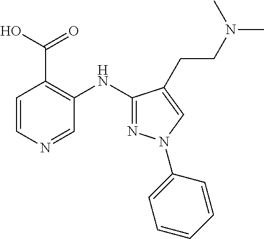 [SMILES: CN(C)CCc1cn(-c2ccccc2)nc1Nc1cnccc1C(=O)O] | ESI-LCMS (m/z): 352.2 [M + 1]; ¹H-NMR (400 MHz, Methanol-d4): δ 9.74 (s, 1H), 8.15 (s, 1H), 8.05 (d, J = 4.8 Hz, 1H), 7.92 (d, J = 4.8 Hz, 1H), 7.75 (d, J = 8.0 Hz, 2H), 7.47 (t, J = 7.6 Hz, 2H), 7.24 (t, J = 7.6 Hz, 1H), 3.45-3.39 (m, 2H), 3.07- 3.03 (m, 2H), 3.01 (s, 6H) ppm. |

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon mono- or multi-ring (e.g., fused, bridged, or spiro rings) system having 3 to 30 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, and adamantyl. The term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, tetrahydroquinolinyl, tetrahydroisoquinolinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1,4-dioxaspiro[4.5]decan-8-yl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, and the like.

The term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl (benzyl)). An “alkylaryl” moiety is an aryl substituted with an alkyl (e.g., methylphenyl).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, “carbocycle” or “carbocyclic ring” is intended to include any stable monocyclic, bicyclic or tricyclic ring having the specified number of carbons, any of which may be saturated, unsaturated, or aromatic. Carbocycle includes cycloalkyl and aryl. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, and [4.4.0] bicyclodecane and [2.2.2] bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” or “heterocyclic group” includes any ring structure (saturated, unsaturated, or aromatic) which contains at least one ring heteroatom (e.g., N, O or S). Heterocycle includes heterocycloalkyl and heteroaryl. Examples of heterocycles include, but are not limited to, morpholine, pyrrolidine, tetrahydrothiophene, piperidine, piperazine, oxetane, pyran, tetrahydropyran, azetidine, and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl (e.g., benzo[d][1,3]dioxole-5-yl), morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —C(O)OH or its C₁-C₆ alkyl ester.

“Acyl” includes moieties that contain the acyl radical (R—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen bonded to two carbon atoms or heteroatoms. For example, the term includes “alkoxyalkyl,” which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls, and alkthioalkynyls. The term “alkthioalkyls” include moieties with an alkyl, alkenyl, or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” refers to —NH₂. “Alkylamino” includes groups of compounds wherein the nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to two alkyl groups. Examples of dialkylamino groups include, but are not limited to, dimethylamino and diethylamine. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

Compounds of the present disclosure that contain nitrogens can be converted to N-oxides by treatment with an oxidizing agent (e.g., 3-chloroperoxybenzoic acid (mCPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

Furthermore, the structures and other compounds discussed herein include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. Examples of lactam-lactim tautomerism are as shown below.

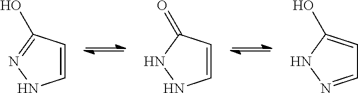 [SMILES: Oc1cc[nH]n1 ; O=c1cc[nH][nH]1 ; Oc1ccn[nH]1]

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of any Formula described herein include the compounds themselves, as well as their salts, and their solvates, if applicable. A salt, for example, can be formed between an anion and a positively charged group (e.g., amino) on a heterocycle substituted amino-pyridine compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a heterocycle substituted amino-pyridine compound. Suitable cations include sodium ion, potassium ion, magnesium ion, calcium ion, and an ammonium cation such as tetramethylammonium ion. The heterocycle substituted amino-pyridine compounds also include those salts containing quaternary nitrogen atoms.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are heterocycle substituted amino-pyridine compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The present disclosure provides methods for the synthesis of the compounds of any of the Formulae described herein. The present disclosure also provides detailed methods for the synthesis of various disclosed compounds of the present disclosure according to the following schemes as shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

Compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

Compounds of the present disclosure can be conveniently prepared by a variety of methods familiar to those skilled in the art. The compounds of this disclosure having any of the Formulae described herein may be prepared according to the procedures illustrated in Schemes 1-6 below, from commercially available starting materials or starting materials which can be prepared using literature procedures. Unless otherwise specified, the variables (e.g., R₂, R₃, and R₄ etc.) in the schemes are as defined in Formula (III).

One of ordinary skill in the art will note that, during the reaction sequences and synthetic schemes described herein, the order of certain steps may be changed, such as the introduction and removal of protecting groups.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

Preferred protecting groups include, but are not limited to:

for a hydroxyl moiety: TBS, benzyl, THP, Ac;

for carboxylic acids: benzyl ester, methyl ester, ethyl ester, allyl ester;

for amines: Cbz, BOC, DMB;

for diols: Ac (x2) TBS (x2), or when taken together acetonides;

for thiols: Ac;

for benzimidazoles: SEM, benzyl, PMB, DMB

For aldehydes: di-alkyl acetals such as dimethoxy acetal or diethyl acetyl.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

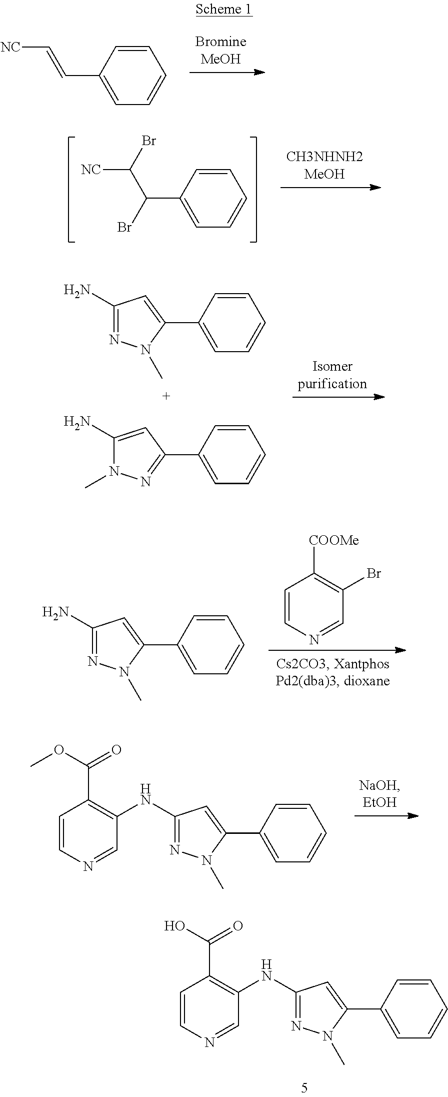

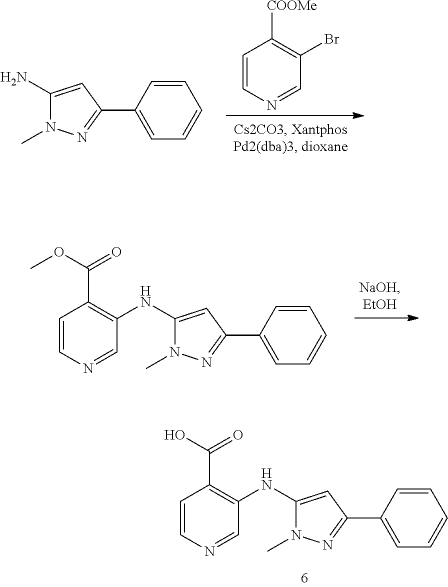

Scheme 1 shows an example of synthesizing Compounds 5 and 6 in Table 1, following a general route that utilizes well-established chemistry. See, e.g., “Aminopyrazoles. IV. Pyrazol-3- and 5-amines from 2,3-dihaloalkanenitriles or 3-chloroacrylonitriles and hydrazines”, Journal of Heterocyclic Chemistry, 19(6), 1267-73; 1982, which is hereby incorporated by reference in its entirety.

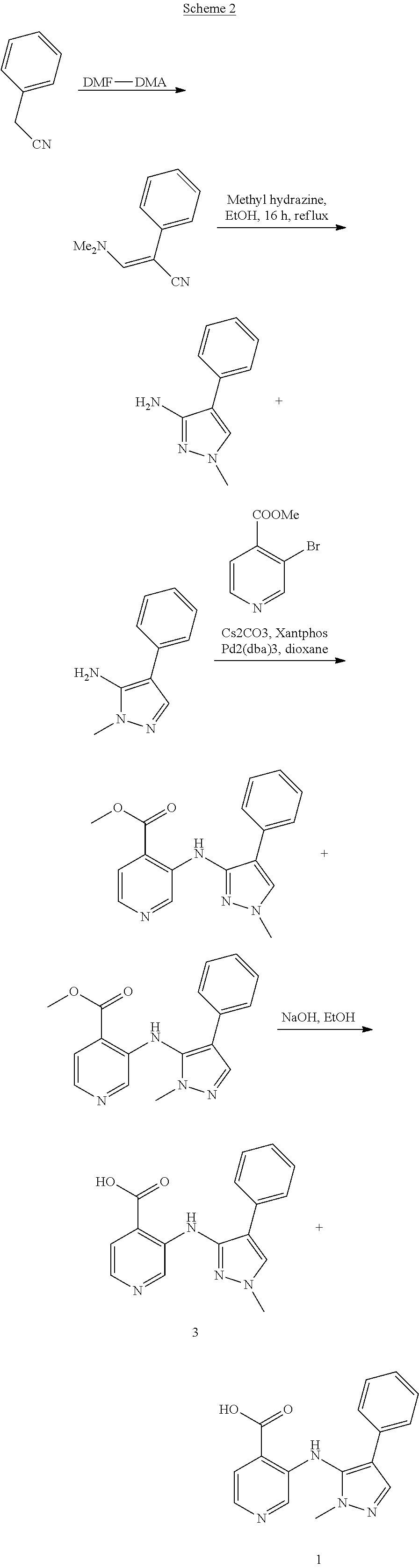

Scheme 2 shows an exemplary route of synthesizing Compounds 1 and 3 in Table 1.

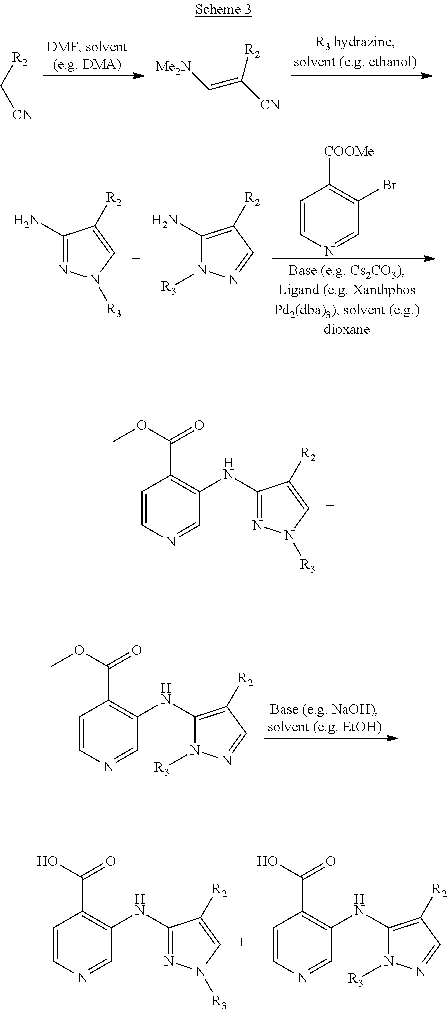

Scheme 3 shows an exemplary route of synthesizing compounds disclosed herein, e.g., Compounds 7-12, 14, 15, 30, 31, 34, and 35 in Table 1, wherein R₂ is C₆-C₁₀ aryl.

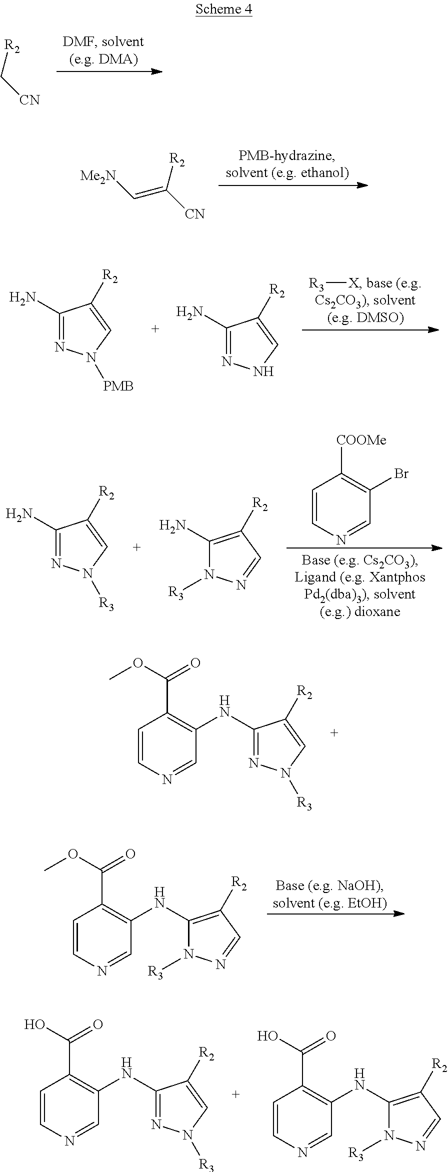

Scheme 4 shows another exemplary route of synthesizing compounds disclosed herein, for example, Compounds 14, 15, 30, 31, 34, and 35.

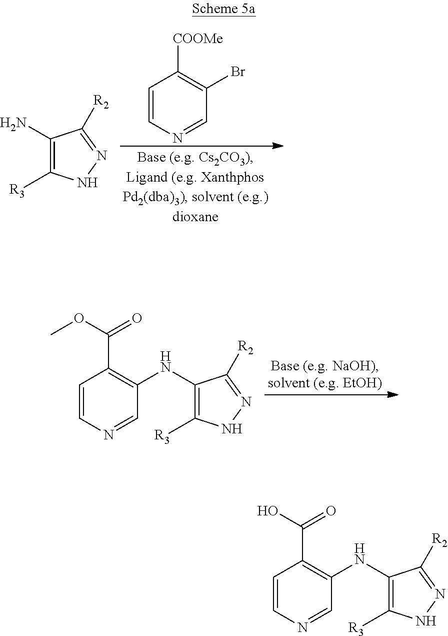

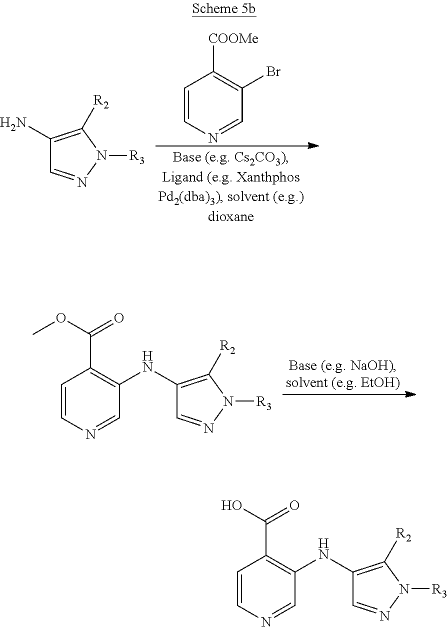

Schemes 5a and 5b show exemplary routes of synthesizing compounds disclosed herein, e.g., Compounds 16, 50, 55, and 59.

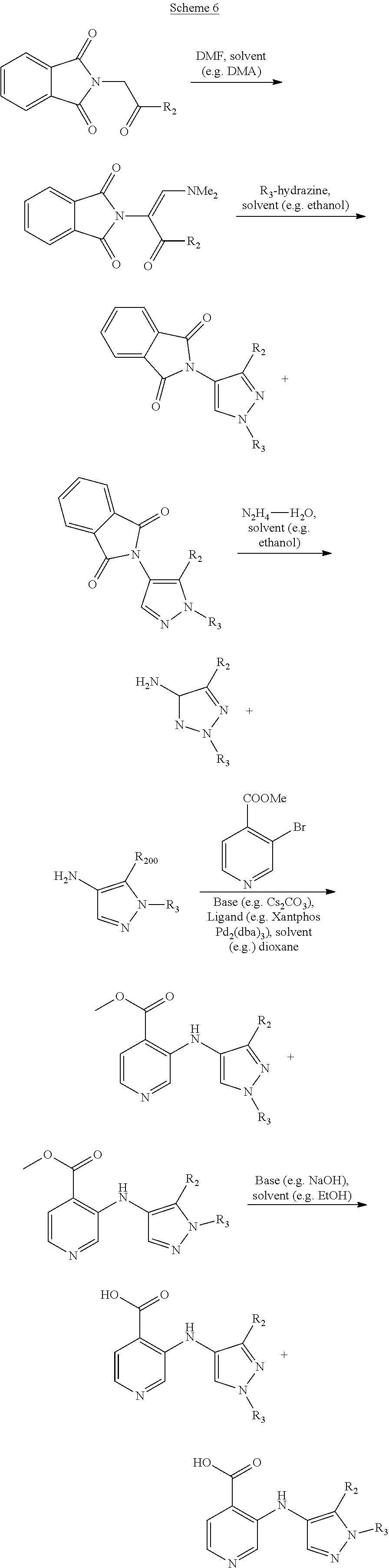

Scheme 6 shows yet another exemplary route of synthesizing compounds disclosed herein, e.g., Compounds 19, 20, 28, 29, 32, 47, and 48.

A person of ordinary skill in the art will recognize that in the above schemes the order of many of the steps is interchangeable.

Compounds of the present disclosure modulate (e.g., inhibit) the activity of a demethylase (e.g., histone demethylase) comprising a JmjC domain, or a mutant thereof and, accordingly, in one aspect of the disclosure, certain compounds disclosed herein are candidates for treating, or preventing certain conditions and diseases, in which a demethylase comprising a JmjC domain plays a role. The present disclosure provides methods for treating conditions and diseases the course of which can be influenced by modulating the methylation status of histones or other proteins, wherein said methylation status is mediated at least in part by the activity of a demethylase comprising a JmjC domain (e.g., a histone demethylase such as JHDM protein(s), JMJD2 protein(s), and JARID protein(s). Modulation of the methylation status of histones can in turn influence the level of expression of target genes activated by methylation, and/or target genes suppressed by methylation. The method includes administering to a subject in need of such treatment, a therapeutically effective amount of a compound of the present disclosure, or a pharmaceutically acceptable salt, polymorph, solvate, or stereoisomeror thereof.

Also disclosed herein are methods of modulating demethylation in a cell or in a subject, either generally or with respect to one or more specific target genes. Demethylation can be modulated to control a variety of cellular functions, including without limitation: differentiation; proliferation; apoptosis; tumorigenesis, leukemogenesis or other oncogenic transformation events; hair loss; or sexual differentiation.

In a further embodiment is the method for treating cancer in a subject, wherein the cancer is selected from prostate cancer, breast cancer, bladder cancer, lung cancer or melanoma.

A “demethylase” as used herein, refers to an enzyme that removes at least one methyl group from an amino acid side chain. Some demethylases act on histones, e.g., act as a histone H3 or H4 demethylase. For example, an H3 demethylase may demethylate one or more of H3K4, H3K9, H3K27, H3K36, and/or H3K79. Alternately, an H4 demethylase may demethylate histone H4K20. Demethylases are known which can demethylate either a mono-, di- and/or a trimethylated substrate. Further, histone demethylases can act on a methylated core histone substrate, a mononucleosome substrate, a dinucleosome substrate and/or an oligonucleosome substrate, peptide substrate and/or chromatin (e.g., in a cell-based assay).

The first lysine demethylase discovered was lysine specific demethylase 1 (LSD1/KDM1), which demethylates both mono- and di-methylated H3K4 or H3K9, using flavin as a cofactor. A second class of Jumonji C (JmjC) domain containing histone demethylases were predicted, and confirmed when a H3K36 demethylase was found using a formaldehyde release assay, which was named JmjC domain containing histone demethylase 1 (JHDM1/KDM2A).

More JmjC domain-containing proteins were subsequently identified and they can be phylogenetically clustered into seven subfamilies: JHDM1, JHDM2, JHDM3, JMJD2, JARID, PHF2/PHF8, UTX/UTY, and JmjC domain only.

The JMJD2 family of proteins are a family of histone-demethylases known to demethylate tri- and di-methylated H3-K9, and were the first identified histone tri-methyl demethylases. In particular, ectopic expression of JMJD2 family members was found to dramatically decrease levels of tri- and di-methylated H3-K9, while increasing levels of mono-methylated H3-K9, which delocalized Heterochromatin Protein 1 (HP1) and reduced overall levels of heterochromatin in vivo. Members of the JMJD2 subfamily of Jumonji proteins include JMJD2C and its homologues JMJD2A, JMJD2B, JMJD2D and JMJD2E. Common structural features found in the JMJD2 subfamily of Jumonji proteins include the JmjN, JmjC, PHD and Tdr sequences.

JMJD2C, also known as GASC1 and KDM4C, is known to demethylate tri-methylated H3K9 and H3K36. Histone demethylation by JMJD2C occurs via a hydroxylation reaction dependent on iron and a-ketoglutarate, wherein oxidative decarboxylation of a-ketoglutarate by JMJD2C produces carbon dioxide, succinate, and ferryl and ferryl subsequently hydroxylates a methyl group of lysine H3K9, releasing formaldehyde. JMJD2C is known to modulate regulation of adipogenesis by the nuclear receptor PPARγ and is known to be involved in regulation of self-renewal in embryonic stem cells.

As used herein, a “JARID protein” includes proteins in the JARID1 subfamily (e.g., JARID1A, JARID1B, JARID1C and JARID1D proteins) and the JARID2 subfamily, as well as homologues thereof. A further description and listing of JARID proteins can be found in Klose et al. (2006) Nature Reviews/Genetics 7:715-727. The JARID1 family contains several conserved domains: JmjN, ARID, JmjC, PHD and a C5HC2 zing finger.

JARID1A, also called KDMSA or RBP2, was initially found as a binding partner of retinoblastoma (Rb) protein. JARID1A was subsequently found to function as a demethylase of tri- and di-methylated H3K4, and has been found to promote cell growth, while inhibiting senescence and differentiation. For instance, abrogation of JARID1A from mouse cells inhibits cell growth, induces senescence and differentiation, and causes loss of pluripotency of embryonic stem cells in vitro. JARID1A has been found to be overexpressed in gastric cancer and the loss of JARID1A has been found to reduce tumorigenesis in a mouse cancer model. Additionally, studies have demonstrated that loss of the retinoblastome binding protein 2 (RBP2) histone demethylase suppresses tumorigenesis in mice lacking Rbl or Menl (Lin et al. Proc. Natl. Acad. Sci. USA, Aug. 16, 2011, 108(33),13379-86) and the authors of the study concluded that RBP2-inhibitory drugs would have anti-cancer activity.

Unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, a “subject” is interchangeable with a “subject in need thereof”, both of which refer to a subject having a disorder in which a JmjC-domain containing demethylase-mediated protein methylation and/or demethylation plays a part, or a subject having an increased risk of developing such disorder relative to the population at large. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having cancer or a precancerous condition. A subject in need thereof can also be one who has (e.g., is suffering from) cancer or a precancerous condition. Alternatively, a subject in need thereof can be one who has an increased risk of developing such disorder relative to the population at large (i.e., a subject who is predisposed to developing such disorder relative to the population at large). A subject in need thereof can have a precancerous condition. A subject in need thereof can have refractory or resistant cancer (i.e., cancer that doesn't respond or hasn't yet responded to treatment). The subject may be resistant at start of treatment or may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In a preferred embodiment, the subject has cancer or a cancerous condition.

As used herein, “candidate compound” refers to a compound of the present disclosure, or a pharmaceutically acceptable salt, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present disclosure, or a pharmaceutically acceptable salt, polymorph or solvate thereof. The biological or medical response can be the treatment of cancer. The biological or medical response can be treatment or prevention of a cell proliferative disorder. The biological response or effect can also include a change in cell proliferation or growth that occurs in vitro or in an animal model, as well as other biological changes that are observable in vitro. In vitro or in vivo biological assays can include, but are not limited to, enzymatic activity assays, electrophoretic mobility shift assays, reporter gene assays, in vitro cell viability assays, and the assays described herein. For example, an in vitro biological assay that can be used includes the steps of (1) mixing a test compound with one of the KDM4A, KDM4C or KDMSC enzyme constructs (e.g., N-terminal GST-tagged KDM4C²⁻³⁷², N-terminal His-tagged KDM4A¹⁻³⁵⁰, or C-terminal FLAG-tagged KDM5A¹⁻¹⁰⁹⁰) (2) adding a histone substrate (e.g., an isolated histone sample, an isolated histone peptide representative of trimethylated H3K9 and H3K36) to this mixture; (3) adding formic acid to stop the reaction; (4) plotting a dose-response curve which associates the amount of inhibition of the enzyme relative to the concentration of the test compound to determine the IC₅₀ value.

As used herein, “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

A compound of the present disclosure, or a pharmaceutically acceptable salt, polymorph or solvate thereof, can or may also be used to prevent a relevant disease, condition or disorder, or used to identify suitable candidates for such purposes. As used herein, “preventing,” “prevent,” or “protecting against” describes reducing or eliminating the onset of the symptoms or complications of such disease, condition or disorder.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

As used herein, “combination therapy” or “co-therapy” includes the administration of a compound of the present disclosure, or a pharmaceutically acceptable salt, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

The present disclosure also provides pharmaceutical compositions comprising a compound of any of the Formulae described herein in combination with at least one pharmaceutically acceptable excipient or carrier.

A “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

The compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds, or pharmaceutically acceptable salts thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

In the synthetic schemes described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described herein (e.g., such as those in Example 2), to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Example 1: Syntheses of Compounds 1-144 in Table 1

Compounds 1-6 were synthesized following the methods as depicted in or similar to those as shown in Schemes 1 and 2.

### Synthesis of 3-((4-(4-fluorophenyl)-1-methyl-1H-pyrazol-3-yl)amino) isonicotinic acid and 3-((4-(4-Fluorophenyl)-1-methyl-1H-pyrazol-5-yl)amino) isonicotinic acid (Compounds 7 and 8)

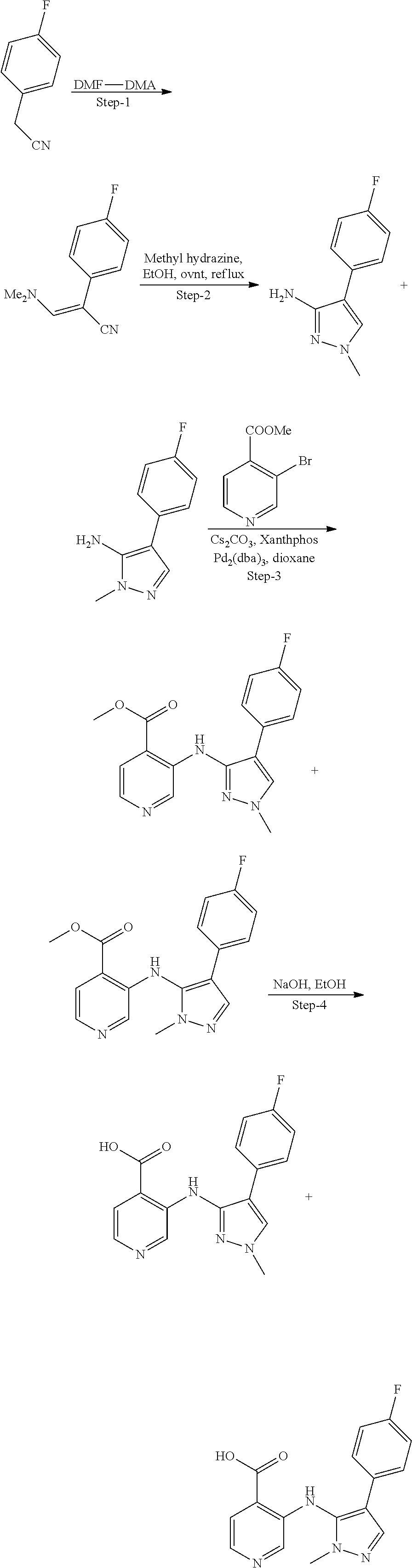

### Synthesis of (E)-3-(dimethylamino)-2-(4-fluorophenyl)acrylonitrile

To a stirred solution of 2-(4-fluorophenyl)acetonitrile (5 g, 37.04 mmol) in toluene (5 mL), DMF-DMA (20 mL) was added and the reaction mixture was refluxed for 12 h. Progress of the reaction was monitored by TLC. Upon completion the reaction mixture was evaporated under reduced pressure resulting in a crude compound which was purified by column chromatography to afford the title compound (3 g, 43%).

### Synthesis of 4-(4-fluorophenyl)-1-methyl-1H-pyrazol-3-amine and 4-(4-fluorophenyl)-1-methyl-1H-pyrazol-5-amine

To a stirred solution of (E)-3-(dimethylamino)-2-(4-fluorophenyl)acrylonitrile (2 g, 10.52 mmol) in EtOH (5 mL), methyl hydrazine (10 mL) was added and the reaction mixture was stirred at 90° C. for 12 h. Progress of the reaction was monitored by TLC. Upon completion the reaction mixture was evaporated under reduced pressure resulting in a crude compound which was purified by column chromatography to afford the mixture of title compounds (1.5 g, 74%).

### Synthesis of methyl 3-((4-(4-fluorophenyl)-1-methyl-1H-pyrazol-3-yl)amino) isonicotinate and methyl 3-((4-(4-fluorophenyl)-1-methyl-1H-pyrazol-5-yl) amino)isonicotinate

To a stirred solution of mixture of 4-(4-fluorophenyl)-1-methyl-1H-pyrazol-3-amine and 4-(4-fluorophenyl)-1-methyl-1H-pyrazol-5-amine (1.5 g, 7.85 mmol) in 1,4-dioxane (20 mL), methyl 3-aminoisonicotinate (3.39 g, 15.70 mmol) and cesium carbonate (3.57 g, 10.99 mmol) was added and purged with argon for 10 min, followed by the addition of xantphos (1.36 g, 2.35 mmol) and purged with argon for an additional 5 min. Pd₂(dba)₃ (0.719 g, 0.785 mmol) was added and stirred at 100° C. for 12 h. Progress of the reaction was monitored by TLC. Upon completion the reaction mixture was filtered through a bed of celite and evaporated to dryness. The residue was taken in ethyl acetate, washed with water and brine, dried over anhydrous sodium sulfate, and evaporated under reduced pressure. The crude product was purified by preparative HPLC to afford methyl 3-((4-(4-fluorophenyl)-1-methyl-1H-pyrazol-3-yl)amino)isonicotinate (0.1 g, 4%) and 3 methyl 3-((4-(4-fluorophenyl)-1-methyl-1H-pyrazol-5-yl)amino) isonicotinate (0.1 g, 4%).

### Synthesis of 3-((4-(4-Fluorophenyl)-1-methyl-1H-pyrazol-3-yl)amino) isonicotinic acid (Compound 7)

To a stirred solution of methyl 3-((4-(4-fluorophenyl)-1-methyl-1H-pyrazol-3-yl)amino)isonicotinate (0.1 g, 0.306 mmol) in EtOH (2 mL), 1N NaOH (2 mL) was added and the reaction mixture was stirred at room temperature for 1 h. Progress of the reaction was monitored by TLC. Upon completion, the reaction mixture was evaporated under reduced pressure. The residue was acidified with 1N HCl and extracted with 10% MeOH/DCM. The combined organic layers were dried over anhydrous sodium sulfate and concentrated under reduced pressure to obtain a residue which was triturated with diethyl ether and pentane, filtered, and dried under reduced pressure to afford the title compound (0.052 g, 55.4%).

### Synthesis of 3-((4-(4-fluorophenyl)-1-methyl-1H-pyrazol-5-yl)amino) isonicotinic acid (Compound 8)

The standard NaOH hydrolysis procedure from paragraph [0307] above was followed.

### Synthesis of 3-((4-(3-Fluorophenyl)-1-methyl-1H-pyrazol-3-yl)amino) isonicotinic acid and 3-((4-(3-Fluorophenyl)-1-methyl-1H-pyrazol-5-yl)amino) isonicotinic acid (Compounds 9 and 10)

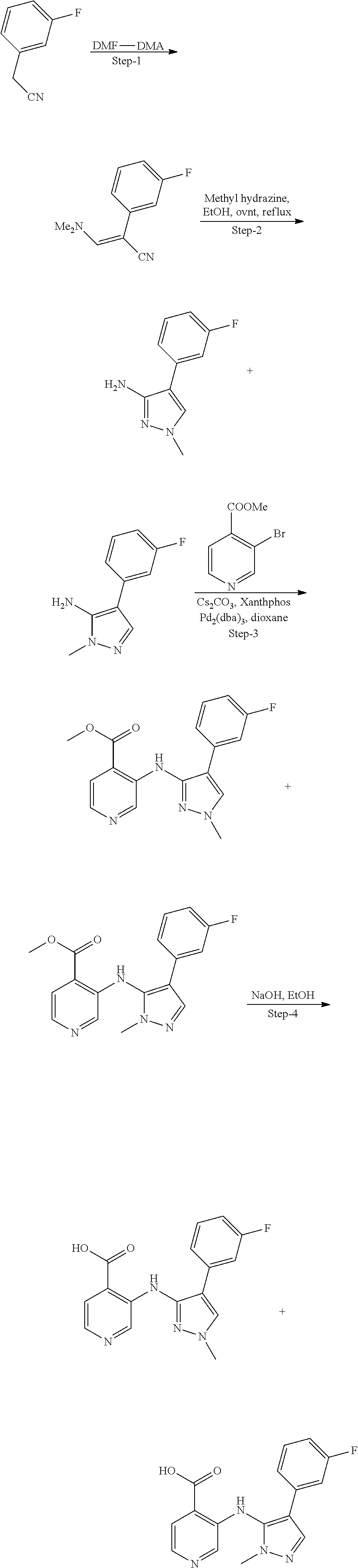

Compounds 9 and 10 were synthesized by a method similar to that illustrated in paragraph [0303] above except that the starting compound was 2-(3-fluorophenyl)acetonitrile.

### Synthesis of 3-((4-(2-Fluorophenyl)-1-methyl-1H-pyrazol-3-yl)amino) isonicotinic acid and 3-((4-(2-fluorophenyl)-1-methyl-1H-pyrazol-5-yl)amino) isonicotinic acid (Compounds 11 and 12)

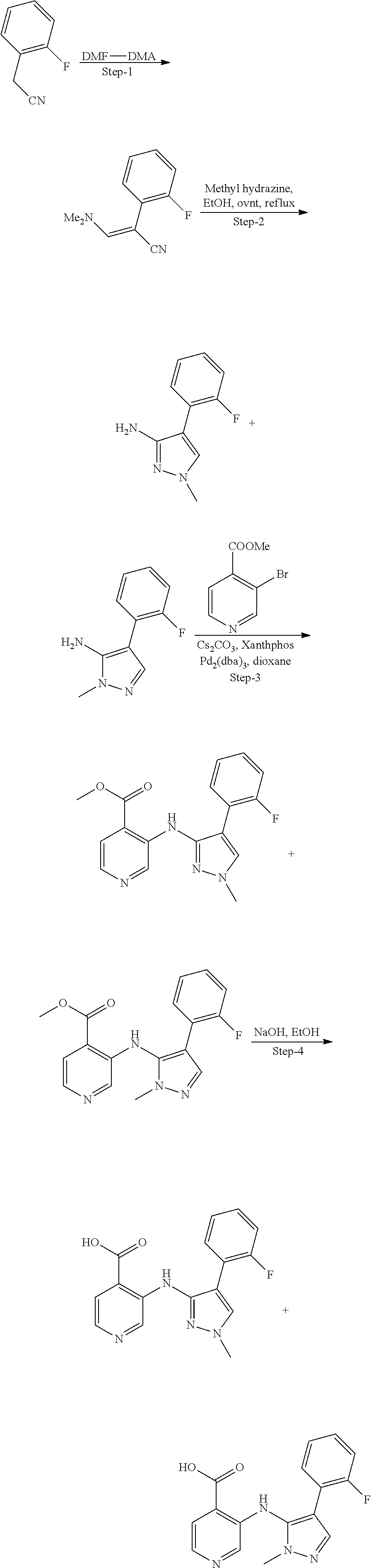

Compounds 11 and 12 were synthesized by a method similar to that illustrated in paragraph [0303] above except that the starting compound was 2-(2-fluorophenyl)acetonitrile.

### Synthesis of 3-((1-methyl-3-phenyl-1H-pyrazol-4-yl)amino) isonicotinic acid (Compound 13)

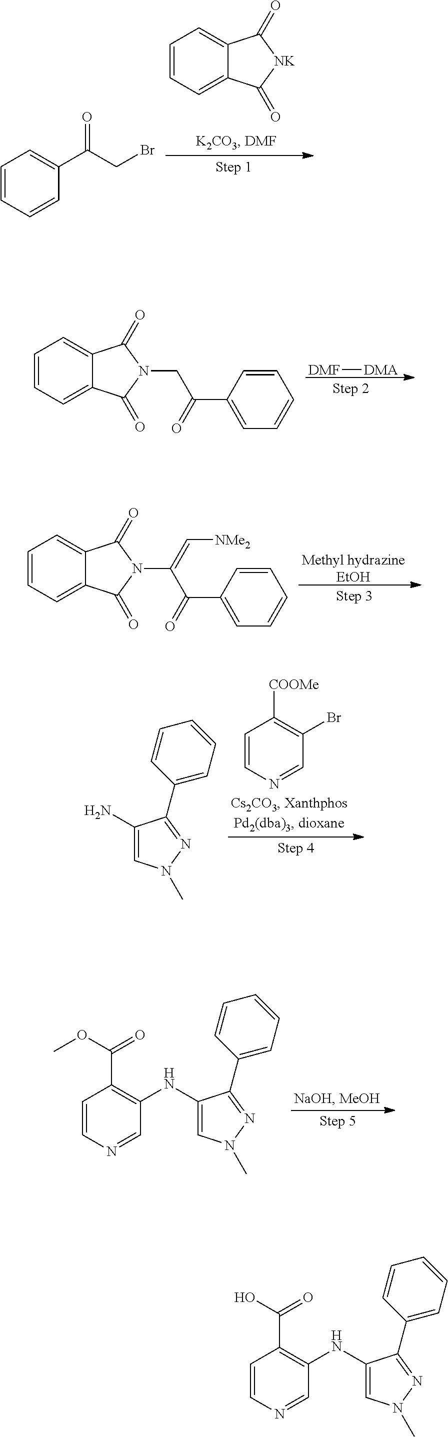

### Synthesis of 2-(2-oxo-2-phenylethyl) isoindoline-1, 3-dione

To a stirred solution of 2-bromo-1-phenylethanone (15 g, 75.37 mmol) in DMF (60 mL), potassium 1,3-dioxoisoindolin-2-ide (15.3 g, 82.91 mmol) was added and the reaction mixture was stirred at 40° C. for 4 h. Progress of the reaction was monitored by TLC. Upon completion the reaction mixture was diluted with water and extracted with dichloromethane. The combined organic layers were washed with 0.2 N NaOH, brine, and water. The organic layer was dried over anhydrous sodium sulfate and concentrated under reduced pressure to afford the title compound. The crude compound was used as such for next step (15 g, 75.37%).

### Synthesis of (E)-2-(1-(dimethylamino)-3-oxo-3-phenylprop-1-en-2-yl) isoindoline-1, 3-dione

To a stirred solution of 2-(2-oxo-2-phenylethyl) isoindoline-1, 3-dione (15 g, 56.60 mmol) and DMF-DMA (30 mL g, 2.24 mmol) in toluene (50 mL) was added and stirred at 100° C. for 16 h. Progress of the reaction was monitored by TLC. Upon completion, the reaction mixture was evaporated under reduced pressure to obtain a residue which was triturated with diethyl ether and pentane, filtered, and dried under reduced pressure to afford the title compound (14.5 g, 80%).

### Synthesis of 1-methyl-3-phenyl-1H-pyrazol-4-amine

To a stirred solution of (E)-2-(1-(dimethylamino)-3-oxo-3-phenylprop-1-en-2-yl) isoindoline-1, 3-dione (4 g, 12.25 mmol) in EtOH (40 mL), methyl hydrazine (1.39 g, 25 mmol) was added and the reaction mixture was stirred at 90° C. for 12 h. Progress of the reaction was monitored by TLC. Upon completion the reaction mixture was evaporated under reduced pressure resulting in a crude compound which was purified by column chromatography to afford the mixture of title compounds (1.5 g, 74%).

### Synthesis of methyl 3-((1-methyl-3-phenyl-1H-pyrazol-4-yl) amino) isonicotinate

The standard Buchwald coupling protocol from paragraph [0306] above was followed.

### Synthesis of 3-((1-methyl-3-phenyl-1H-pyrazol-4-yl) amino) isonicotinic acid (Compound 13)

The standard NaOH hydrolysis procedure from paragraph [0307] above was followed.

### Synthesis of 3-((1-(2-(4,4-difluoropiperidin-1-yl)ethyl)-4-phenyl-1H-pyrazol-3-yl)amino) isonicotinic acid and 3-((1-(2-(4,4-difluoropiperidin-1-yl)ethyl)-4-phenyl-1H-pyrazol-5-yl)amino) isonicotinic acid (Compounds 14 and 15)

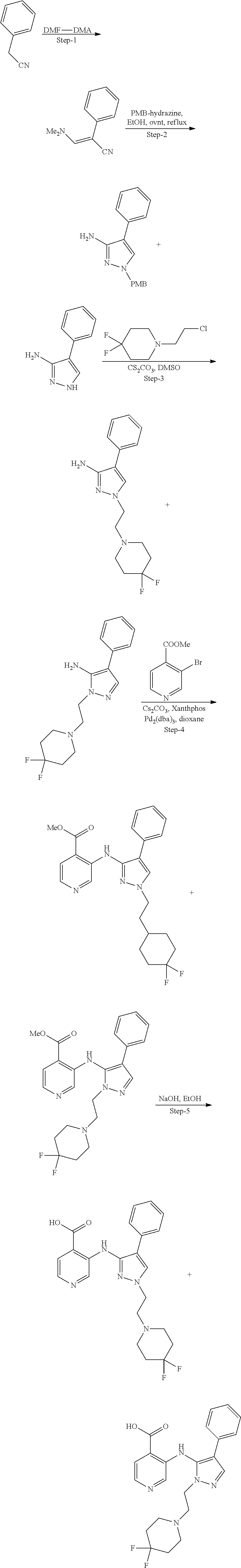

### Synthesis of (E)-3-(dimethylamino)-2-phenylacrylonitrile

To a stirred solution of 2-phenylacetonitrile (10 g, 85.47 mmol) in toluene (50 mL), DMF-DMA (45 mL) was added and the reaction mixture was refluxed for 12 h. Progress of the reaction was monitored by TLC. Upon completion the reaction mixture was evaporated under reduced pressure resulting in a crude compound which was purified by column chromatography to afford the title compound (12 g, 81.6%).

### Synthesis of 1-(4-methoxybenzyl)-4-phenyl-1H-pyrazol-3-amine and 4-phenyl-1H-pyrazol-3-amine

To a stirred solution of (E)-3-(dimethylamino)-2-phenylacrylonitrile (5 g, 28.90 mmol) in EtOH (150 mL), PMB-hydrazine (8.1 g, 43.35 mmol) was added and the reaction mixture was stirred at room temperature for 12 h. Progress of the reaction was monitored by TLC. Upon completion the reaction mixture was evaporated under reduced pressure resulting in a crude compound which was purified by column chromatography to afford 4-phenyl-1H-pyrazol-3-amine (1 g, 21.6%) and 1-(4-methoxybenzyl)-4-phenyl-1H-pyrazol-3-amine (1 g, 12.3%).

### Synthesis of 1-(2-(4,4-difluoropiperidin-1-yl)ethyl)-4-phenyl-1H-pyrazol-3-amine and 1-(2-(4,4-difluoropiperidin-1-yl)ethyl)-4-phenyl-1H-pyrazol-5-amine

To a stirred solution of 4-phenyl-1H-pyrazol-3-amine (0.9 g, 5.66 mmol) and 1-(2-chloroethyl)-4,4-difluoropiperidine hydrochloride (1.76 g, 9.62 mmol) in DMSO (15 mL), Cs₂CO₃ (4.59 g, 14.15 mmol) was added and stirred at room temperature for 16 h. Progress of the reaction was monitored by TLC. Upon completion the reaction mixture was diluted with water and extracted with ethyl acetate. The combined organic layers were dried over anhydrous sodium sulfate and concentrated under reduced pressure. The crude compound was purified by column chromatography to afford 1-(2-(4,4-difluoropiperidin-1-yl)ethyl)-4-phenyl-1H-pyrazol-3-amine (0.65 g, 76.5%) and 1-(2-(4,4-difluoropiperidin-1-yl)ethyl)-4-phenyl-1H-pyrazol-5-amine (0.65 g, 76.5%)

### Synthesis of methyl 3-((1-(2-(4,4-difluoropiperidin-1-yl)ethyl)-4-phenyl-1H-pyrazol-3-yl)amino)isonicotinate

The standard Buchwald coupling protocol from paragraph [0306] above was followed.

### Synthesis of methyl 3-((1-(2-(4,4-difluoropiperidin-1-yl)ethyl)-4-phenyl-1H-pyrazol-5-yl)amino)isonicotinate

The standard Buchwald coupling protocol from paragraph [0306] above was followed.

### Synthesis of 3-((1-(2-(4,4-Difluoropiperidin-1-yl)ethyl)-4-phenyl-1H-pyrazol-3-yl)amino)isonicotinic acid (Compound 14)

The standard NaOH hydrolysis procedure from paragraph [0307] above was followed.

### Synthesis of 3-((1-(2-(4,4-difluoropiperidin-1-yl)ethyl)-4-phenyl-1H-pyrazol-5-yl)amino)isonicotinic acid (Compound 15)

The standard NaOH hydrolysis procedure from paragraph [0307] above was followed.

### Synthesis of 3-((3,5-Dimethyl-1H-pyrazol-4-yl)amino)isonicotinic acid (Compound 16)

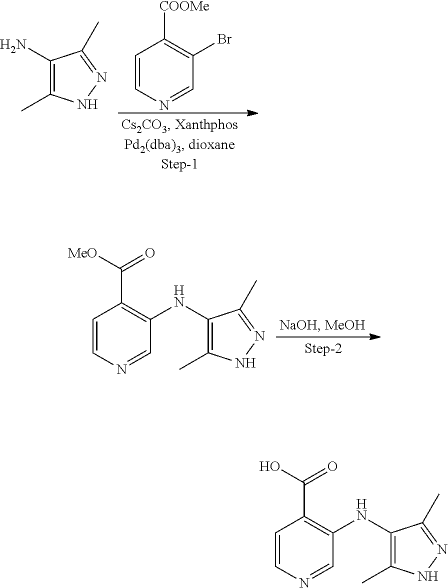

### Synthesis of Methyl 3-((3,5-dimethyl-1H-pyrazol-4-yl)amino)isonicotinate

The standard Buchwald coupling protocol from paragraph [0306] above was followed.

### Synthesis of 3-((3,5-Dimethyl-1H-pyrazol-4-yl)amino)isonicotinic acid (Compound 16)

The standard NaOH hydrolysis procedure from paragraph [0307] above was followed.

### Synthesis of 3-((1-(2-(4,4-Difluoropiperidin-1-yl)ethyl)-3-phenyl-1H-pyrazol-4-yl)amino)isonicotinic acid and 3-((1-(2-(4,4-difluoropiperidin-1-yl) ethyl)-5-phenyl-1H-pyrazol-4-yl)amino)isonicotinic acid (Compounds 17 and 18)

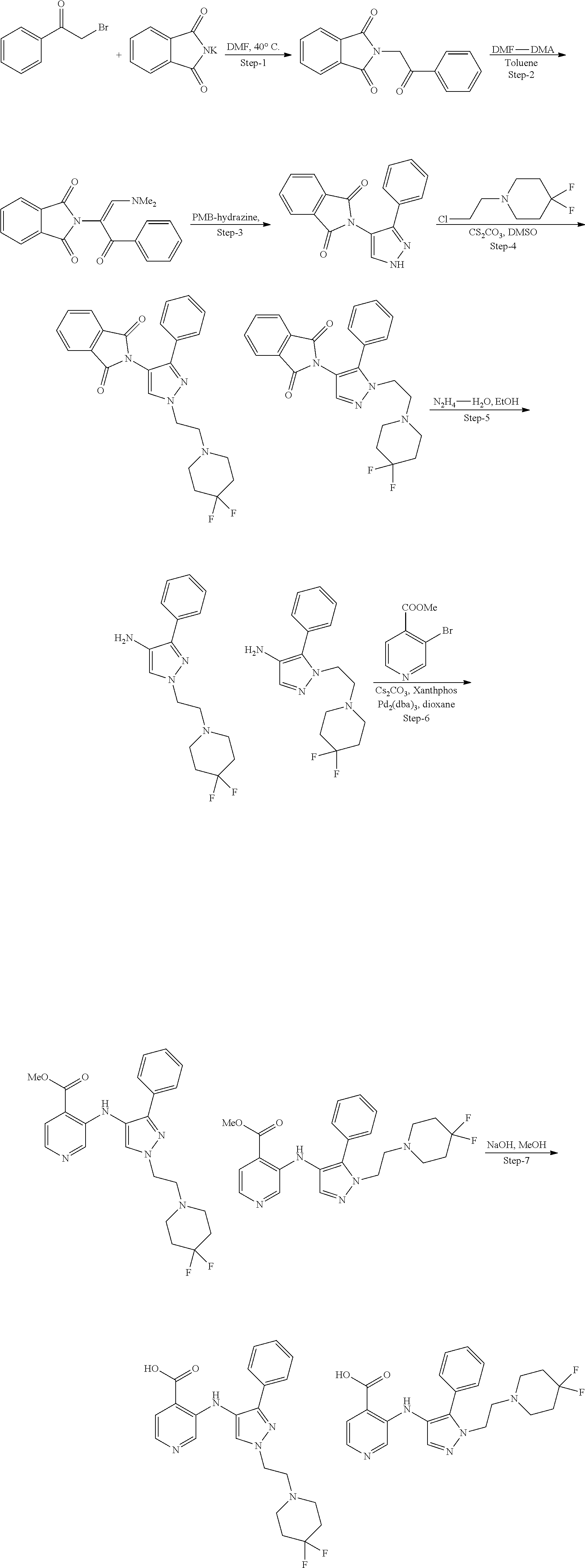

### Synthesis of 2-(2-Oxo-2-phenylethyl)isoindoline-1,3-dione

To a stirred solution of 2-bromo-1-phenylethanone (15 g, 75.37 mmol) in DMF (60 mL), potassium 1,3-dioxoisoindolin-2-ide (15.3 g, 82 mmol) was added and stirred at 40° C. for 4 h. Progress of the reaction was monitored by TLC. Upon completion the reaction mixture was diluted with water and extracted with ethyl acetate. The combined organic layers were washed with 0.2 N NaOH; water; dried over anhydrous Na₂SO₄ and concentrated under reduced pressure to afford the title compound (15 g, 75.4%).

### Synthesis of (E)-2-(1-(Dimethylamino)-3-oxo-3-phenylprop-1-en-2-yl) isoindoline-1,3-dione

To a stirred solution of 2-(2-oxo-2-phenylethyl)isoindoline-1,3-dione (15 g, 56 mmol) in toluene (50 mL), DMF-DMA (27 g, 224 mmol) was added. The resulting reaction mixture was stirred at 100° C. for 15 h. Progress of the reaction was monitored by TLC. Upon completion the reaction mixture was concentrated to dryness under reduced pressure. The crude compound was purified by column chromatography to afford the title compound (14.5 g, 80%).

### Synthesis of 2-(3-Phenyl-1H-pyrazol-4-yl)isoindoline-1,3-dione

To a stirred solution of (E)-2-(1-(dimethylamino)-3-oxo-3-phenylprop-1-en-2-yl)isoindoline-1,3-dione (7.5 g, 23.36 mmol) in EtOH (90 mL), PMB hydrazine (6.5 g, 35.04 mmol) was added. The resulting reaction mixture was stirred at room temperature for 15 h. Progress of the reaction was monitored by TLC. Upon completion the reaction mixture was concentrated to dryness under reduced pressure. The crude compound was purified by column chromatography to afford the title compound (4 g, 59%).

### Synthesis of 2-(1-(2-(4,4-Difluoropiperidin-1-yl)ethyl)-3-phenyl-1H-pyrazol-4-yl) isoindoline-1,3-dione and 2-(1-(2-(4,4-difluoropiperidin-1-yl)ethyl)-5-phenyl-1H-pyrazol-4-yl)isoindoline-1,3-dione

To a stirred solution of 2-(3-phenyl-1H-pyrazol-4-yl)isoindoline-1,3-dione (4 g, 13.84 mmol) and 1-(2-chloroethyl)-4,4-difluoropiperidine (5 g, 27.68 mmol) in DMSO (25 mL), Cs₂CO₃ (13.5 g, 41.52 mmol) was added and reaction was stirred at room temperature for 16 h. Progress of the reaction was monitored by TLC. Upon completion the reaction mixture was diluted with water and extracted with ethyl acetate. Combined organic layers were washed with brine, dried over anhydrous Na₂SO₄ and concentrated under reduced pressure. The crude compound was purified by column chromatography to afford the mixture of title compounds (2.2 g, 37%).

### Synthesis of 1-(2-(4,4-Difluoropiperidin-1-yl)ethyl)-3-phenyl-1H-pyrazol-4-amine and 1-(2-(4,4-difluoropiperidin-1-yl)ethyl)-5-phenyl-1H-pyrazol-4-amine

To a mixture of 2-(1-(2-(4,4-difluoropiperidin-1-yl)ethyl)-3-phenyl-1H-pyrazol-4-yl)isoindoline-1,3-dione and 2-(1-(2-(4,4-difluoropiperidin-1-yl)ethyl)-5-phenyl-1H-pyrazol-4-yl)isoindoline-1,3-dione (2.2 g, 5.05 mmol) in EtOH (20 mL), and hydrazine hydrate (12 mL) were added. The resulting reaction mixture was stirred at 90° C. for 16 h. Progress of the reaction was monitored by TLC. Upon completion the reaction mixture was concentrated to dryness under reduced pressure. The crude compound was purified by column chromatography to afford the mixture of title compounds (0.9 g, 60%).

### Synthesis of methyl 3-((1-(2-(4,4-difluoropiperidin-1-yl)ethyl)-3-phenyl-1H-pyrazol-4-yl)amino)isonicotinate and methyl 3-((1-(2-(4,4-difluoropiperidin-1-yl)ethyl)-5-phenyl-1H-pyrazol-4-yl)amino)isonicotinate

The standard Buchwald coupling protocol from paragraph [0306] above was followed (0.45 g, 37.5%).

### Synthesis of 3-((1-(2-(4,4-Difluoropiperidin-1-yl)ethyl)-3-phenyl-1H-pyrazol-4-yl)amino)isonicotinic acid (Compound 17)

The standard NaOH hydrolysis procedure from paragraph [0307] above was followed.

### Synthesis of 3-((3-Methyl-1-phenyl-1H-pyrazol-4-yl)amino)isonicotinic acid and 3-((5-methyl-1-phenyl-1H-pyrazol-4-yl)amino)isonicotinic acid (Compounds 19 and 20)

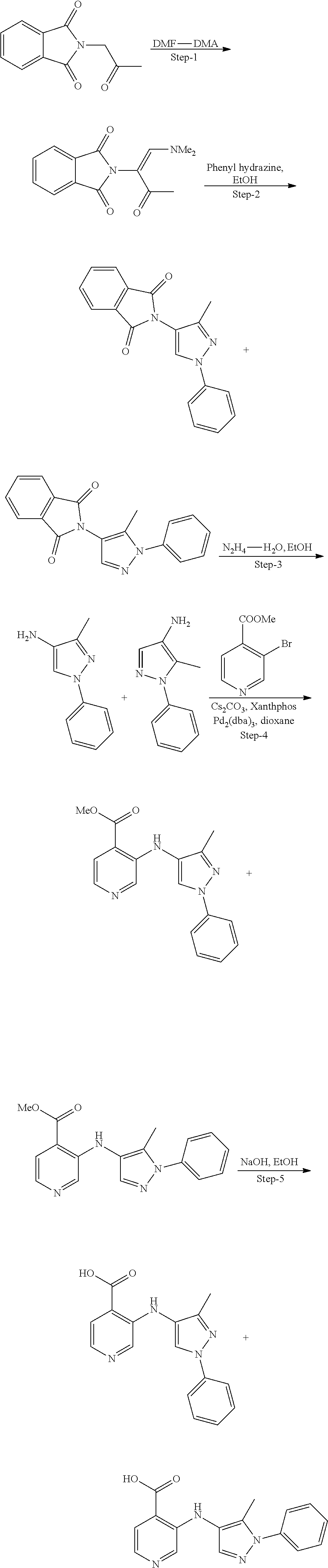

Compounds 19 and 20 were synthesized by a method similar to that illustrated in paragraph [0313] above except that the starting compound was 2-(2-oxopropyl)isoindoline-1,3-dione.

### Synthesis of 3-((1,3-Diphenyl-1H-pyrazol-5-yl)amino)isonicotinic acid (Compound 21)

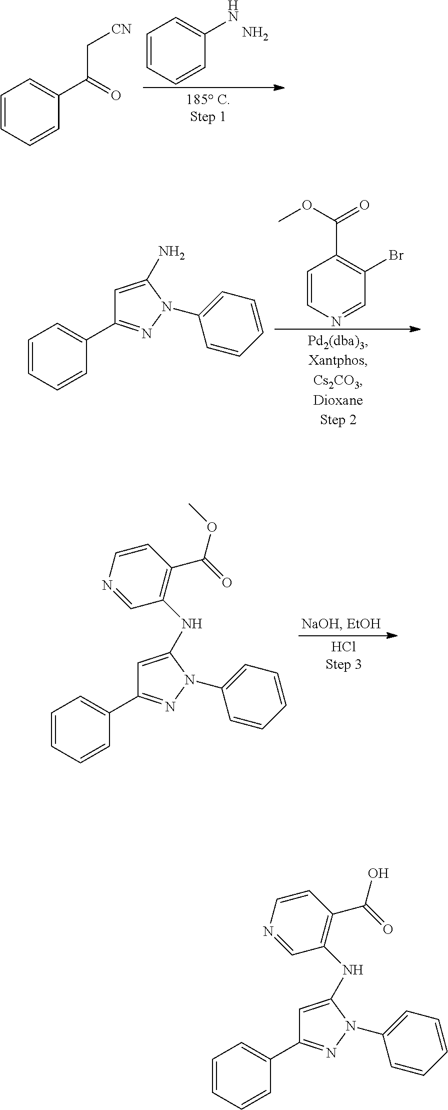

### Synthesis of 1,3-Diphenyl-1H-pyrazol-5-amine

A mixture of 3-oxo-3-phenylpropanenitrile (1 g, 6.89 mmol) and phenylhydrazine (0.745 g, 6.89 mmol) was heated at 185° C. for 4 h. Progress of the reaction was monitored by TLC. Upon completion the reaction mixture was cooled to room temperature and the solid was triturated with diethyl ether, filtered and dried under reduced pressure to afford the title compound (1.2 g, 75%).

### Synthesis of Methyl 3-((1,3-diphenyl-1H-pyrazol-5-yl)amino)isonicotinate

The standard Buchwald coupling protocol from paragraph [0306] above was followed (0.2 g, 32%).

### Synthesis of 3-((1,3-Diphenyl-1H-pyrazol-5-yl)amino)isonicotinic acid (Compound 21)

The standard NaOH hydrolysis procedure from paragraph [0307] above was followed. (0.07 g, 36.4%).

### Synthesis of 3-((1-Isopropyl-3-phenyl-1H-pyrazol-5-yl)amino)isonicotinic acid (Compound 22)

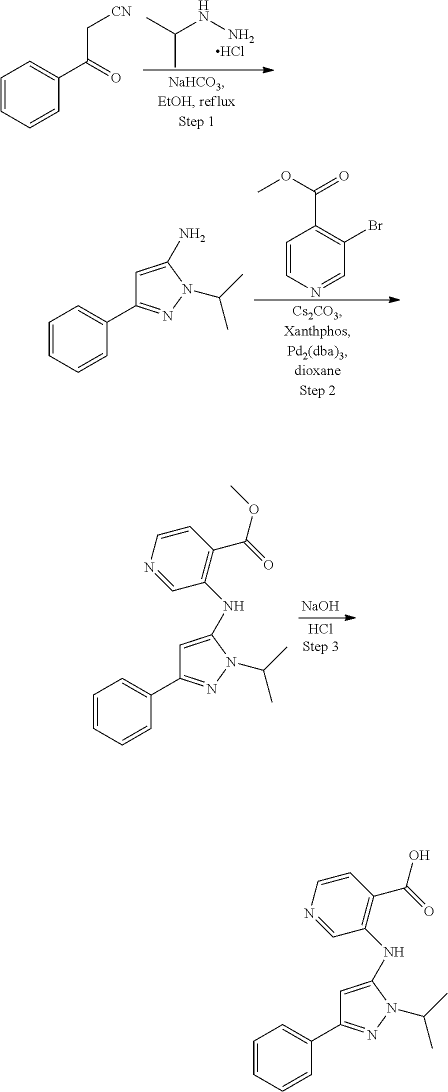

Compound 22 was synthesized by a method similar to that illustrated in paragraph [0340] above except that the starting compound was isopropyl hydrazine.

### Synthesis of 3-((3-Phenyl-1-(tetrahydro-2H-pyran-4-yl)-1H-pyrazol-5-yl) amino)isonicotinic acid (Compound 23)

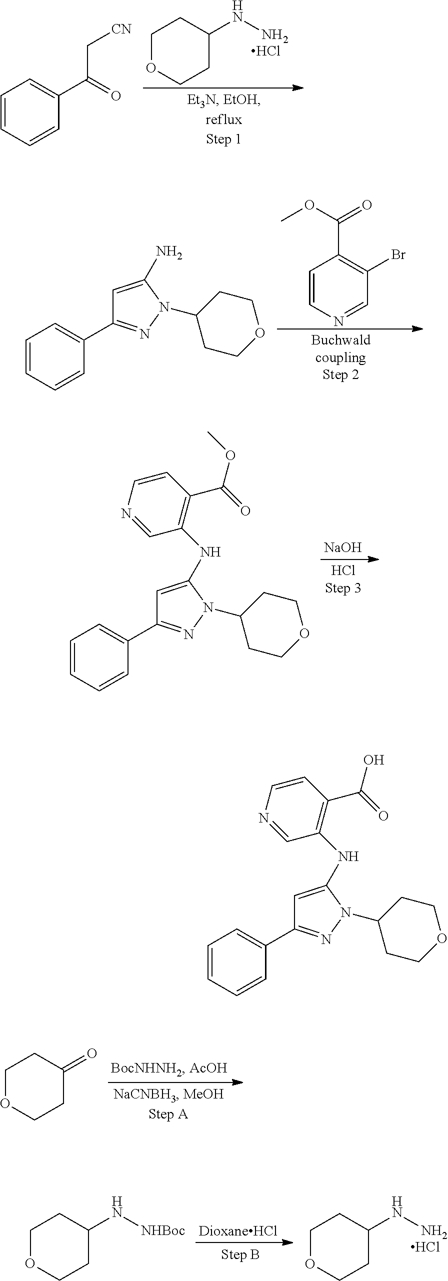

Compound 23 was synthesized by a method similar to that illustrated in paragraph [0340] above except that the starting compound was (tetrahydro-2H-pyran-4-yl)hydrazine.

### Synthesis of (tetrahydro-2H-pyran-4-yl)hydrazine

To stirred solution of tert-butyl 2-(tetrahydro-2H-pyran-4-yl)hydrazinecarboxylate (1.8 g, 8.33 mmol) in 1,4 dioxane (15 mL), HCl.dioxane (4 M, 10 mL) was added and stirred at room temperature for 1 h. Progress of the reaction was monitored by TLC. Upon completion the reaction mixture was evaporated under reduced pressure to obtain a residue which was triturated with diethyl ether and pentane, filtered and dried under reduced pressure to afford the title compound.

### Synthesis of 3-((3-(4-Chlorophenyl)-1-methyl-1H-pyrazol-5-yl)amino) isonicotinic acid (Compound 24)

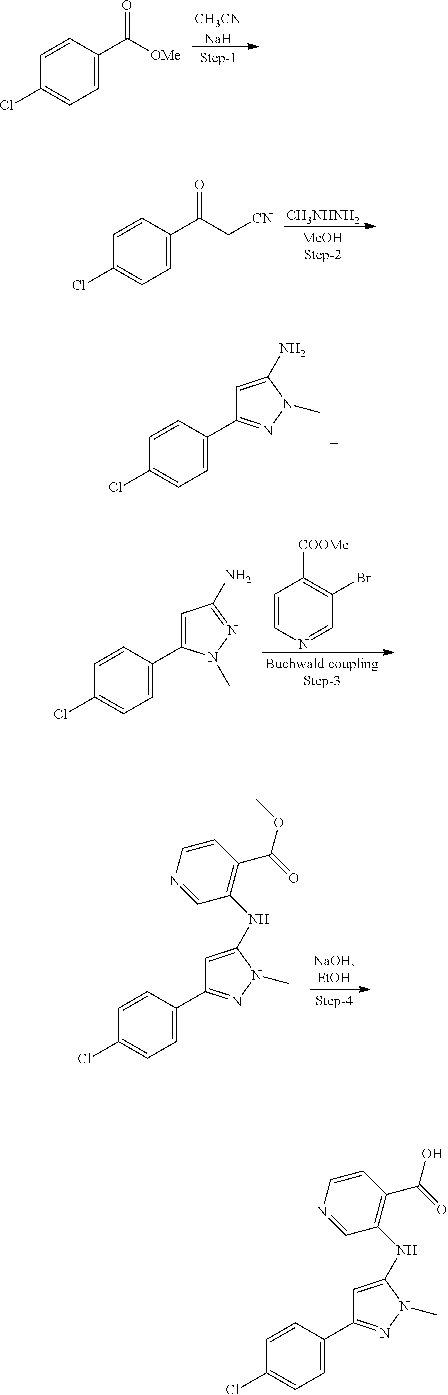

Compound 24 was synthesized by a method similar to that illustrated in paragraph [0340] above except that the starting compound was 3-(4-Chlorophenyl)-3-oxopropanenitrile.

### Synthesis of 3-(4-Chlorophenyl)-3-oxopropanenitrile

To a stirred solution of methyl 4-chlorobenzoate (5 g, 29.41 mmol) in toluene (50 mL), ACN (4.6 mL, 88.23 mmol) and NaH (60%, 3.3 g, 88.23 mmol) were added and stirred at 100° C. for 15 h. Progress of the reaction was monitored by TLC. Upon completion the reaction mixture was diluted with ice cold water, quenched with 2N HCl up to pH=2 and extracted with ethyl acetate. The combined organic layers were dried over anhydrous Na₂SO₄ and concentrated under reduced pressure resulting in a crude compound which was purified by column chromatography to afford the title compound (3.2 g, 62%).

### Synthesis of 3-((3-(3-chlorophenyl)-1-methyl-1H-pyrazol-5-yl)amino) isonicotinic acid (Compound 25)

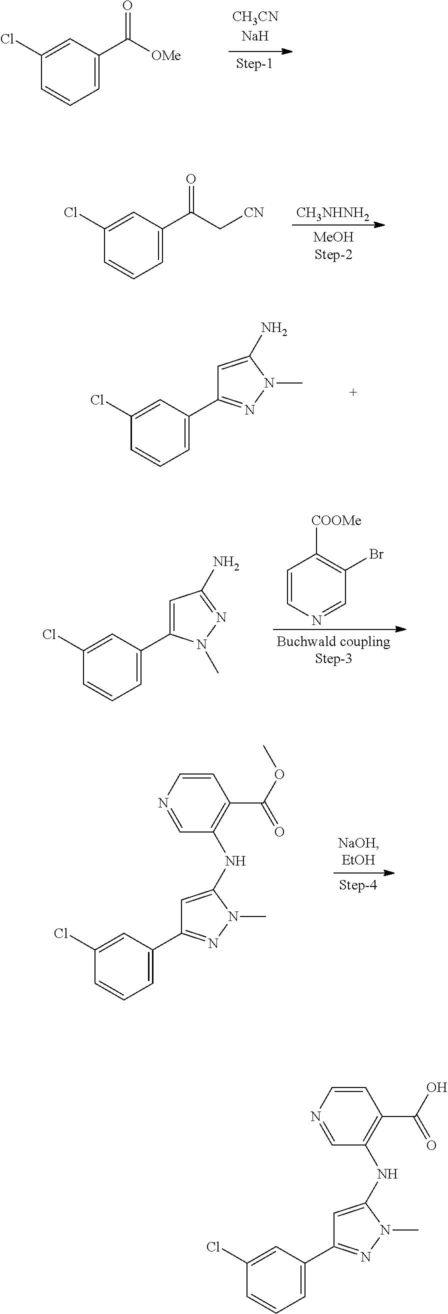

### Synthesis of 3-(3-Chlorophenyl)-3-oxopropanenitrile

Compound 25 was synthesized by a method similar to that illustrated in paragraph [0340] above except that the starting compound was 3-(3-chlorophenyl)-3-oxopropanenitrile.

### Synthesis of 3-(3-Chlorophenyl)-3-oxopropanenitrile

3-(3-Chlorophenyl)-3-oxopropanenitrile was synthesized by a method similar to that illustrated in paragraph [0351] above (7 g, 73%).

### Synthesis of 3-((1-(2-Methoxyethyl)-3-phenyl-1H-pyrazol-5-yl)amino) isonicotinic acid (Compound 26)

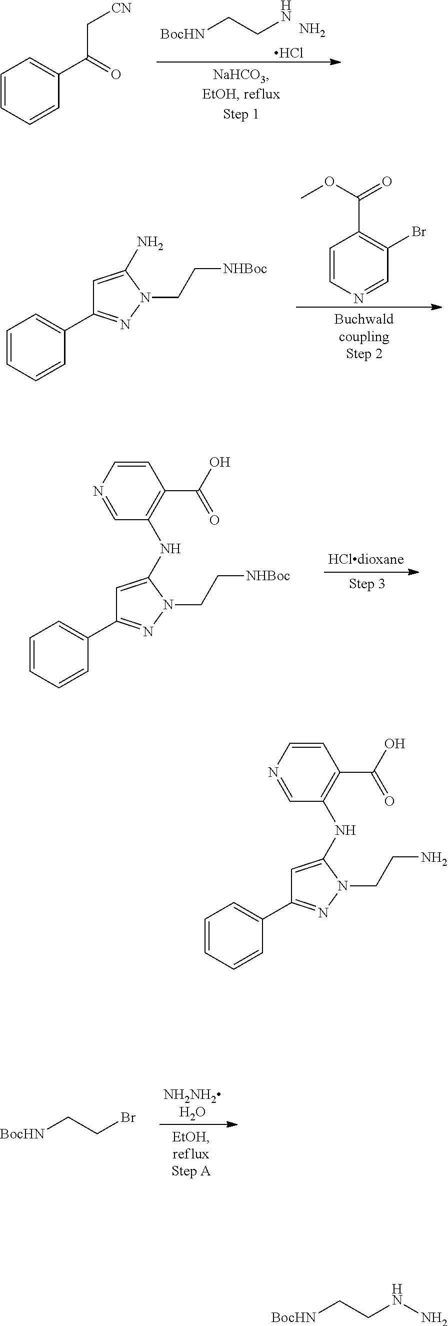

Compound 26 was synthesized by a method similar to that illustrated in paragraph [0340] above except that the starting compound was tert-Butyl (2-hydrazinylethyl)carbamate.

### Synthesis of tert-Butyl (2-hydrazinylethyl)carbamate

To a stirred solution of tert-butyl (2-bromoethyl)carbamate (2 g, 8.93 mmol) in EtOH (15 mL), hydrazine hydrate (6.69 g, 133.9 mmol) was added and refluxed for 15 h. Progress of the reaction was monitored by TLC. Upon completion the reaction mixture was concentrated to dryness under reduced pressure to afford the title compound (1.6 g, crude).

### Synthesis of 3-((1-(2-Methoxyethyl)-3-phenyl-1H-pyrazol-5-yl)amino) isonicotinic acid (Compound 27)

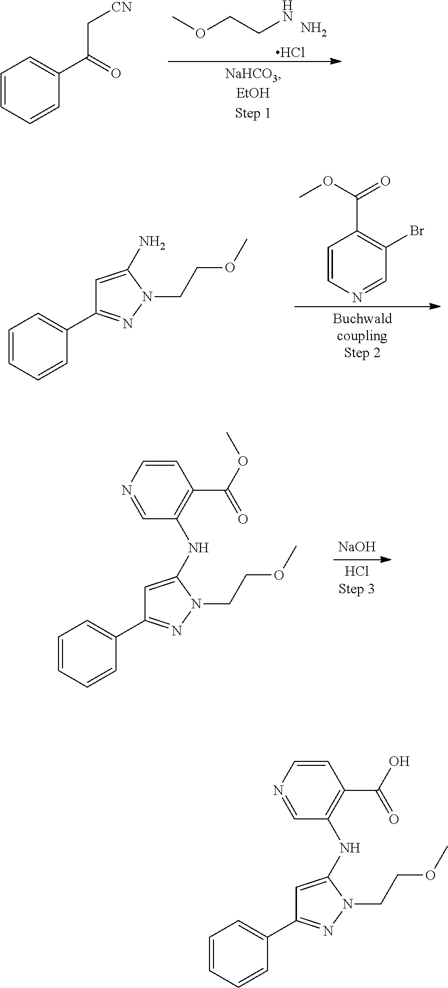

Compound 27 was synthesized by a method similar to that illustrated in paragraph [0340] above except that the starting compound was (2-methoxyethyl)hydrazine.

### Synthesis of 3-((1-(3-bromophenyl)-3-methyl-1H-pyrazol-4-yl)amino) isonicotinic acid and 3-((1-(3-bromophenyl)-5-methyl-1H-pyrazol-4-yl)amino) isonicotinic acid (Compounds 28 and 29)

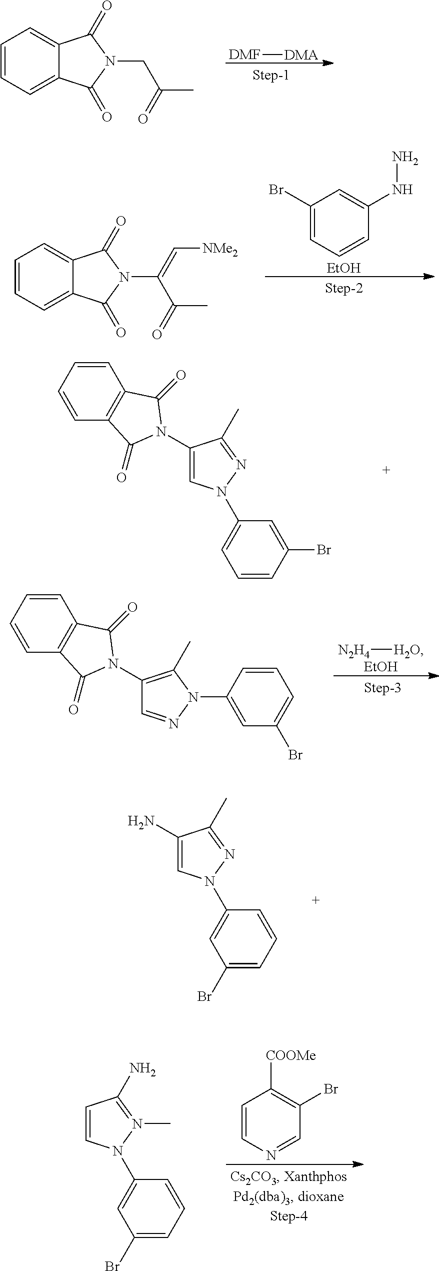

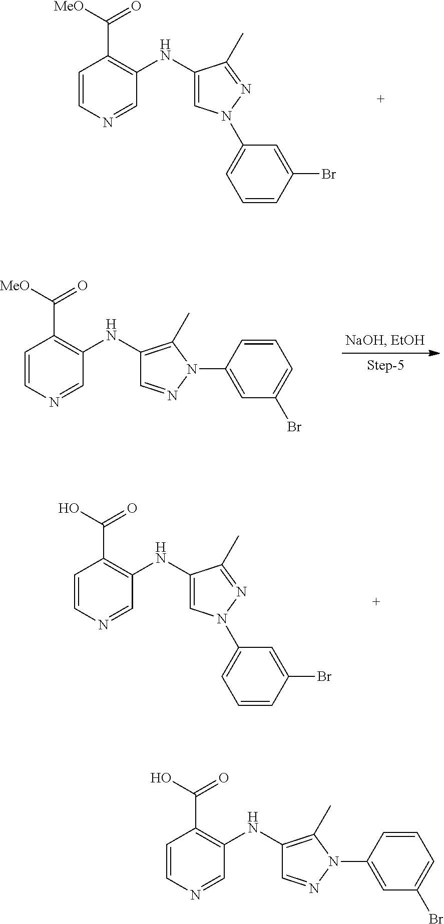 [SMILES: COC(=O)c1ccncc1Nc1cn(-c2cccc(Br)c2)nc1C ; COC(=O)c1ccncc1Nc1cnn(-c2cccc(Br)c2)c1C ; Cc1nn(-c2cccc(Br)c2)cc1Nc1cnccc1C(=O)O ; Cc1c(Nc2cnccc2C(=O)O)cnn1-c1cccc(Br)c1]

Compounds 28 and 29 were synthesized by a method similar to that illustrated in paragraph [0313] above except that the starting compound was (3-bromophenyl)hydrazine.

### Synthesis of 3-((1-(2-Morpholinoethyl)-4-phenyl-1H-pyrazol-3-yl)amino) isonicotinic acid and 3-((1-(2-morpholinoethyl)-4-phenyl-1H-pyrazol-5-yl) amino)isonicotinic acid (Compounds 30 and 31)

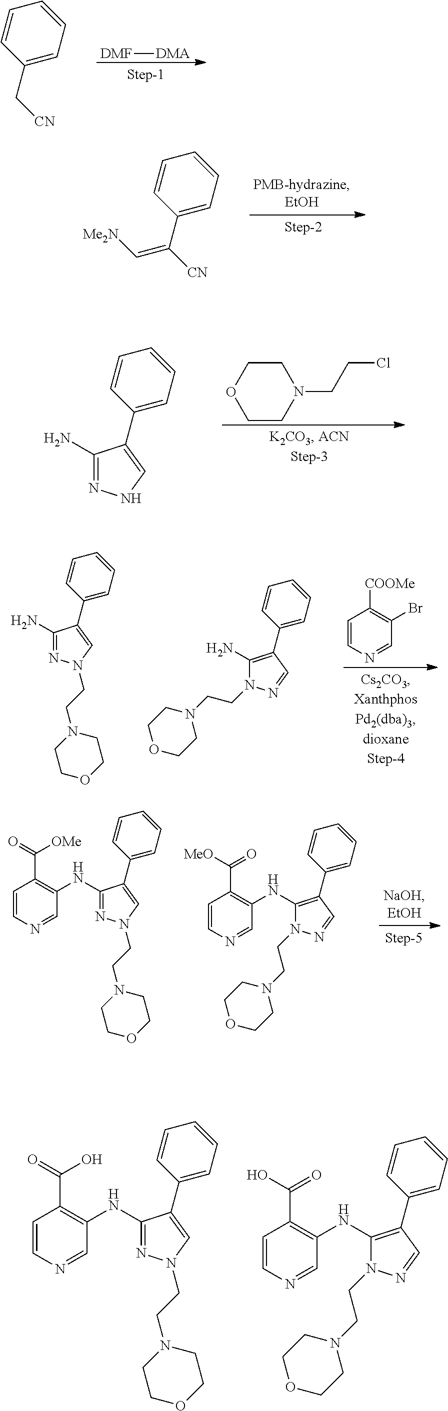

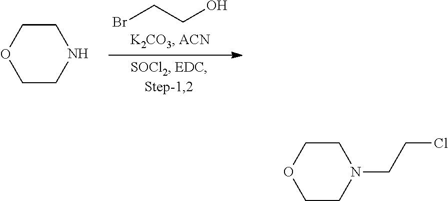

Compounds 30 and 31 were synthesized by a method similar to that illustrated in paragraph [0319] above except that the starting compound was 4-(2-Chloroethyl)morpholine.

### Synthesis of 4-(2-chloroethyl)morpholine

To a stirred solution of morpholine (3 g, 34.48 mmol) and 2-bromoethanol (8.62 g, 68.96 mmol) in ACN (30 mL), K₂CO₃ (14.1 g, 103.4 mmol) was added and reaction was stirred at 75° C. for 16 h. Progress of the reaction was monitored by TLC. Upon completion the reaction mixture was filtered and the filtrate was concentrated under reduced pressure. The crude compound was dissolved in dichloroethane (60 mL), and SOCl₂ (15 mL) was added and stirred at 80° C. for 16 h. Progress of the reaction was monitored by TLC. Upon completion the reaction mixture was concentrated under reduced pressure to obtain a residue which was triturated with diethyl ether and pentane, filtered and dried under reduced pressure to afford the title compound (2 g, crude).

### Synthesis of 3-((3-methyl-1-(tetrahydro-2H-pyran-4-yl)-1H-pyrazol-4-yl) amino)isonicotinic acid (Compound 32)

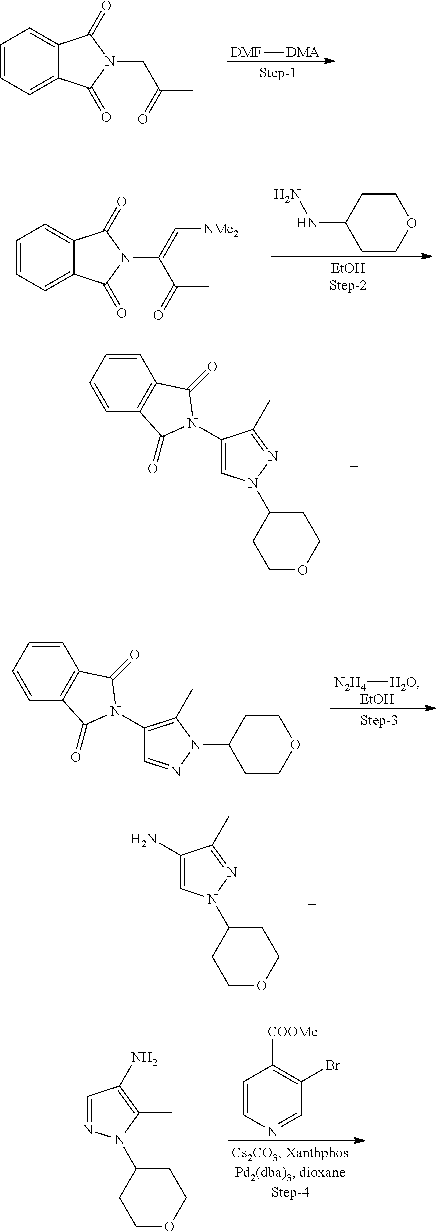

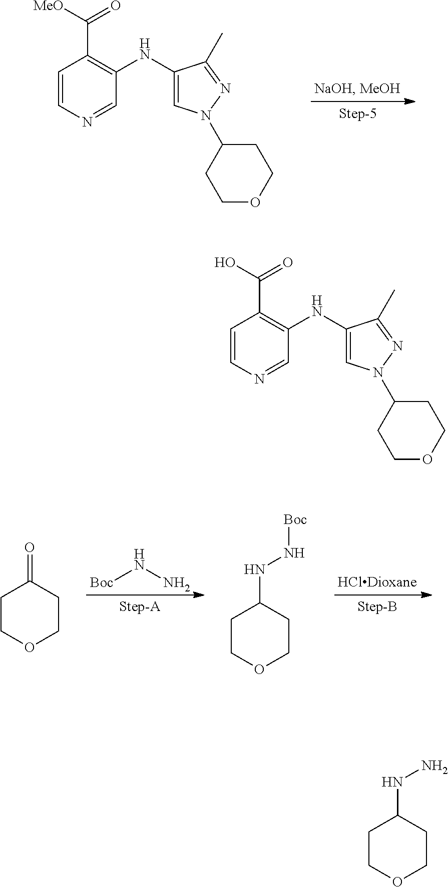 [SMILES: COC(=O)c1ccncc1Nc1cn(C2CCOCC2)nc1C ; Cc1nn(C2CCOCC2)cc1Nc1cnccc1C(=O)O ; O=C1CCOCC1 ; CC(C)(C)OC(=O)NN ; CC(C)(C)OC(=O)NNC1CCOCC1 ; NNC1CCOCC1]

Compound 32 was synthesized by a method similar to that illustrated in paragraph [0313] above except that the starting compound was (tetrahydro-2H-pyran-4-yl)hydrazine.

### Synthesis of 3-((1-benzyl-5-methyl-1H-pyrazol-4-yl)amino)isonicotinic acid (Compound 33)

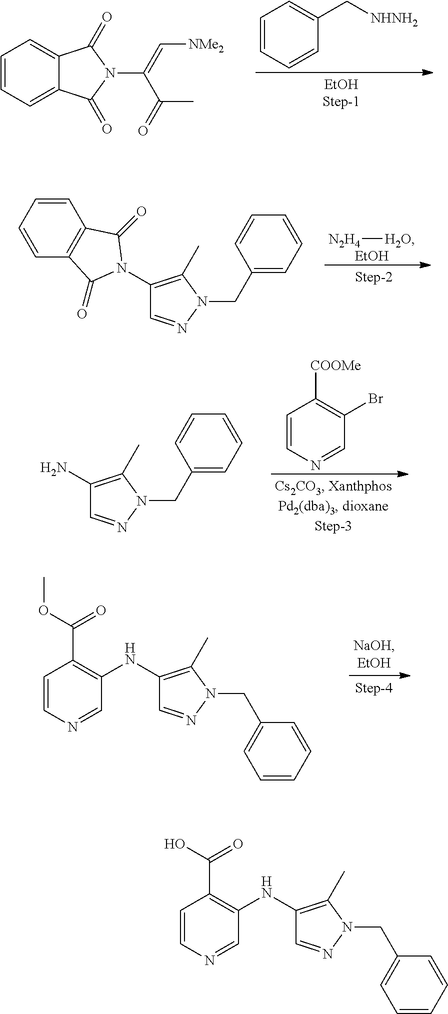

Compound 33 was synthesized by a method similar to that illustrated in paragraph [0313] above except that the starting compounds were 2-(2-oxopropyl)isoindoline-1,3-dione and benzyl hydrazine.

### Synthesis of 3-((1-(2-(4-Methylpiperazin-1-yl)ethyl)-4-phenyl-1H-pyrazol-3-yl)amino)isonicotinic acid and 3-((1-(2-(4-methylpiperazin-1-yl)ethyl)-4-phenyl-1H-pyrazol-5-yl)amino)isonicotinic acid (Compounds 34 and 35)

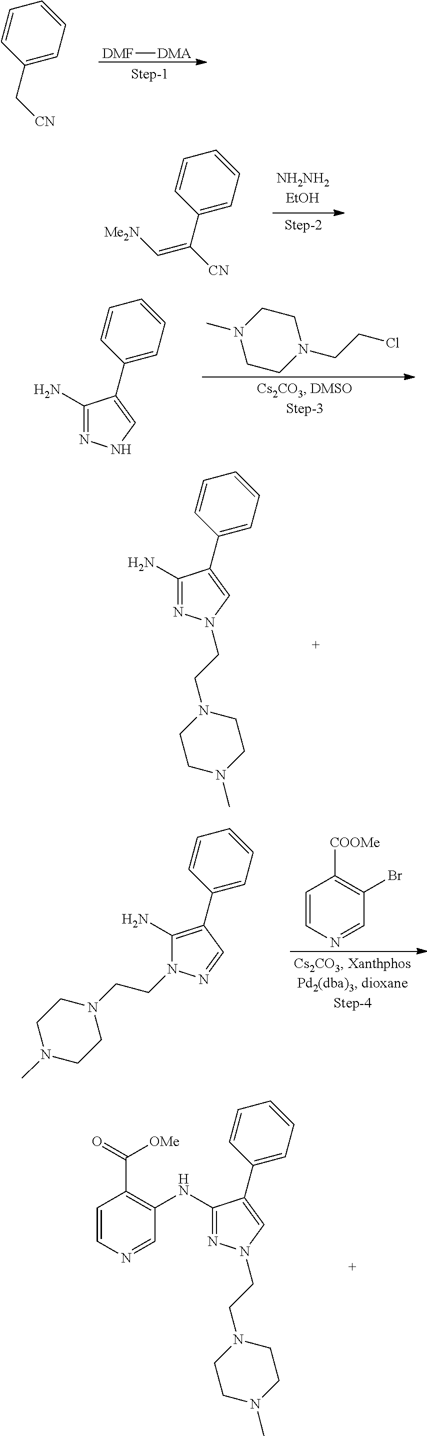

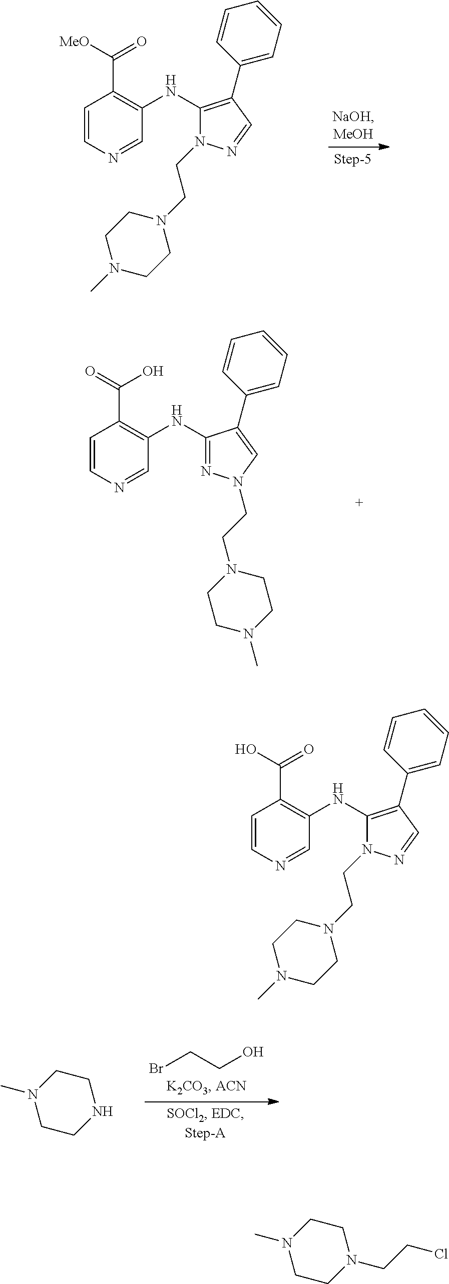

Compounds 34 and 35 were synthesized by a method similar to that illustrated in paragraph [0319] above except that the starting compound was 1-(2-chloroethyl)-4-methylpiperazine.

### Synthesis of 1-(2-chloroethyl)-4-methylpiperazine

To a stirred solution of 1-methylpiperazine (3 g, 30 mmol) and 2-bromoethanol (7.5 g, 60 mmol) in ACN (30 mL), K₂CO₃ (12.44 g, 90 mmol) was added and reaction was stirred at 75° C. for 16 h. Progress of the reaction was monitored by TLC. Upon completion the reaction mixture was filtered and the filtrate was concentrated under reduced pressure. The crude compound was dissolved in dichloroethane (30 mL), and SOCl₂ (15 mL) was added and stirred at 80° C. for 16 h. Progress of the reaction was monitored by TLC. Upon completion the reaction mixture was concentrated under reduced pressure to obtain a residue which was triturated with diethyl ether and pentane, filtered and dried under reduced pressure to afford the title compound (3.5 g, crude).

### Synthesis of 3-((1-Ethyl-3-phenyl-1H-pyrazol-5-yl)amino)isonicotinic acid (Compound 36)

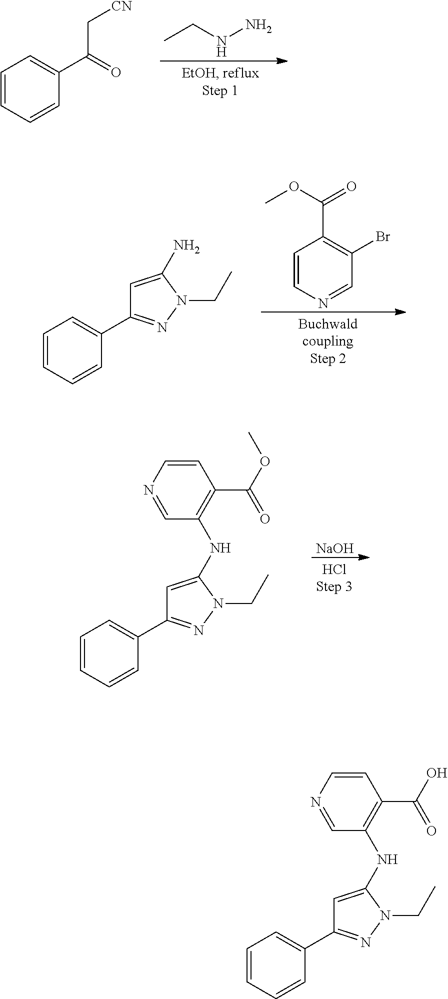

Compound 36 was synthesized by a method similar to that illustrated in paragraph [0340] above except that the starting compound was ethyl hydrazine.

### Synthesis of 3-((1-((cis)-4-aminocyclohexyl)-3-phenyl-1H-pyrazol-5-yl) amino)isonicotinic acid and 3-((1-((trans)-4-aminocyclohexyl)-3-phenyl-1H-pyrazol-5-yl)amino)isonicotinic acid (Compounds 37 and 38)

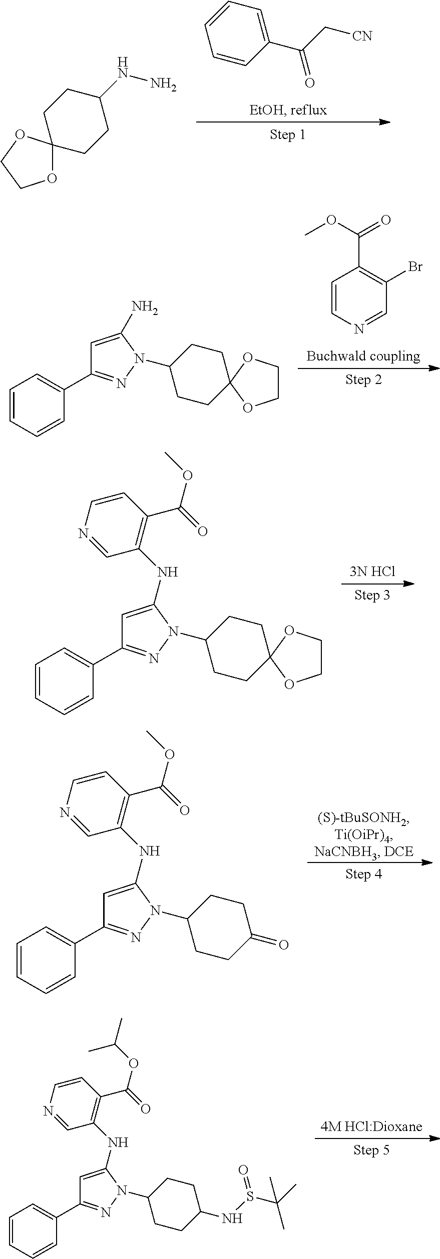

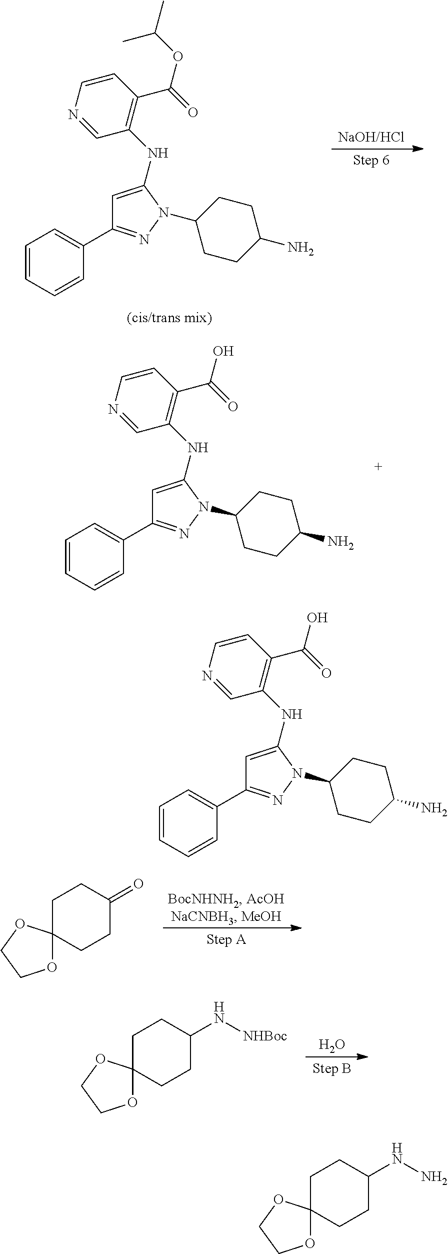

### Synthesis of tert-butyl 2-(1, 4-dioxaspiro [4.5] decan-8-yl) hydrazinecarboxylate

To a stirred solution of 1, 4-dioxaspiro [4.5] decan-8-one (5 g, 32.01 mmol), tert-butyl carbazate (4.23 g, 32.01 mmol), in methanol (140 mL) was added and stirred at room temperature for 20 h. After 20 h reaction mixture was concentrated under reduced pressure. To this residue Acetic acid (23.42 g, 390.59), H₂O (5 mL) and sodium cyanoborohydride (2 g, 32.01) was added and stirred at room temperature for 3 h. Progress of the reaction was monitored by TLC. Upon completion; the reaction was concentrated under reduced pressure and residue was basified by using 1N NaOH to PH=7 to 8. The residue was diluted with 10% MeOH/DCM. The organic layer was separated; aqueous layer was extracted with 10% MeOH/DCM. The combined organic layers were dried over anhydrous sodium sulfate and concentrated under reduced pressure. The crude compound was purified by column chromatography to afford the title compound (7.5 g, 86.1%).

### Synthesis of 1, 4-dioxaspiro [4.5] decan-8-ylhydrazine

To a stirred solution of tert-butyl 2-(1, 4-dioxaspiro [4.5] decan-8-yl) hydrazinecarboxylate (3 g, 11.01 mmol) in H₂O (25 mL) was added. The resulting reaction mixture was stirred at 100° C. for 12 h. Progress of the reaction was monitored by TLC and LCMS. Upon completion the reaction mixture was evaporated to dryness under reduced pressure. To this crude residue 10% MeOH/DCM was added, and the organic layer was evaporated to dryness under reduced pressure to afford title compound (1.2 g, 63.49%).

### Synthesis of 3-phenyl-1-(1, 4-dioxaspiro [4.5] decan-8-yl)-1H-pyrazol-5-amine

To a stirred solution of 1, 4-dioxaspiro [4.5] decan-8-ylhydrazine (1.1 g, 6.38 mmol), 3-oxo-3-phenylpropanenitrile (0.927 g, 6.38 mmol) in EtOH (20 mL) was added. The resulting reaction mixture was stirred at 100° C. for 12 h. Progress of the reaction was monitored by TLC and LCMS. Upon completion the reaction mixture was evaporated to dryness under reduced pressure, water was added and solid precipitated was filtered dried to afford title compound (1 g, 52.63%).

### Synthesis of methyl 3-((3-phenyl-1-(1, 4-dioxaspiro [4.5] decan-8-yl)-1H-pyrazol-5-yl) amino) isonicotinate

The standard Buchwald coupling protocol from paragraph [0306] above was followed (0.950 g, 65.51%).

### Synthesis of methyl 3-((1-(4-oxocyclohexyl)-3-phenyl-1H-pyrazol-5-yl) amino) isonicotinate

To a stirred solution of methyl 3-((3-phenyl-1-(1, 4-dioxaspiro [4.5] decan-8-yl)-1H-pyrazol-5-yl) amino) isonicotinate (1 g, 2.56 mmol), 3 M HCl (10 mL) in THF (20 mL) was added. The resulting reaction mixture was stirred at room temperature for 4 h. Progress of the reaction was monitored by TLC and LCMS. Upon completion the reaction mixture was neutralized with NaHCO₃ and the residue was taken in ethyl acetate, washed with water, dried over anhydrous sodium sulfate and evaporated under reduced pressure to afford the title compound as crude which was used as such for next step (0.915 g).

### Synthesis of Isopropyl 3-((1-(4-(1, 1-dimethylethylsulfinamido)cyclohexyl)-3-phenyl-1H-pyrazol-5-yl) amino) isonicotinate

To a stirred solution of methyl 3-((3-phenyl-1-(1,4-dioxaspiro[4.5]decan-8-yl)-1H-pyrazol-5-yl)amino) isonicotinate (0.1 g, 0.25 mmol), (S)-tBuSONH₂ (0.046 g, 0.38 mmol), in DCE (2 mL) was added and Ti(OiPr)₄ (0.37 mL, 1.28 mmol) at 0° C. and stirred at 60° C. for 12 h. Sodium triacetoxyborohydride (0.162 g, 0.76 mmol) was added at 0° C. and stirred at 60° C. for 1 h; LCMS showed complete imine formation. To this reaction mixture sodium cyanoborohydride (0.48 g, 0.76 mmol) was added and stirred at room temperature to 60° C. for 12 h. Progress of the reaction was monitored by TLC. Upon completion the reaction was quenched with saturated solution of NaHCO₃ and filtered through a bed of celite. The filtrate was diluted with 10% MeOH/DCM. The organic layer was separated; aqueous layer was extracted with 10% MeOH/DCM. The combined organic layers were dried over anhydrous sodium sulfate and concentrated under reduced pressure. The crude compound was purified by column chromatography to afford the title compound (0.1 g, 74.6%).

### Synthesis of isopropyl 3-((1-(4-aminocyclohexyl)-3-phenyl-1H-pyrazol-5-yl) amino) isonicotinate

To a stirred solution of isopropyl 3-((1-(4-(1,1-dimethylethylsulfinamido)cyclohexyl)-3-phenyl-1H-pyrazol-5-yl)amino)isonicotinate (0.5 g, 0.95 mmol) in dioxane (3 mL), HCl.dioxane (4M, 10 mL) was added and the reaction mixture was stirred at room temperature for 1 h. Progress of the reaction was monitored by TLC. Upon completion the reaction mixture was evaporated under reduced pressure to obtain a residue which was triturated with diethyl ether and pentane, filtered and dried under reduced pressure to afford the title compound (0.300 g, 75%).

### Synthesis of 3-((1-((cis)-4-aminocyclohexyl)-3-phenyl-1H-pyrazol-5-yl) amino)isonicotinic acid and 3-((1-((trans)-4-aminocyclohexyl)-3-phenyl-1H-pyrazol-5-yl)amino)isonicotinic acid (Compounds 37 and 38)

The standard NaOH hydrolysis procedure from paragraph [0307] above was followed. The crude compound was purified by prep HPLC to afford two isomers.

### Synthesis of 3-((1-((cis)-4-Hydroxycyclohexyl)-3-phenyl-1H-pyrazol-5-yl) amino)isonicotinic acid and 3-((1-((trans)-4-hydroxycyclohexyl)-3-phenyl-1H-pyrazol-5-yl) amino)isonicotinic acid (Compounds 40 and 39)

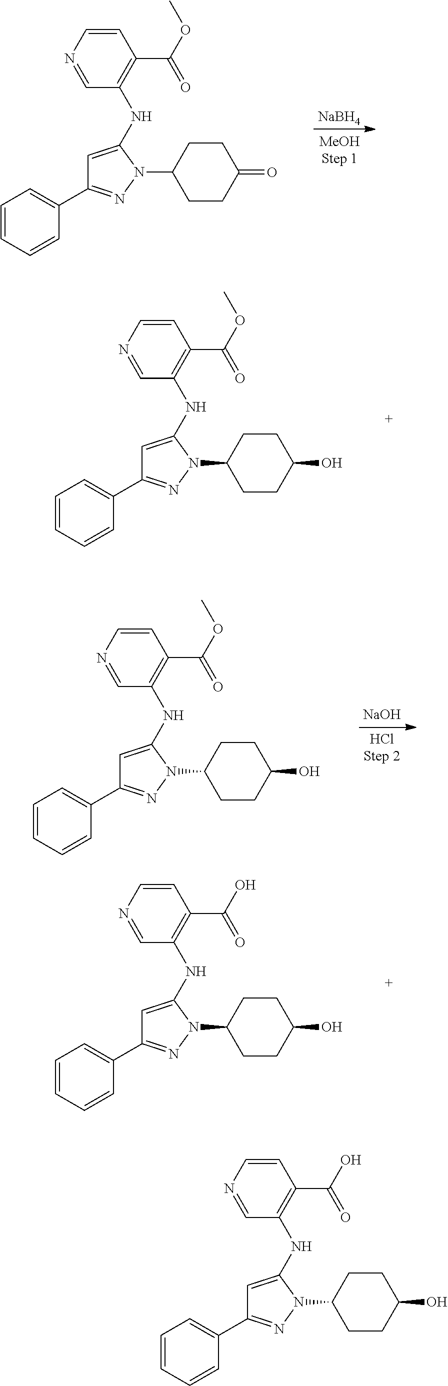

### Synthesis of methyl 3-((1-(4-hydroxycyclohexyl)-3-phenyl-1H-pyrazol-5-yl) amino)isonicotinate

To a stirred solution of methyl 3-((1-(4-oxocyclohexyl)-3-phenyl-1H-pyrazol-5-yl)amino)isonicotinate (0.45 g, 1.15 mmol) in MeOH (10 mL) at 0° C., NaBH₄ (0.022 g, 0.576 mmol) was added and the reaction was stirred at room temperature for 45 min. Progress of the reaction was monitored by TLC. Upon completion the reaction mixture was quenched with aqueous sat. NH₄Cl solution and concentrated to dryness under reduced pressure. The residue was diluted with water and extracted with ethyl acetate. Combined organic layers were dried over anhydrous sodium sulfate and concentrated under reduced pressure. The crude compound was purified by column chromatography to afford the title compound (cis isomer: 0.08 g, 18%, and trans isomer: 44.2%).

### Synthesis of 3-((1-((cis)-4-Hydroxycyclohexyl)-3-phenyl-1H-pyrazol-5-yl) amino)isonicotinic acid and 3-((1-((trans)-4-Hydroxycyclohexyl)-3-phenyl-1H-pyrazol-5-yl)amino)isonicotinic acid (Compounds 40 and 39)

The standard NaOH hydrolysis procedure from paragraph [0307] above was followed. (cis isomer: 0.034 g, 43%; trans isomer: 0.062 g).

### Synthesis of 3-((1,3-Diphenyl-1H-pyrazol-4-yl)amino)isonicotinic acid (Compound 41)

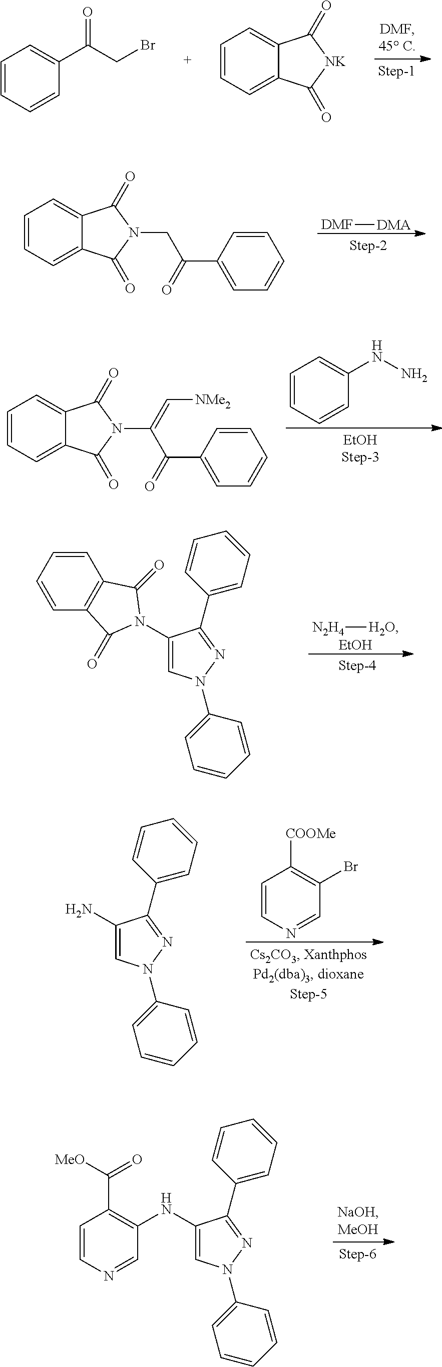

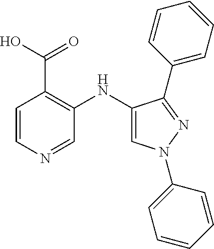 [SMILES: O=C(O)c1ccncc1Nc1cn(-c2ccccc2)nc1-c1ccccc1]

Compound 41 was synthesized by a method similar to that illustrated in paragraph [0313] above except that the starting compound was phenyl hydrazine.

### Synthesis of 3-((1, 4-diphenyl-1H-pyrazol-3-yl)amino)isonicotinic acid (Compound 42)

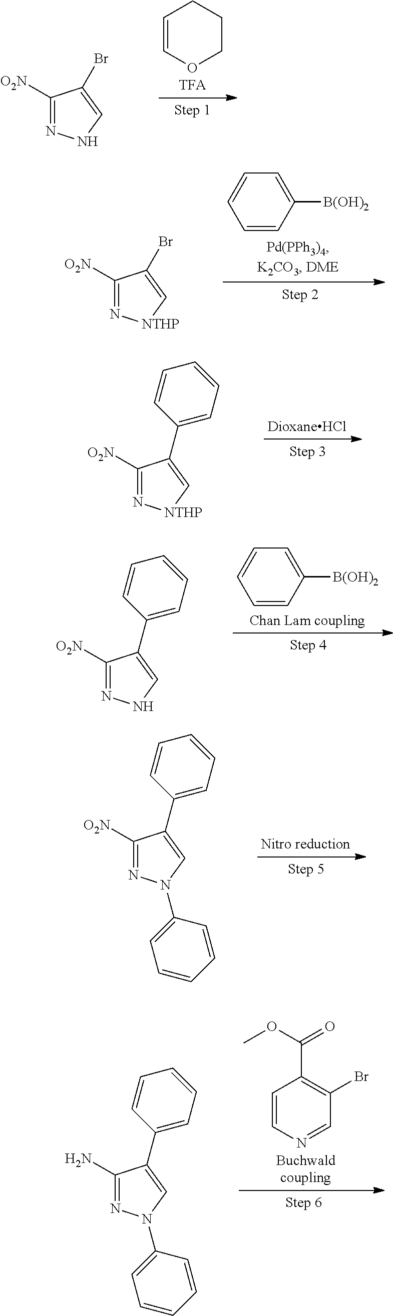

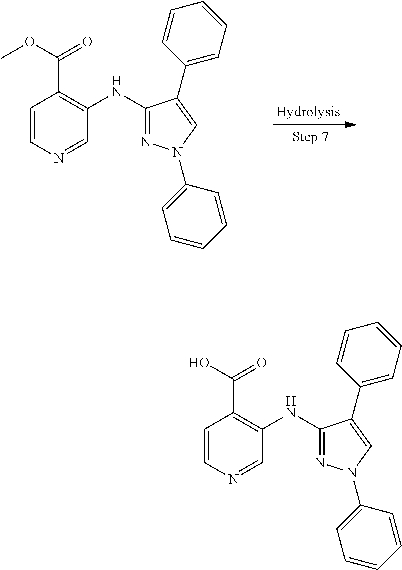 [SMILES: COC(=O)c1ccncc1Nc1nn(-c2ccccc2)cc1-c1ccccc1 ; O=C(O)c1ccncc1Nc1nn(-c2ccccc2)cc1-c1ccccc1]

### Synthesis of 4-bromo-3-nitro-1-(tetrahydro-2H-pyran-2-yl)-1H-pyrazole

To a stirred solution of 4-bromo-3-nitro-1H-pyrazole (5 g, 26.17 mmol), 3, 4-dihydro-2H-pyran (2.41 g, 28.79 mmol) in toluene (50 mL) and TFA (0.289 g, 2.61 mmol) was added and stirred at 110° C. for 4 h. Progress of the reaction was monitored by TLC. Upon completion the reaction mixture was diluted with ethyl acetate and saturated sodium bicarbonate and organic layer separated. The combined organic layers were dried over anhydrous sodium sulfate and concentrated under reduced pressure. The crude compound was purified by column chromatography to afford the title compound (6.62 g, 92.07%).

### Synthesis of 3-nitro-4-phenyl-1-(tetrahydro-2H-pyran-2-yl)-1H-pyrazole

To a stirred solution of 4-methyl-3-nitro-1H-pyrazole (1.5 g, 5.45 mmol), phenylboronic acid (0.864 g, 7.09 mmol) in 1,2-DME (10 mL) was added and purged with argon for 10 min. Pd [(C₆H₅)₃P]₄ (0.944 g, 0.87 mmol) and K₂CO₃ (6 mL, 1.5 g, 10.9 mmol) was added and the solution was purged with argon for additional 15 min and stirred at 85° C. for 15 h. Progress of the reaction was monitored by TLC. Upon completion the reaction mixture was filtered through a bed of celite and evaporated to dryness. The residue was taken in ethyl acetate, washed with water, brine, dried over anhydrous sodium sulfate and evaporated under reduced pressure. The crude product was purified by column chromatography to afford the title compound (1.3 g, 87.83%).

### Synthesis of 3-nitro-4-phenyl-1H-pyrazole hydrochloride

To a stirred solution of 3-nitro-4-phenyl-1-(tetrahydro-2H-pyran-2-yl)-1H-pyrazole (1.2 g, 4.39 mmol) in minimum dioxane was added HCl.dioxane (4M, 15 mL) and the solution was stirred at room temperature for 2 h. Progress of the reaction was monitored by TLC. Upon completion the reaction mixture was evaporated to dryness. The crude compound was used without purification for next step (0.520 g).

### Synthesis of 3-nitro-1, 4-diphenyl-1H-pyrazole

To a stirred solution of 3-nitro-4-phenyl-1H-pyrazole hydrochloride (0.5 g, 2.26 mmol) in DCM (10 mL), pyridine (0.822 g, 9.04 mmol), phenylboronic acid (0.359 g, 2.94 mmol) and Cu(OAc)₂ (0.615 g, 3.39 mmol) were added and the reaction was stirred at room temperature for 16 h. Progress of the reaction was monitored by TLC. Upon completion the reaction mixture was filtered through a bed of celite and evaporated to dryness. The residue was taken in ethyl acetate, washed with water, brine, dried over anhydrous sodium sulfate and evaporated under reduced pressure. The crude product was purified by column chromatography to afford impure compound (0.4 g, 66.56%).

### Synthesis of 1, 4-diphenyl-1H-pyrazol-3-amine

To a stirred solution of 3-nitro-1, 4-diphenyl-1H-pyrazole (0.5 g, 1.88 mmol) in MeOH (15 mL), 10% Pd—C (0.1 g) was added and the reaction was stirred under hydrogen atmosphere (balloon pressure) at room temperature for 3 h. Progress of the reaction was monitored by TLC. Upon completion the reaction mixture was filtered through a bed of celite and evaporated to dryness to afford the crude compound which was used without purification for next step (0.4 g).

### Synthesis of methyl 3-((1, 4-diphenyl-1H-pyrazol-3-yl) amino) isonicotinate

The standard Buchwald coupling protocol from paragraph [0306] above was followed (0.2 g, 31.75%).

### Synthesis of 3-((1, 4-diphenyl-1H-pyrazol-3-yl) amino) isonicotinic acid (Compound 42)

The standard NaOH hydrolysis procedure from paragraph [0307] above was followed (0.13 g, 76.91%).

### Synthesis of 3-((1,4-Diphenyl-1H-pyrazol-5-yl)amino)isonicotinic acid (Compound 43)

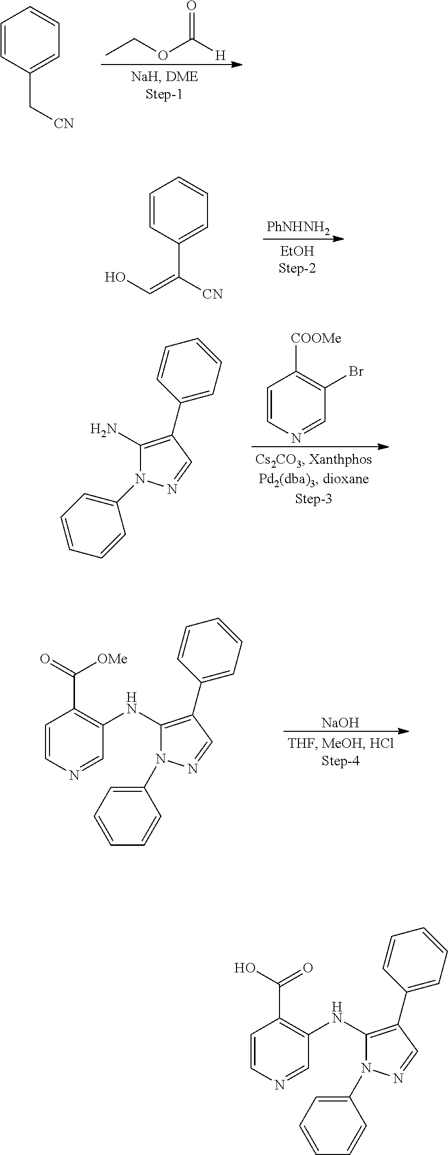

### Synthesis of (E)-3-Hydroxy-2-phenylacrylonitrile

To a stirred solution of NaH (10.2 g, 256 mmol) in 1,2 dimethoxy ethane (150 mL) at 0° C., (2-phenylacetonitrile (20 g, 170 mmol) and ethyl formate (18 mL, 221 mmol) was added slowly. The resulting reaction mixture was stirred at 60° C. for 4 h. Progress of the reaction was monitored by TLC. Upon completion the reaction mixture was quenched with water and acidified with 1N HCl. The solid was filtered, dried over anhydrous sodium sulfate, and concentrated under reduced pressure to afford the title compound (15 g, 60.5%).

### Synthesis of 1,4-Diphenyl-1H-pyrazol-5-amine

To a stirred solution of (E)-3-hydroxy-2-phenylacrylonitrile (0.5 g, 3.44 mmol) in EtOH (10 mL), phenyl hydrazine (0.67 mL, 6.89 mmol) and acetic acid (0.29 mL, 5.16 mmol) was added. The resulting reaction mixture was stirred at room temperature for 15 h. Progress of the reaction was monitored by TLC. Upon completion the reaction mixture was concentrated to dryness under reduced pressure. The crude compound was purified by column chromatography to afford the title compound (0.6 g, 74%).

### Synthesis of Methyl 3-((1,4-diphenyl-1H-pyrazol-5-yl)amino)isonicotinate

The standard Buchwald coupling protocol from paragraph [0306] above was followed (0.41 g, 47.4%).

### Synthesis of 3-((1,4-diphenyl-1H-pyrazol-5-yl)amino)isonicotinic acid (Compound 43)

The standard NaOH hydrolysis procedure from paragraph [0307] above was followed (0.187 g, 65.1%).

### Synthesis of 3-((1-methyl-3-(pyridin-4-yl)-1H-pyrazol-5-yl)amino) isonicotinic acid (Compound 44)

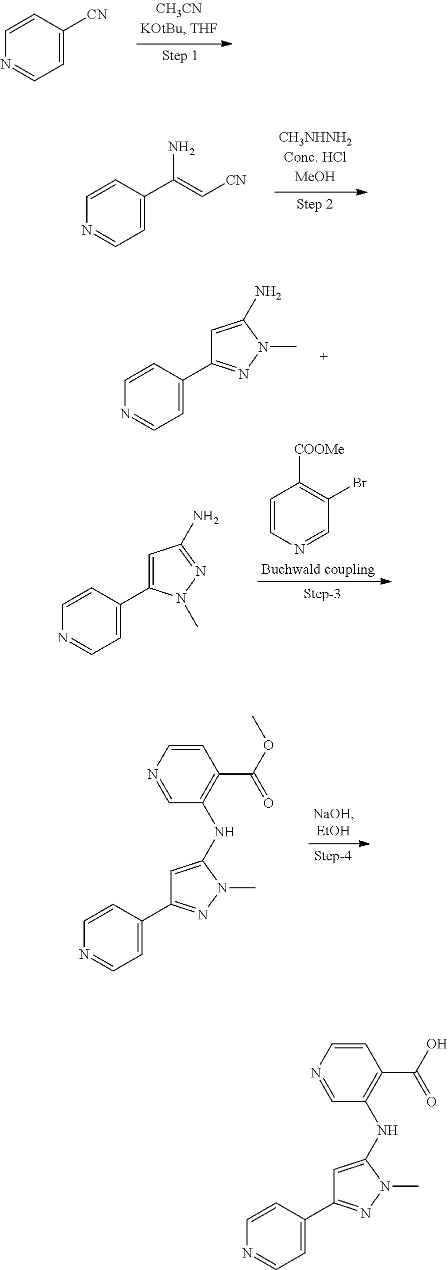

### Synthesis of (Z)-3-amino-3-(pyridin-4-yl) acrylonitrile

To a stirred solution of isonicotinonitrile (4 g, 38.46 mmol) in acetonitrile (5.2 mL, 96.15 mmol) and THF (30 mL) was added potassium tert-butoxide (17.2 g, 153.84 mmol) at 0° C. portionwise, in 5 lots over a period of 20 min. Upon complete addition, the reaction was stirred at room temperature for 30 min. Progress of the reaction was monitored by TLC. Upon completion the reaction mixture was quenched with water and taken in dichloromethane, washed with water, dried over anhydrous sodium sulfate and evaporated under reduced pressure. The crude product was purified by column chromatography to afford title compound (2 g, 36.36%).

### Synthesis of 1-methyl-3-(pyridin-4-yl)-1H-pyrazol-5-amine and 1-methyl-5-(pyridin-4-yl)-1H-pyrazol-3-amine

To a stirred solution of (Z)-3-amino-3-(pyridin-4-yl) acrylonitrile (2 g, 13.79 mmol) in MeOH (10 mL), HCl was added drop wise at 0° C. followed by addition of Methylhydrazine (3.8 g, 82.75 mmol) and reaction mixture was stirred at 80 0° C. for 2 h. Progress of the reaction was monitored by TLC. Upon completion the reaction mixture was evaporated under reduced pressure resulting in a crude compound which was purified by column chromatography to afford mixture of title compound (0.5 g), (Note: Bias towards one isomer).

### Synthesis of Methyl 3-((1-methyl-3-(pyridin-4-yl)-1H-pyrazol-5-yl) amino) isonicotinate

The standard Buchwald coupling protocol from paragraph [0306] above was followed (0.140 g, 39.54%).

### Synthesis of 3-((1-methyl-3-(pyridin-4-yl)-1H-pyrazol-5-yl) amino) isonicotinic acid (Compound 44)

The standard NaOH hydrolysis procedure from paragraph [0307] above was followed (0.054 g, 56.84%).

### Synthesis of 3-((1-(2-(4, 4-difluoropiperidin-1-yl) ethyl)-4-(4-fluorophenyl)-1H-pyrazol-3-yl) amino)isonicotinic acid and 3-((1-(2-(4,4-difluoropiperidin-1-yl) ethyl)-4-(4-fluorophenyl)-1H-pyrazol-5-yl) amino) isonicotinic acid

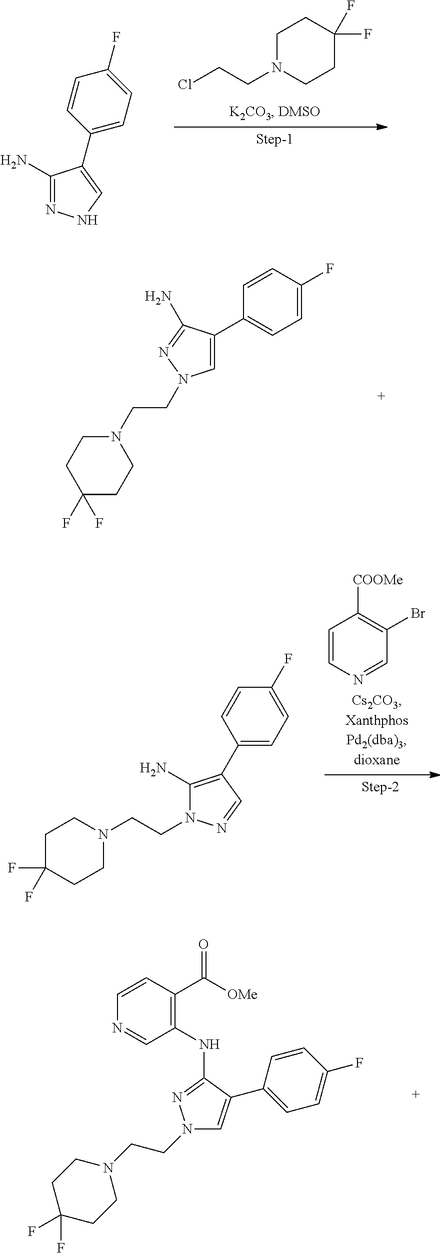

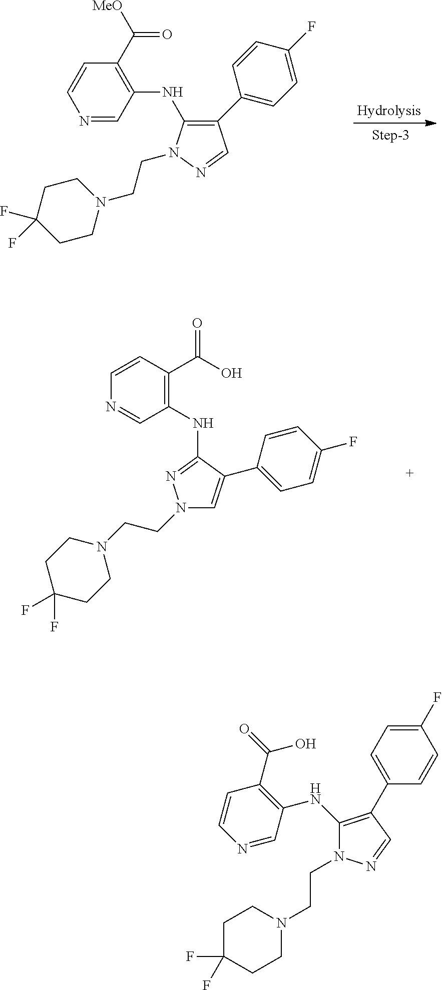 [SMILES: COC(=O)c1ccncc1Nc1c(-c2ccc(F)cc2)cnn1CCN1CCC(F)(F)CC1 ; O=C(O)c1ccncc1Nc1nn(CCN2CCC(F)(F)CC2)cc1-c1ccc(F)cc1 ; O=C(O)c1ccncc1Nc1c(-c2ccc(F)cc2)cnn1CCN1CCC(F)(F)CC1]

3-((1-(2-(4, 4-difluoropiperidin-1-yl) ethyl)-4-(4-fluorophenyl)-1H-pyrazol-3-yl) amino)isonicotinic acid and 3-((1-(2-(4,4-difluoropiperidin-1-yl) ethyl)-4-(4-fluorophenyl)-1H-pyrazol-5-yl) amino) isonicotinic acid were synthesized by a method similar to that illustrated in paragraph [0319] above except that the starting compound was 1-(2-chloroethyl)-4,4-difluoropiperidine.

### Synthesis of 3-((5-methyl-1-(p-tolyl)-1H-pyrazol-4-yl)amino) isonicotinic acid (Compound 47)

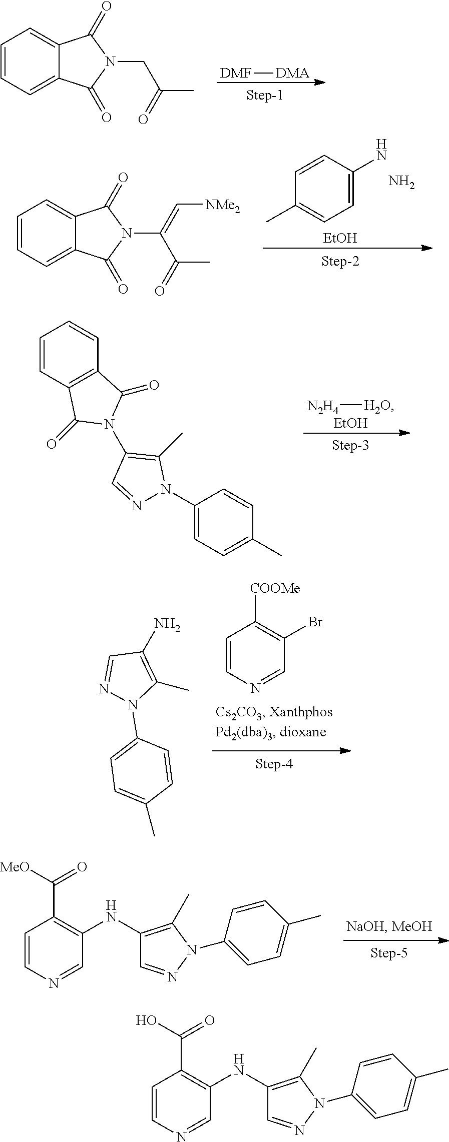

Compound 47 was synthesized by a method similar to that illustrated in paragraph [0313] above except that the starting compounds were 2-(2-oxopropyl)isoindoline-1,3-dione and p-tolylhydrazine.

### Synthesis of 3-((5-methyl-1-(m-tolyl)-1H-pyrazol-4-yl)amino)isonicotinic acid and 3-((3-methyl-1-(m-tolyl)-1H-pyrazol-4-yl) amino) isonicotinic acid (Compounds 49 and 48)

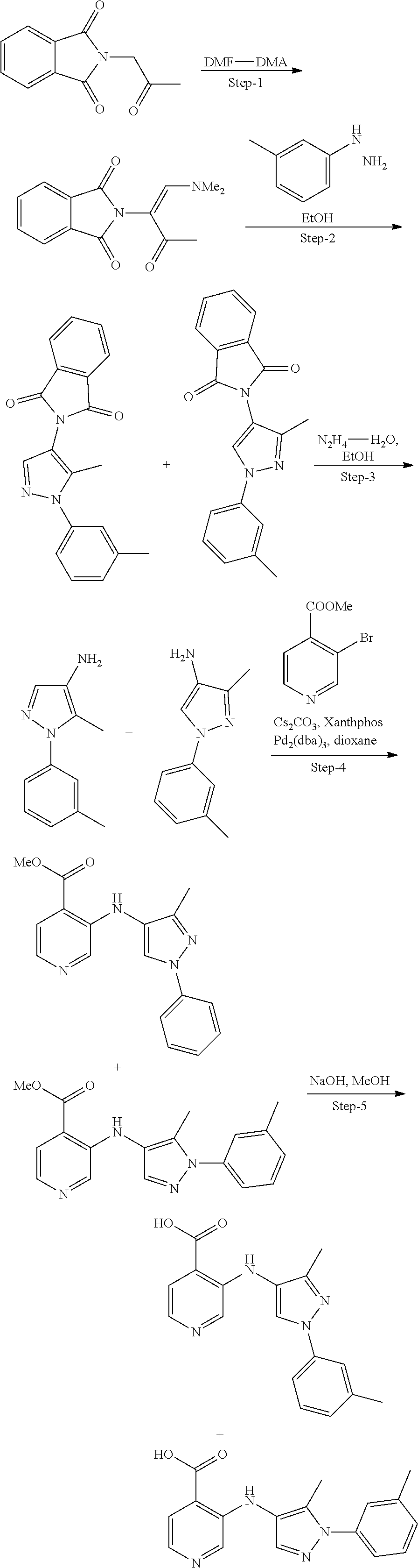

Compounds 48 and 49 were synthesized by a method similar to that illustrated in paragraph [0313] above except that the starting compounds were 2-(2-oxopropyl)isoindoline-1,3-dione and m-tolylhydrazine.

### Synthesis of 3-((5-methyl-1-(o-tolyl)-1H-pyrazol-4-yl)amino)isonicotinic acid (Compound 50)

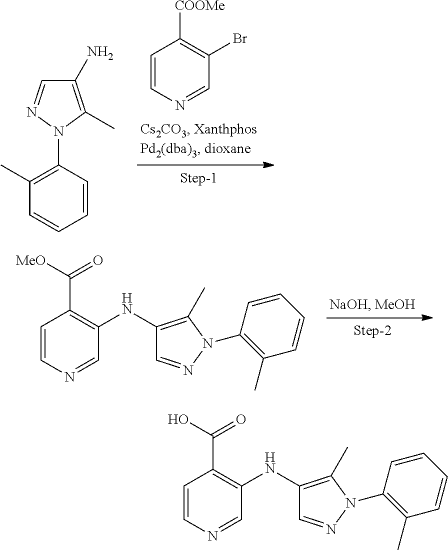

Compound 50 was synthesized by a method similar to that illustrated in paragraph [0313] above except that the starting compounds were 2-(2-oxopropyl)isoindoline-1,3-dione and o-tolylhydrazine.

### Synthesis of 3-((1-(3-(dimethylamino) propyl)-4-phenyl-1H-pyrazol-3-yl) amino) isonicotinic acid and 3-((1-(3-(dimethylamino) propyl)-4-phenyl-1H-pyrazol-5-yl) amino) isonicotinic acid (Compounds 51 and 52)

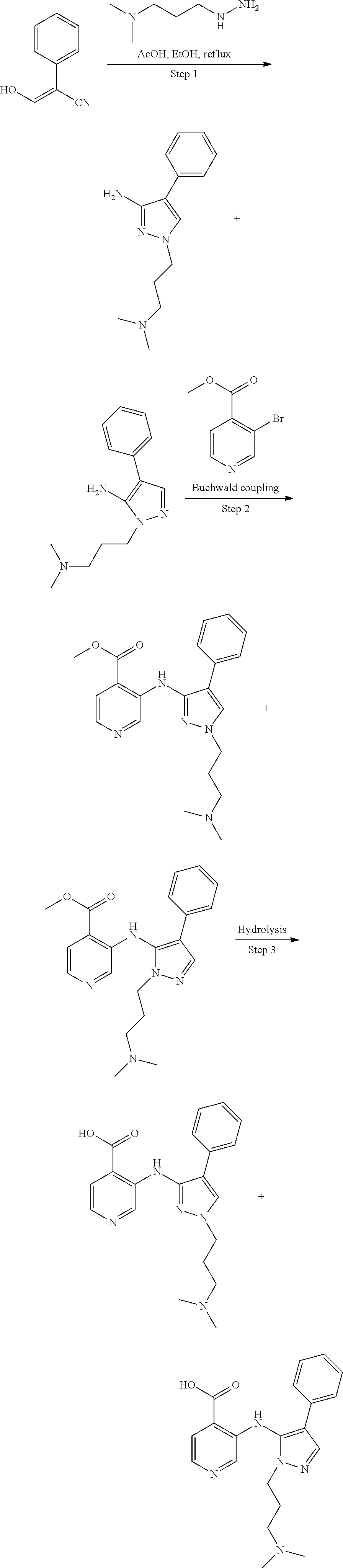

Compounds 51 and 52 were synthesized by a method similar to that illustrated in paragraph [0396] above except that the starting compound was 3-hydrazinyl-N,N-dimethylpropan-1-amine.

### Synthesis of 3-((1, 4-dimethyl-3-phenyl-1H-pyrazol-5-yl)amino)isonicotinic acid and 3-((1, 4-dimethyl-5-phenyl-1H-pyrazol-3-yl)amino)isonicotinic acid (Compounds 53 and 54)

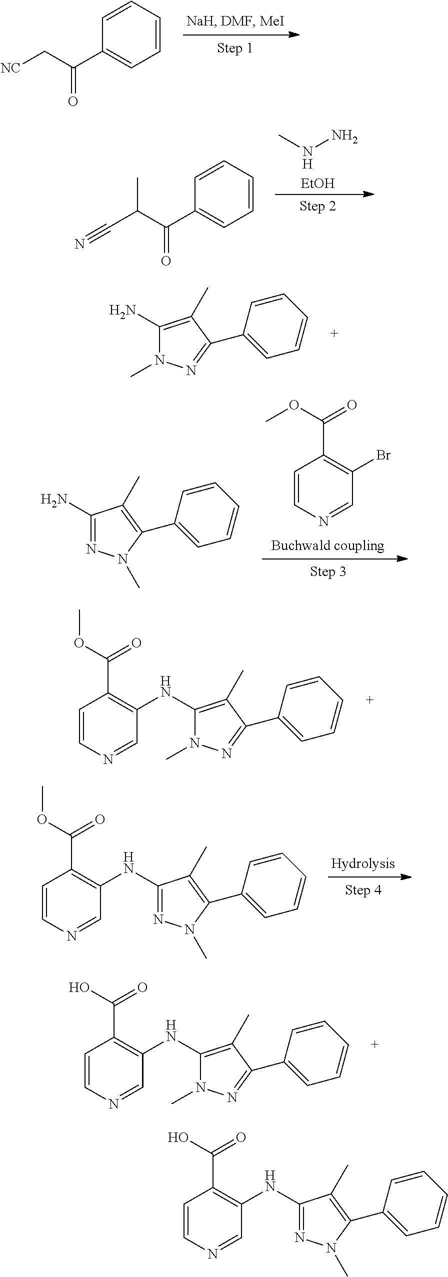

### Synthesis of 2-methyl-3-oxo-3-phenylpropanenitrile

To a solution of 3-oxo-3-phenylpropanenitrile (3 g, 20.68 mmol) in DMF, NaH (0.827 g, 20.68 mmol) was added at 0° C. and stirred for 15 min. Methyl iodide (2.93 g, 20.68 mmol) added at 0° C. and stirred at room temperature for 1 h. Progress of the reaction was monitored by TLC. Upon completion the reaction mixture was diluted with water and ethyl acetate. The combined organic layer was dried over anhydrous sodium sulfate and evaporated under reduced pressure. The crude product was purified by column chromatography to afford the title compound (1.2 g, 36.58%).

### Synthesis of 1, 4-dimethyl-3-phenyl-1H-pyrazol-5-amine and 1, 4-dimethyl-5-phenyl-1H-pyrazol-3-amine

To a stirred solution of 2-methyl-3-oxo-3-phenylpropanenitrile (0.6 g, 3.77 mmol) in EtOH (2 mL), methyl hydrazine (1.73 g, 3.77 mmol) was added and the reaction mixture was stirred at 90° C. for 12 h. Progress of the reaction was monitored by TLC. Upon completion the reaction mixture was evaporated under reduced pressure resulting in a crude compound which was purified by column chromatography to afford the mixture of title compounds (0.4 g).

### Synthesis of methyl 3-((1, 4-dimethyl-3-phenyl-1H-pyrazol-5-yl) amino) isonicotinate and methyl 3-((1, 4-dimethyl-5-phenyl-1H-pyrazol-3-yl) amino) isonicotinate

The standard Buchwald coupling protocol from paragraph [0306] above was followed (0.035 g, 5.07%).

### Synthesis of 3-((1, 4-dimethyl-3-phenyl-1H-pyrazol-5-yl) amino) isonicotinic acid and 3-((1,4-dimethyl-5-phenyl-1H-pyrazol-3-yl)amino) isonicotinic acid

The standard NaOH hydrolysis procedure from paragraph [0307] above was followed (0.09 g, 72.5%) and (0.03 g, 80.4%).

### Synthesis of 3-((5-methyl-3-phenyl-1H-pyrazol-4-yl)amino)isonicotinic acid (Compound 55)

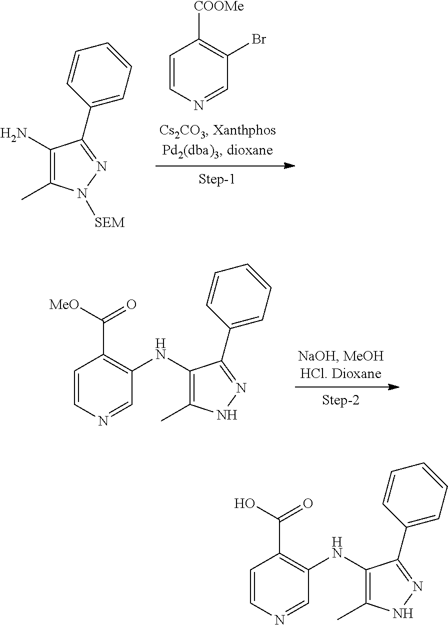

### Synthesis of methyl 3-((5-methyl-3-phenyl-1H-pyrazol-4-yl) amino) isonicotinate

The standard Buchwald coupling protocol from paragraph [0306] above was followed (0.035 g, 12.11%).

### Synthesis of 3-((5-methyl-3-phenyl-1H-pyrazol-4-yl) amino) isonicotinic acid

The standard NaOH hydrolysis procedure from paragraph [0307] above was followed (30 mg 90.9%).

### Synthesis of 3-((3-phenyl-1-(pyridin-4-yl)-1H-pyrazol-5-yl)amino) isonicotinic acid (Compound 56)

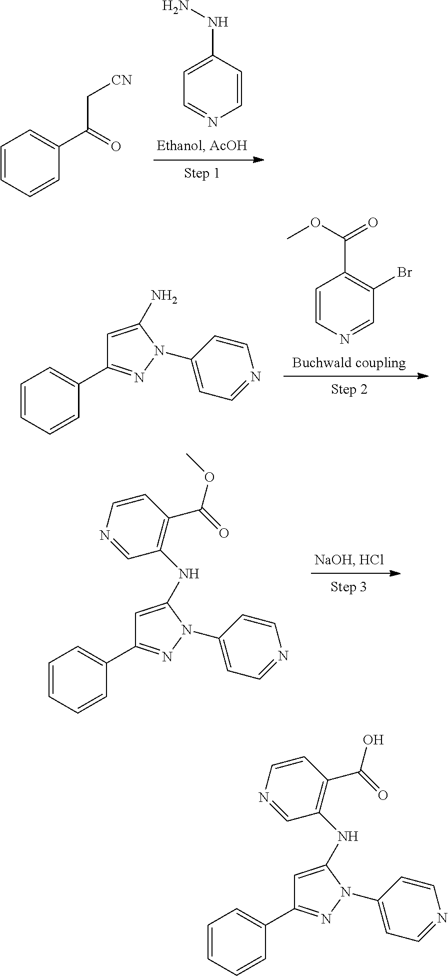 [SMILES: N#CCC(=O)c1ccccc1 ; NNc1ccncc1 ; Nc1cc(-c2ccccc2)nn1-c1ccncc1 ; COC(=O)c1ccncc1Br ; COC(=O)c1ccncc1Nc1cc(-c2ccccc2)nn1-c1ccncc1 ; O=C(O)c1ccncc1Nc1cc(-c2ccccc2)nn1-c1ccncc1]

Compound 56 was synthesized by a method similar to that illustrated in paragraph [0340] above except that the starting compound was 4-hydrazinylpyridine.

### Synthesis of 3-((4-cyclohexyl-1-(2-(4,4-difluoropiperidin-1-yl)ethyl)-1H-pyrazol-3-yl)amino)isonicotinic acid (Compound 57)

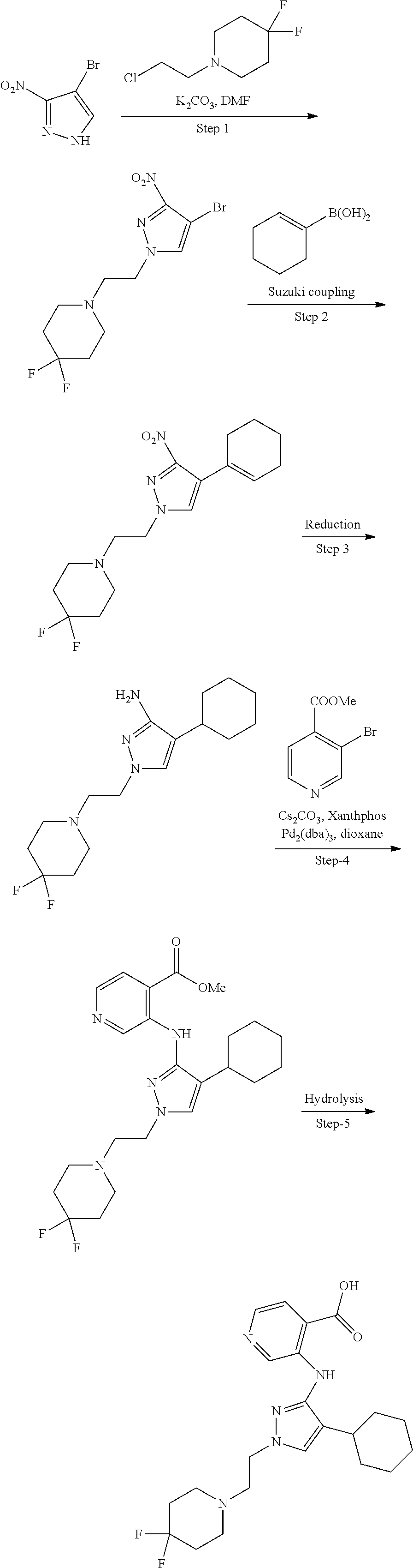

Compound 57 was synthesized by a method similar to that illustrated in paragraph [0388] above except that the starting compound was 1-(2-chloroethyl)-4,4-difluoropiperidine instead of THP protection.

### Synthesis of 3-((5-methyl-1-(4-(methylcarbamoyl) phenyl)-1H-pyrazol-4-yl) amino) isonicotinic acid (Compound 58)

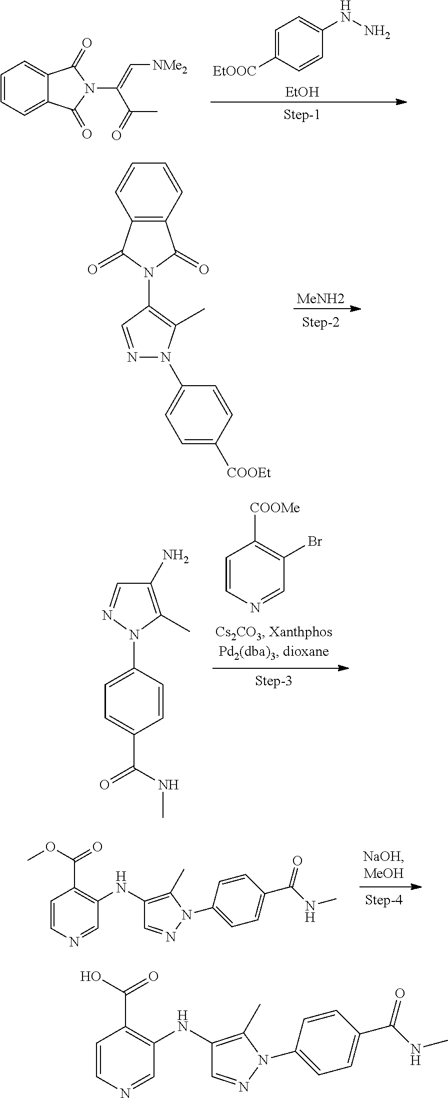

Compound 58 was synthesized by a method similar to that illustrated in paragraph [0313] above except that the starting compounds were 2-(2-oxopropyl)isoindoline-1,3-dione and ethyl 4-hydrazinylbenzoate.

### Synthesis of 3-((5-methyl-1-(3-(methylcarbamoyl) phenyl)-1H-pyrazol-4-yl) amino) isonicotinic acid (Compound 59)

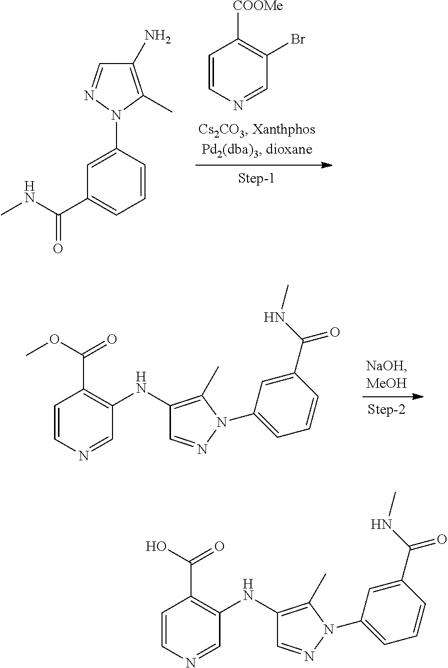

Compound 59 was synthesized by a method similar to that illustrated in paragraph [0313] above except that the starting compounds were 2-(2-oxopropyl)isoindoline-1,3-dione and ethyl 3-hydrazinylbenzoate.

### Synthesis of 3-((5-methyl-1-phenyl-1H-pyrazol-3-yl)amino)isonicotinic acid and 3-((3-methyl-1-phenyl-1H-pyrazol-5-yl) amino) isonicotinic acid (Compounds 60 and 61)

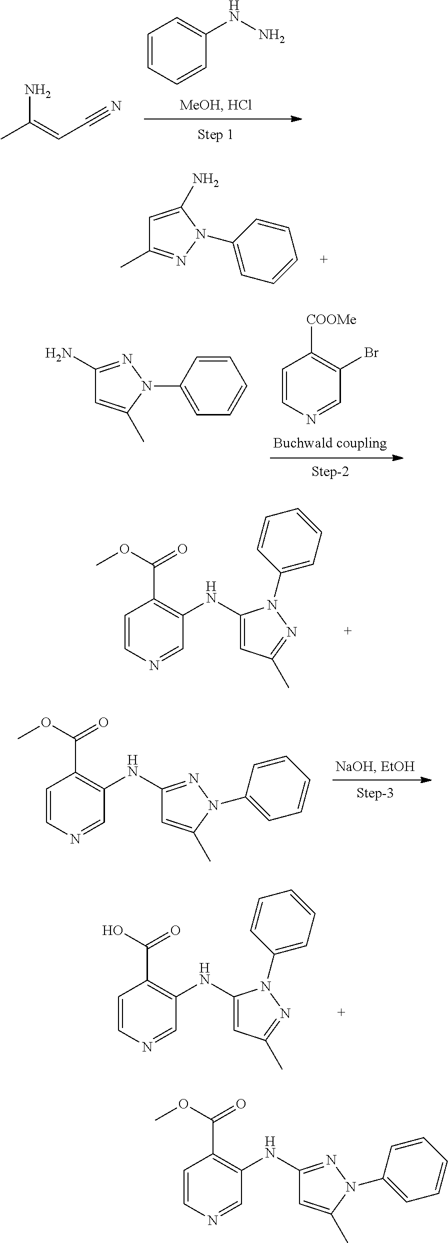

### Synthesis of 3-methyl-1-phenyl-1H-pyrazol-5-amine and 5-methyl-1-phenyl-1H-pyrazol-3-amine

To a solution of (Z)-3-aminobut-2-enenitrile (4 g, 48.78 mmol) in MeOH (20 mL), HCl (10 mL) at 0° C. and phenylhydrazine (3.1 g, 29.26 mmol) was added and heated at 90° C. for 2 h. Progress of the reaction was monitored by TLC. Upon completion the reaction mixture was evaporated under reduced pressure to obtain a residue. The crude product was purified by column chromatography to afford 3-methyl-1-phenyl-1H-pyrazol-5-amine (1 g, 15.62%) and 5-methyl-1-phenyl-1H-pyrazol-3-amine (0.9 g, 14.06%).

### Synthesis of methyl 3-((3-methyl-1-phenyl-1H-pyrazol-5-yl)amino) isonicotinate and methyl 3-((5-methyl-1-phenyl-1H-pyrazol-3-yl) amino)isonicotinate

The standard Buchwald coupling protocol from paragraph [0306] above was followed (0.14 g, 32.78%), (Note: Confirmed by NOE).

### Synthesis of 3-((3-methyl-1-phenyl-1H-pyrazol-5-yl) amino) isonicotinic acid and 3-((3-methyl-1-phenyl-1H-pyrazol-5-yl) amino) isonicotinic acid

The standard NaOH hydrolysis procedure from paragraph [0307] above was followed (0.075 g, 78.94%).

### Synthesis of 3-((4-methyl-1-phenyl-1H-pyrazol-3-yl) amino) isonicotinic acid (Compound 62)

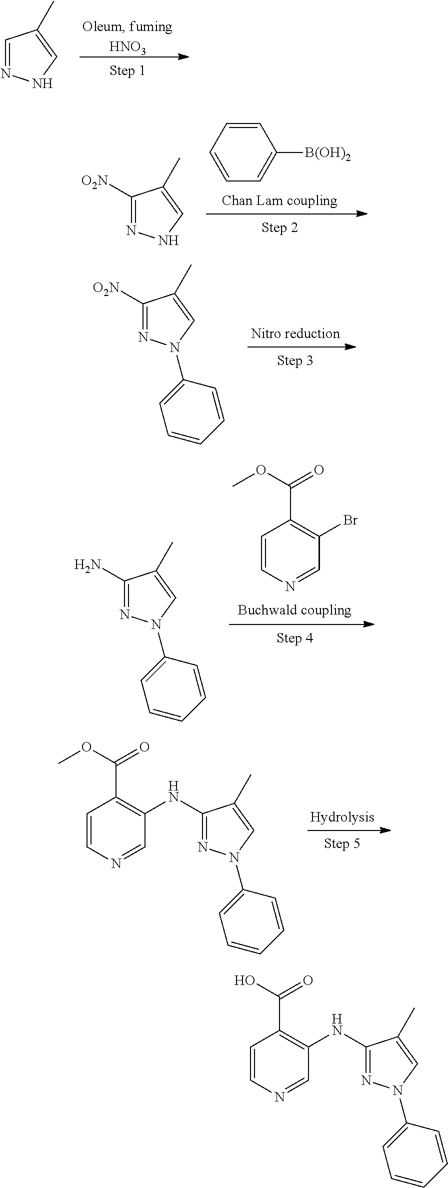

Compound 62 was synthesized by a method similar to that illustrated in paragraph [0388] above except that the starting compound was 4-methyl-3-nitro-1H-pyrazole.

### Synthesis of 4-methyl-3-nitro-1H-pyrazole

To a stirred solution of 4-methyl-1H-pyrazole (1 g, 12.19 mmol), H₂SO₄(15 mL), oleum (4.4 mL) at 30-40° C. and fuming nitric acid (0.64 g, 15.23 mmol) was added and stirred at 105° C. for 2 h. Progress of the reaction was monitored by TLC. Upon completion the reaction mixture was diluted with water and purged with air for 30 min, and extracted with ethyl acetate, and washed with water and brine. Combined organic layers were dried over anhydrous sodium sulfate and concentrated under reduced pressure. The crude compound was purified by column chromatography to afford the title compound (0.85 g, 55%).

### Synthesis of 3-((1-(2-methoxyphenyl)-4-methyl-1H-pyrazol-3-yl) amino) isonicotinic acid (Compound 63)

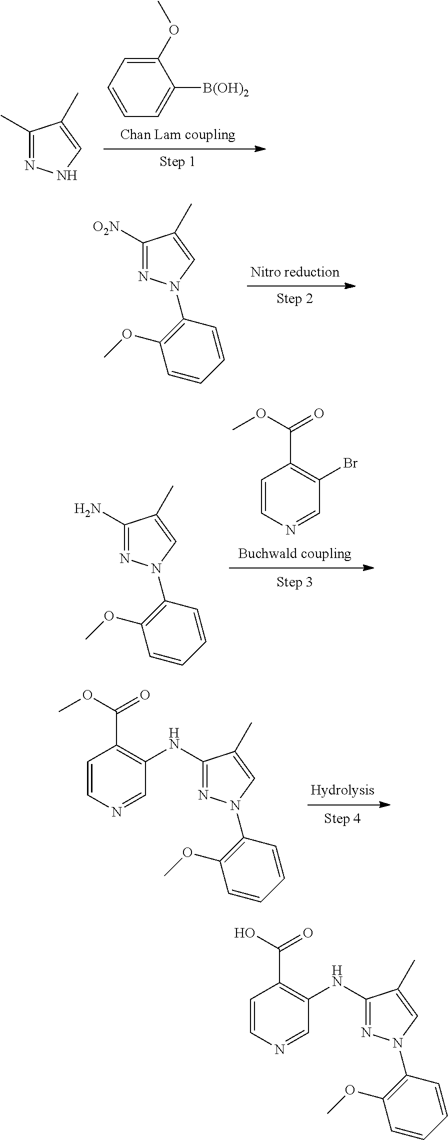

Compound 63 was synthesized by a method similar to that illustrated in paragraph [0388] above except that the starting compounds were (2-methoxyphenyl)boronic acid and 4-methyl-3-nitro-1H-pyrazole.

### Synthesis of 3-((1-(2-(dimethylamino) ethyl)-4-phenyl-1H-pyrazol-3-yl) amino)isonicotinic acid and 3-((1-(2-(dimethylamino) ethyl)-4-phenyl-1H-pyrazol-5-yl) amino)isonicotinic acid (Compounds 64 and 65)

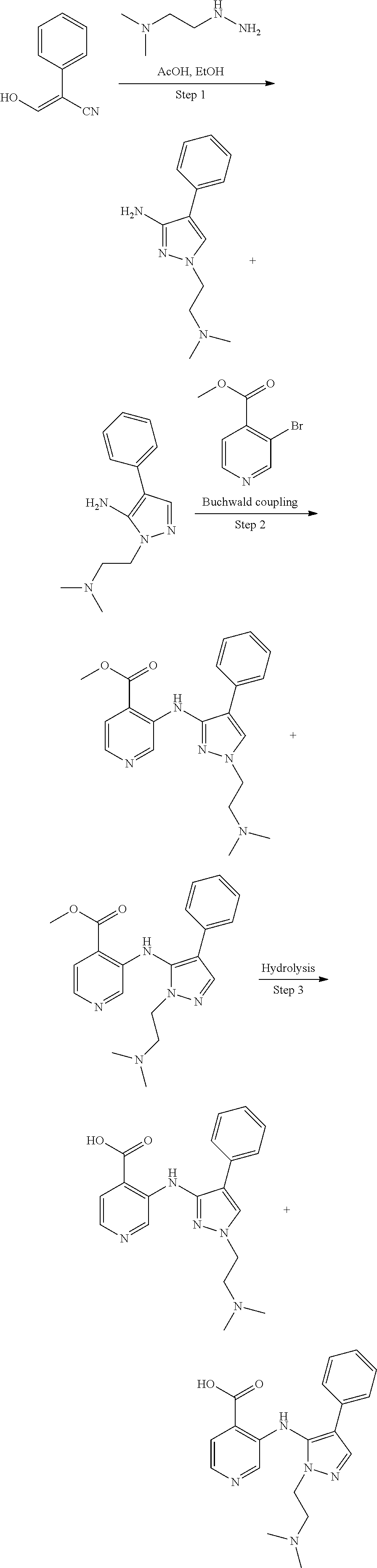

Compounds 64 and 65 were synthesized by a method similar to that illustrated in paragraph [0396] above except that the starting compound was 2-hydrazinyl-N,N-dimethylethan-1-amine.

### Synthesis of 3-((1-(2-(4,4-difluoropiperidin-1-yl) ethyl)-4-methyl-3-phenyl-1H-pyrazol-5-yl)amino)isonicotinic acid and 3-((1-(2-(4,4-difluoropiperidin-1-yl) ethyl)-4-methyl-5-phenyl-1H-pyrazol-3-yl)amino)isonicotinic acid (Compounds 66 and 67)

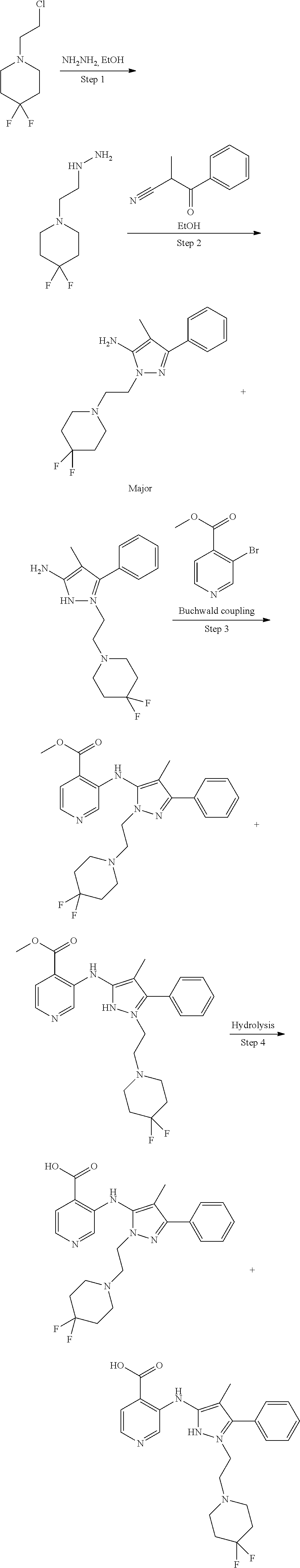

Compounds 66 and 67 were synthesized by a method similar to that illustrated in paragraph [0416] above except that the starting compound was 4,4-difluoro-1-(2-hydrazinylethyl) piperidine.

### Synthesis of 3-((3-methyl-1-(piperidin-4-yl)-1H-pyrazol-4-yl)amino) isonicotinic acid and 3-((5-methyl-1-(piperidin-4-yl)-1H-pyrazol-4-yl) amino)isonicotinic acid (Compounds 69 and 68)

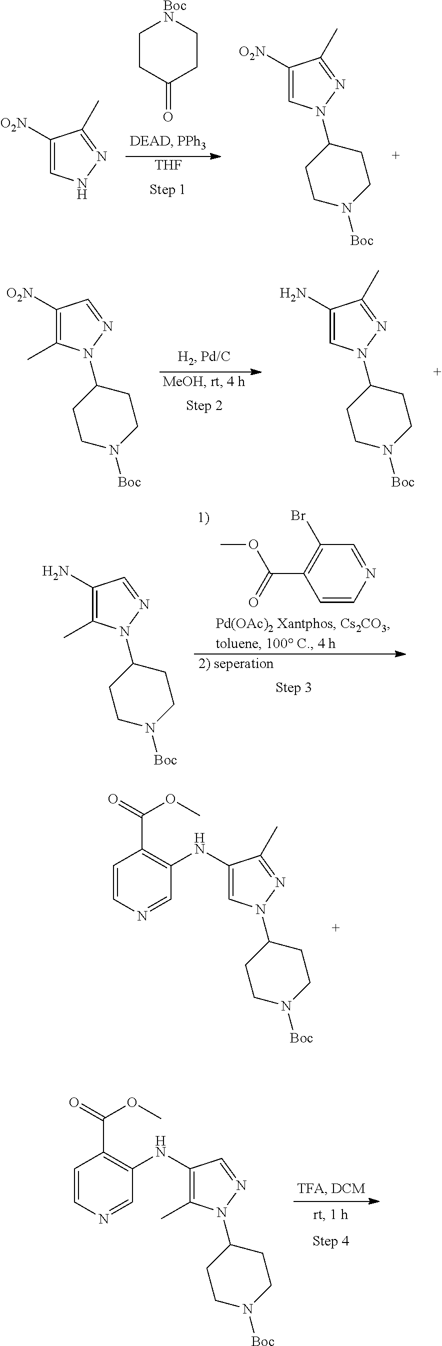

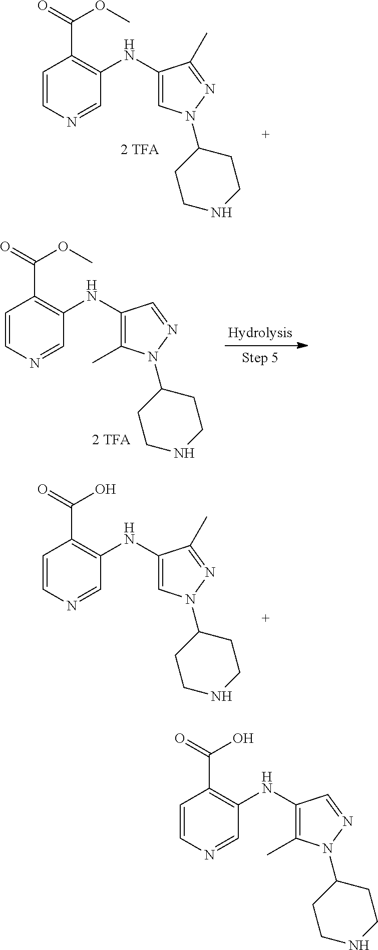 [SMILES: COC(=O)c1ccncc1Nc1cn(C2CCNCC2)nc1C ; COC(=O)c1ccncc1Nc1cnn(C2CCNCC2)c1C ; Cc1nn(C2CCNCC2)cc1Nc1cnccc1C(=O)O ; Cc1c(Nc2cnccc2C(=O)O)cnn1C1CCNCC1]

### Synthesis of the mixture of tert-butyl 4-(3-methyl-4-nitro-1H-pyrazol-1-yl) piperidine-1-carboxylate and tert-butyl 4-(5-methyl-4-nitro-1H-pyrazol-1-yl) piperidine-1-carboxylate

To a solution of 3-methyl-4-nitro-1H-pyrazole (5.08 g, 40 mmol) in anhydrous THF (60 mL) was added tert-butyl 4-hydroxypiperidine-1-carboxylate (8.84 g, 44 mmol) and PPh₃ (15.7 g, 60 mmol). The resulting solution was stirred at 0° C. for 5 min, then DEAD (10.4 g, 60 mmol) was added dropwise at 0° C. After addition the resulting mixture was stirred at room temperature for 3 hours, and the LCMS indicated the starting material was consumed. After concentration, the residue was purified by silica gel chromatography (eluted with EtOAc/petroleum ether=4/1 to 2/1) to give a mixture of two regioisomers (totally 8.9 g, Yield: 72%, isomeric ratio: around 7/3 based on HNMR): tert-butyl 4-(3-methyl-4-nitro-1H-pyrazol-1-yl)piperidine-1-carboxylate (major) and tert-butyl 4-(5-methyl-4-nitro-1H-pyrazol-1-yl)piperidine-1-carboxylate (minor) as light yellow oil. ESI-LCMS (m/z): 255.1 [M−55].

### Synthesis of the mixture of tert-butyl 4-(4-amino-3-methyl-1H-pyrazol-1-yl)piperidine-1-carboxylate and tert-butyl 4-(4-amino-5-methyl-1H-pyrazol-1-yl)piperidine-1-carboxylate

To a solution of tert-butyl 4-(3-methyl-4-nitro-1H-pyrazol-1-yl)piperidine-1-carboxylate and tert-butyl 4-(5-methyl-4-nitro-1H-pyrazol-1-yl)piperidine-1-carboxylate (4.8 g, 15.5 mmol, form step 1) in MeOH (20 mL) was added 10% Pd/C (480 mg), and the resulting mixture was stirred at room temperature under hydrogen atmosphere for 4 hours. The LCMS indicated the starting materials were consumed. After filtration, the filtrate was concentrated to give a mixture of tert-butyl 4-(4-amino-3-methyl-1H-pyrazol-1-yl)piperidine-1-carboxylate and tert-butyl 4-(4-amino-5-methyl-1H-pyrazol-1-yl) piperidine-1-carboxylate (4.1 g, Yield: 94%, isomeric ratio: around 5/3 based on HNMR) as light yellow oil. ESI-LCMS (m/z): 281.2 [M+1].

### Synthesis of methyl 3-(1-(1-(tert-butoxycarbonyl)piperidin-4-yl)-3-methyl-1H-pyrazol-4-ylamino)isonicotinate and methyl 3-(1-(1-(tert-butoxy carbonyl)piperidin-4-yl)-5-methyl-1H-pyrazol-4-ylamino)isonicotinate

The standard Buchwald coupling protocol from paragraph [0306] above was followed (500 mg, Yield: 9%).

### Synthesis of methyl 3-(3-methyl-1-(piperidin-4-yl)-1H-pyrazol-4-yl-amino) isonicotinate and methyl 3-((5-methyl-1-(piperidin-4-yl)-1H-pyrazol-4-yl)amino) isonicotinate

To a solution of methyl 3-((1-(1-(tert-butoxycarbonyl)piperidin-4-yl)-3-methyl-1H-pyrazol-4-yl)amino)isonicotinate (1.0 g, 2.40 mmol) in DCM (10 mL) was added trifluoroacetic acid (3.07 g, 2.0 mL). The reaction mixture was stirred at 15° C. for 1 h. Then concentrated to give the methyl 3-(3-methyl-1-(piperidin-4-yl)-1H-pyrazol-4-yl amino)isonicotinate (1.2 g, Yield: 92%) as 2 TFA salt which was used directly in next reaction without further purification. ESI-LCMS (m/z): 316.1 [M+1]. Same protocol used for methyl 3-((5-methyl-1-(piperidin-4-yl)-1H-pyrazol-4-yl)amino) isonicotinate.

### Synthesis of 3-((3-methyl-1-(piperidin-4-yl)-1H-pyrazol-4-yl)amino) isonicotinic acid and 3-((5-methyl-1-(piperidin-4-yl)-1H-pyrazol-4-yl) amino)isonicotinic acid

The standard NaOH hydrolysis procedure from paragraph [0307] above was followed.

### Synthesis of 3-((1-(2-cyanophenyl)-3-methyl-1H-pyrazol-4-yl)amino) isonicotinic acid (Compound 70)

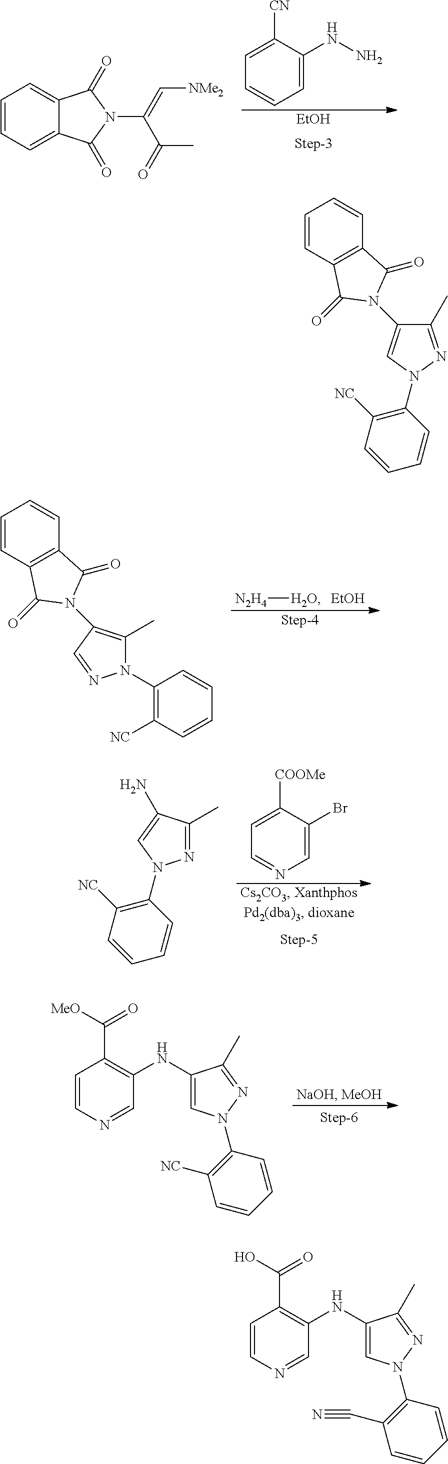

Compound 70 was synthesized by a method similar to that illustrated in paragraph [0313] above except that the starting compounds were 2-(2-oxopropyl)isoindoline-1,3-dione and 2-hydrazinylbenzonitrile.

### Synthesis of 3-((1-(2-cyanophenyl)-4-methyl-1H-pyrazol-3-yl)amino) isonicotinic acid (Compound 71)

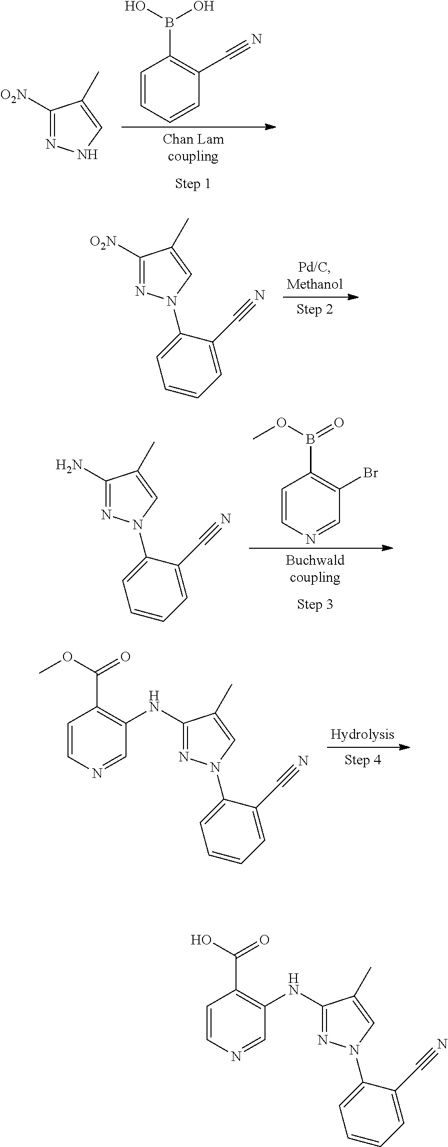

Compound 71 was synthesized by a method similar to that illustrated in paragraph above except that the starting compound was (2-cyanophenyl)boronic acid.

### Synthesis of 3-((4-phenyl-1-(tetrahydro-2H-pyran-4-yl)-1H-pyrazol-3-yl) amino)isonicotinic acid (Compound 72)

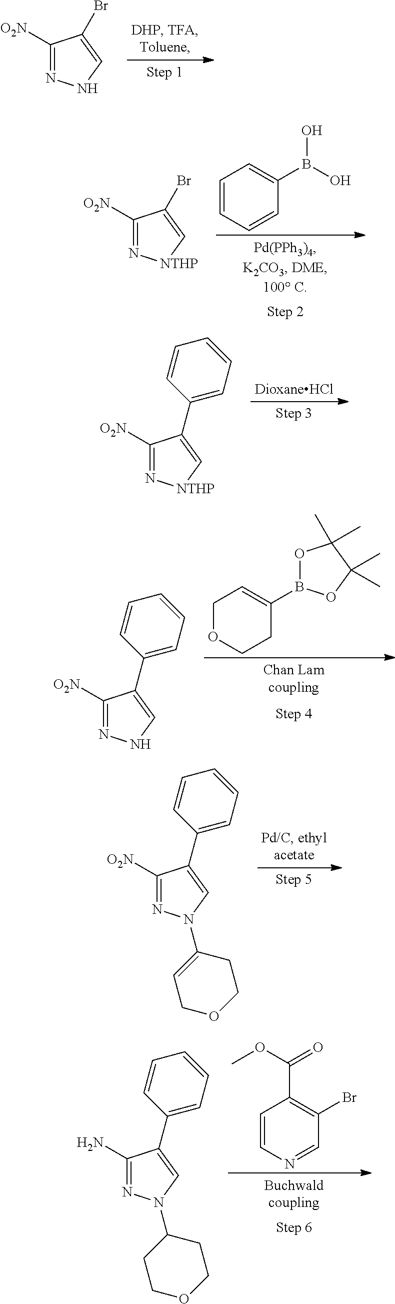

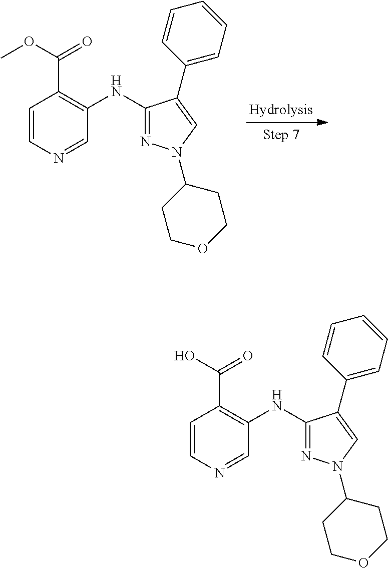 [SMILES: COC(=O)c1ccncc1Nc1nn(C2CCOCC2)cc1-c1ccccc1 ; O=C(O)c1ccncc1Nc1nn(C2CCOCC2)cc1-c1ccccc1]

Compound 72 was synthesized by a method similar to that illustrated in paragraph [0388] above except that 2-(3,6-dihydro-2H-pyran-4-yl)-4,4,5,5-tetramethyl-1,3,2-dioxaborolane was used in the Chan-Lam coupling step.

### Synthesis of 3-((4-phenyl-1-(3, 3, 3-trifluoropropyl)-1H-pyrazol-3-yl) amino) isonicotinic acid (Compound 73)

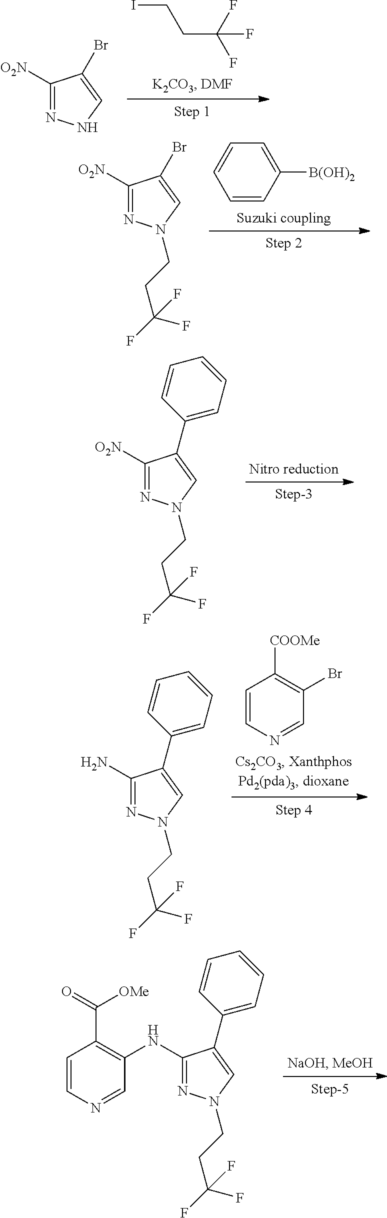

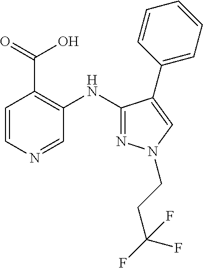 [SMILES: O=C(O)c1ccncc1Nc1nn(CCC(F)(F)F)cc1-c1ccccc1]

Compound 73 was synthesized by a method similar to that illustrated in paragraph [0388] above starting from 1,1,1-trifluoro-3-iodopropane instead of THP protection.

### Synthesis of 3-((4-(pyridin-4-yl)-1-(3, 3, 3-trifluoropropyl)-1H-pyrazol-3-yl) amino)isonicotinic acid (Compound 74)

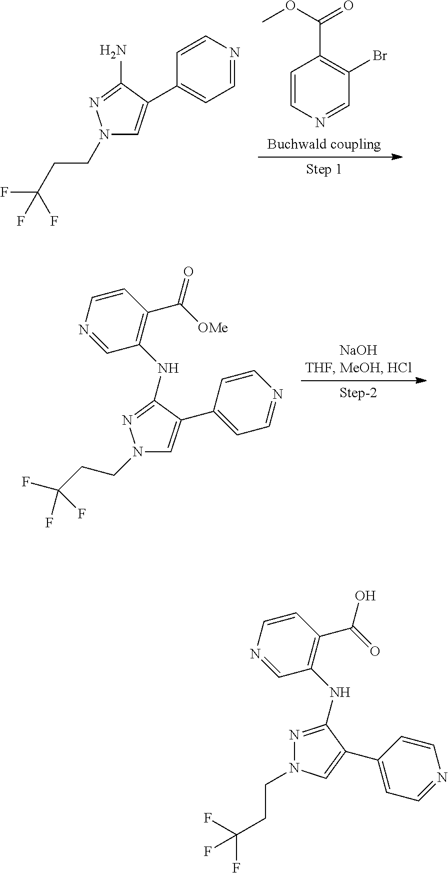

Compound 74 was synthesized by a method similar to that illustrated in paragraph [0457] above starting from pyridin-4-ylboronic acid.

### Synthesis of 3-((1-(4-methoxyphenyl)-3,5-dimethyl-1H-pyrazol-4-yl) amino)isonicotinic acid and 3-((1-(4-hydroxyphenyl)-3,5-dimethyl-1H-pyrazol-4-yl)amino)isonicotinic acid (Compounds 75 and 76)

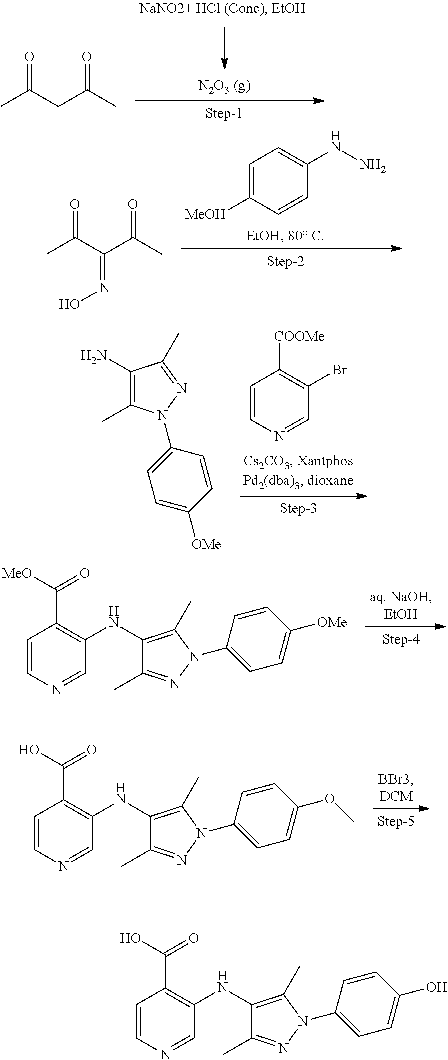

### Synthesis of 3-(hydroxyimino)pentane-2,4-dione

To a stirred solution of pentane-2,4-dione (20 g, 199 mmol) in ethanol (150 mL), N₂O₃ (gas) was purged for 1 h at 0° C. The reaction mixture was stirred at room temperature for 2 h. The progress of the reaction was monitored by TLC. Upon completion the reaction was concentrated to dryness under reduced pressure. The residue was washed with (10%) diethyl ether and (90%) n-hexane to afford the title compound (20 g, 77.6%).

### Synthesis of 1-(4-methoxyphenyl)-3,5-dimethyl-1H-pyrazol-4-amine

To a stirred solution of 3-(hydroxyimino)pentane-2,4-dione (2.6 g, 320.14 mmol) in EtOH (100 mL), (4-methoxyphenyl)hydrazine (3.85 g, 22.16 mmol) was added at 0° C. The reaction mixture was stirred at 70° C. for 4 h. The progress of the reaction was monitored by TLC and LCMS. Upon completion the reaction was concentrated to dryness under reduced pressure to obtain crude residue. The crude product was purified by column chromatography on silica gel to afford the title compounds (0.3 g, 6.86%).

### Synthesis of methyl 3-((1-(4-methoxyphenyl)-3,5-dimethyl-1H-pyrazol-4-yl) amino)isonicotinate

The standard Buchwald coupling protocol from paragraph [0306] above was followed (0.2 g, 24.6%).

### Synthesis of 3-((1-(4-methoxyphenyl)-3,5-dimethyl-1H-pyrazol-4-yl) amino)isonicotinic acid

The standard NaOH hydrolysis procedure from paragraph [0307] above was followed (0.07 g, 73%).

### Synthesis of 3-((1-(4-hydroxyphenyl)-3,5-dimethyl-1H-pyrazol-4-yl)amino) isonicotinic acid

3-((1-(4-methoxyphenyl)-3,5-dimethyl-1H-pyrazol-4-yl) amino)isonicotinic acid was demethylated using boron tribromide.

### Synthesis of 3-((3,5-dimethyl-1-phenyl-1H-pyrazol-4-yl)amino)isonicotinic acid (Compound 77)

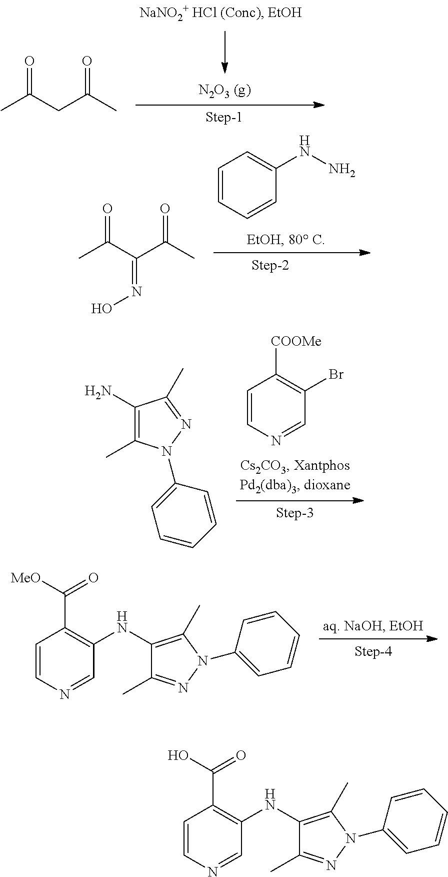

Compound 77 was synthesized using a method similar to that illustrated in paragraph [0461] above except that the starting compound was phenyl hydrazine.

### Synthesis of 3-((3-methyl-1-(tetrahydro-2H-pyran-4-yl)-1H-pyrazol-5-yl) amino) isonicotinic acid (Compound 78)

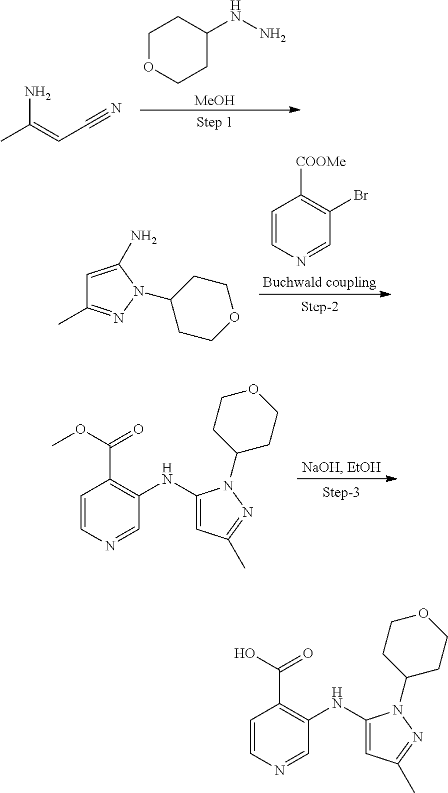

Compound 78 was synthesized by a method similar to that illustrated in paragraph [0432] above except that the starting compound was (tetrahydro-2H-pyran-4-yl)hydrazine.

### Synthesis of 3-((1-((cis)-4-aminocyclohexyl)-5-methyl-1H-pyrazol-4-yl) amino)isonicotinic acid and 3-((1-((cis)-4-aminocyclohexyl)-3-methyl-1H-pyrazol-4-yl)amino)isonicotinic acid (Compounds 79 and 80)

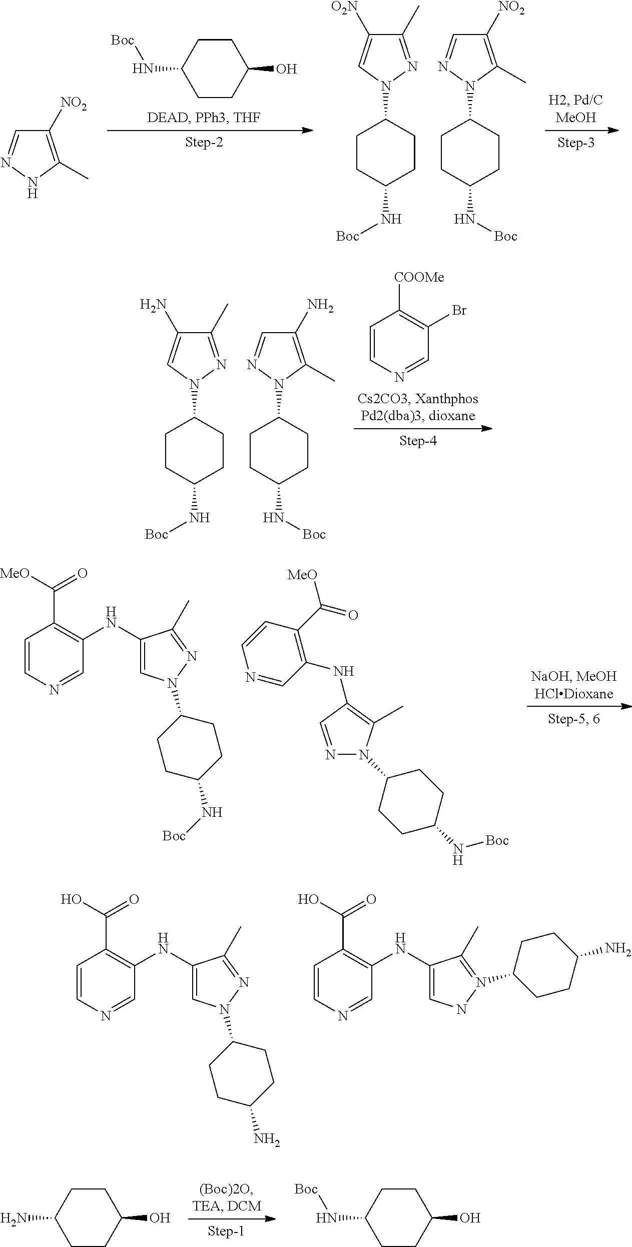

Compounds 79 and 80 were synthesized by a method similar to that illustrated in paragraph [0445] above except that the first step was a Mitsunobu reaction using tert-butyl ((trans)-4-hydroxycyclohexyl)carbamate.

### Synthesis of tert-butyl ((cis)-4-(3-methyl-4-nitro-1H-pyrazol-1-yl) cyclohexyl)carbamate and tert-butyl ((cis)-4-(5-methyl-4-nitro-1H-pyrazol-1-yl)cyclohexyl)carbamate

To a stirred solution of 5-methyl-4-nitro-1H-pyrazole (1.6 g, 7.44 mmol) in THF (20 mL), tert-butyl ((trans)-4-hydroxycyclohexyl)carbamate (0.945 g, 7.44 mmol) and triphenyl phosphine (2.33 g, 8.93 mmol) were added at 0° C. and stirred at room temperature for 30 min. After that DIAD (1.8 g, 8.93 mmol) was added dropwise and stirred at room temperature for 12 h. The progress of the reaction was monitored by TLC. Upon completion the reaction was diluted with water and extracted with 10% methanol in DCM. The combined organic layers were washed with sodium bicarbonate solution, dried over anhydrous Na₂SO₄ and concentrated under reduced pressure to obtain a crude compound. The crude product was purified by column chromatography on (neutral) silica gel to afford the mixture of title compounds (1.2 g, 49.7%).

### Synthesis of 3-((5-ethyl-1-phenyl-1H-pyrazol-4-yl)amino)isonicotinic acid (Compound 81)

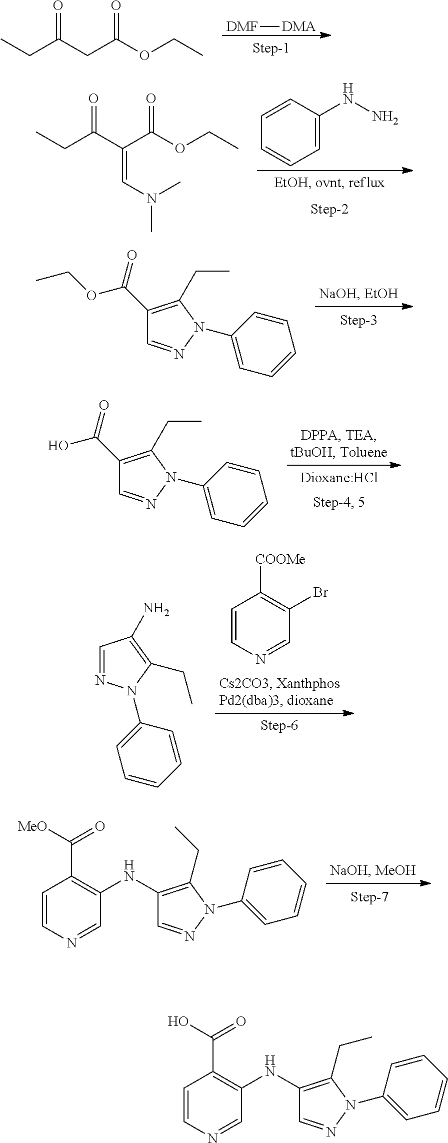

### Synthesis of ethyl (Z)-2-((dimethylamino)methylene)-3-oxopentanoate

To a stirred solution of ethyl 3-oxopentanoate (0.5 g, 3.47 mmol) in DMF-DMA (1.2 g, 10.41 mmol), methanol (10 mL) was added and the solution was heated at 60° C. for 2 h. The progress of the reaction was monitored by TLC and LCMS. Upon completion the reaction was concentrated under reduced pressure to obtain a crude residue. The crude product was purified by column chromatography on silica gel to afford the title compound (0.5 g, 72.46%).

### Synthesis of ethyl 5-ethyl-1-phenyl-1H-pyrazole-4-carboxylate

To a stirred solution of ethyl (Z)-2-((dimethylamino)methylene)-3-oxopentanoate (0.5 g, 2.51 mmol) and phenylhydrazine (0.325 g, 3.01 mmol) in methanol (10 mL) and the solution was heated at 100° C. for 12 h. The progress of the reaction was monitored by TLC and LCMS. Upon completion the reaction was concentrated under reduced pressure to obtain a crude residue. The crude product was purified by column chromatography on silica gel to afford the compound (0.7 g, 81%).

### Synthesis of 5-ethyl-1-phenyl-1H-pyrazole-4-carboxylic acid

To a stirred solution of ethyl 5-ethyl-1-phenyl-1H-pyrazole-4-carboxylate (0.7 g, 2.86 mmol) in ethanol (5 mL), NaOH (0.2 g, 7.17 mmol) in water (5 mL) was added and stirred at room temperature for 12 h. The progress of the reaction was monitored by TLC. Upon completion the reaction was concentrated to dryness under reduced pressure. The residue was acidified with 1N HCl under cool condition and the precipitated solid was filtered and dried to afford the title compound confirmed by NOE (0.5 g, 80.7%).

### Synthesis of 5-ethyl-1-phenyl-1H-pyrazol-4-amine

To a stirred solution of 5-ethyl-1-phenyl-1H-pyrazole-4-carboxylic acid (0.4 g, 1.85 mmol) in t-BuOH:toluene (1:1, 20 mL), DPPA (0.763 g, 2.77 mmol) and TEA (0.28 g, 2.77 mmol) was added. The reaction was heated at 100° C. for overnight. The progress of the reaction was monitored by TLC and LCMS. Upon completion the reaction mixture was diluted with water and extracted with ethyl acetate. The combined organic layers were dried over anhydrous Na₂SO₄ and concentrated under reduced pressure resulting in a crude compound which was purified by column chromatography to afford the compound. The compound was dissolved in dioxane: HCl (5 mL) and stirred at room temperature for 1 h. Upon completion the reaction was concentrated to dryness under reduced pressure, and was purified by washing with hexane to obtain compound (0.34 g, 88.5%).

### Synthesis of methyl 3-((5-ethyl-1-phenyl-1H-pyrazol-4-yl)amino) isonicotinate

The standard Buchwald coupling protocol from paragraph [0306] above was followed (0.15 g, 34%).

### Synthesis of 3-((5-ethyl-1-phenyl-1H-pyrazol-4-yl)amino)isonicotinic acid

The standard NaOH hydrolysis procedure from paragraph [0307] above was followed (0.085 g, 88%).

### Synthesis of Synthesis of 3-((5-methyl-1-phenyl-1H-pyrazol-4-yl)amino)isonicotinic acid (Compound 82)

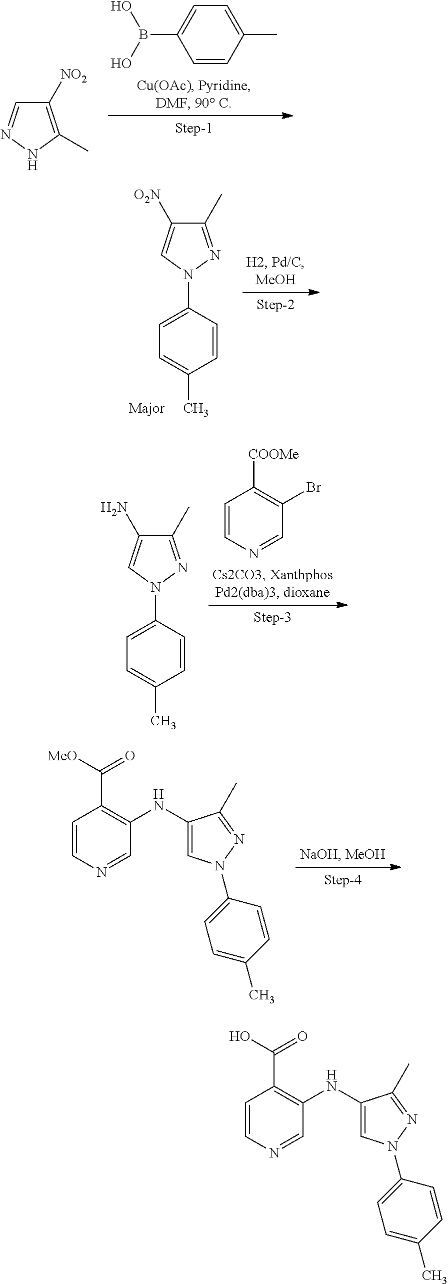

Compound 82 was synthesized by a method similar to that illustrated in paragraph [0388] above except that the starting compound was p-tolylboronic acid.

### Synthesis of 3-((3-methyl-1-(1-methylpiperidin-4-yl)-1H-pyrazol-4-yl)amino) isonicotinic acid and 3-((5-methyl-1-(1-methylpiperidin-4-yl)-1H-pyrazol-4-yl) amino)isonicotinic acid (Compounds 83 and 84)

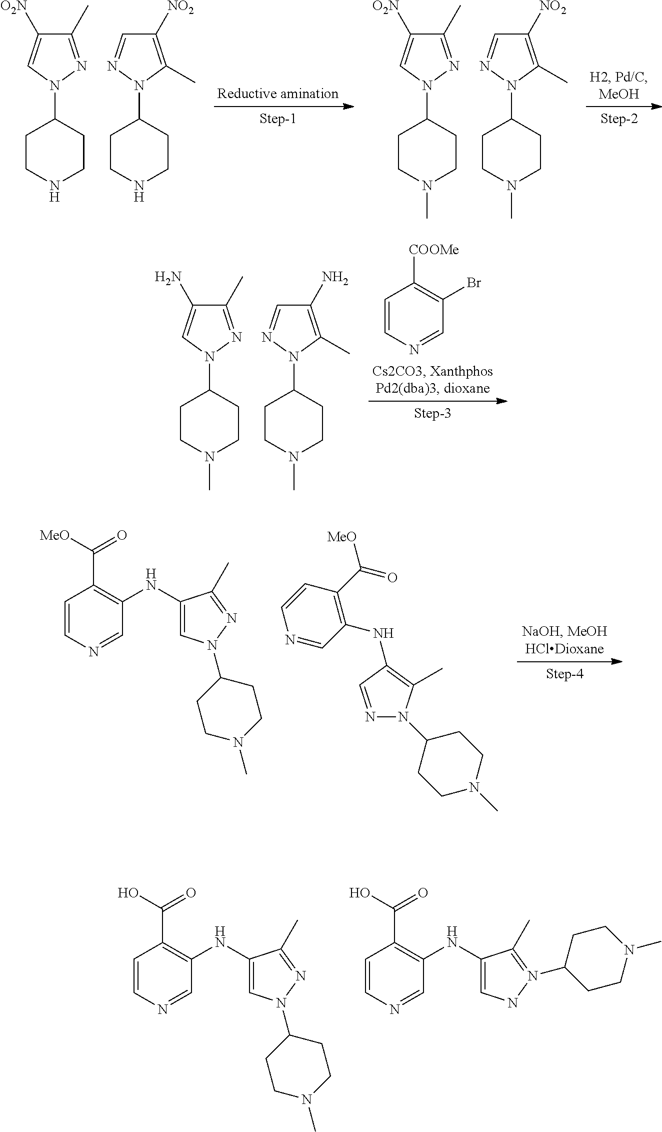

Compounds 83 and 84 were synthesized by a method similar to that illustrated in paragraph [0445] above except that the first step was an Eschweiler-Clarke reaction.

### Synthesis of 1-methyl-4-(3-methyl-4-nitro-1H-pyrazol-1-yl)piperidine and 1-methyl-4-(5-methyl-4-nitro-1H-pyrazol-1-yl)piperidine

To a mixture of 4-(3-methyl-4-nitro-1H-pyrazol-1-yl)piperidine and 4-(5-methyl-4-nitro-1H-pyrazol-1-yl)piperidine (2 g, 9.52 mmol) in 1,2 DCE (30 mL), formalin (2.1 mL, 28.57 mmol) and DIPEA (2.6 mL, 14.28 mmol) were added and stirred at room temperature for 10 min. Acetic acid (0.8 mL, 14.28 mmol) was added dropwise and stirred at room temperature for 20 min. After that sodium triacetoxyborohydride (4 g, 19.04 mmol) was added and stirred at room temperature for 16 h. The progress of the reaction was monitored by TLC. Upon completion the reaction was diluted with sodium bicarbonate and extracted with 10% methanol in DCM. The combined organic layers were washed with brine, dried over anhydrous Na₂SO₄ and concentrated under reduced pressure to obtain a crude compound. The crude product was purified by column chromatography on silica gel to afford the mixture of title compounds (1.8 g, 85.7%).

### Synthesis of 3-((1-(4-aminocyclohexyl)-3-(3-chlorophenyl)-1H-pyrazol-5-yl) amino)isonicotinic acid and 3-((1-(4-aminocyclohexyl)-5-(3-chlorophenyl)-1H-pyrazol-3-yl)amino)isonicotinic acid (Compounds 85 and 87)

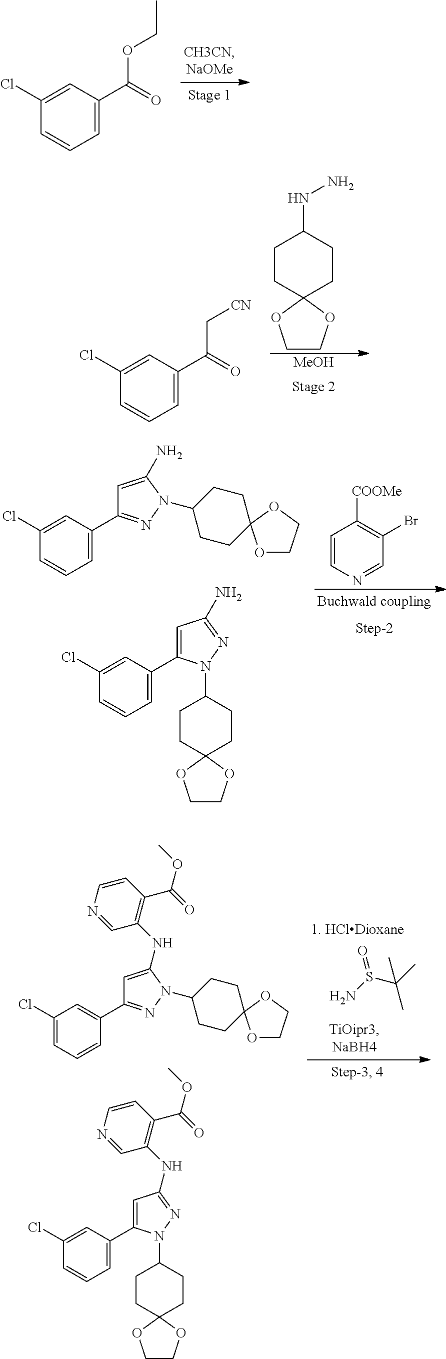

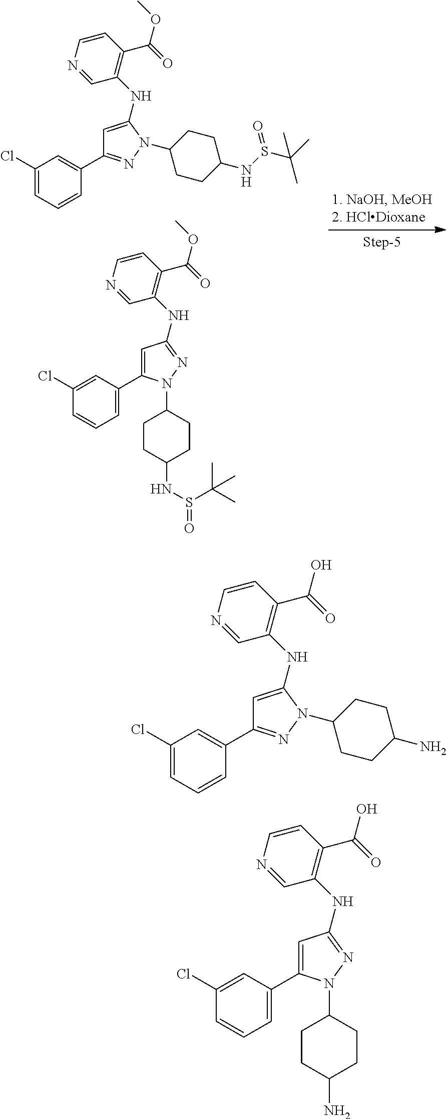 [SMILES: COC(=O)c1ccncc1Nc1cc(-c2cccc(Cl)c2)nn1C1CCC(NS(=O)C(C)(C)C)CC1 ; COC(=O)c1ccncc1Nc1cc(-c2cccc(Cl)c2)n(C2CCC(NS(=O)C(C)(C)C)CC2)n1 ; NC1CCC(n2nc(-c3cccc(Cl)c3)cc2Nc2cnccc2C(=O)O)CC1 ; NC1CCC(n2nc(Nc3cnccc3C(=O)O)cc2-c2cccc(Cl)c2)CC1]

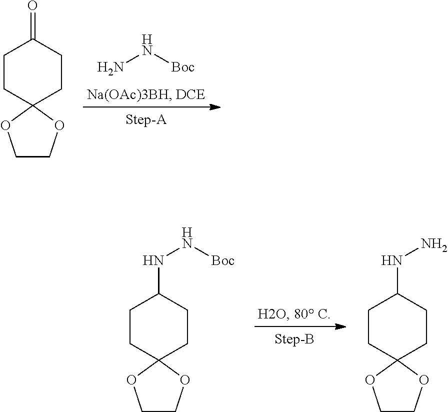 [SMILES: O=C1CCC2(CC1)OCCO2 ; CC(C)(C)OC(=O)NN ; CC(C)(C)OC(=O)NNC1CCC2(CC1)OCCO2 ; NNC1CCC2(CC1)OCCO2]

### Synthesis of 3-(3-chlorophenyl)-3-oxopropanenitrile

To a stirred solution of ethyl 3-chlorobenzoate (5 g, 27.16 mmol) and sodium methoxide (2.02 g, 40.75 mmol) in acetonitrile (50 mL) and reaction was heated to reflux for 3 h. The progress of the reaction was monitored by TLC and LCMS. Upon completion the reaction was filtered and solid dissolved in water. The residue was acidified with 3M HCl solution and extracted with DCM. The combined organic layers were washed with sodium bicarbonate solution, dried over anhydrous Na₂SO₄ and concentrated under reduced pressure to obtain a crude compound. The crude product was purified by washing with DCM and ether to afford the mixture of two isomers compound (1.2 g, 49.7%).

### Synthesis of tert-butyl 2-(1,4-dioxaspiro[4.5]decan-8-yl)hydrazine-1-carboxylate

To a stirred solution of 1,4-dioxaspiro[4.5]decan-8-onein dichloroethane (5 g, 32.01 mmol) in hexane (70 mL), tert-butyl hydrazine carboxylate (3.66 g, 35.21 mmol) was added and the solution was heated to reflux for 5 h. The progress of the reaction was monitored by TLC. The precipitated solid was filtered and dried. The residue was dissolved in THF (60 mL) and methanol (10 mL), sodium borohydride (2.3 g, 48.02 mol) was added portion wise and stirred at room temperature for 12 h. Upon completion the reaction was diluted with ice water and extracted with ethyl acetate. The combined organic layers were washed with sodium bicarbonate solution, dried over anhydrous Na₂SO₄ and concentrated under reduced pressure to obtain a crude compound (6 g, 69%).

### Synthesis of (1,4-dioxaspiro[4.5]decan-8-yl)hydrazine

To a stirred solution of tert-butyl 2-(1,4-dioxaspiro[4.5]decan-8-yl)hydrazine-1-carboxylate (6 g, 22.05 mmol) in water (50 mL) and the reaction was stirred at 100° C. for 8 h. The progress of the reaction was monitored by TLC. After completion of the reaction, the reaction mixture was evaporated to dryness. The crude product was purified by washing with toluene to afford the title compounds (3.5 g, 92.3%).

### Synthesis of 3-(3-chlorophenyl)-1-(1,4-dioxaspiro[4.5]decan-8-yl)-1H-pyrazol-5-amine and 5-(3-chlorophenyl)-1-(1,4-dioxaspiro[4.5]decan-8-yl)-1H-pyrazol-3-amine

To a stirred solution of 3-(3-chlorophenyl)-3-oxopropanenitrile (3.5 g, 19.55 mmol) and (1,4-dioxaspiro[4.5]decan-8-yl)hydrazine (3.36 g, 19.55 mmol) in ethanol (50 mL) in sealed tube. The reaction mixture was stirred at 90° C. for 3 h. The progress of the reaction was monitored by TLC. Upon completion the reaction was concentrated to dryness under reduced pressure. The crude product was purified by column chromatography on silica gel to afford the mixture of two isomers compound (3 g, 46.08%).

### Synthesis of methyl 3-((3-(3-chlorophenyl)-1-(1,4-dioxaspiro[4.5]decan-8-yl)-1H-pyrazol-5-yl)amino)isonicotinate and methyl 3-((5-(3-chlorophenyl)-1-(1,4-dioxaspiro [4.5]decan-8-yl)-1H-pyrazol-3-yl)amino)isonicotinate

The standard Buchwald coupling protocol from paragraph [0306] above was followed (2.5 g, 59.3%).

### Synthesis of methyl 3-((3-(3-chlorophenyl)-1-(4-(1,1-dimethylethylsulfinamido)cyclohexyl)-1H-pyrazol-5-yl)amino)isonicotinate and methyl 3-((5-(3-chlorophenyl)-1-(4-(1,1-dimethylethylsulfinamido)cyclohexyl)-1H-pyrazol-3-yl)amino)isonicotinate

To a mixture of methyl 3-((3-(3-chlorophenyl)-1-(1,4-dioxaspiro[4.5]decan-8-yl)-1H-pyrazol-5-yl)amino) isonicotinate and methyl 34(5-(3-chlorophenyl)-1-(1,4-dioxaspiro[4.5]decan-8-yl)-1H-pyrazol-3-yl)amino)isonicotinate (2.5 g, 5.34 mmol) in dioxane:HCl (25 mL) and reaction was stirred at room temperature for 12 h. The progress of the reaction was monitored by TLC and LCMS. Upon completion the reaction was quenched with sodium bicarbonate and extracted with ethyl acetate. The combined organic layers were washed with sodium bicarbonate solution, dried over anhydrous Na₂SO₄ and concentrated under reduced pressure to obtain a crude residue. The residue (1 g, 2.358 mmol) and 2-methylpropane-2-sulfinamide (0.428 g, 3.53 mmol) in DCE (10 mL), TiOipr3(4.02 g, 14.1 mmol) was added and stirred at room temperature for 30 min. Sodium borohydride (0.178 g, 4.71 mmol) was added and stirred at room temperature for 12 h. Upon completion the reaction was diluted with ice water and extracted with ethyl acetate. The combined organic layers were washed with sodium bicarbonate solution, dried over anhydrous Na₂SO₄ and concentrated under reduced pressure. The crude product was purified by column chromatography on silica gel to afford the mixture of two isomers compound (0.5 g, 38.16%).

### Synthesis of 3-((1-(4-aminocyclohexyl)-3-(3-chlorophenyl)-1H-pyrazol-5-yl) amino)isonicotinic acid and 3-((1-(4-aminocyclohexyl)-5-(3-chlorophenyl)-1H-pyrazol-3-yl) amino)isonicotinic acid

The standard NaOH hydrolysis procedure from paragraph [0307] above was followed (0.1 g, 27%).

### Synthesis of 3-((5-(3-chlorophenyl)-1-(1,4-dioxaspiro[4.5]decan-8-yl)-1H-pyrazol-3-yl)amino)isonicotinic acid (Compound 86)

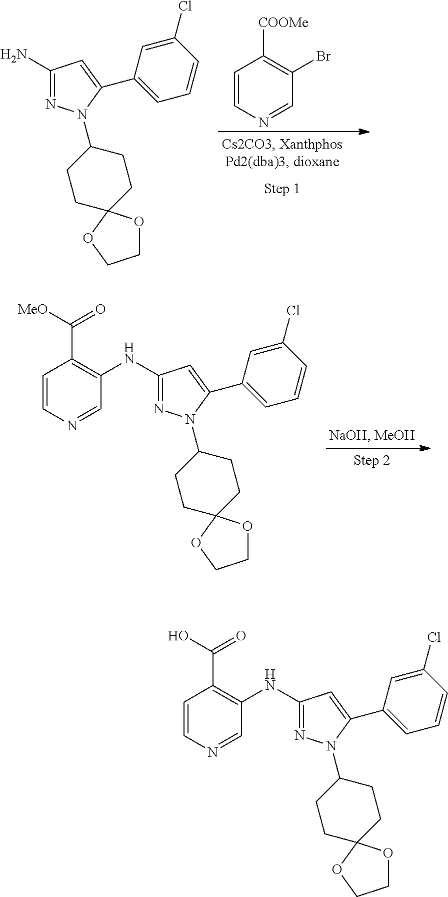

The standard Buchwald coupling protocol from paragraph [0306] and the standard NaOH hydrolysis protocol from paragraph [0307] above were followed.

### Synthesis of 3-((1-phenyl-4-(pyridin-4-yl)-1H-pyrazol-3-yl)amino) isonicotinic acid (Compound 88)

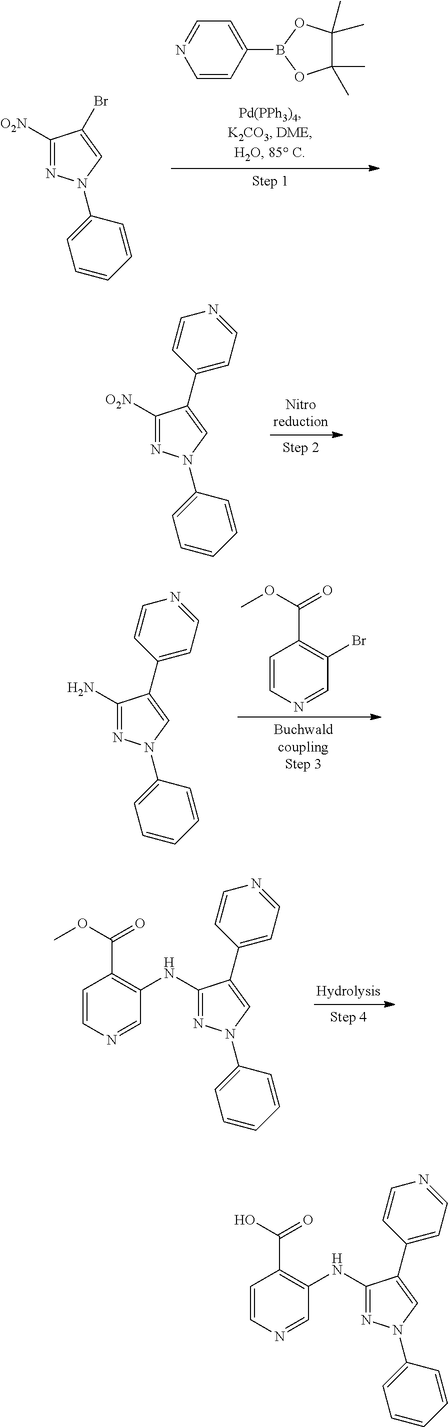

Compound 88 was synthesized by a method similar to that illustrated in paragraph [0388] above except that the starting compound was pyridin-4-ylboronic acid.

### Synthesis of 3-(1-(1-(cyclopropylmethyl)piperidin-4-yl)-3-methyl-1H-pyrazol-4-ylamino)isonicotinic acid and 3-(1-(1-(cyclopropylmethyl)piperidin-4-yl)-5-methyl-1H-pyrazol-4-ylamino) isonicotinic acid (Compounds 89 and 90)

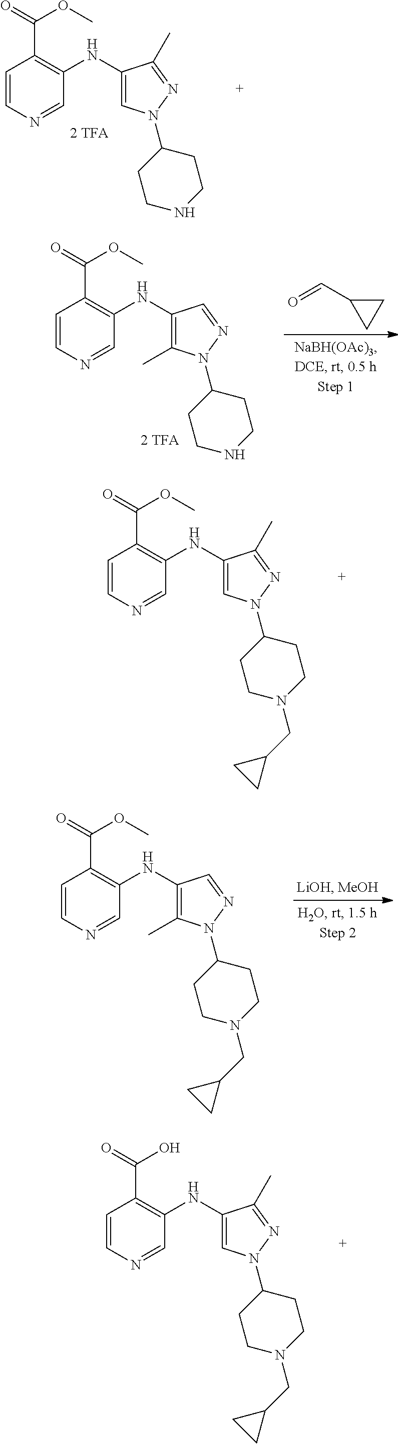 [SMILES: COC(=O)c1ccncc1Nc1cn(C2CCNCC2)nc1C ; COC(=O)c1ccncc1Nc1cnn(C2CCNCC2)c1C ; O=CC1CC1 ; COC(=O)c1ccncc1Nc1cn(C2CCN(CC3CC3)CC2)nc1C ; COC(=O)c1ccncc1Nc1cnn(C2CCN(CC3CC3)CC2)c1C ; Cc1nn(C2CCN(CC3CC3)CC2)cc1Nc1cnccc1C(=O)O]

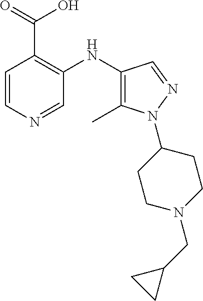 [SMILES: Cc1c(Nc2cnccc2C(=O)O)cnn1C1CCN(CC2CC2)CC1]

### Synthesis of methyl 3-((1-(1-(cyclopropylmethyl)piperidin-4-yl)-3-methyl-1H-pyrazol-4-yl)amino)isonicotinate and methyl 3-((1-(1-(cyclopropylmethyl) piperidin-4-yl)-5-methyl-1H-pyrazol-4-yl)amino)isonicotinate

To a solution of methyl 3-(3-methyl-1-(piperidin-4-yl)-1H-pyrazol-4-ylamino) isonicotinate (180 mg, 0.33 mmol, 2 TFA salt from step 4) in dichloroethane (6 mL) was added cyclopropanecarbaldehyde (117 mg, 1.67 mmol). The mixture was stirred at room temperature for 5 min then NaBH(OAc)₃ (264 mg, 1.25 mmol) was added. The resulting mixture was stirred at room temperature for 30 min, quenched with H₂O (10 mL) and extracted with dichloroethane (3×25 mL). The combined organic layers were dried over Na₂SO₄, filtered and concentrated. The residue was purified by prep-TLC (DCM/MeOH=8/1) to give methyl 3-((1-(1-(cyclopropylmethyl) piperidin-4-yl)-3-methyl-1H-pyrazol-4-yl)amino)isonicotinate (85 mg, Yield: 69%) as a yellow solid. ESI-LCMS (m/z): 370.2 [M+1]. The same protocol was used for the reductive amination of methyl 3-((5-methyl-1-(piperidin-4-yl)-1H-pyrazol-4-yl)amino)isonicotinate.

### Synthesis of 3-((1-(1-(cyclopropylmethyl)piperidin-4-yl)-3-methyl-1H-pyrazol-4-yl)amino)isonicotinic acid and 3-((1-(1-(cyclopropylmethyl) piperidin-4-yl)-5-methyl-1H-pyrazol-4-yl)amino)isonicotinic acid

To a solution of methyl 3-((1-(1-(cyclopropylmethyl)piperidin-4-yl)-3-methyl-1H-pyrazol-4-yl) amino)isonicotinate (75 mg, 0.20 mmol) in H₂O/MeOH (v/v=1/4, 5 mL) was added lithium hydroxide hydrate (30 mg, 1.28 mmol). The reaction mixture was stirred at room temperature for 1.5 h, and the LCMS indicated the starting material was consumed. The mixture was adjusted to pH=6-7 with 2N HCl aqueous solution then concentrated. The residue was purified by Prep-HPLC to give 3-((1-(1-(cyclo-propylmethyl)piperidin-4-yl)-3-methyl-1H-pyrazol-4-yl) amino)isonicotinic acid (15 mg, Yield: 20%) as yellow solid. The same protocol was used for the hydrolysis of methyl 3-((1-(1-(cyclopropylmethyl)piperidin-4-yl)-5-methyl-1H-pyrazol-4-yl)amino)isonicotinate.

### Synthesis of 3-(3-methyl-1-(1-(1-methylazetidin-3-yl)piperidin-4-yl)-1H-pyrazol-4-ylamino)isonicotinic acid and 3-(5-methyl-1-(1-(1-methylazetidin-3-yl)piperidin-4-yl)-1H-pyrazol-4-ylamino)isonicotinic acid (Compounds 91 and 92)

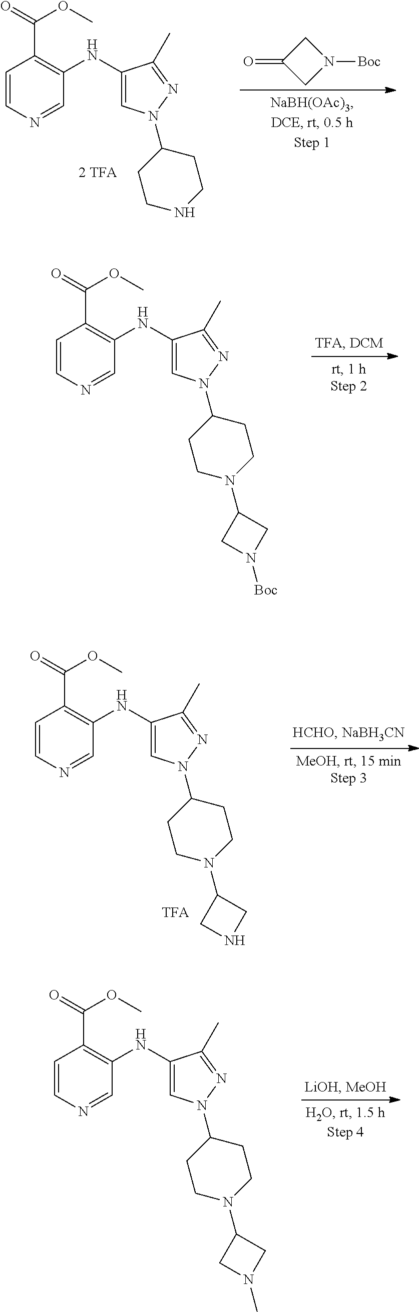 [SMILES: COC(=O)c1ccncc1Nc1cn(C2CCNCC2)nc1C ; CC(C)(C)OC(=O)N1CC(=O)C1 ; COC(=O)c1ccncc1Nc1cn(C2CCN(C3CN(C(=O)OC(C)(C)C)C3)CC2)nc1C ; COC(=O)c1ccncc1Nc1cn(C2CCN(C3CNC3)CC2)nc1C ; COC(=O)c1ccncc1Nc1cn(C2CCN(C3CN(C)C3)CC2)nc1C]

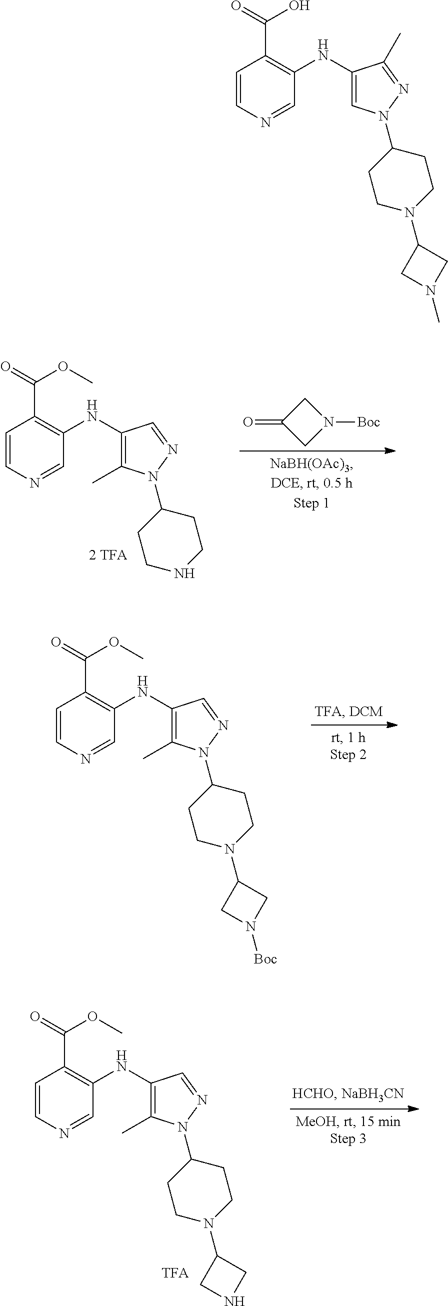 [SMILES: Cc1nn(C2CCN(C3CN(C)C3)CC2)cc1Nc1cnccc1C(=O)O ; COC(=O)c1ccncc1Nc1cnn(C2CCNCC2)c1C ; CC(C)(C)OC(=O)N1CC(=O)C1 ; COC(=O)c1ccncc1Nc1cnn(C2CCN(C3CN(C(=O)OC(C)(C)C)C3)CC2)c1C ; COC(=O)c1ccncc1Nc1cnn(C2CCN(C3CNC3)CC2)c1C]

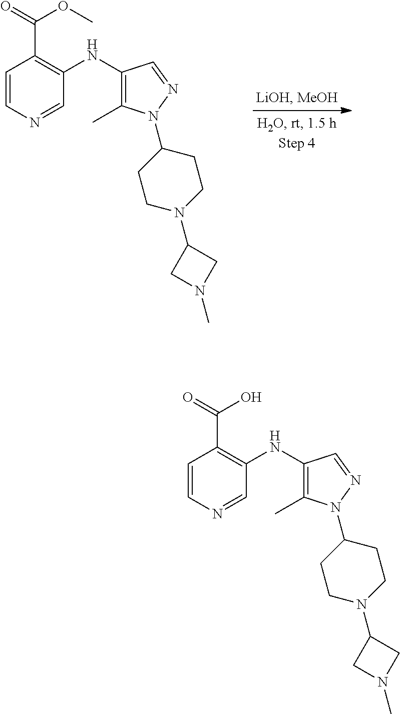 [SMILES: COC(=O)c1ccncc1Nc1cnn(C2CCN(C3CN(C)C3)CC2)c1C ; Cc1c(Nc2cnccc2C(=O)O)cnn1C1CCN(C2CN(C)C2)CC1]

Compounds 91 and 92 were synthesized by a method similar to that illustrated in paragraph [0498] above except that the starting compound was tert-butyl 3-oxoazetidine-1-carboxylate.

### Synthesis of methyl 3-((1-(1-cyclobutylpiperidin-4-yl)-3-methyl-1H-pyrazol-4-yl)amino)isonicotinate, 3-(1-(1-cyclobutylpiperidin-4-yl)-3-methyl-1H-pyrazol-4-ylamino)isonicotinic acid, methyl 3-((1-(1-cyclobutylpiperidin-4-yl)-5-methyl-1H-pyrazol-4-yl)amino)isonicotinate, and 3-(1-(1-cyclobutylpiperidin-4-yl)-5-methyl-1H-pyrazol-4-yl) amino)isonicotinic acid (Compounds 93-96)

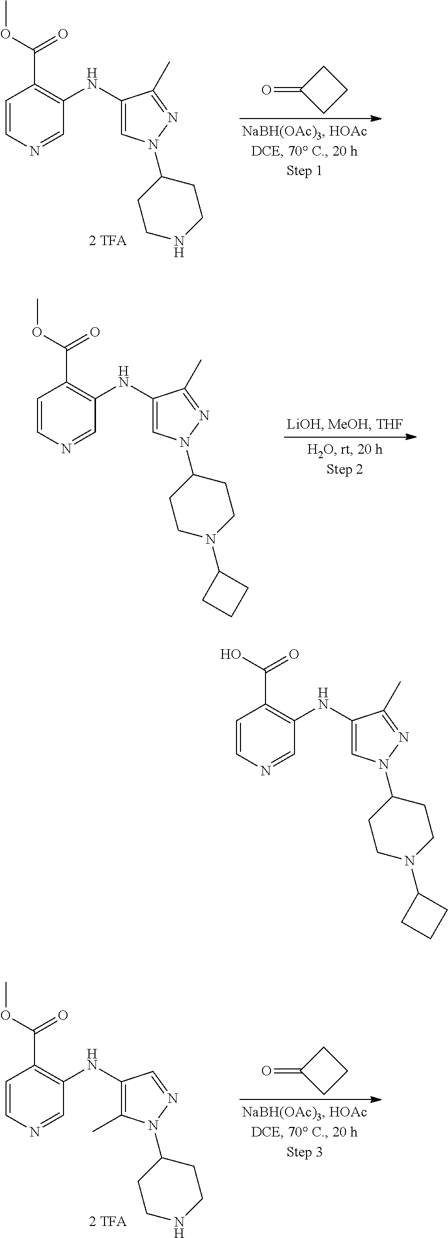

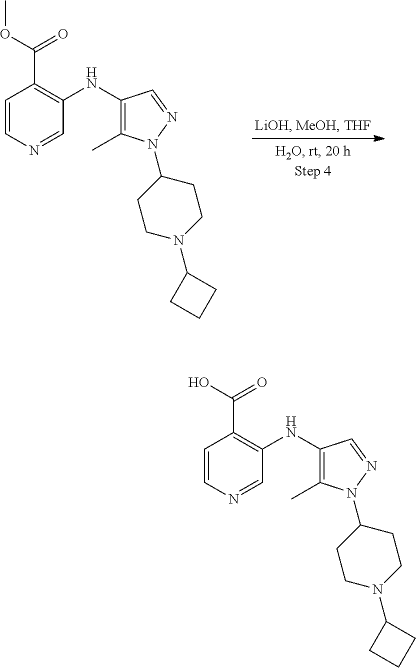 [SMILES: COC(=O)c1ccncc1Nc1cnn(C2CCN(C3CCC3)CC2)c1C ; Cc1c(Nc2cnccc2C(=O)O)cnn1C1CCN(C2CCC2)CC1]

Compounds 93-96 were synthesized by a method similar to that illustrated in paragraph [0498] above except that the starting compound was cyclobutanone.

### Synthesis of 3-(1-(1-benzoylpiperidin-4-yl)-3-methyl-1H-pyrazol-4-ylamino) isonicotinic acid and 3-(1-(1-benzoylpiperidin-4-yl)-5-methyl-1H-pyrazol-4-ylamino) isonicotinic acid (Compounds 97 and 98)

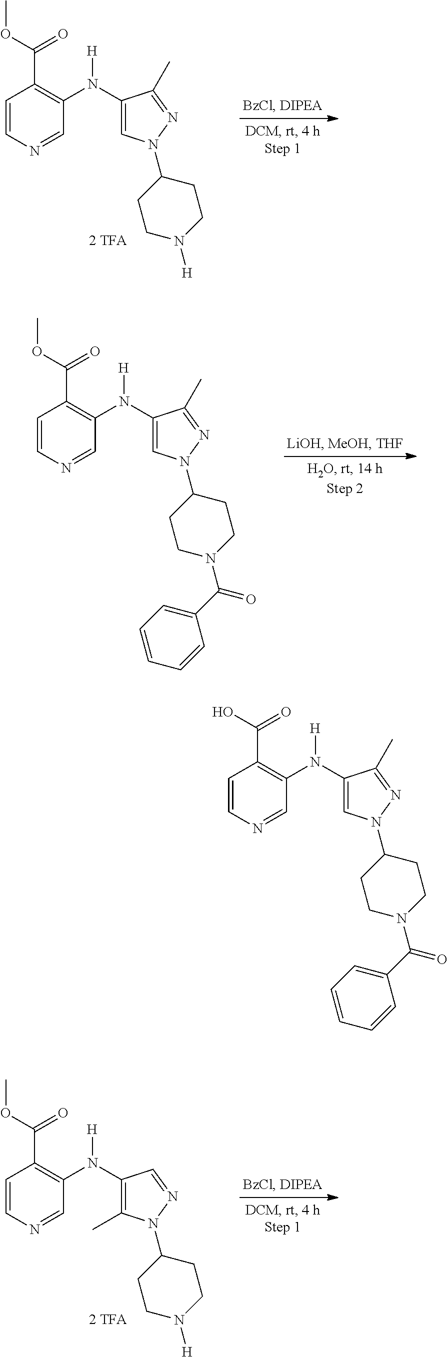 [SMILES: COC(=O)c1ccncc1Nc1cn(C2CCNCC2)nc1C ; COC(=O)c1ccncc1Nc1cn(C2CCN(C(=O)c3ccccc3)CC2)nc1C ; Cc1nn(C2CCN(C(=O)c3ccccc3)CC2)cc1Nc1cnccc1C(=O)O ; COC(=O)c1ccncc1Nc1cnn(C2CCNCC2)c1C]

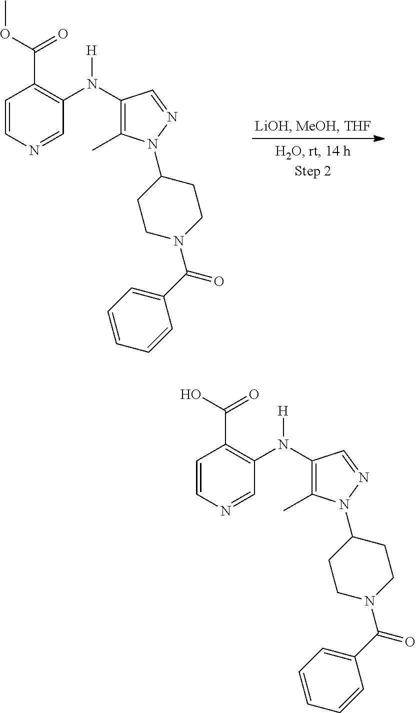 [SMILES: COC(=O)c1ccncc1Nc1cnn(C2CCN(C(=O)c3ccccc3)CC2)c1C ; Cc1c(Nc2cnccc2C(=O)O)cnn1C1CCN(C(=O)c2ccccc2)CC1]

### Synthesis of methyl 3-(1-(1-benzoylpiperidin-4-yl)-3-methyl-1H-pyrazol-4-ylamino)isonicotinate and methyl 3-(5-methyl-1-(piperidin-4-yl)-1H-pyrazol-4-yl amino)isonicotinate

To a solution of methyl 3-((3-methyl-1-(piperidin-4-yl)-1H-pyrazol-4-yl)amino) isonicotinate (180 mg, 0.33 mmol, 2 TFA salt) and DIPEA (122 mg, 0.95 mmol) in DCM (10 mL) was added benzoyl chloride (53 mg, 0.38 mmol) at 0° C. The reaction mixture was stirred at room temperature for 4 h, and LCMS indicated the starting material was consumed. Saturated NH₄Cl aqueous solution (10 mL) was added, the organic phase was separated and washed with brine, dried over Na₂SO₄, filtered and concentrated to afford methyl 3-((1-(1-benzoylpiperidin-4-yl)-3-methyl-1H-pyrazol-4-yl)amino)isonicotinate (135 mg, crude) as a yellow oil which was used directly into next step without further purification. ESI-LCMS (m/z): 420.2 [M+1]. The same protocol was used for the synthesis of methyl 3-(5-methyl-1-(piperidin-4-yl)-1H-pyrazol-4-yl amino)isonicotinate.

### Synthesis of 3-(1-(1-benzoylpiperidin-4-yl)-3-methyl-1H-pyrazol-4-yl amino)isonicotinic acid and 3-(1-(1-benzoylpiperidin-4-yl)-5-methyl-1H-pyrazol-4-yl-amino)isonicotinic acid

Standard LiOH hydrolysis conditions as described in paragraph [0500] above were followed.

### Synthesis of 3-(3-methyl-1-(1-(2,2,2-trifluoroethyl)piperidin-4-yl)-1H-pyrazol-4-ylamino)isonicotinic acid and 3-(5-methyl-1-(1-(2,2,2-tri-fluoro-ethyl)piperidin-4-yl)-1H-pyrazol-4-ylamino)isonicotinic acid (Compounds 99 and 100)

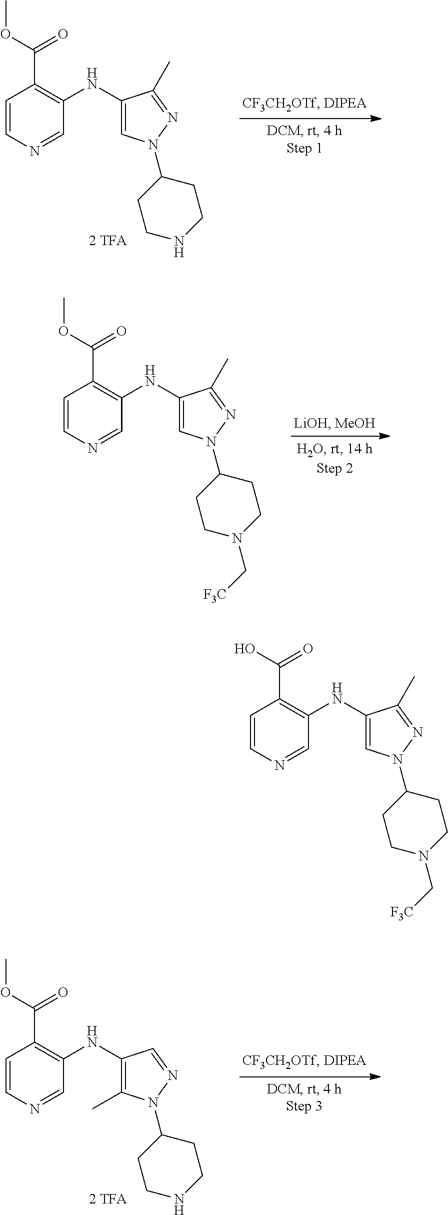 [SMILES: COC(=O)c1ccncc1Nc1cn(C2CCNCC2)nc1C ; COC(=O)c1ccncc1Nc1cn(C2CCN(CC(F)(F)F)CC2)nc1C ; Cc1nn(C2CCN(CC(F)(F)F)CC2)cc1Nc1cnccc1C(=O)O ; COC(=O)c1ccncc1Nc1cnn(C2CCNCC2)c1C]

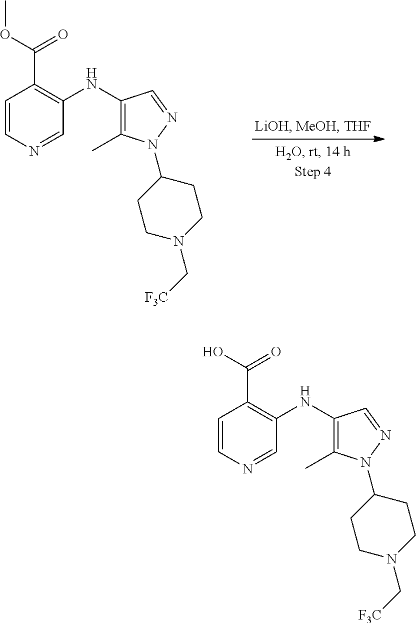 [SMILES: COC(=O)c1ccncc1Nc1cnn(C2CCN(CC(F)(F)F)CC2)c1C ; Cc1c(Nc2cnccc2C(=O)O)cnn1C1CCN(CC(F)(F)F)CC1]

### Synthesis of methyl 3-(3-methyl-1-(1-(2,2,2-trifluoroethyl)piperidin-4-yl)-1H-pyrazol-4-ylamino)isonicotinate and methyl 3-(5-methyl-1-(1-(2,2,2-trifluoroethyl)piperidin-4-yl)-1H-pyrazol-4-ylamino)isonicotinate

To a solution of methyl 3-(3-methyl-1-(piperidin-4-yl)-1H-pyrazol-4-ylamino)isonicotinate (100 mg, 0.18 mmol, 2 TFA salt) and DIPEA (122 mg, 0.94 mmol) in DCM (5 mL) was added 2,2,2-trifluoroethyl trifluoromethanesulfonate (64 mg, 0.28 mmol) at 0° C. The reaction mixture was stirred at room temperature for 4 h, and LCMS indicated the starting material was consumed. Saturated NH₄Cl aqueous solution (10 mL) was added, the organic phase was separated and washed with brine, dried over Na₂SO₄, filtered and concentrated to afford concentrated to afford methyl 3-(3-methyl-1-(1-(2,2,2-trifluoroethyl)piperidin-4-yl)-1H-pyrazol-4-yl amino)isonicotinate (155 mg, crude) as a yellow oil which was used directly into next step without further purification. ESI-LCMS (m/z): 398.2 [M+1]. The same protocol was used to make methyl 3-(5-methyl-1-(1-(2,2,2-trifluoroethyl) piperidin-4-yl)-1H-pyrazol-4-ylamino)isonicotinate.

### Synthesis of 3-(3-methyl-1-(1-(2,2,2-trifluoroethyl)piperidin-4-yl)-1H-pyrazol-4-ylamino)isonicotinic acid and 3-(5-methyl-1-(1-(2,2,2-trifluoroethyl) piperidin-4-yl)-1H-pyrazol-4-ylamino)isonicotinic acid

Standard LiOH hydrolysis conditions as described in paragraph [0500] above.

### Synthesis of methyl 3-((1-(1-isopropylpiperidin-4-yl)-3-methyl-1H-pyrazol-4-yl)amino)isonicotinate, 3-((1-(1-isopropylpiperidin-4-yl)-3-methyl-1H-pyrazol-4-yl)amino)isonicotinic acid, methyl 3-((1-(1-isopropylpiperidin-4-yl)-5-methyl-1H-pyrazol-4-yl)amino)isonicotinate and 3-(1-(1-isopropylpiperidin-4-yl)-5-methyl-1H-pyrazol-4-yl)amino)isonicotinic acid (Compounds 101-104)

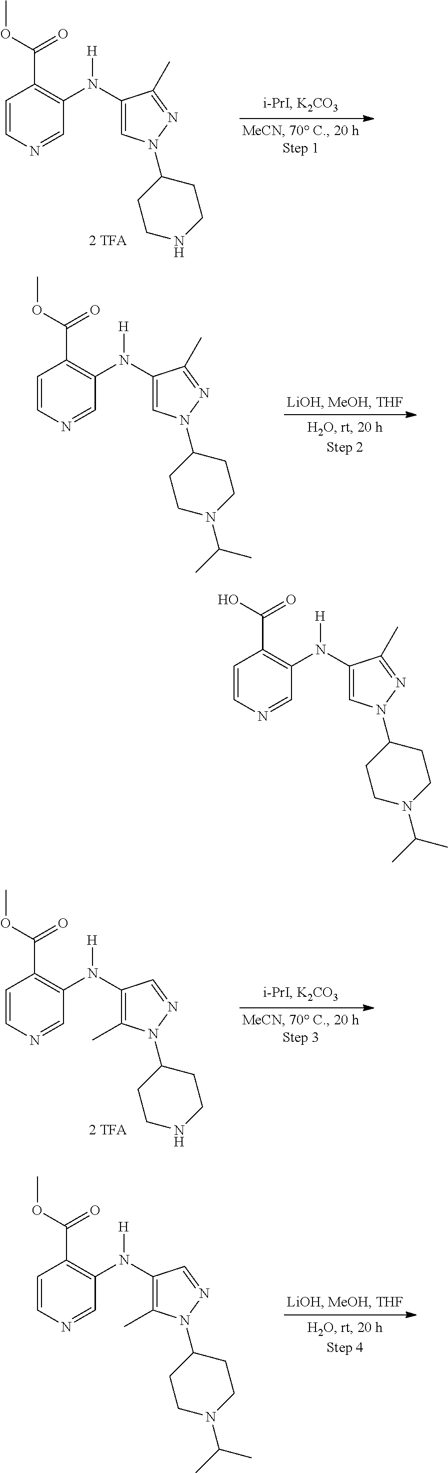

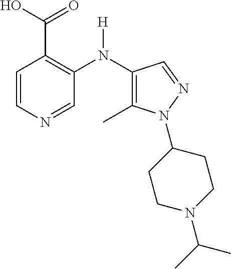 [SMILES: Cc1c(Nc2cnccc2C(=O)O)cnn1C1CCN(C(C)C)CC1]

Compounds 101-104 were synthesized by a method similar to that illustrated in paragraph [0508] above using iPrI and K₂CO₃ as base.

### Synthesis of 3-(3-methyl-1-(1-phenylpiperidin-4-yl)-1H-pyrazol-4-ylamino) isonicotinic acid and 3-(5-methyl-1-(1-phenylpiperidin-4-yl)-1H-pyrazol-4-ylamino)isonicotinic acid (Compounds 105 and 106)

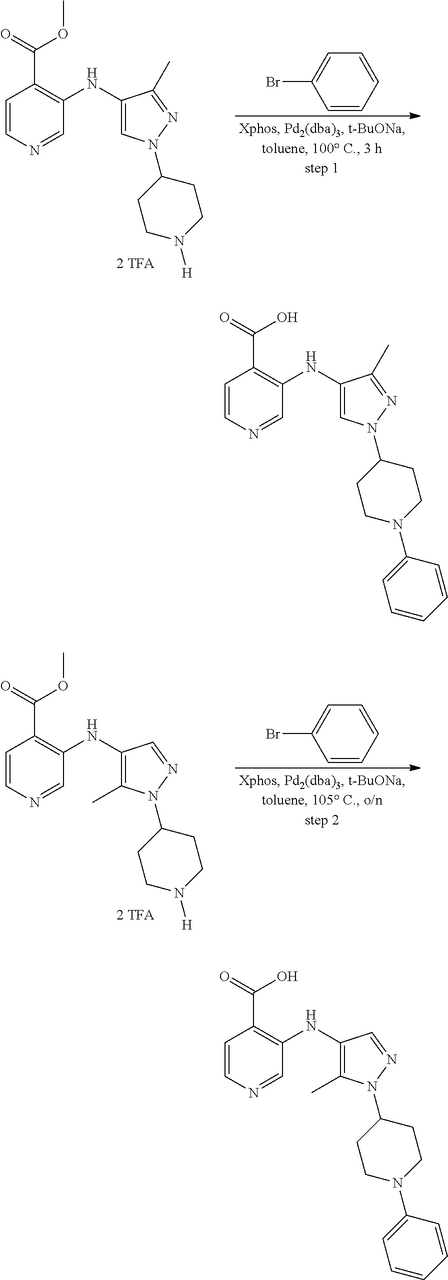 [SMILES: COC(=O)c1ccncc1Nc1cn(C2CCNCC2)nc1C ; Brc1ccccc1 ; Cc1nn(C2CCN(c3ccccc3)CC2)cc1Nc1cnccc1C(=O)O ; COC(=O)c1ccncc1Nc1cnn(C2CCNCC2)c1C ; Brc1ccccc1 ; Cc1c(Nc2cnccc2C(=O)O)cnn1C1CCN(c2ccccc2)CC1]

### Synthesis of 3-(3-methyl-1-(1-phenylpiperidin-4-yl)-1H-pyrazol-4-yl amino)isonicotinic acid and 3-(5-methyl-1-(1-phenylpiperidin-4-yl)-1H-pyrazol-4-yl-amino)isonicotinic acid

The standard Buchwald coupling protocol from paragraph [0306] above was followed (55 mg, Yield: 76%).

### Synthesis of 3-(1-(2-(dimethylamino)ethyl)-3-methyl-1H-pyrazol-4-ylamino) isonicotinic acid and 2-(1-(2-(dimethylamino)ethyl)-5-methyl-1H-pyrazol-4-ylamino)benzoic acid (Compounds 107 and 108)

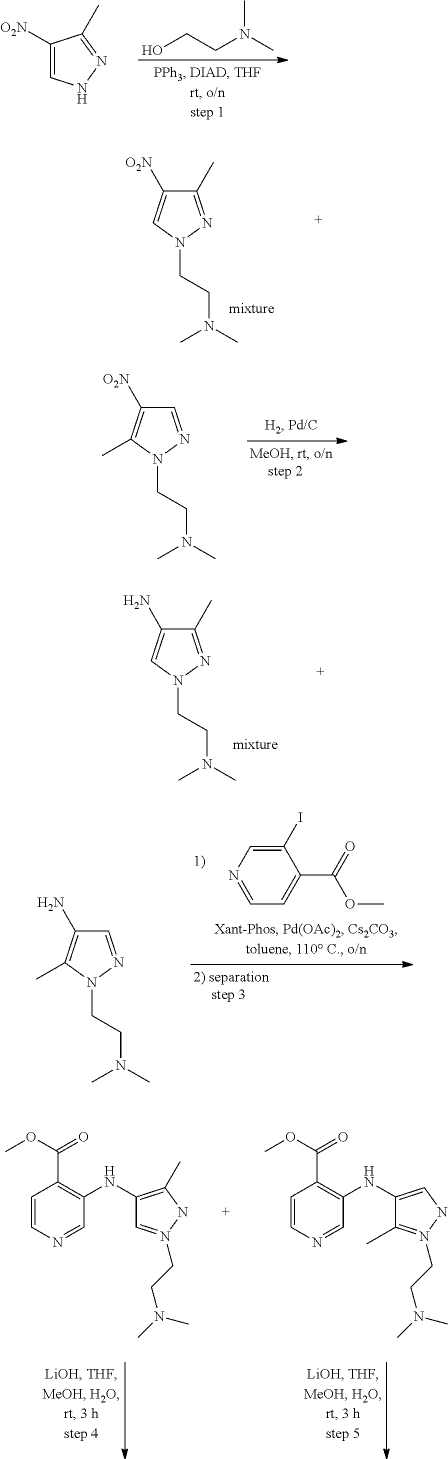

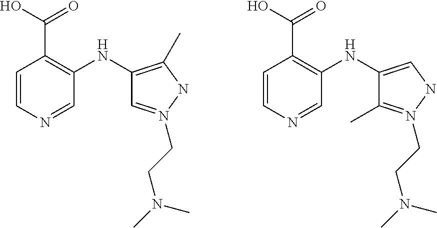

### Synthesis of the mixture of N,N-dimethyl-2-(3-methyl-4-nitro-1H-pyrazol-1-yl)ethanamine and N,N-dimethyl-2-(5-methyl-4-nitro-1H-pyrazol-1-yl) ethanamine

To a solution of 3-methyl-4-nitro-1H-pyrazole (2.54 g, 20 mmol) in anhydrous THF (40 mL) under nitrogen atmosphere was added 2-(dimethylamino)ethanol (1.8 g, 20 mmol), PPh₃ (7.86 g, 30 mmol). The solution was cooled to 0° C. and DIAD (6.06 g, 30 mmol) was added dropwise. After the addition the reaction mixture was stirred for 15 min at 0° C., warmed to room temperature and stirred overnight. The resulting solution was diluted with EtOAc (100 mL) and washed with water (20 mL×3), the organic phase was dried over Na₂SO₄, filtered and concentrated. The residue was purified by silica gel chromatography (eluted with petroleum ether/EtOAc=6/1) to give an inseparable mixture of regioisomers: N,N-dimethyl-2-(3-methyl-4-nitro-1H-pyrazol-1-yl)ethanamine and N,N-dimethyl-2-(5-methyl-4-nitro-1H-pyrazol-1-yl) ethanamine (937 mg, Yield: 23%) as yellow oil. ESI-LCMS (m/z): 199.1 [M+1].

### Synthesis of the mixture of 1-(2-(dimethylamino)ethyl)-3-methyl-1H-pyrazol-4-mine and 1-(2-(dimethylamino)ethyl)-5-methyl-1H-pyrazol-4-amine

To a mixture of N,N-dimethyl-2-(3-methyl-4-nitro-1H-pyrazol-1-yl)ethanamine and N,N-dimethyl-2-(5-methyl-4-nitro-1H-pyrazol-1-yl) ethanamine (868 mg, 4.37 mmol) in MeOH (14 mL) was added Pd/C (10%, 90 mg) with nitrogen protected. The system was degassed, recharged with H₂ and stirred at room temperature overnight. The reaction mixture was filtered through Celite and filtrate was concentrated to give a mixture of 1-(2-(dimethylamino)ethyl)-3-methyl-1H-pyrazol-4-amine and 1-(2-(di-methylamino)ethyl)-5-methyl-1H-pyrazol-4-amine (740 mg, 100%) as white solid. ESI-LCMS (m/z): 169.2 [M+1].

### Synthesis of methyl 3-(1-(2-(dimethylamino)ethyl)-3-methyl-1H-pyrazol-4-ylamino)isonicotinate and methyl 3-(1-(2-(dimethylamino)ethyl)-5-methyl-1H-pyrazol-4-ylamino)isonicotinate

The standard Buchwald coupling protocol from paragraph [0306] above was followed.

### Synthesis of 3-(1-(2-(dimethylamino)ethyl)-3-methyl-1H-pyrazol-4-yl amino)isonicotinic acid and 3-(1-(2-(dimethylamino)ethyl)-5-methyl-1H-pyrazol-4-yl)amino)isonicotinic acid

Standard LiOH hydrolysis conditions as described in paragraph [0500] above were followed.

### Synthesis of 3-(3-methyl-1-((3S,4S)-4-methylpyrrolidin-3-yl)-1H-pyrazol-4-yl-amino)isonicotinic acid and 3-(3-methyl-1-((3R,4R)-4-methyl-pyrrolidin-3-yl)-1H-pyrazol-4-yl-amino)isonicotinic acid (Compounds 109 and 110)

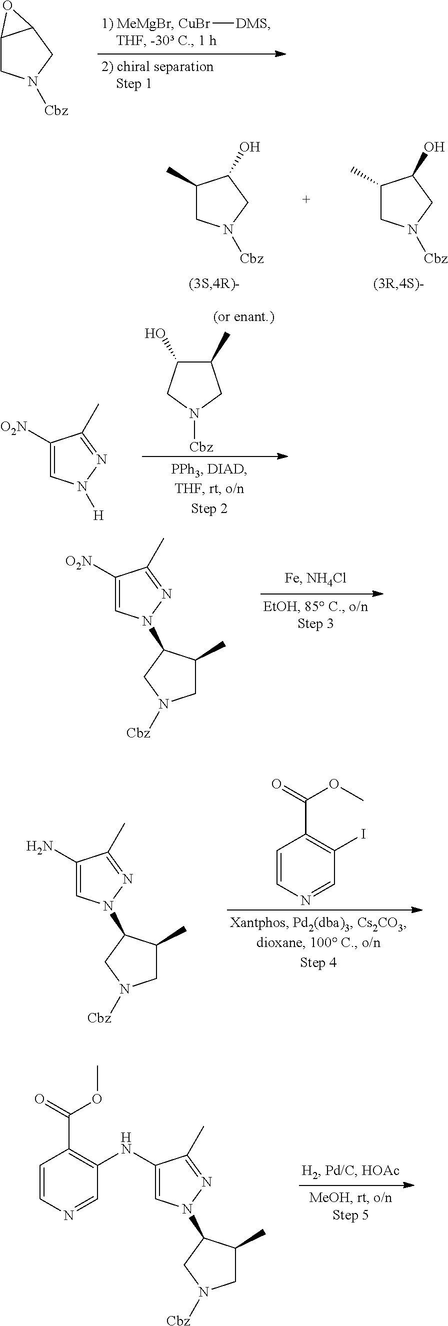

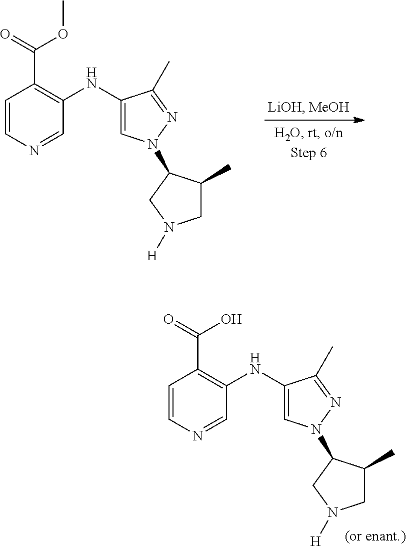 [SMILES: COC(=O)c1ccncc1Nc1cn([C@@H]2CNC[C@@H]2C)nc1C ; Cc1nn([C@@H]2CNC[C@@H]2C)cc1Nc1cnccc1C(=O)O]

### Synthesis of (3S,4R)-benzyl 3-hydroxy-4-methylpyrrolidine-1-carboxylate and (3R,4S)-benzyl 3-hydroxy-4-methylpyrrolidine-1-carboxylate

To a mixture of benzyl 6-oxa-3-aza-bicyclo[3.1.0]hexane-3-carboxylate (10.0 g, 46 mmol) and CuBr-DMS (1.21 g, 5.9 mmol) in 150 mL of anhydrous THF at −30° C. was added MeMgBr (46 mL, 3.0 M in hexane) and the mixture was stirred at −30° C. for 1 h. The mixture was quenched with saturated NH₄Cl aqueous solution (100 mL) and the mixture was extracted with EtOAc (3×150 mL). The combined organic layers were dried over Na₂SO₄, filtered and concentrated to give trans-benzyl 3-hydroxy-4-methylpyrrolidine-1-carboxylate (10.6 g, Yield: 94%) as oil. ¹HNMR (400 MHz, CDCl3): δ 7.26-7.37 (m, 5H), 5.12 (s, 2H), 3.72-3.65 (m, 2H), 3.36-3.30 (m, 1H), 3.15-3.08 (m, 1H), 1.97-1.91 (m, 1H), 1.02 (d, J=7.5 Hz, 3H) ppm; ESI-LCMS (m/z): 236 [M+1].

The racemic sample was further separated by chiral prep-HPLC [AD-H column, 20*250 mm, 5 μm (Dacel); mobile phase: CO2/MeOH (0.5% NH3)=60/40] to give P1 [4.65 g, first peak, arbitrarily assigned as (3S,4R)-configuration] and P2 [4.5 g, second peak, arbitrarily assigned as (3R,4S)-configuration].

### Synthesis of (3S,4S)-benzyl 3-methyl-4-(5-methyl-4-nitro-1H-pyrazol-1-yl) pyrrolidine-1-carboxylate or enantiomer

To a solution of 3-methyl-4-nitro-1H-pyrazole (500 mg, 3.93 mmol) in dry THF (10 mL) was added (3R,4S)-benzyl-3-hydroxyl-4-methylpyrrolidine-1-carboxylate (924 mg, 3.93 mmol), and PPh₃ (2.06 g, 7.86 mmol). The solution was stirred at room temperature for 5 min, and then DIAD (1.58 g, 7.86 mmol) was added dropwise. After addition the reaction mixture was stirred at room temperature overnight. The reaction mixture was concentrated and the residue was purified by silica gel chromatography (eluted with EtOAc/petroleum ether=1/5 to 1/1) to give (3S,4S)-benzyl 3-methyl-4-(5-methyl-4-nitro-1H-pyrazol-1-yl)pyrrolidine-1-carboxylate (1.2 g, Yield: 88% yield). ESI-LCMS (m/z): 345.2 [M+1].

### Synthesis of (3S,4S)-benzyl 3-(4-amino-5-methyl-1H-pyrazol-1-yl)-4-methylpyrrolidine-1-carboxylate or enantiomer

A mixture of (3S,4S)-benzyl 3-methyl-4-(5-methyl-4-nitro-1H-pyrazol-1-yl)pyrrolidine-1-carboxylate (0.7 g, 2.03 mmol), iron powder (453 mg, 8.12 mmol) and ammonium chloride (325 mg, 6.08 mmol) in 75% ethanol (50 mL) was stirred at 85° C. overnight. After cooling down to room temperature, the mixture was filtered through a pad of Celite and the filtrate was concentrated to give (3S,4S)-benzyl 3-(4-amino-5-methyl-1H-pyrazol-1-yl)-4-methylpyrrolidine-1-carboxylate (950 mg, Yield: 87%). ESI-LCMS (m/z): 315.2 [M+1].

### Synthesis of methyl 3-((1-((3S,4S)-1-((benzyloxy)carbonyl)-4-methyl pyrrolidin-3-yl)-5-methyl-1H-pyrazol-4-yl)amino)isonicotinate or enantiomer

The standard Buchwald coupling protocol from paragraph [0306] above was followed (390 mg, Yield: 39%) as a yellow oil. ESI-LCMS (m/z): 450.3 [M+1].

### Synthesis of methyl 3-((5-methyl-1-((3S,4S)-4-methylpyrrolidin-3-yl)-1H-pyrazol-4-yl)amino)isonicotinate or enantiomer

A mixture of methyl 3-((1-((3S,4S)-1-((benzyloxy)carbonyl)-4-methylpyrrolidin-3-yl)-5-methyl-1H-pyrazol-4-yl)amino) isonicotinate (390 mg, 0.88 mmol), 10% Pd/C (189 mg) and HOAc (1 mL) in MeOH (30 mL) was degassed under vacuum and purged with H₂ several times. The mixture was stirred under H₂ atmosphere at room temperature overnight, then filtered through a pad of Celite and the cake was washed with MeOH. The combined filtrates were concentrated and purified by silica gel chromatography (eluted with EtOAc/petroleum ether=1/2 to 2/1) to give methyl 3-(3-methyl-1-((3S,4S)-4-methylpyrrolidin-3-yl)-1H-pyrazol-4-ylamino) isonicotinate (180 mg, Yield: 64%) as yellow oil. ESI-LCMS (m/z): 316.2 [M+1].

### Synthesis of 3-((5-methyl-1-((3S,4S)-4-methylpyrrolidin-3-yl)-1H-pyrazol-4-yl)amino) isonicotinic acid or enantiomer

Standard LiOH hydrolysis conditions as described in paragraph [0500] above were followed.

### Synthesis of 3-(3-methyl-1-(1-methylazepan-4-yl)-1H-pyrazol-4-ylamino) isonicotinic acid and 3-((5-methyl-1-(1-methylazepan-4-yl)-1H-pyrazol-4-yl)amino)isonicotinic acid (Compounds 111 and 112)

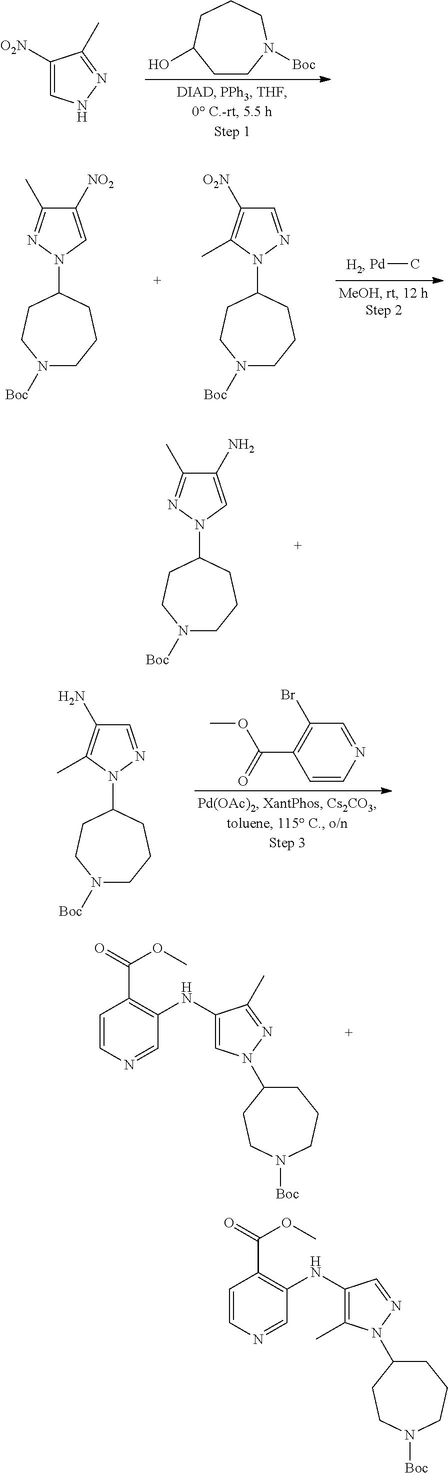

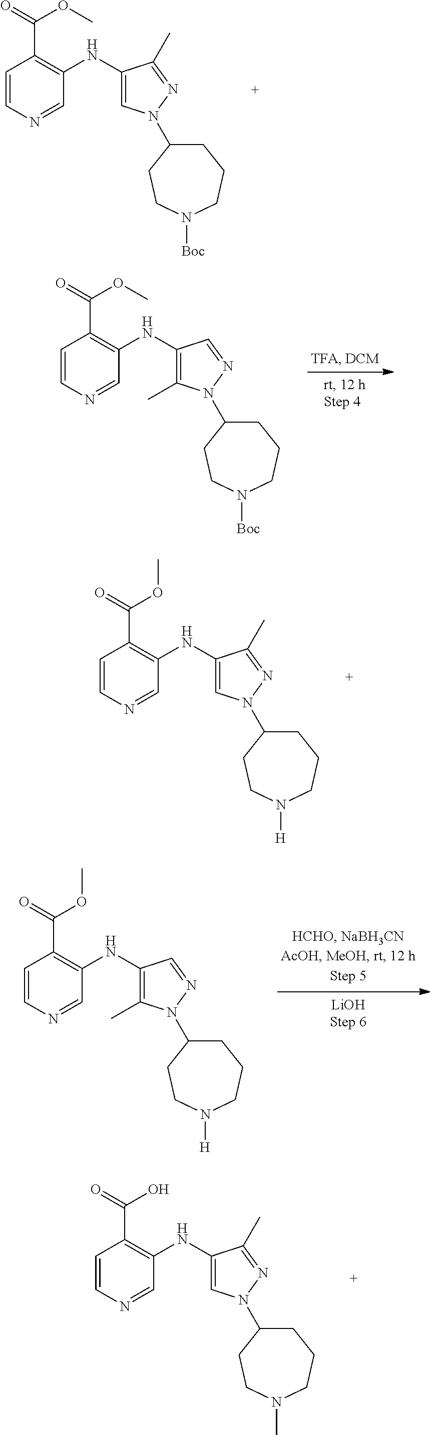 [SMILES: COC(=O)c1ccncc1Nc1cn(C2CCCN(C(=O)OC(C)(C)C)CC2)nc1C ; COC(=O)c1ccncc1Nc1cnn(C2CCCN(C(=O)OC(C)(C)C)CC2)c1C ; COC(=O)c1ccncc1Nc1cn(C2CCCNCC2)nc1C ; COC(=O)c1ccncc1Nc1cnn(C2CCCNCC2)c1C ; Cc1nn(C2CCCN(C)CC2)cc1Nc1cnccc1C(=O)O]

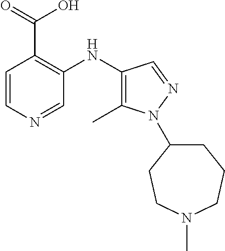 [SMILES: Cc1c(Nc2cnccc2C(=O)O)cnn1C1CCCN(C)CC1]

Compounds 111 and 112 were synthesized by a method similar to that illustrated in paragraph [0445] above except that the starting compound was tert-butyl 4-hydroxyazepane-1-carboxylate.

### Synthesis of (S)-3-(1-(azepan-4-yl)-5-methyl-1H-pyrazol-4-yl)amino) iso nicotinic acid, (R)-3-(1-(azepan-4-yl)-5-methyl-1H-pyrazol-4-yl)amino)isonicotinic acid, (S)-3-((1-(azepan-4-yl)-3-methyl-1H-pyrazol-4-yl)amino)isonicotinic acid and (R)-3-((1-(azepan-4-yl)-3-methyl-1H-pyrazol-4-yl)amino) isonicotinic acid (Compounds 113-116)

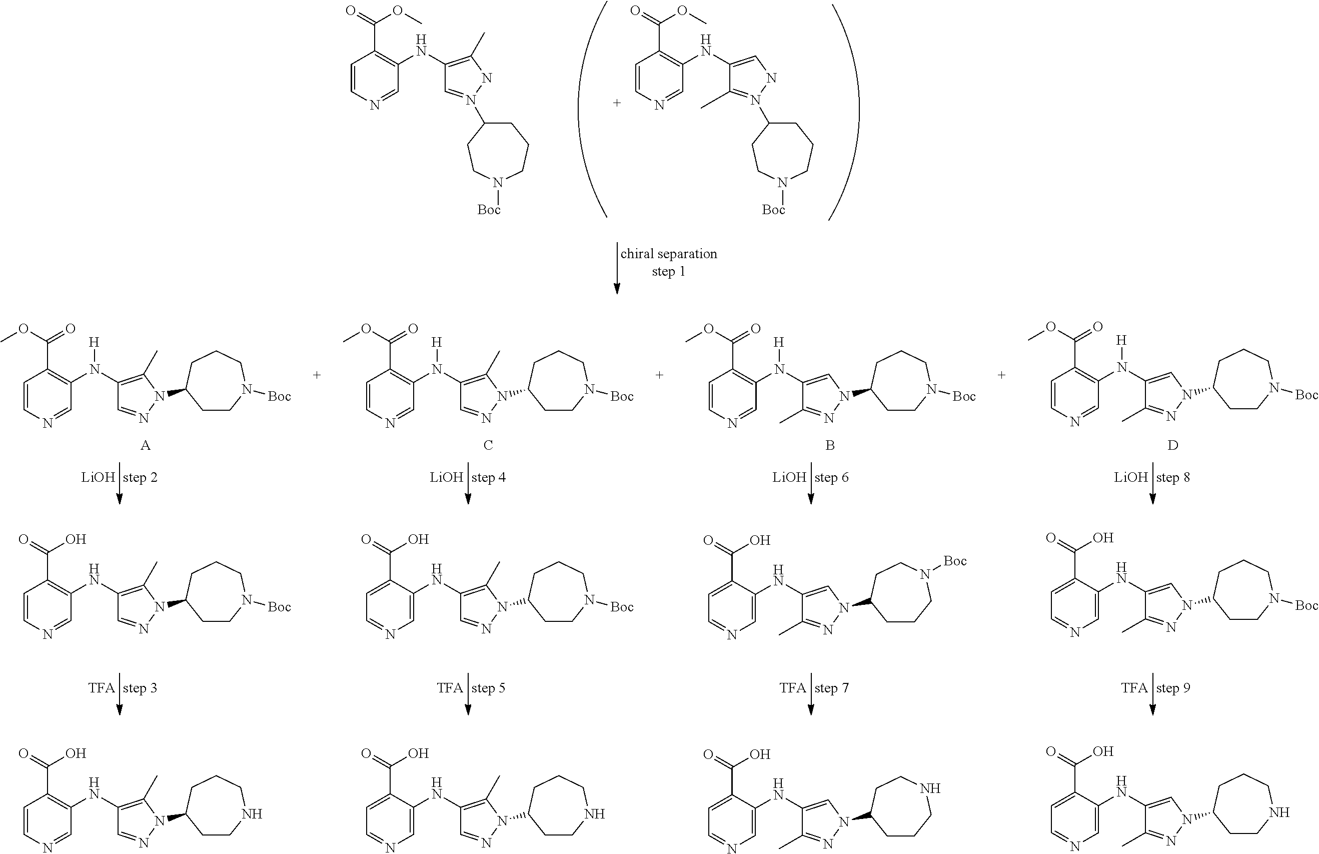

Chiral Separation:

The mixture of tert-butyl 4-(4-(4-(methoxycarbonyl) pyridin-3-ylamino)-3-methyl-1H-pyrazol-1-yl)azepane-1-carboxylate and tert-butyl 4-(4-(4-(methoxycarbonyl)pyridin-3-ylamino)-5-methyl-1H-pyrazol-1-yl)azepane-1-carboxylate (4.22 g, 9.8 mmol) was separated by chiral HPLC [AD-H (4.6*250 mm, Sum); MeOH (0.2% Methanol Ammonia)], and four fractions were obtained: P1, P2, P3 and P4. After concentration and structural analysis, P1 and P3 have identical HNMR and were confirmed as a pair of enantiomers of tert-butyl 4-(4-((4-(methoxycarbonyl)pyridine-3-yl)amino)-5-methyl-1H-pyrazol-1-yl)azepane-1-carboxylate. While the P2 and P4 were enantiomers of tert-butyl 4-(4-((4-(methoxycarbonyl)pyridin-3-yl)amino)-3-methyl-1H-pyrazol-1-yl)azepane-1-carboxylate. The absolute configuration of each compound was temporarily assumed to be (R)— and (S)—.

P1: (S)-tert-butyl 4-(4-((4-(Methoxycarbonyl)pyridin-3-yl)amino)-5-methyl-1H-pyrazol-1-yl)azepane-1-carboxylate (340 mg). ESI-LCMS (m/z): 430.2 [M+1]⁺; ¹HNMR (400 MHz, CDCl₃): δ 8.41 (s, 1H), 8.08 (s, 1H), 7.96 (d, J=5.2 Hz, 1H), 7.65 (d, J=5.2 Hz, 1H), 7.45 (s, 1H), 4.20-4.10 (m, 1H), 3.95 (s, 3H), 3.80-3.20 (m, 4H), 2.35-2.25 (m, 1H), 2.24-2.12 (m, 5H), 2.10-1.90 (m, 2H), 1.80-1.65 (m, 2H), 1.50 (s, 9H) ppm.

P2: (S)-tert-butyl 4-(4-((4-(methoxycarbonyl)pyridin-3-yl)amino)-3-methyl-1H-pyrazol-1-yl)azepane-1-carboxylate (717 mg). ESI-LCMS (m/z): 430.2 [M+1]⁺; ¹HNMR (400 MHz, CDCl₃): δ 8.44 (s, 1H), 8.14 (s, 1H), 7.96 (d, J=5.2 Hz, 1H), 7.65 (d, J=5.2 Hz, 1H), 7.37 (s, 1H), 4.24-4.14 (m, 1H), 3.95 (s, 3H), 3.80-3.26 (m, 4H), 2.33-2.25 (m, 1H), 2.24-1.68 (m, 9H), 1.49 (s, 9H) ppm.

P3: (R)-tert-butyl 4-(4-((4-(methoxycarbonyl)pyridin-3-yl)amino)-5-methyl-1H-pyrazol-1-yl)azepane-1-carboxylate (294 mg). ESI-LCMS (m/z): 430.2 [M+1]⁺; ¹HNMR (400 MHz, CDCl₃): δ 8.41 (s, 1H), 8.08 (s, 1H), 7.96 (d, J=5.2 Hz, 1H), 7.65 (d, J=5.2 Hz, 1H), 7.45 (s, 1H), 4.20-4.10 (m, 1H), 3.95 (s, 3H), 3.80-3.20 (m, 4H), 2.35-2.25 (m, 1H), 2.24-2.12 (m, 5H), 2.10-1.90 (m, 2H), 1.80-1.65 (m, 2H), 1.50 (s, 9H) ppm.

P4: (R)-tert-butyl 4-(4-((4-(methoxycarbonyl)pyridin-3-yl)amino)-3-methyl-1H-pyrazol-1-yl)azepane-1-carboxylate (591 mg). ESI-LCMS (m/z): 430.2 [M+1]⁺; ¹HNMR (400 MHz, CDCl₃): δ 8.44 (s, 1H), 8.14 (s, 1H), 7.96 (d, J=5.2 Hz, 1H), 7.65 (d, J=5.2 Hz, 1H), 7.37 (s, 1H), 4.24-4.14 (m, 1H), 3.95 (s, 3H), 3.80-3.26 (m, 4H), 2.33-2.25 (m, 1H), 2.24-1.68 (m, 9H), 1.49 (s, 9H) ppm.

### Synthesis of (S)-3-((1-(1-(tert-butoxycarbonyl)azepan-4-yl)-5-methyl-1H-pyrazol-4-yl)amino)isonicotinic acid and 3 other isomers

Standard LiOH hydrolysis conditions as described in paragraph [0500] above.

### Synthesis of (S)-3-((1-(azepan-4-yl)-5-methyl-1H-pyrazol-4-yl)amino)iso nicotinic acid and 3 additional isomers

The standard TFA BOC deprotection protocol as described in paragraph [0448] was used.

### Synthesis of (S)-3-(1-(1-cyclobutylazepan-4-yl)-5-methyl-1H-pyrazol-4-yl amino)isonicotinic acid, (S)-3-((1-(1-cyclobutylazepan-4-yl)-3-methyl-1H-pyrazol-4-yl)amino)isonicotinic acid, (R)-3-((1-(1-cyclobutyl-azepan-4-yl)-5-methyl-1H-pyrazol-4-yl)amino) isonicotinic acid and (R)-3-((1-(1-cyclo-butylazepan-4-yl)-3-methyl-1H-pyrazol-4-yl)amino)isonicotinic acid (Compounds 117-120)

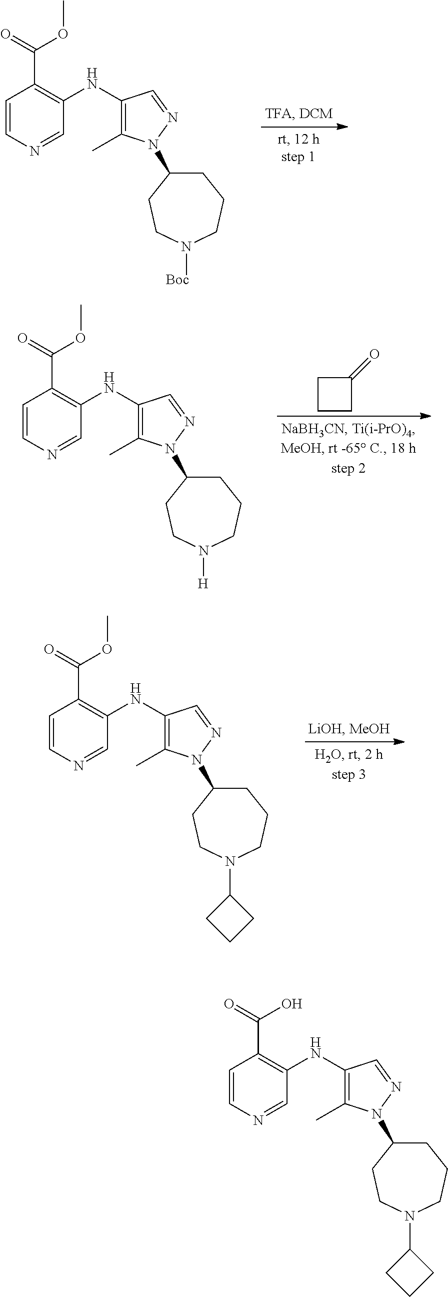 [SMILES: COC(=O)c1ccncc1Nc1cnn([C@H]2CCCN(C(=O)OC(C)(C)C)CC2)c1C ; COC(=O)c1ccncc1Nc1cnn([C@H]2CCCNCC2)c1C ; O=C1CCC1 ; COC(=O)c1ccncc1Nc1cnn([C@H]2CCCN(C3CCC3)CC2)c1C ; Cc1c(Nc2cnccc2C(=O)O)cnn1[C@H]1CCCN(C2CCC2)CC1]

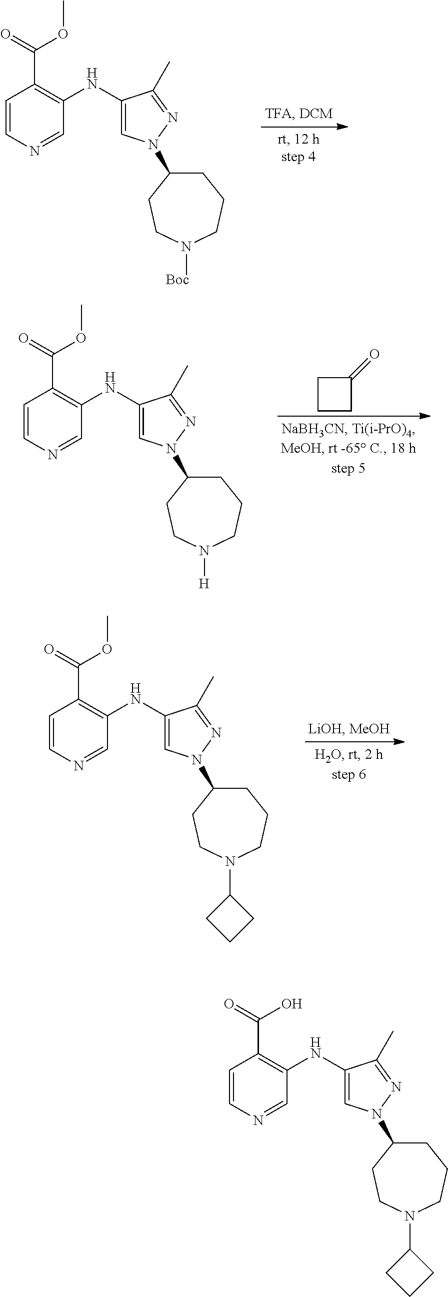 [SMILES: COC(=O)c1ccncc1Nc1cn([C@H]2CCCN(C(=O)OC(C)(C)C)CC2)nc1C ; COC(=O)c1ccncc1Nc1cn([C@H]2CCCNCC2)nc1C ; O=C1CCC1 ; COC(=O)c1ccncc1Nc1cn([C@H]2CCCN(C3CCC3)CC2)nc1C ; Cc1nn([C@H]2CCCN(C3CCC3)CC2)cc1Nc1cnccc1C(=O)O]

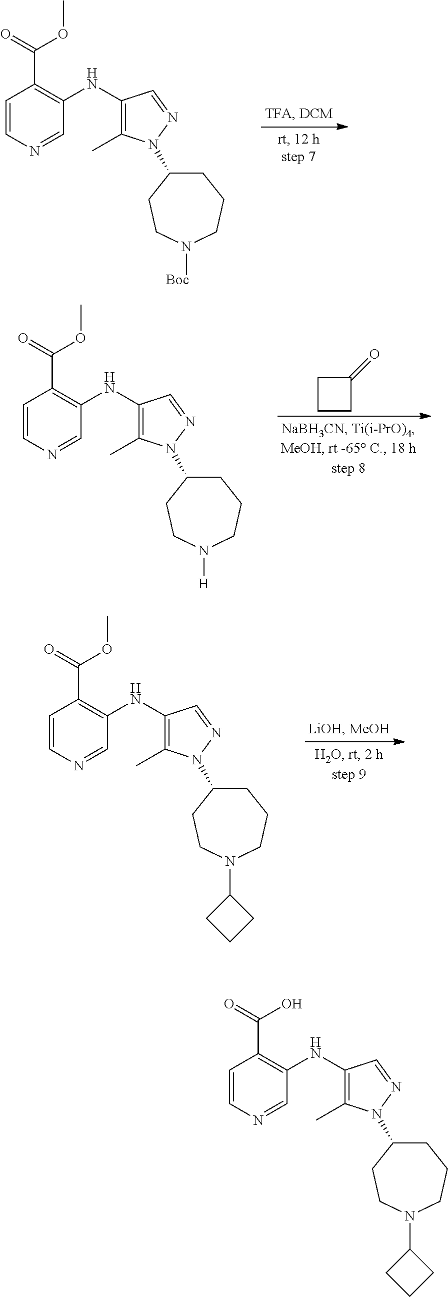 [SMILES: COC(=O)c1ccncc1Nc1cnn([C@@H]2CCCN(C(=O)OC(C)(C)C)CC2)c1C ; COC(=O)c1ccncc1Nc1cnn([C@@H]2CCCNCC2)c1C ; O=C1CCC1 ; COC(=O)c1ccncc1Nc1cnn([C@@H]2CCCN(C3CCC3)CC2)c1C ; Cc1c(Nc2cnccc2C(=O)O)cnn1[C@@H]1CCCN(C2CCC2)CC1]

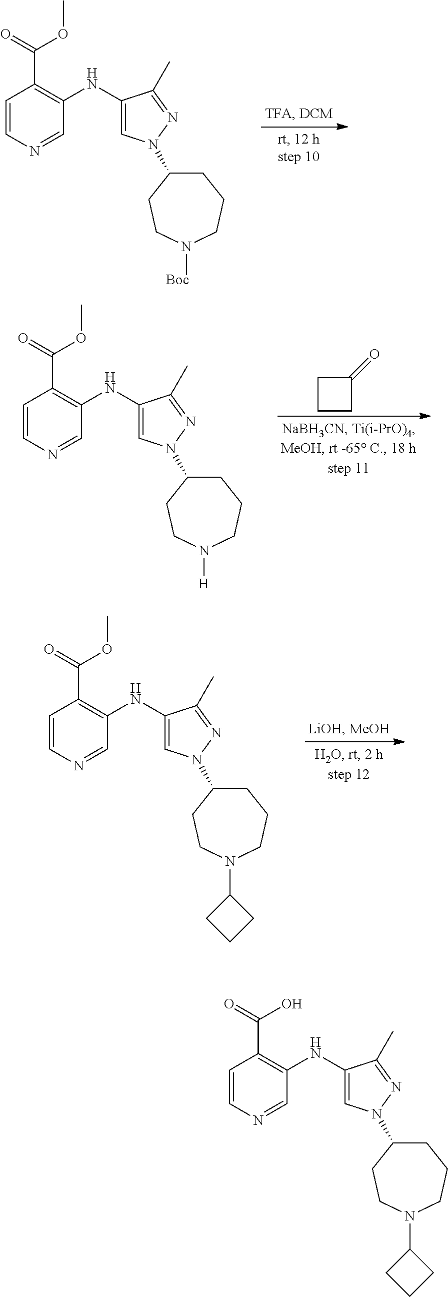 [SMILES: COC(=O)c1ccncc1Nc1cn([C@@H]2CCCN(C(=O)OC(C)(C)C)CC2)nc1C ; COC(=O)c1ccncc1Nc1cn([C@@H]2CCCNCC2)nc1C ; O=C1CCC1 ; COC(=O)c1ccncc1Nc1cn([C@@H]2CCCN(C3CCC3)CC2)nc1C ; Cc1nn([C@@H]2CCCN(C3CCC3)CC2)cc1Nc1cnccc1C(=O)O]

Compounds 117-120 were synthesized using the protocols as described in paragraphs [0449], [0498], and [0500] above.

### Synthesis of (S)—N-(2-hydroxyethyl)-3-((3-methyl-1-(1-methylpyrrolidin-3-yl)-1H-pyrazol-4-yl)amino)isonicotinamide and (R)—N-(2-hydroxy-ethyl)-3-((3-methyl-1-(1-methylpyrrolidin-3-yl)-1H-pyrazol-4-yl) amino)isonicotin-amide (Compounds 121 and 122)

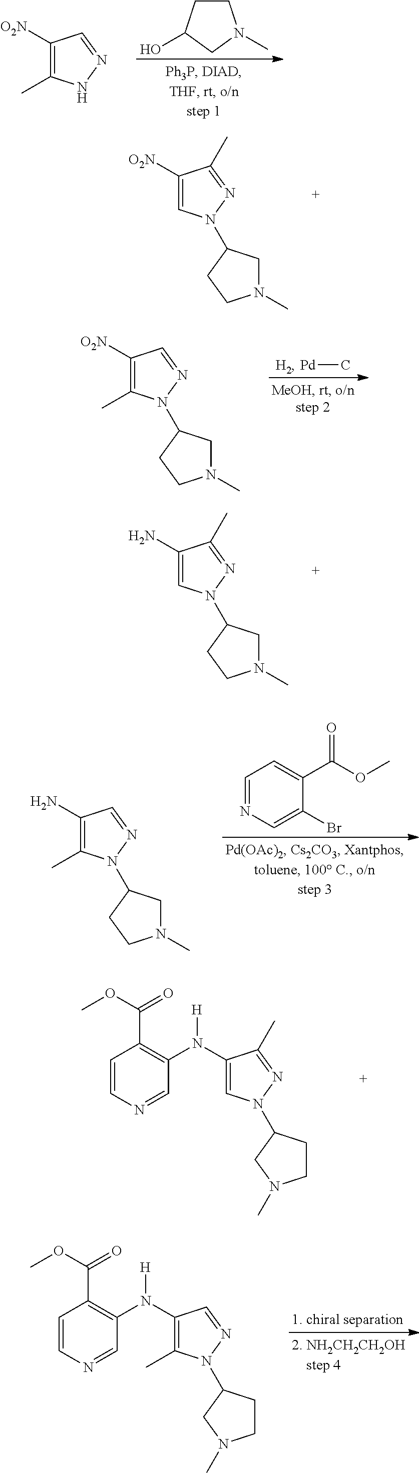

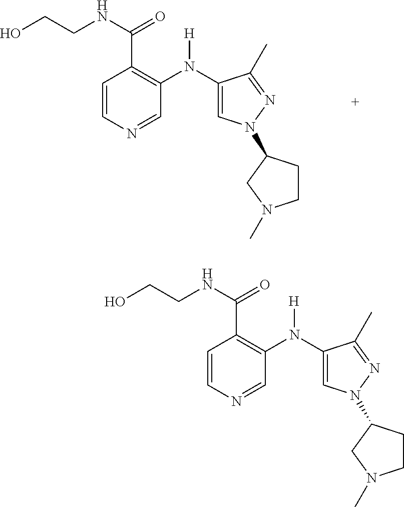 [SMILES: Cc1nn([C@H]2CCN(C)C2)cc1Nc1cnccc1C(=O)NCCO ; Cc1nn([C@@H]2CCN(C)C2)cc1Nc1cnccc1C(=O)NCCO]

Compounds 121 and 122 were synthesized by a method similar to that illustrated in paragraph [0445] above except that the starting compound was 1-methylpyrrolidin-3-ol and the reaction was followed by chiral purification and in situ amide formation.

### Synthesis of (S)—N-(2-hydroxyethyl)-3-((3-methyl-1-(1-methylpyrrolidin-3-yl)-1H-pyrazol-4-yl)amino)isonicotinamide and (R)—N-(2-hydroxyethyl)-3-((3-methyl-1-(1-methylpyrrolidin-3-yl)-1H-pyrazol-4-yl)amino)isonicotinamide

The sample of methyl-3-((3-methyl-1-(1-methylpyrrolidin-3-yl)-1H-pyrazol-4-yl) amino)isonicotinate and methyl 3-(5-methyl-1-(1-methylpyrrolidin-3-yl)-1H-pyrazol-4-yl)isonicotinate (130 mg) was separated by Chiral-HPLC [Column: AY-H (250*4.6 mm, Sum), Mobile Phase: n-Hexane (0.1% ethanolamine): EtOH (0.1% ethanolamine)=20:80]. Two separated major isomers were reacted with ethanolamine when the solvent was concentrated in vacuo with heating, and a pair of enantiomeric by-products was obtained.

### (S)—N-(2-hydroxyethyl)-3-((3-methyl-1-(1-methylpyrrolidin-3-yl)-1H-pyrazol-4-yl)amino)isonicotinamide

20 mg, Yield: 14%. ESI-LCMS (m/z): 345.2 [M+1]; ¹H NMR (400 MHz, Methanol-d₄): δ 7.94 (s, 1H), 7.89 (d, J=5.2 Hz, 1H), 7.76 (s, 1H), 7.52 (d, J=5.1 Hz, 1H), 4.88-4.83 (m, 1H), 3.74 (t, J=5.7 Hz, 2H), 3.53 (t, J=5.8 Hz, 2H), 3.10-3.00 (m, 1H), 2.96-2.87 (m, 2H), 2.75-2.65 (m, 1H), 2.56-2.46 (m, 1H), 2.44 (s, 3H), 2.29-2.16 (m, 1H), 2.11 (s, 3H).

### (R)—N-(2-hydroxyethyl)-3-((3-methyl-1-(1-methylpyrrolidin-3-yl)-1H-pyrazol-4-yl)amino)isonicotinamide

16 mg, Yield: 11%. ESI-LCMS (m/z): 345.2 [M+1]; ¹H NMR (400 MHz, Methanol-d₄): δ 7.94 (s, 1H), 7.90 (d, J=5.2 Hz, 1H), 7.76 (s, 1H), 7.52 (d, J=5.1 Hz, 1H), 4.88-4.84 (m, 1H), 3.74 (t, J=5.8 Hz, 2H), 3.53 (t, J=5.8 Hz, 2H), 3.07-3.01 (m, 1H), 2.94-2.85 (m, 2H), 2.74-2.65 (m, 1H), 2.54-2.45 (m, 1H), 2.43 (s, 3H), 2.28-2.17 (m, 1H), 2.11 (s, 3H).

### Synthesis of 3-(5-ethyl-1-(1-methylpiperidin-4-yl)-1H-pyrazol-4-ylamino) isonicotinic acid (Compound 123)

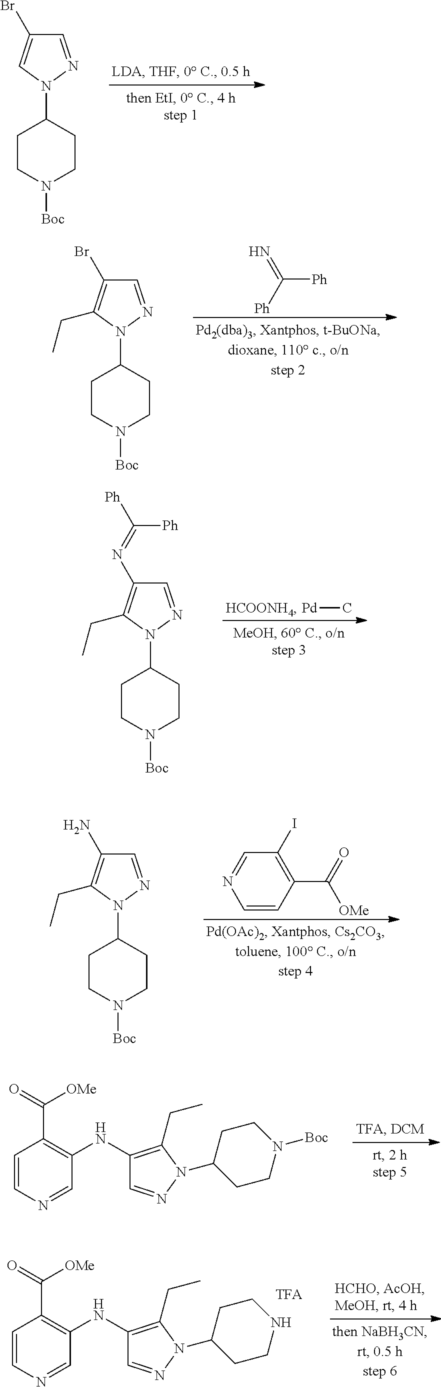

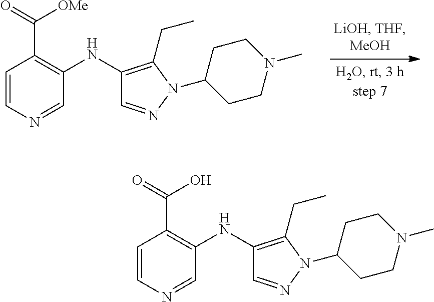 [SMILES: CCc1c(Nc2cnccc2C(=O)OC)cnn1C1CCN(C)CC1 ; CCc1c(Nc2cnccc2C(=O)O)cnn1C1CCN(C)CC1]

### Synthesis of tert-butyl 4-(4-bromo-5-ethyl-1H-pyrazol-1-yl)piperidine-1-carboxylate

To a solution of tert-butyl 4-(4-bromo-1H-pyrazol-1-yl)piperidine-1-carboxylate (990 mg, 2.99 mmol) in anhydrous THF (10 mL) under nitrogen atmosphere was added LDA (2M, 4.5 mL, 9.0 mmol) slowly at 0° C. while stirring. After 0.5 h, iodoethane (1.39 g, 8.97 mmol) was added slowly at 0° C., the mixture was stirred at the same temperature for 4 h. Then saturated NH₄Cl aqueous solution (20 mL) was added and the mixture was extracted with EtOAc (20 mL×4). The combined organic layers was dried over MgSO4, filtered and concentrated. The residue was purified by prep-HPLC to give tert-butyl 4-(4-bromo-5-ethyl-1H-pyrazol-1-yl)piperidine-1-carboxylate (630 mg, Yield: 59%) as a white solid. ESI-LCMS (m/z): 304.0 [M−56]⁺.

### Synthesis of tert-butyl 4-(4-(diphenylmethyleneamino)-5-ethyl-1H-pyrazol-1-yl)piperidine-1-carboxylate

The standard Buchwald coupling protocol from paragraph [0306] above was followed (340 mg, Yield: 50%).

### Synthesis of tert-butyl 4-(4-amino-5-ethyl-1H-pyrazol-1-yl)piperidine-1-carboxylate

To a mixture of tert-butyl 4-(4-(diphenylmethyleneamino)-5-ethyl-1H-pyrazol-1-yl)piperidine-1-carboxylate (340 mg, 0.74 mmol), HCOONH₄ (467 mg, 7.41 mmol) in MeOH (6 mL) was added 10% Pd/C (35 mg). The reaction mixture was warmed to 60° C. and stirred overnight. After cooled down to room temperature, the mixture was filtered through Celite and the filtrate was concentrated. The residue was diluted with water (10 mL), extracted with EtOAc (10 mL×5). The combined organic layers was dried over Na₂SO₄, filtered and concentrated. The residue was purified with prep-TLC (petroleum ether/EtOAc=6/1) to give the title compound as white solid (71 mg, Yield: 32%). ESI-LCMS (m/z): 295.2 [M+1].

### Synthesis of 3-(5-ethyl-1-(1-methylpiperidin-4-yl)-1H-pyrazol-4-ylamino) isonicotinic acid

The standard Buchwald coupling protocol from paragraph [0306] above was followed. The standard TFA BOC deprotection protocol as described in paragraph was used. Standard Eschweiler-Clarke conditions as described in paragraph [0484] above were employed. Standard LiOH hydrolysis conditions as described in paragraph [0500] above were used.

### Synthesis of 3-(1-(1-methylpyrrolidin-3-yl)-5-phenyl-1H-pyrazol-4-yl) amino)isonicotinic acid (Compound 124)

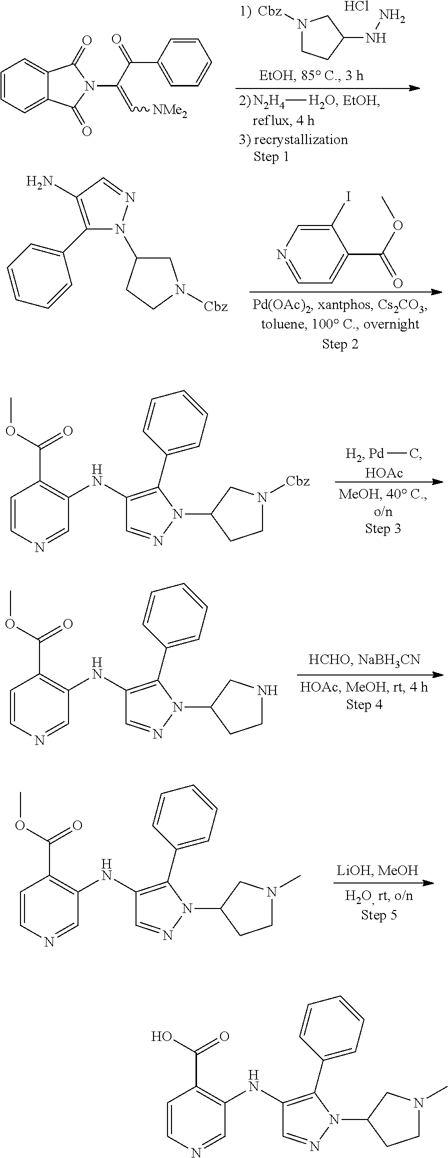

Compound 124 was synthesized by a method similar to that illustrated in paragraph [0330] above except that the starting compound was benzyl 3-hydrazinylpyrrolidine-1-carboxylate.

### Synthesis of 3-(1-(1-benzylpyrrolidin-3-yl)-5-phenyl-1H-pyrazol-4-ylamino) isonicotinic acid (Compound 125)

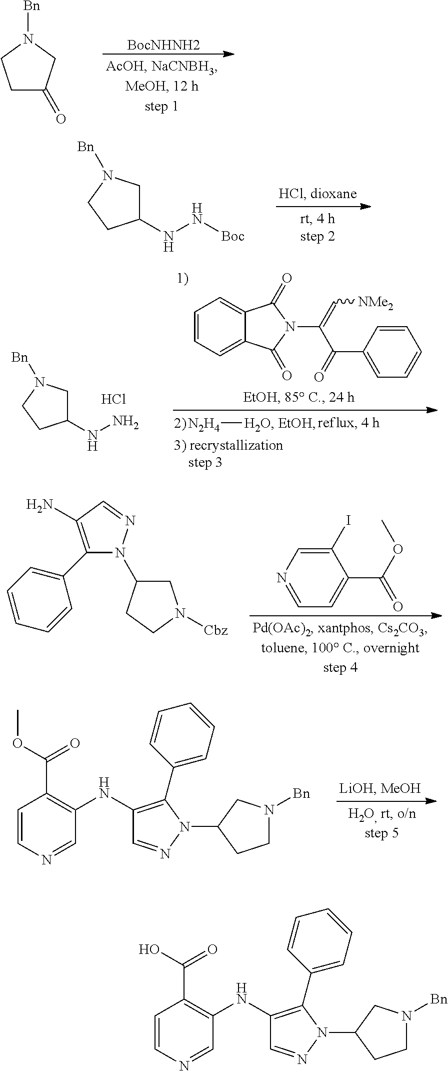

Compound 125 was synthesized by a method similar to that illustrated in paragraph [0330] above except that the starting compound was 1-benzyl-3-hydrazinylpyrrolidine hydrochloride.

### Synthesis of tert-butyl 2-(1-benzylpyrrolidin-3-yl)hydrazinecarboxylate

The mixture of 1-benzylpyrrolidin-3-one (2.62 g, 15 mmol), tert-butyl hydrazine carboxylate (1.98 g, 15.0 mmol) and acetic acid (900 mg, 15.0 mmol) in methanol (80 mL) was stirred at room temperature for 1 h, then sodium cyanoborohydride (1.41 g, 22.5 mmol) was added. The resulting mixture was stirred for another 12 h at room temperature. After concentration, the residue was diluted with water (100 mL), extracted with EtOAc (100 mL×2). The combined organic layers was dried over Na₂SO₄, filtered and concentrated to afford tert-butyl 2-(1-benzylpyrrolidin-3-yl) hydrazinecarboxylate (2.5 g, Yield: 57%) as a pale yellow oil. ESI-LCMS (m/z): 292.2 [M+1].

### Synthesis of 1-benzyl-3-hydrazinylpyrrolidine hydrochloride

To a solution of tert-butyl 2-(1-benzylpyrrolidin-3-yl)hydrazinecarboxylate (2.5 g, 8.57 mmol) in DCM (5 mL) was added 4N HCl in dioxane (21.4 mL, 85.7 mmol). The reaction mixture was stirred at room temperature for 4 h, then concentrated to afford 1-benzyl-3-hydrazinylpyrrolidine hydrochloride (2.0 g, crude) as a pale yellow oil which was used directly in next step without further purification. ESI-LCMS (m/z): 192.2 [M+1].

### Synthesis of 3-(1-(1-methylpiperidin-4-yl)-5-phenyl-1H-pyrazol-4-ylamino) isonicotinic acid and 3-(1-(1-methylpiperidin-4-yl)-3-phenyl-1H-pyrazol-4-ylamino)isonicotinic acid (Compounds 126 and 127)

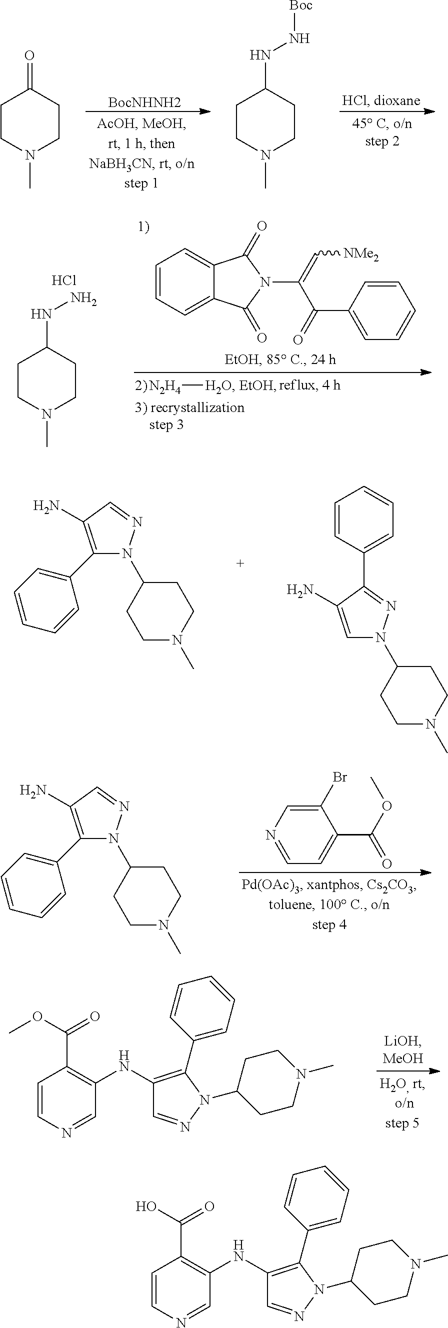

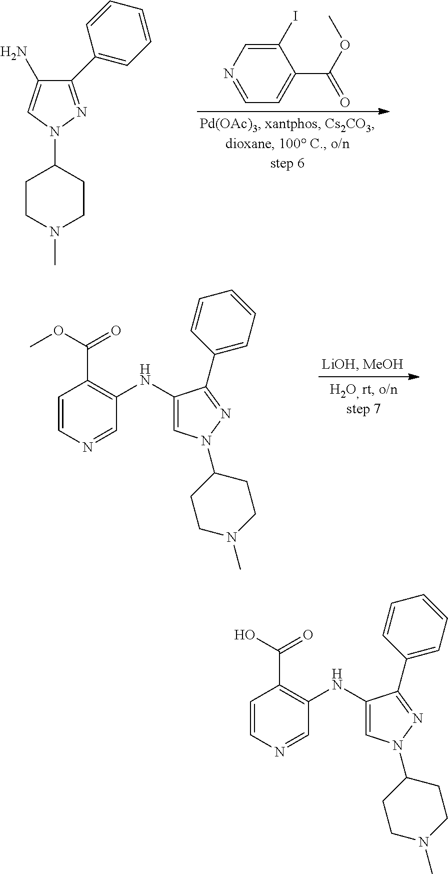 [SMILES: CN1CCC(n2cc(N)c(-c3ccccc3)n2)CC1 ; COC(=O)c1ccncc1I ; COC(=O)c1ccncc1Nc1cn(C2CCN(C)CC2)nc1-c1ccccc1 ; CN1CCC(n2cc(Nc3cnccc3C(=O)O)c(-c3ccccc3)n2)CC1]

Compounds 126 and 127 were synthesized by a method similar to that illustrated in paragraph [0330] above except that the starting compound was 4-hydrazinyl-1-methylpiperidine hydrochloride.

### Synthesis of tert-butyl 2-(1-methylpiperidin-4-yl)hydrazinecarboxylate

A mixture of 1-methylpiperidin-4-one (1.13 g, 10 mmol), tert-butyl hydrazine carboxylate (1.32 g, 10 mmol) and acetic acid (1.20 g, 20 mmol) in methanol (50 mL) was stirred at room temperature for 1 h, then Sodium cyanoborohydride (1.25 g, 20 mmol) was added. The resulting mixture was stirred at room temperature overnight. After the reaction was complete, the mixture was diluted with water (100 mL) and extracted with EtOAc (100 mL×2). The combined organic layers were washed with brine (50 mL), dried over Na₂SO₄, filtered and concentrated to give tert-butyl 2-(1-methylpiperidin-4-yl)hydrazinecarboxylate (2.0 g, Yield: 87%) as pale yellow oil. ESI-LCMS (m/z): 230.2 [M+1].

### Synthesis of 4-hydrazinyl-1-methylpiperidine hydrochloride

The tert-butyl 2-(1-methylpiperidin-4-yl)hydrazinecarboxylate (2.0 g, 8.72 mmol) in dioxane was treated with 4N Hydrochloric acid in dioxane (21.8 mL, 87.2 mmol) at room temperature. The reaction mixture was stirred at 45° C. overnight, and then concentrated to afford 4-hydrazinyl-1-methylpiperidine hydrochloride (1.2 g, crude) as white solid which was used directly in next step without further purification. ESI-LCMS (m/z): 130.2 [M+1].

### Synthesis of 3-(1-benzyl-4-methyl-1H-pyrazol-3-ylamino)isonicotinic acid (Compound 128)

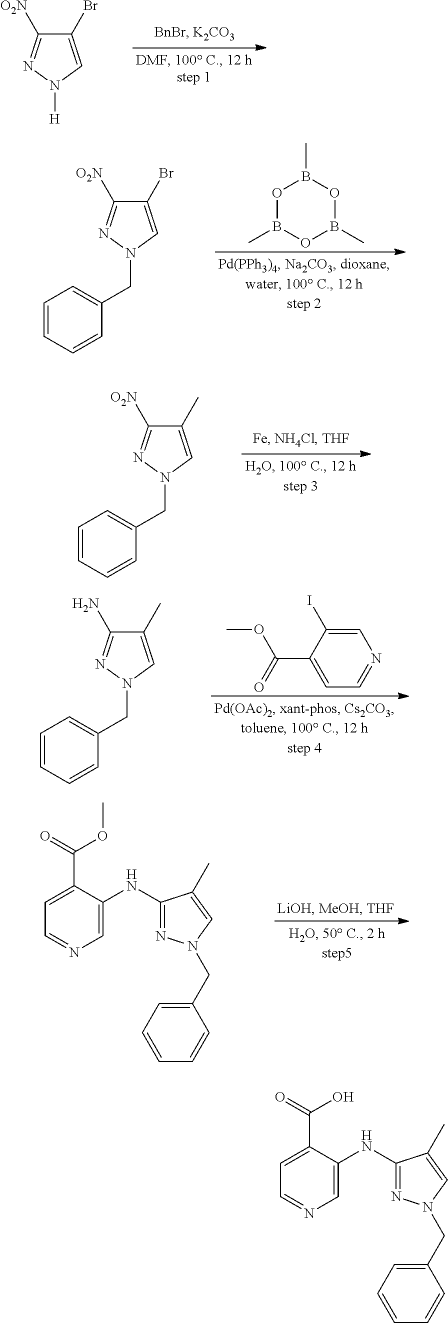

### Synthesis of 1-benzyl-4-bromo-3-nitro-1H-pyrazole

To a solution of 4-bromo-3-nitro-1H-pyrazole (1.3 g, 6.8 mmol) in DMF (15 mL) was added benzyl bromide (1.72 g, 10.1 mmol) and K₂CO₃ (1.86 g, 13.5 mmol) at room temperature. The reaction mixture was stirred at 80° C. for 12 h. After cooling down to room temperature, the mixture was diluted with water (40 mL) and extracted with EtOAc (40 mL×2). The combined organic layers were washed with water (30 mL) and brine (30 mL), dried over Na₂SO₄, filtered and concentrated. The residue was purified by silica gel chromatography (eluted with petroleum ether/EtOAc from 0% to 20%) to give the 1-benzyl-4-bromo-3-nitro-1H-pyrazole (1.5 g, Yield: 78%) as a yellow solid. ESI-LCMS (m/z): 282.0 [M+1]; ¹HNMR (400 MHz, CD₃OD): δ 8.05 (s, 1H), 7.43-7.32 (m, 5H), 5.41 (s, 2H) ppm.

### Synthesis of 1-benzyl-4-methyl-3-nitro-1H-pyrazole

To a solution of 1-benzyl-4-bromo-3-nitro-1H-pyrazole (800 mg, 2.83 mmol) in dioxane/water (16 mL/4 mL) was added trimethylboroxine (1.1 g, 8.5 mmol), Pd(PPh₃)₄ (344 mg, 0.28 mmol) and Na₂CO₃ (599 mg, 5.6 mmol). The mixture was heated at 100° C. for 12 h under nitrogen atmosphere. After cooling down to room temperature, the mixture was diluted with water (50 mL) and extracted with EtOAc (40 mL×3). The combined organic layers were washed with water (30 mL) and brine (50 mL), dried over Na₂SO₄, filtered and concentrated. The residue was purified by Prep-TLC (DCM/MeOH=5/1) to give 1-benzyl-4-methyl-3-nitro-1H-pyrazole (600 mg, Yield: 80%) as yellow solid. ESI-LCMS (m/z): 218.2 [M+1].

### Synthesis of 1-benzyl-4-methyl-1H-pyrazol-3-amine

A mixture of 1-benzyl-4-methyl-3-nitro-1H-pyrazole (550 mg, 2.53 mmol), iron powder (1.48 g, 25.2 mmol) and NH₄Cl (1.40 g, 25.2 mmol) in MeOH/water (55 mL/15 mL) was stirred at 80° C. for 12 h. After cooling down to room temperature, the mixture was filtered through Celite, and the filtrate was concentrated. The resulting mixture was diluted with water (30 mL), and extracted with EtOAc (25 mL×2). The combined organic layers were washed with water (30 mL) and brine (30 mL), dried over Na₂SO₄, filtered and concentrated to give the title compound (500 mg, Yield: 87%) as a brown solid. ESI-LCMS (m/z): 188.1 [M+1].

### Synthesis of 3-(1-benzyl-4-methyl-1H-pyrazol-3-ylamino)isonicotinic acid

The standard Buchwald coupling protocol from paragraph [0306] above was followed. Standard LiOH hydrolysis conditions as described in paragraph [0500] above were used.

### Synthesis of 3-(4-phenyl-1-(piperidin-4-yl)-1H-pyrazol-3-ylamino) isonicotinic acid and 3-(1-(1-methylpiperidin-4-yl)-4-phenyl-1H-pyrazol-3-ylamino) isonicotinic acid (Compounds 129 and 130)

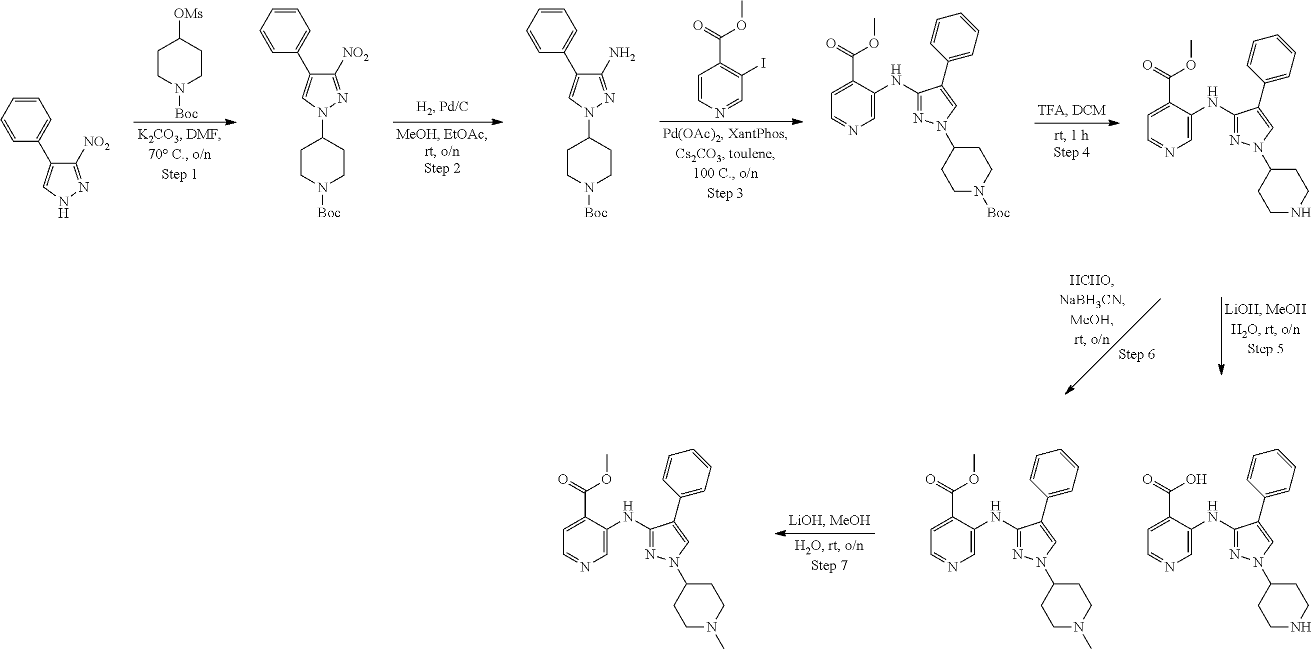

### Synthesis of tert-butyl 4-(3-nitro-4-phenyl-1H-pyrazol-1-yl)piperidine-1-carboxylate

A mixture of 3-nitro-4-phenyl-1H-pyrazole (200 mg, 1.05 mmol), tert-butyl 4-(methylsulfonyloxy)piperidine-1-carboxylate (438 mg, 1.57 mmol) and potassium carbonate (290 mg, 2.10 mmol) in DMF (5 mL) was stirred at 70° C. overnight. After cooling down to room temperature, the mixture was diluted with water (30 mL), extracted with EtOAc (30 mL×3). The combined organic layers was washed with brine (20 mL), dried over Na₂SO₄, filtered and concentrated to give a residue which was purified by prep-HPLC to give two regioisomers.

### tert-Butyl 4-(3-nitro-4-phenyl-1H-pyrazol-1-yl)piperidine-1-carboxylate

(110 mg, Yield: 28%, yellow solid). ESI-LCMS (m/z): 317.1 [M−55]; ¹HNMR (400 MHz, CDCl₃): δ 7.53 (s, 1H), 7.46-7.38 (m, 5H), 4.43-4.25 (m, 3H), 3.00-2.85 (m, 2H), 2.28-2.20 (m, 2H), 2.07-1.95 (m, 2H), 1.50 (s, 9H) ppm.

### tert-Butyl 4-(5-nitro-4-phenyl-1H-pyrazol-1-yl)piperidine-1-carboxylate

(60 mg, Yield: 15% yield, yellow solid). ESI-LCMS (m/z): 317.1 [M−55]; ¹HNMR (400 MHz, CDCl₃): δ 7.61 (s, 1H), 7.47-7.39 (m, 5H), 5.02-4.93 (m, 1H), 4.40-4.25 (m, 2H), 3.00-2.86 (m, 2H), 2.24-2.06 (m, 4H), 1.51 (s, 9H) ppm.

### Synthesis of tert-butyl 4-(3-amino-4-phenyl-1H-pyrazol-1-yl)piperidine-1-carboxylate

A mixture of tert-butyl 4-(3-nitro-4-phenyl-1H-pyrazol-1-yl) piperidine-1-carboxylate (720 mg, 1.93 mmol) and 10% Pd/C (500 mg) in MeOH/EtOAc (60 mL, v/v=1/1) was degassed under vacuum and purged with H₂ several times. The mixture was stirred under H₂ atmosphere at room temperature overnight, then filtered and concentrated to give tert-butyl 4-(3-amino-4-phenyl-1H-pyrazol-1-yl)piperidine-1-carboxylate (580 mg, Yield: 88%) as a yellow solid. ESI-LCMS (m/z): 343.2 [M+1].

### Synthesis of 3-(4-phenyl-1-(piperidin-4-yl)-1H-pyrazol-3-ylamino) isonicotinic acid

The standard Buchwald coupling protocol from paragraph [0306] above was followed. The standard TFA BOC deprotection protocol as described in paragraph [0448] was used. Standard LiOH hydrolysis conditions as described in paragraph [0500] above were employed.

### Synthesis of 3-(1-(1-methylpiperidin-4-yl)-4-phenyl-1H-pyrazol-3-yl amino)isonicotinic acid

Standard Eschweiler-Clarke conditions as described in paragraph [0484] above were employed. Standard LiOH hydrolysis conditions as described in paragraph [0500] above were employed.

### Synthesis of 3-(1-(trans-4-methylpyrrolidin-3-yl)-4-phenyl-1H-pyrazol-5-yl-amino)isonicotinic acid and 3-(1-(trans-1,4-dimethylpyrrolidin-3-yl)-4-phenyl-1H-pyrazol-5-ylamino)isonicotinic acid (Compounds 131 and 132)

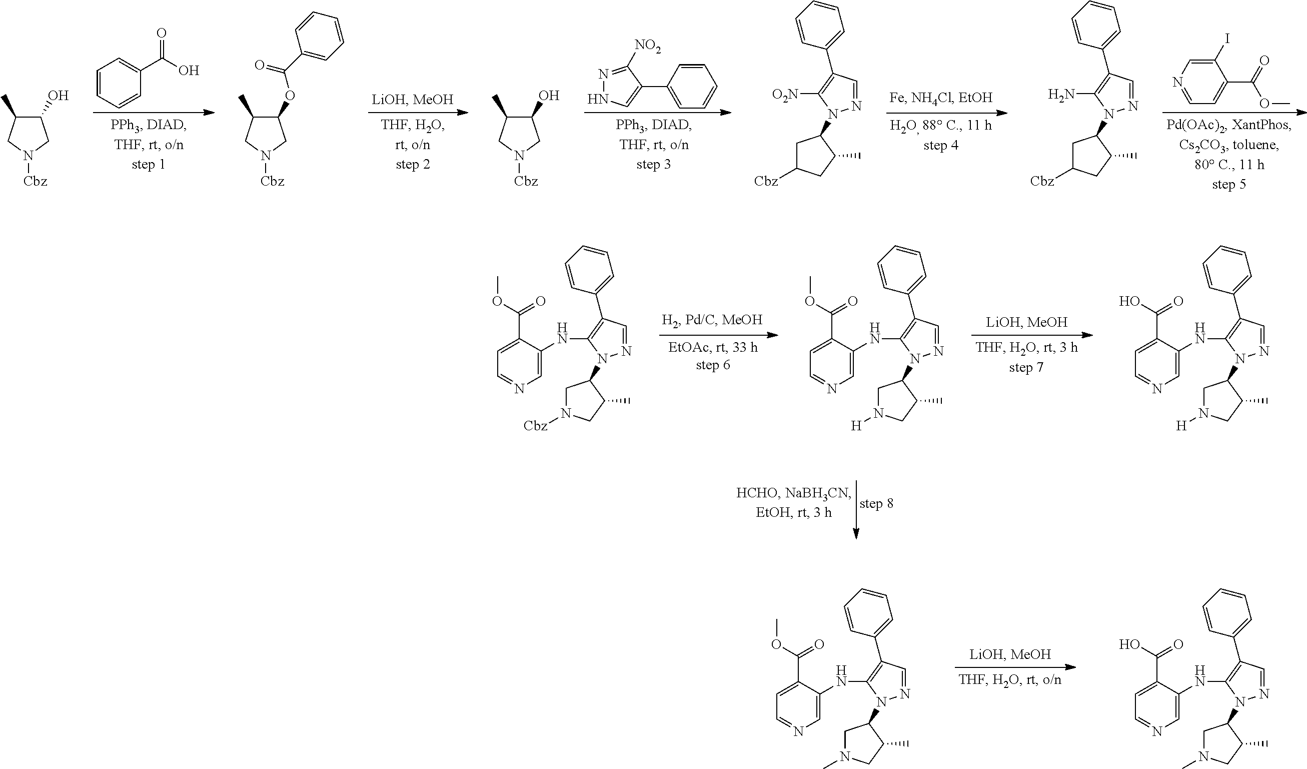

Compounds 131 and 132 were synthesized by a method similar to that illustrated in paragraph [0445] above except that the starting compound was benzyl (cis)-3-hydroxy-4-methylpyrrolidine-1-carboxylate.

### Synthesis of cis-benzyl 3-(benzoyloxy)-4-methylpyrrolidine-1-carboxylate

To a solution of trans-benzyl 3-hydroxy-4-methylpyrrolidine-1-carboxylate (1.06 g, 4.49 mmol), benzoic acid (711.6 mg, 6.56 mmol) and PPh₃ (1.52 g, 5.83 mmol) in anhydrous THF (50 mL) was added DIAD (1.35 g, 6.73 mmol). The mixture was stirred at room temperature for 18 h. The mixture was concentrated and the residue was purified by chromatographic column on silica gel (eluted with 0% to 40% of EtOAc in petroleum ether) to give cis-benzyl 3-(benzoyloxy)-4-methylpyrrolidine-1-carboxylate (1.3 g, Yield: 85%) as a colorless oil. ESI-LCMS (m/z): 340.1 [M+1].

### Synthesis of cis-benzyl 3-hydroxy-4-methylpyrrolidine-1-carboxylate

A solution of cis-benzyl 3-(benzoyloxy)-4-methylpyrrolidine-1-carboxylate (1.3 g, 3.83 mmol) and LiOH—H₂O (322 mg, 7.67 mmol) in MeOH/THF/H₂O (22 mL, v/v/v=10/10/1) was stirred at room temperature for 18 h. After concentration, the residue was re-dissolved in DCM (100 mL) and washed with water (50 mL), 5% Na₂CO₃ aqueous solution (50 mL), water (50 mL) and brine (50 mL). The organic layer was dried over Na₂SO₄, filtered and concentrated to give cis-benzyl 3-hydroxy-4-methylpyrrolidine-1-carboxylate (830 mg, Yield: 92%) as yellow oil. ESI-LCMS (m/z): 236.2 [M+1].

### Synthesis of 3-(4-phenyl-1-(piperidin-3-yl)-1H-pyrazol-5-ylamino) isonicotinic acid and 3-(1-(1-ethylpiperidin-3-yl)-4-phenyl-1H-pyrazol-5-yl-amino)isonicotinic acid (Compounds 133 and 134)

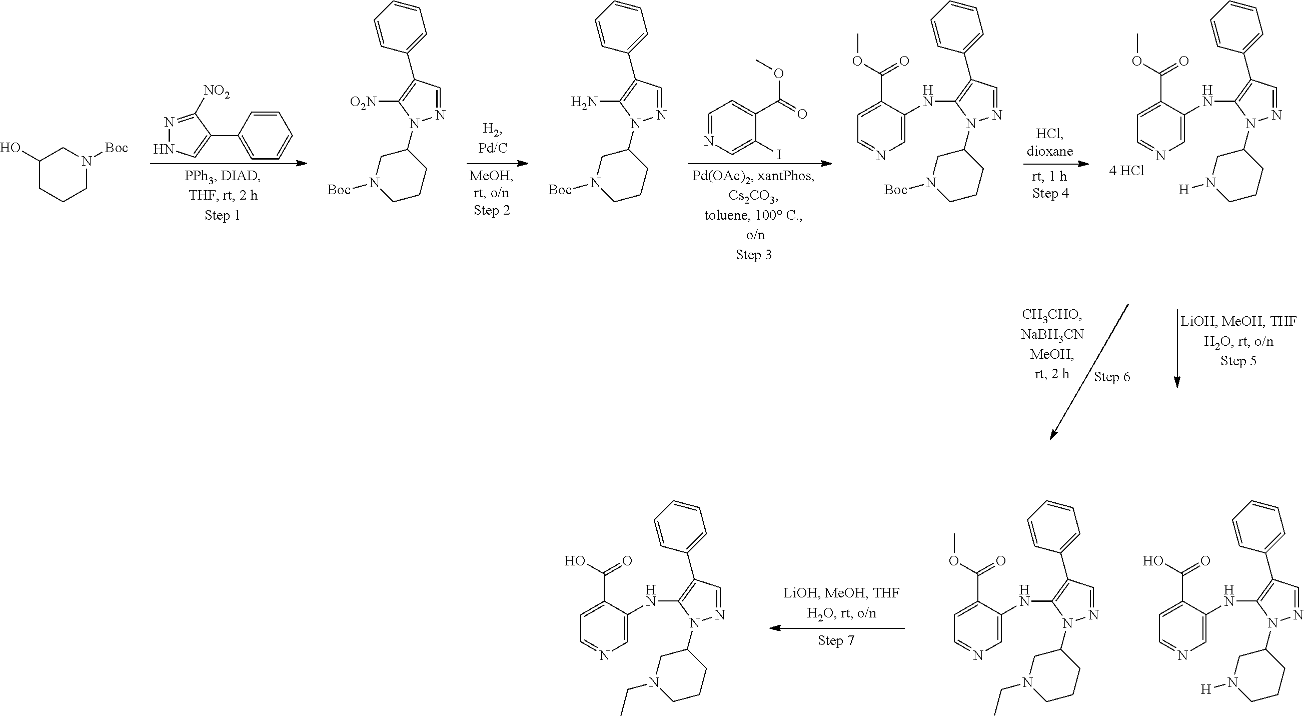

Compounds 133 and 134 were synthesized by a method similar to that illustrated in paragraph [0445] employing standard Mitsunobu conditions with tert-butyl 3-hydroxypiperidine-1-carboxylate. Acetaldehyde was employed in step 6 (reductive amination).

### Synthesis of 3-(1-(1-(2-(dimethylamino)ethyl)piperidin-3-yl)-4-phenyl-1H-pyrazol-5-ylamino) isonicotinic acid and 3-(1-(1-(2-(methylamino)ethyl)piperidin-3-yl)-4-phenyl-1H-pyrazol-5-yl-amino) isonicotinic acid (Compounds 135 and 136)

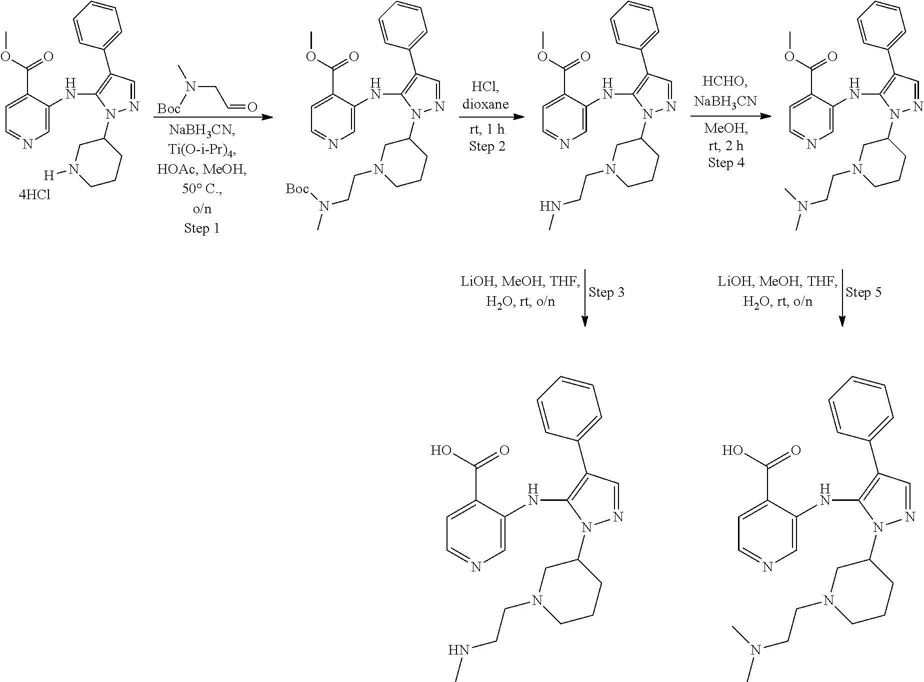

Compounds 135 and 136 were synthesized by a method similar to that illustrated in paragraph [0575] with a methyl ester of Compound 133 and tert-butyl methyl(2-oxoethyl) carbamate as starting materials.

### Synthesis of (R)-3-(4-phenyl-1-(1,2,3,4-tetrahydroquinolin-3-yl)-1H-pyrazol-5-ylamino)isonicotinic acid (Compound 137)

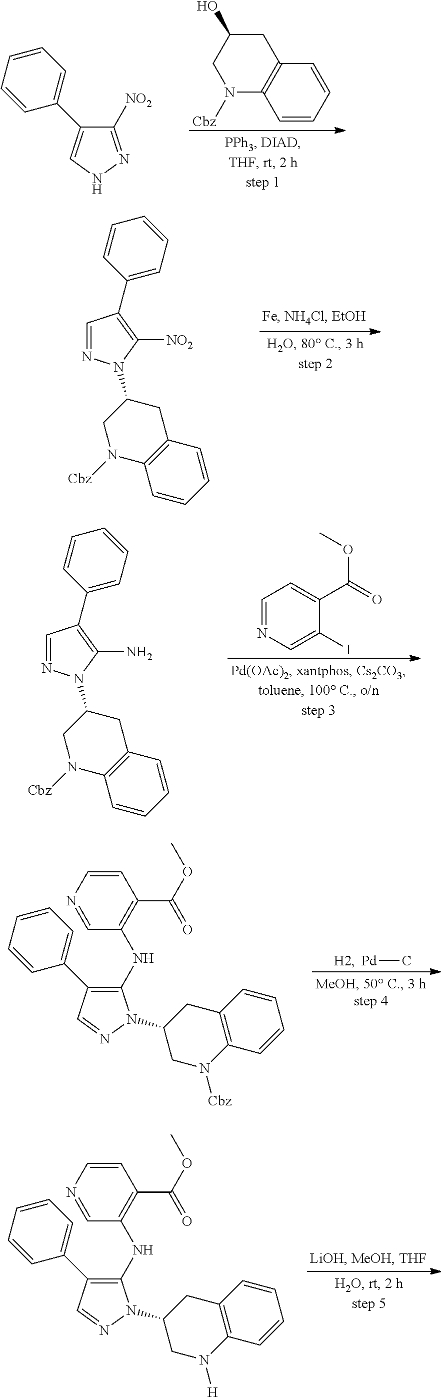

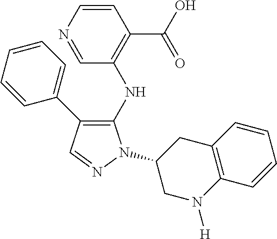 [SMILES: O=C(O)c1ccncc1Nc1c(-c2ccccc2)cnn1[C@H]1CNc2ccccc2C1]

Compound 137 was synthesized by a method similar to that illustrated in paragraph [0445] above except that the starting compound was benzyl (S)-3-hydroxy-3,4-dihydroquinoline-1(2H)-carboxylate.

### Synthesis of 3-(1-(cis-4-aminocyclohexyl)-4-phenyl-1H-pyrazol-5-ylamino) isonicotinic acid and 3-(1-(cis-4-(dimethylamino)cyclohexyl)-4-phenyl-1H-pyrazol-5-ylamino)isonicotinic acid (Compounds 138 and 139)

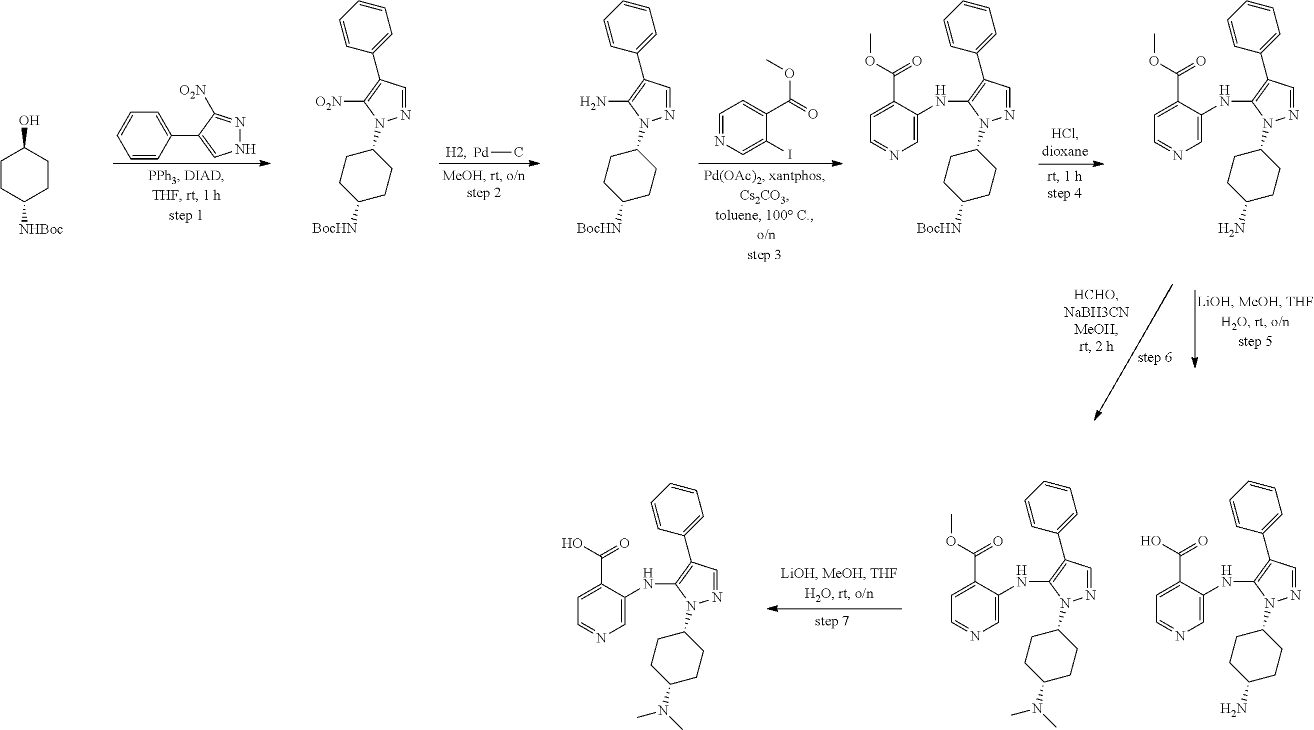

Compounds 138 and 139 were synthesized by a method similar to that illustrated in paragraph [0445] above except that the starting compound was tert-butyl ((trans)-4-hydroxycyclohexyl) carbamate.

### Synthesis of 3-(1-(1-(2-aminoethyl)piperidin-3-yl)-4-methyl-1H-pyrazol-5-yl-amino)isonicotinic acid (Compound 140)

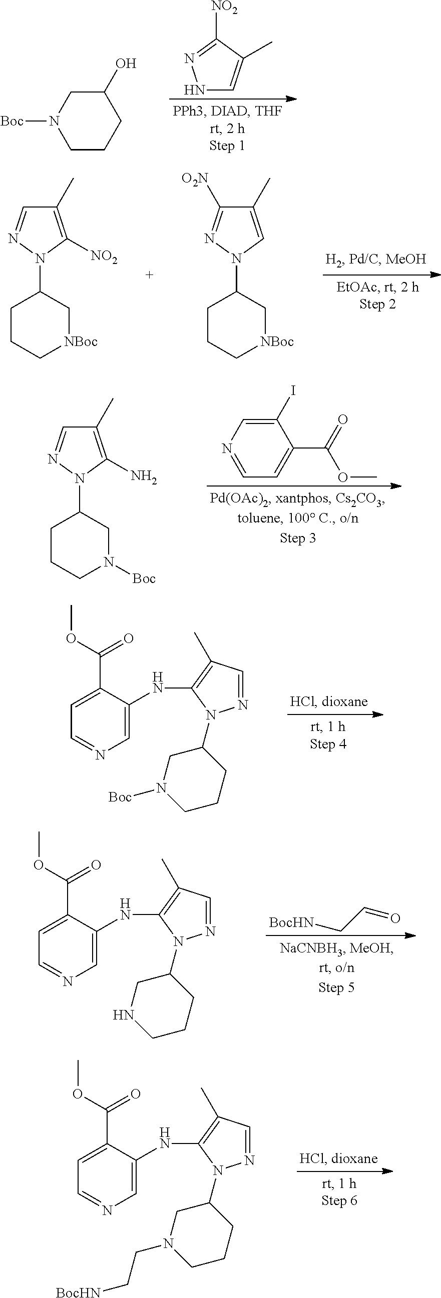

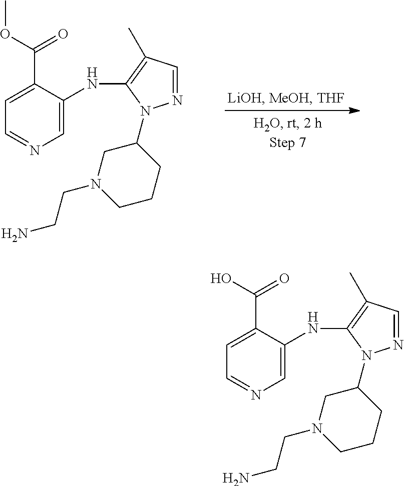 [SMILES: COC(=O)c1ccncc1Nc1c(C)cnn1C1CCCN(CCN)C1 ; Cc1cnn(C2CCCN(CCN)C2)c1Nc1cnccc1C(=O)O]

Compound 140 was synthesized by a method similar to that illustrated in paragraph [0445] above except that the starting compound was tert-butyl (2-oxoethyl)carbamate.

### Synthesis of 3-(4-methyl-1-(piperidin-3-yl)-1H-pyrazol-3-ylamino) isonicotinic acid (Compound 141)

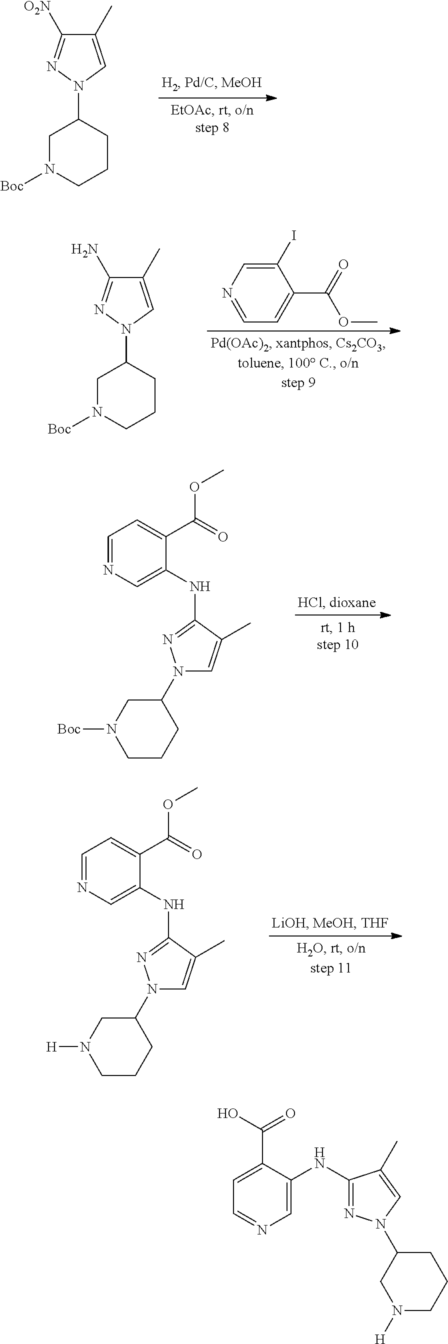 [SMILES: Cc1cn(C2CCCN(C(=O)OC(C)(C)C)C2)nc1[N+](=O)[O-] ; Cc1cn(C2CCCN(C(=O)OC(C)(C)C)C2)nc1N ; COC(=O)c1ccncc1I ; COC(=O)c1ccncc1Nc1nn(C2CCCN(C(=O)OC(C)(C)C)C2)cc1C ; COC(=O)c1ccncc1Nc1nn(C2CCCNC2)cc1C ; Cc1cn(C2CCCNC2)nc1Nc1cnccc1C(=O)O]

Compound 141 was synthesized by a method similar to that illustrated in paragraph [0445] above.

### Synthesis of 3-(4-methyl-1-(piperidin-4-yl)-1H-pyrazol-5-ylamino) isonicotinic acid (Compound 142)

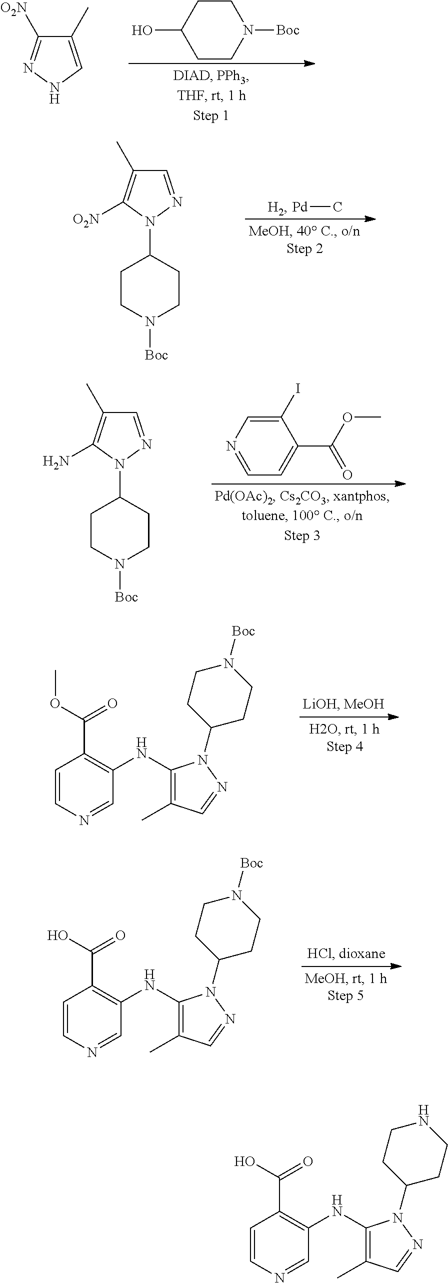

Compound 142 was synthesized by a method similar to that illustrated in paragraph [0445] above except that the starting compound was tert-butyl 4-hydroxypiperidine-1-carboxylate.

### Synthesis of 3-(4-methyl-1-(1,2,3,4-tetrahydroisoquinolin-5-yl)-1H-pyrazol-3-ylamino)isonicotinic acid (Compound 143)

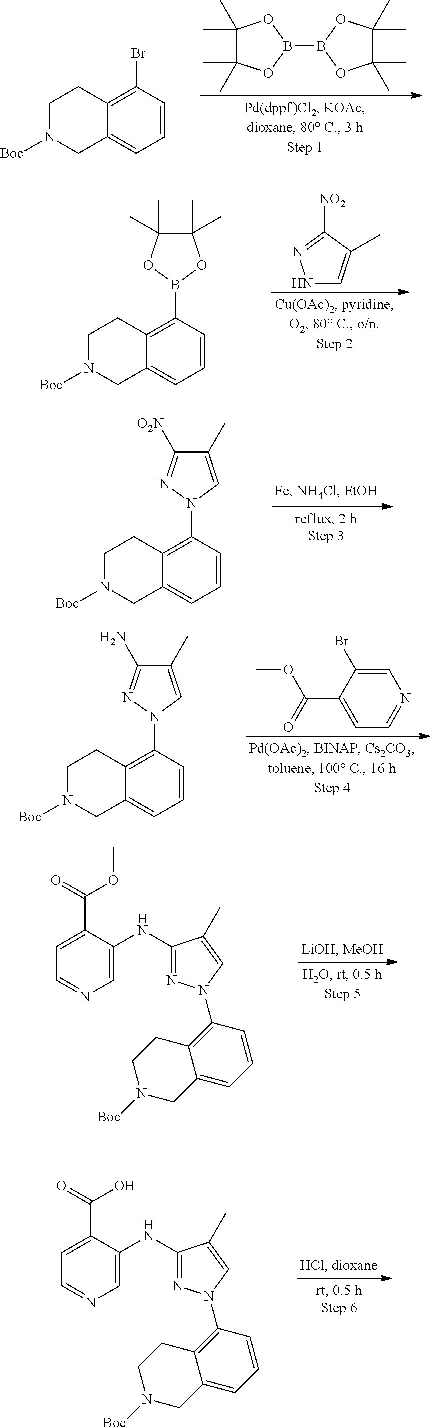

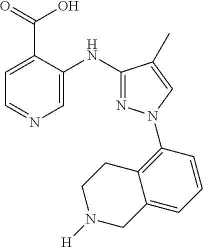 [SMILES: Cc1cn(-c2cccc3c2CCNC3)nc1Nc1cnccc1C(=O)O]

Compound 143 was synthesized by a method similar to that illustrated in paragraph [0388] above except that tert-butyl 5-(4,4,5,5-tetramethyl-1,3,2-dioxaborolan-2-yl)-3,4-dihydroisoquinoline-2(1H)-carboxylate was used in the Chan-Lam coupling step.

### Synthesis of tert-butyl 5-(4,4,5,5-tetramethyl-1,3,2-dioxaborolan-2-yl)-3, 4-dihydroisoquinoline-2(1H)-carboxylate

To a solution of tert-butyl 5-bromo-3,4-dihydroisoquinoline-2(1H)-carboxylate (4.0 g, 12.8 mmol) in dioxane (60 mL) was added bis(pinacolato)diboron (6.5 g, 25.6 mmol), KOAc (3.76 g, 38.4 mmol) and Pd(dppf)Cl₂ (936 mg, 1.15 mmol), and the mixture was stirred at 80° C. for 3 hours under Ar atmosphere. After cooling down to room temperature, the mixture was filtered through Celite. The filtrate was diluted with water (30 mL), extracted with EtOAc (30 mL×3). The combined organic layers was washed with brine (20 mL), dried over Na₂SO₄ filtered and concentrated. The residue was purified by silica gel chromatography (petroleum ether/EtOAc=9/1) to afford tert-butyl 5-(4,4,5,5-tetramethyl-1,3,2-dioxaborolan-2-yl)-3,4-dihydro-isoquinoline-2(1H)—F

### Synthesis of 3-(4-(2-(dimethylamino)ethyl)-1-phenyl-1H-pyrazol-3-ylamino) isonicotinic acid (Compound 144)

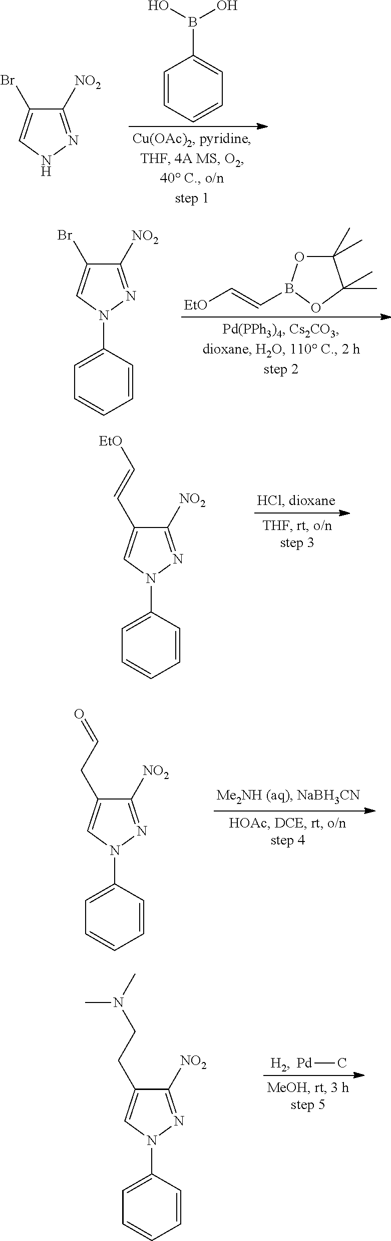

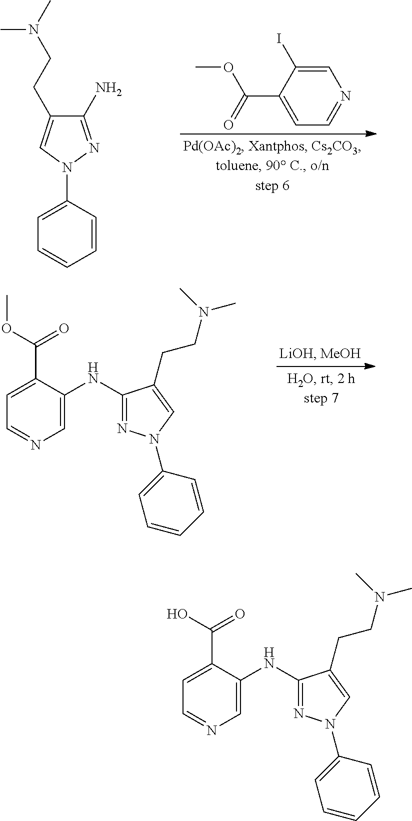 [SMILES: CN(C)CCc1cn(-c2ccccc2)nc1N ; COC(=O)c1ccncc1I ; COC(=O)c1ccncc1Nc1nn(-c2ccccc2)cc1CCN(C)C ; CN(C)CCc1cn(-c2ccccc2)nc1Nc1cnccc1C(=O)O]

### Synthesis of 4-bromo-3-nitro-1-phenyl-1H-pyrazole

To a solution of 4-bromo-3-nitro-1H-pyrazole (1.5 g, 7.8 mmol) in anhydrous THF (50 mL) was added phenylboronic acid (0.95 g, 7.8 mmol), copper(II) acetate (2.1 g, 11.7 mmol) and pyridine (2.5 g, 31.2 mmol. The mixture was stirred overnight at 40° C. under oxygen atmosphere. After removing the insolubles by filtration, the filtrate was diluted with EtOAc (50 mL), washed with water (50 mL) and brine (50 mL), dried over Na₂SO₄, filtered and concentrated in vacuo to obtain a residue which was purified by silica gel chromatography (eluting with hexane/EtOAc=1/1) to afford 4-bromo-3-nitro-1-phenyl-1H-pyrazole as a yellow solid (2.0 g, Yield: 95%). ESI-LCMS (m/z): 268.0 [M+1].

### Synthesis of 4-(2-ethoxyvinyl)-3-nitro-1-phenyl-1H-pyrazole

To a solution of 4-bromo-3-nitro-1-phenyl-1H-pyrazole (800 mg, 2.98 mmol) in dioxane (20 mL) and water (5 mL) was added 2-(2-ethoxyvinyl)-4,4,5,5-Tetramethyl-1,3,2-dioxaborolane (1.18 g, 5.96 mmol), Pd(PPh₃)₄ (344 mg, 0.29 mmol) and cesium carbonate (1.94 g, 5.96 mmol). The mixture was heated to 110° C. for 3 h under nitrogen atmosphere. After cooling down to room temperature, the mixture was diluted with water (50 mL), extracted with EtOAc (25 mL×2). The combined organic layers were washed with brine (100 mL), dried over Na₂SO₄, filtered and concentrated. The residue was purified by silica gel chromatography (petroleum ether/EtOAc=3/1) to afford the title compound as a yellow solid (700 mg, Yield: 81%). ESI-LCMS (m/z): 260.1 [M+1].

### Synthesis of 2-(3-nitro-1-phenyl-1H-pyrazol-4-yl)acetaldehyde

To a solution of 4-(2-ethoxyvinyl)-3-nitro-1-phenyl-1H-pyrazole (700 mg, 2.69 mmol) in THF (10 mL) was added 4N HCl in dioxane (6.7 mL, 26.9 mmol) dropwise at 15° C. The reaction was stirred at 15° C. overnight. After concentration, the residue was diluted with EtOAc (50 mL), washed with brine (50 mL), dried over Na₂SO₄, filtered and concentrated to afford the title compound (600 mg, crude), which was used in next step directly without further purification. ESI-LCMS (m/z): 232.1 [M+1].

### Synthesis of N,N-dimethyl-2-(3-nitro-1-phenyl-1H-pyrazol-4-yl) ethanamine

To a solution of 2-(3-nitro-1-phenyl-1H-pyrazol-4-yl)acetaldehyde (600 mg, 2.59 mmol) in DCE (25 mL) was added dimethylamine aqueous solution (40 wt. %, 4.4 mL) and acetic acid (2 drops). The solution was stirred at 25° C. for 1 h. Then Sodium triacetoxyborohydride (2.18 g, 10.3 mmol) was added in portions. The reaction mixture was stirred at 25° C. overnight. Water (100 mL) was added, and the mixture was extracted with DCM (100 mL×2). The combined organic layers were washed with brine (100 mL), dried over Na₂SO₄, filtered and concentrated to afford the title compound (0.6 g, crude) which was used into next step without further purification. ESI-LCMS (m/z): 261.1 [M+1]⁺.

### Synthesis of 4-(2-(dimethylamino)ethyl)-1-phenyl-1H-pyrazol-3-amine

To a solution of N,N-dimethyl-2-(3-nitro-1-phenyl-1H-pyrazol-4-yl)ethanamine (600 mg, crude from step 4) in MeOH (50 mL) was added 10% Pd/C (122 mg) in one portion with nitrogen protected. The system was exchanged with hydrogen gas three times, and stirred under hydrogen atmosphere at room temperature for 3 h. The mixture was filtered and the filtrate was concentrated to afford the title compound (600 mg, crude) which was used into next step without further purification. ESI-LCMS (m/z): 231.2 [M+1].

### Synthesis of methyl 3-(4-(2-(dimethylamino)ethyl)-1-phenyl-1H-pyrazol-3-ylamino)isonicotinate

The standard Buchwald coupling protocol from paragraph [0306] above was followed (300 mg, Yield: 30%).

### Synthesis of 3-(4-(2-(dimethylamino)ethyl)-1-phenyl-1H-pyrazol-3-yl amino)isonicotinic acid

Standard LiOH hydrolysis conditions as described in paragraph [0500] above were followed (45 mg, Yield: 16%).

### Example 2: Bioassay Protocol and General Methods

KDM4C Assay

N-terminal GST-tagged KDM4C²⁻³⁷² was purchased from BPS Biosciences. Peptides were synthesized by BioPeptide and all other reagents were purchased from Sigma-Aldrich at the highest level of purity possible. Assays were performed in a 50 μL volume in 384-well V-bottom polypropylene microplates (Greiner) at 25° C. Optimized 1× assay buffer was 50 mM HEPES (pH=8.0), 100 μM sodium ascorbate, 20 mM NaCl, 10 μM ammonium iron sulfate, 1 mM tris(2-carboxyethyl)phosphine (TCEP), 0.002% Tween-20, and 0.005% BSG. For compound screening, 35 μL of KDM4C²⁻³⁷² (final concentration, f.c.=5 nM) was added using a Multidrop Combi and preincubated with 1 μL of test compound (f.c.=10 μM) for 30 minutes and subsequently 15 μL of peptide with the following sequence NH₂-ARTKQTAR(Kme3)STGGKAPRKQLA(K-Ahx-Biotin)-amide (f.c.=500 nM) and 2-oxoglutarate (f.c.=15 μM) was added by Multidrop Combi to begin the reaction. Reactions proceeded for 150 minutes and were stopped by the addition of 5 μL of formic acid (f.c.=0.5%) using a Multidrop Combi and then analyzed by SAMDI Tech Inc.

KDM4A Assay

N-terminal His-tagged KDM4A¹⁻³⁵⁰ was purchased from BPS Biosciences. Peptides were synthesized by BioPeptide and all other reagents were purchased from Sigma-Aldrich at the highest level of purity possible. Assays were performed in a 50 μL volume in 384-well V-bottom polypropylene microplates (Greiner) at 25° C. Optimized 1× assay buffer was 50 mM HEPES (pH=8.0), 100 μM sodium ascorbate, 20 mM NaCl, 10 μM ammonium iron sulfate, 1 mM tris(2-carboxyethyl)phosphine (TCEP), 0.002% Tween-20, and 0.005% BSG. For compound screening, 35 μL of KDM4A¹⁻³⁵⁰ (final concentration, f.c.=5 nM) was added using a Multidrop Combi and preincubated with 1 μL of test compound (f.c.=10 μM) for 30 minutes and subsequently 15 μL of peptide with the following sequence NH₂-ARTKQTAR(Kme2)STGGKAPRKQLA(K-Ahx-Biotin)-amide (f.c.=600 nM) and 2-oxoglutarate (f.c.=5 μM) was added by Multidrop Combi to begin the reaction. Reactions proceeded for 120 minutes and were stopped by the addition of 5 μL of formic acid (f.c.=0.5%) using a Multidrop Combi and then analyzed by SAMDI Tech Inc.

KDM5A Assay

C-terminal FLAG-tagged KDM5A¹⁻¹⁰⁹⁰ was purchased from BPS Biosciences. Peptides were synthesized by BioPeptide and all other reagents were purchased from Sigma-Aldrich at the highest level of purity possible. Assays were performed in a 50 μL volume in 384-well V-bottom polypropylene microplates (Greiner) at 25° C. Optimized 1× assay buffer was 50 mM HEPES (pH=8.0), 100 μM sodium ascorbate, 20 mM NaCl, 10 μM ammonium iron sulfate, 1 mM tris(2-carboxyethyl)phosphine (TCEP), 0.002% Tween-20, and 0.005% BSG. For compound screening, 35 μL of KDM5A¹⁻¹⁰⁹° (final concentration, f.c.=20 nM) was added using a Multidrop Combi and preincubated with 1 μL of test compound (f.c.=10 μM) for 30 minutes and subsequently 15 μL of peptide with the following sequence NH₂-ART[Kme-3]QTARKSTGGKA[K-Ahx-Biot]-amide (f.c.=250 nM) and 2-oxoglutarate (f.c.=15 μM) was added by Multidrop Combi to begin the reaction. Reactions proceeded for 150 minutes and were stopped by the addition of 5 μL of formic acid (f.c.=0.5%) using a Multidrop Combi and then analyzed by SAMDI Tech Inc.

General SAMDI Procedure

SAMDI Tech analyzed all reactions using self-assembled monoloayer desorption/ionization technology as previously described (Gerard-Levin Z A, Scholle M D, Eisenberg A H, et al.: High-throughput screening of small molecule libraries using SAMDI mass spectrometry. ACS Comb Sci 2011; 13:347-350). Briefly, a 2 μL sample of the stopped demethylase reactions were transferred to a SAMDI array coated with 384 biotin-neutravidin spots using a 384-channel pipet station. The SAMDI arrays were incubated for 1 h in a humidified chamber, followed by washing of the surface with deionized ultra-filtered water (DIUF) and drying under nitrogen. A matrix of 30 mg/ml 2′,4′,6′-Trihydroxyacetophenone monohydrate (THAP) in acetone was applied using a TPP Mosquito 1.2 μL tip by dispensing 50 nL on each of the 384 spots on the array. SAMDI-MS was run using acquisition method ReflectorPositiveUTX. The % conversion of substrate to product was calculated using the AUC of the substrate and product peptide peaks.

An IC₅₀ value was determined by contacting various concentrations of a test compound with one of the KDM4A, KDM4C or KDMSC enzyme constructs and plotting a dose-response curve which associates the amount of inhibition of the enzyme relative to the concentration of the test compound. From the curve, the IC₅₀ value (which represents the concentration of the test compound necessary to inhibit maximal enzymatic activity by 50%) was determined. Additionally, the slope of the linear range of the plotted curve, which indicates the speed with which the curve rises between the minimum and maximum, was calculated.

IC₅₀ values of various compounds disclosed herein are presented in Table 2 below. * denotes 100 μM>IC₅₀>10 μM; ** denotes 10 μM>IC₅₀>1 μM; *** denotes 1 μM>IC₅₀>0.1 μM; and **** denotes 0.1 μM>IC₅₀>0.001 μM.

TABLE 2

| Compound | 4A IC₅₀ | 4C IC₅₀ | 5A IC₅₀ |
|---|---|---|---|
| Number | (μM) | (μM) | (μM) |
| 1 | *** | *** | ** |
| 2 | *** | *** | ** |
| 3 | ** | ** | ** |
| 4 | ** | ** | >100 |
| 5 | *** | *** | *** |
| 6 | *** | ** | **** |
| 7 | *** | *** | *** |
| 8 | ** | ** | * |
| 9 | *** | *** | *** |
| 10 | ** | ** | * |
| 11 | *** | *** | ** |
| 12 | *** | **** | *** |
| 13 | ** | *** | ** |
| 14 | *** | *** | **** |
| 15 | * | * | ** |
| 16 | ** | * | * |
| 17 | ** | ** | *** |
| 18 | *** | *** | ** |
| 19 | **** | *** | **** |
| 20 | **** | **** | ** |
| 21 | ** | *** | *** |
| 22 | *** | *** | *** |
| 23 | ** | *** | ** |
| 24 | *** | ** | **** |
| 25 | *** | ** | **** |
| 26 | ** | ** | ** |
| 27 | *** | *** | *** |
| 28 | **** | **** | ** |
| 29 | *** | *** | *** |
| 30 | * | * | ** |
| 31 | *** | *** | **** |
| 32 | **** | *** | ** |
| 33 | **** | **** | *** |
| 34 | ** | * | * |
| 35 | *** | *** | **** |
| 36 | *** | *** | **** |
| 37 | *** | *** | ** |
| 38 | * | * | ** |
| 39 | ** | *** | * |
| 40 | ** | ** | ** |
| 41 | *** | *** | ** |
| 42 | *** | *** | *** |
| 43 | * | * | * |
| 44 | Insoluble | | |
| 45 | *** | *** | **** |
| 46 | ** | * | ** |
| 47 | Insoluble | | |
| 48 | **** | **** | *** |
| 49 | **** | *** | **** |
| 50 | Insoluble | | |
| 51 | **** | *** | **** |
| 52 | ** | * | ** |
| 53 | *** | *** | ** |
| 54 | Insoluble | | |
| 55 | ** | ** | ** |
| 56 | *** | *** | ** |
| 57 | *** | *** | *** |
| 58 | **** | **** | ** |
| 59 | **** | **** | ** |
| 60 | *** | *** | ** |
| 61 | *** | *** | ** |
| 62 | **** | **** | **** |
| 63 | *** | *** | **** |
| 64 | **** | **** | **** |
| 65 | ** | ** | ** |
| 66 | * | * | * |
| 67 | **** | *** | **** |
| 68 | *** | *** | ** |
| 69 | *** | ** | *** |
| 70 | *** | ** | ** |
| 71 | *** | *** | **** |
| 72 | ** | ** | *** |
| 73 | *** | *** | **** |
| 74 | *** | *** | *** |
| 75 | *** | ** | * |
| 76 | *** | ** | * |
| 77 | **** | *** | * |
| 78 | *** | *** | ** |
| 79 | **** | **** | ** |
| 80 | *** | *** | **** |
| 81 | **** | **** | ** |
| 82 | **** | **** | **** |
| 83 | **** | *** | ** |
| 84 | *** | *** | *** |
| 85 | ** | ** | ** |
| 86 | *** | ** | ** |
| 87 | * | * | ** |
| 88 | *** | **** | *** |
| 89 | **** | *** | *** |
| 90 | *** | *** | ** |
| 91 | *** | ** | *** |
| 92 | *** | *** | ** |
| 93 | * | >100 | * |
| 94 | **** | *** | *** |
| 95 | * | * | * |
| 96 | **** | *** | ** |
| 97 | ** | ** | *** |
| 98 | *** | *** | ** |
| 99 | *** | ** | *** |
| 100 | *** | *** | ** |
| 101 | * | * | * |
| 102 | *** | *** | *** |
| 103 | * | * | * |
| 104 | *** | *** | ** |
| 105 | — | *** | **** |
| 106 | — | **** | ** |
| 107 | *** | ** | *** |
| 108 | — | ** | ** |
| 109 | — | ** | *** |
| 110 | — | *** | **** |
| 111 | *** | *** | * |
| 112 | *** | *** | *** |
| 113 | — | *** | ** |
| 114 | *** | *** | *** |
| 115 | **** | *** | ** |
| 116 | *** | *** | *** |
| 117 | — | *** | ** |
| 118 | — | ** | *** |
| 119 | — | *** | ** |
| 120 | — | *** | *** |
| 121 | — | >100 | >100 |
| 122 | — | >100 | * |
| 123 | — | *** | ** |
| 124 | — | ** | ** |
| 125 | — | *** | *** |
| 126 | 1.49 | ** | ** |
| 127 | — | *** | *** |
| 128 | — | *** | *** |
| 129 | — | *** | **** |
| 130 | — | *** | **** |
| 131 | — | * | * |
| 132 | — | * | >100 |
| 133 | — | >100 | >100 |
| 134 | — | * | * |
| 135 | — | * | ** |
| 136 | — | * | * |
| 137 | — | * | * |
| 138 | — | >100 | ** |
| 139 | — | * | * |
| 140 | — | * | * |
| 141 | — | *** | **** |
| 142 | — | * | * |
| 143 | — | *** | **** |
| 144 | — | ** | ** |

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A compound of Formula (III) or a pharmaceutically acceptable salt thereof: 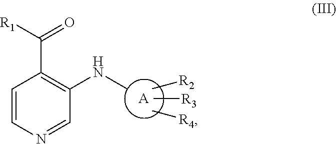
wherein
ring A is 5-membered heteroaryl or 5-membered heterocycloalkyl;
R₁ is hydroxyl, C₁-C₆ alkoxyl, or mono- or di-C₁-C₆-alkylamino and said C₁-C₆ alkoxyl, or mono- or di-C₁-C₆-alkylamino is optionally substituted with one or more substituents selected from the group consisting of halo, hydroxyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl;
R₂ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₂ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₂ is attached to the pyrazolyl via a carbon ring atom thereof;
R₃ is H, halo, cyano, azido, oxo, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more -Q₁-T₁, wherein Q₁ is a bond, C(O), a C₁-C₆ alkyl linker, or a 4- to 6-membered heterocycloalkyl linker and T₁ is selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when T₁ is C₁-C₆ alkyl, C₁-C₆ alkoxyl,C(O)O—C₁-C₆ alkyl, mono-C₁-C₆ alkylamino, di-C₁-C₆ alkylamino, C₃-C₈ cycloalkyl, C₆-C₁₀ aryl, 4 to 12-membered heterocycloalkyl, or 5- or 6-membered heteroaryl, T₁ is optionally substituted with one or more substituents selected from the group consisting of halo, C₁-C₄ alkyl, and C₆-C₁₀ aryl; and
R₄ is H, C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and when R₄ is not H, R₄ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₄ is attached to the pyrazolyl via a carbon ring atom thereof, and when neither R₂ nor R₄ is H, only one of R₂, R₃, and R₄ is aryl or heteroaryl.

2. The compound of claim 1, wherein R₄ is hydrogen.

3. The compound of claim 1 or 2, being of Formula (I) or a pharmaceutically acceptable salt thereof: 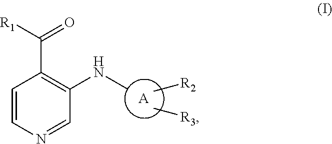
wherein
ring A is 5-membered heteroaryl or 5-membered heterocycloalkyl;
R₁ is hydroxyl, or C₁-C₆ alkoxyl;
R₂ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, oxo, C₆-C₁₀ aryl or 5- or 6-membered heteroaryl and R₂ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, wherein when ring A is pyrazolyl, R₂ is attached to the pyrazolyl via a carbon ring atom thereof; and
R₃ is H, halo, cyano, azido, oxo, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more substituents selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

4. The compound of claim 1, wherein R₄ is C₁-C₆ alkyl.

5. The compound of any one of claims 1-4, wherein ring A is a nitrogen-containing heteroaryl.

6. The compound of any one of claims 1-5, wherein ring A is pyrazolyl, imidazolyl, pyrrolyl, triazolyl, oxazolyl, oxadiazolyl, thiazolyl, isoxazolyl, isothiazolyl, thiadiazolyl, or tetrazolyl.

7. The compound of any one of claims 1-6, wherein R₁ is hydroxyl.

8. The compound of any one of claims 1-7, wherein R₂ is phenyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

9. The compound of any one of claims 1-7, wherein R₂ is pyridinyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

10. The compound of any one of claims 1-7, wherein R₂ is unsubstituted phenyl or pyridinyl.

11. The compound of any one of claims 1-10, wherein the compound is any of Formulae (Ia)-(Ih): 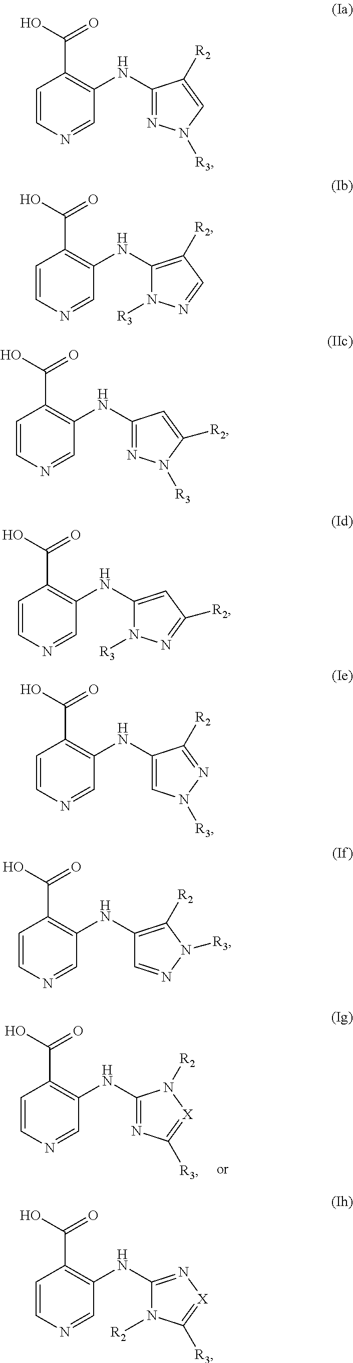
wherein X is N or CR₄.

12. The compound of claim 10, wherein the compound is of Formula (Ig) and X is CH.

13. The compound of any one of claims 1-12, wherein R₃ is C₁-C₆ alkyl optionally substituted with one or more -Q₁-T₁.

14. The compound of claim 13, wherein R₃ is methyl.

15. The compound of any one of claims 1-12, wherein R₃ is C₃-C₈ cycloalkyl, C₆-C₁₀ aryl, 4 to 12-membered heterocycloalkyl, or 5- or 6-membered heteroaryl optionally substituted with one or more -Q₁-T₁.

16. The compound of claim 1, wherein the compound is selected from those in Table 1, and pharmaceutically acceptable salts thereof.

17. A pharmaceutical composition comprising a compound of any of claims 1-16 or a pharmaceutically acceptable salt thereof and a pharmaceutically acceptable carrier.

18. A method for treating cancer in a subject in need thereof comprising administering to the subject a therapeutically effective amount of a compound of any of claims 1-16 or a pharmaceutically acceptable salt thereof.

19. A compound of any of claims 1-16 or a pharmaceutically acceptable salt thereof for use in a method of treating cancer.

20. Use of a compound of any of claims 1-16 or a pharmaceutically acceptable salt thereof in the manufacture of a medicament for treating cancer.

21. A method for treating cancer in a subject in need thereof comprising administering to the subject a therapeutically effective amount of a compound of Formula (II) or a pharmaceutically acceptable salt thereof: 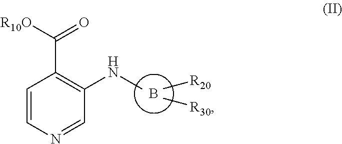
wherein
ring B is pyrazolyl;
R₁₀ is H or C₁-C₆ alkyl;
R₂₀ is C₁-C₆ alkyl, C₂-C₆ alkenyl, C₂-C₆ alkynyl, C₃-C₈ cycloalkyl, C₁-C₆ alkoxyl, C₆-C₁₀ aryloxy, amino, mono-C₁-C₆ alkylamino, di-C₁-C₆ alkylamino, C₆-C₁₀ aryl or 5- or 6-membered heteroaryl and R₂₀ is optionally substituted with one or more substituents selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl, and R₂₀ is attached to ring B via a nitrogen ring atom thereof; and
R₃₀ is H, halo, cyano, azido, ORₐ, —NRₐR_{b}, —C(O)Rₐ, —C(O)ORₐ, —C(O)NRₐR_{b}, —NR_{b}C(O)Rₐ, —S(O)_{b}Rₐ, —S(O)_{b}NRₐR_{b}, or R_{S0}, in which R_{S0} is C₁-C₆ alkyl, C₂-C₆ alkenyl, C₂-C₆ alkynyl, C₃-C₈ cycloalkyl, C₆-C₁₀ aryl, C₆-C₁₀ aryloxy, amino, mono-C₁-C₆ alkylamino, di-C₁-C₆ alkylamino, 4 to 12-membered heterocycloalkyl, or 5- or 6-membered heteroaryl, b is 0, 1, or 2, each of Rₐ and R_{b}, independently is H or R_{S1}, and R_{S1} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl; or Rₐ and R_{b}, together with the N atom to which they are attached, form a 4 to 12-membered heterocycloalkyl ring having 0 or 1 additional heteroatom; and each of R_{S0}, R_{S1}, and the 4 to 12-membered heterocycloalkyl ring formed by Rₐ and R_{b}, is optionally substituted with one or more substituents selected from the group consisting of halo, hydroxyl, oxo, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

22. The method of claim 21, wherein R₂₀ is phenyl or pyridinyl optionally substituted with one substituent selected from the group consisting of halo, hydroxyl, C(O)OH, C(O)O—C₁-C₆ alkyl, cyano, C₁-C₆ alkyl, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl.

23. The method of any one of claims 21-22, wherein R₂₀ is unsubstituted phenyl or pyridinyl.

24. The method of any one of claims 21-23, wherein R₃₀ is C₁-C₆ alkyl.

25. The method of any one of claims 21-24, wherein R₃₀ is methyl.

26. The method of any one of claims 21-25, wherein R₁ is H.
